(19) United States
(12) Patent Application Publication
(10) Pub. No.: US 2004/0121965 A1
(43) Pub. Date: Jun. 24, 2004

(54) Method of treating resistant tumors

(75) Inventors: Lee Martin Greenberger, Montclair, NJ (US); Frank Loganzo JR., New City, NY (US); Carolyn Mary Discafani-Marro, Cortlandt Manor, NY (US); Arie Zask, New York, NY (US); Semiramis Ayral-Kaloustian, Tarrytown, NY (US)

Correspondence Address: WYETH, PATENT LAW GROUP, FIVE GIRALDA FARMS, MADISON, NJ, 07940, US

(73) Assignee: Wyeth Holdings Corporation, Madison, NJ (US)

(21) Appl. No.: 10/666,722

(22) Filed: Sep. 18, 2003

Related U.S. Application Data
(60) Provisional application No. 60/411,883, filed on Sep. 20, 2002.

(51) Int. Cl. A61K 38/04

(52) U.S. Cl. 514/19

(57) ABSTRACT

The invention provides a method of treating or inhibiting the growth of or eradicating a tumor in a mammal in need thereof wherein said tumor is resistant to at least one chemotherapeutic agent which method comprises providing to said mammal an effective amount of a compound of Formula II or a pharmaceutically acceptable salt thereof.

“This application claims priority from copending provisional application No. 60/411,883 filed Sep. 20, 2002 the entire disclosure of which is hereby incorporated by reference.”

### FIELD OF THE INVENTION

The present invention relates to the field of treatments for cancer. More particularly the present invention relates to a method of treating or inhibiting the growth of cancerous tumors in a mammal with inherent or acquired resistance to chemotherapeutic agents used in chemotherapy treatment and in particular antimitotic agents by administering an effective amount of a hemiasterlin derivative and pharmaceutically acceptable salts thereof.

### BACKGROUND OF THE INVENTION

Drug resistance is a major impediment in cancer chemotherapy. Patients may acquire resistance during multiple cycles of therapy. Alternatively, patients may not respond at the onset of therapy (inherent resistance). Resistance to virtually all approved cancer cytotoxic chemotherapy has been reported for chemotherapeutic agents which include antimicrotubule agents (paclitaxel and docetaxel, vinblastine, vincristine or vinorelbine), topoisomerase inhibitors (etoposide, teniposide, topotecan, camptothecin, doxorubicin and duanorubicin), antimetabolities (methotrexate, 5-flurouracil, gemcitabine), alkylating agents (melphalan, chlorambucil), and other DNA damaging agents (cisplatin and its analogs). (Gottesman, M. M.; Goldstein, L. J.; Fojo, A.; Galski and Pastan, I. Expression of the multidrug resistance gene in human cancer. In: I. B. Roninson (ed.), Molecular and Cellular Biology of Multidrug Resistance in Tumor Cells, pp. 291-301. New York: Plenum Press, 1991.) The mechanisms mediating resistance are highly diverse. Clearly, the finding and development of a chemical compound for use in tumors that are resistant to the marketed antimitotic drugs, paclitaxel, docetaxel, vinblastine, or vincristine would be beneficial.

Hemiasterlins are natural products derived from sponges that induce microtubule depolymerization, G₂/M cell cycle arrest, and ultimately cell death. (Anderson, H. J., Coleman, J. E., Andersen, R. J., Roberge, M. Cytotoxic peptides hemiasterlin, hemiasterlin A and hemiasterlin B induce mitotic arrest and abnormal spindle formation, Cancer Chemother. Pharmacol. 39: 223-226, 1997. Talpir, R., Benayahu, Y., Kashman, Y., Pannell, L., Schleyer, M. Hemiasterlin and geodiamolide TA: two new cytotoxic peptides from the marine sponge hemiasterella minor (kirkpatrick), Tetrahedron Left. 35: 4453-4456, 1994.) The total synthesis of hemiasterlin has been reported. (Andersen, R. J., Coleman, J. E., Piers, E., Wallace, D. Total synthesis of (−) hemiasterlin, a structurally novel tripeptide that exhibits potent cytototic activity, Tetrahedron Lett. 38: 317-320, 1997.) Hemiasterlins in cancer therapy have been reported (WO 99/32509, WO 96/33211 and US Pat. No. 6,153,590). (Andersen, R. J., Coleman, J. E., Piers, E., Wallace, D. Total synthesis of (−) hemiasterlin, a structurally novel tripeptide that exhibits potent cytototic activity, Tetrahedron Lett. 38: 317-320, 1997;Andersen, R., Coleman, J., De Silva, D., Kong, F., Piers, E., Wallace, D., Roberge, M, Allen, T. Cytotoxic peptides from marine sponge Cymbastela sp. Tetrahedron. 51: 10653-10662, 1995.)

The mechanisms that may mediate resistance to antimitotics include drug efflux pumps (MDR1 and possibly MXR), tubulin mutations, alternative expression of tubulin isotypes, alteration in the expression or function of genes that mediate apoptosis (e.g. p53 and bcl-2), and overexpression of growth factors such as HER-2. (Dumontet, C. Mechanisms of action and resistance to tubulin-binding agents. Exp. Opin. Invest. Drugs. 9: 779-788, 2000) Resistance mediated by the multidrug drug resistance gene, MDR-1, has been intensively studied, mainly because it is frequently encountered in experimental models. (Greenberger, L. M., Cohen, D., and Horwitz, S. B. In vitro models of multiple drug resistance. In: a. R. F. O. L. J. Goldstein (ed.) Anticancer Drug Resistance, pp. 69-106. Norwell, M A: Kluwer Academic Publishers, 1994) MDR-1 is implicated in resistance to anti-microtubule drugs since: 1) selection of tissue culture cells for resistance to vinca alkaloids or taxanes leads to marked over-expression of MDR-1, 2) cells that over-express MDR-1 have low drug accumulation of taxanes or vinca alkaloids, 3) transfection of cells with MDR-1 induces resistance to these agents, 4) photoaffinity probes for vinca alkaloids or taxanes bind to the MDR-1 gene product, P-glycoprotein, 5) transgenic mice devoid of MDR gene family members have altered pharmacokinetic profiles for taxanes and 6) agents that inhibit P-glycoprotein resensitize resistant cells to taxanes or vinca alkaloids. The clinical relevance of MDR-1 overexpression is not clear in most solid tumor types and its association with lack of patient response or poor prognosis is controversial. (Bradshaw, D. M., Arceci, R. J. Clinical relevance of transmembrane drug efflux as a mechanism of multidrug resistance, J. Clin. Oncol. 16: 3674-3690, 1998; Trock B J, Leonessa F, Clarke R A. Multidrug resistance in breast cancer: a meta-analysis of MDR1/gp170 expression and its possible functional significance. J Natl Cancer Inst,. 89: 917-931, 1997) Nevertheless, overexpression of MDR1 has been clearly associated with response to chemotherapy and prognosis in leukemias. Low level resistance to vinca alkaloids (but not taxanes) has also been found in cells transfected with another efflux pump, MRP. (Breuninger, L. M., Paul, S., Gaughan, K., Miki, T., Chan, A., Aaronson, S. A., Kruh, G. D. Expression of multidrug resistance-associated protein in NIH/3T3 cells confers multidrug resistance associated with increased drug efflux and altered intracellular drug distribution., Cancer Res. 55: 5342-5347, 1995; Zaman, G. J. R., Flens, M. J., van Leusden, M. R., de Haas, M., Mulder, H. S., Lankelma, J., Pinedo, H. M., Scheper, R. J., Baas, F., Broxterman, H. J., and Borst, P. The human multidrug resistance-associated protein MRP is a plasma membrane drug-efflux pump, Proc. Natl. Acad. Sci. USA. 91: 8822-8826, 1994.)

Tubulin mutations have been found in cells selected for resistance to agents that polymerize microtubules, paclitaxel or epothilones. (Giannakakou, P., Gussio, R., Nogales, E., Downing, K. H., Zaharevitz, D., Bolbuck, B., Poy, G., Sackett, D., Nicolauo, K. C., Fojo, T. A common pharmacophore for epithilone and taxanes: molecular basis for drug resistance conferred by tubulin mutations in human cancer cells, Proc. Natl. Acad. Sci. USA. 97: 2904-2909, 2000; Giannakakou, P., Sackett, D. L., Kang, Y.-K., Zhan, A., Buters, J. T., M., Fojo, T., Poruchynsky, M. S. Paclitaxel-resistant human ovarian cancer cells have mutant b-tubulins that exhibit impaired paclitaxel-driven polymerization, J. Biol. Chem. 272: 17118-17125, 1997; He L., Yang C.-P. H., Horwitz S. B. Mutations in β-tubulin map to domains involved in regulation of microtubule stability in epothilone-resistant cell lines. Molecular Cancer Therapeutics. 1:3-10, 2001.) For paclitaxel resistance of this type, selection must be done with paclitaxel in the presence of an MDR-1 inhibitor to avoid the preferential overexpression of MDR1. Based on crystallographic data and molecular modeling of tubulin, the mutations occur in regions of tubulin thought to interact with taxanes. (Giannakakou, P., Gussio, R., Nogales, E., Downing, K. H., Zaharevitz, D., Bolbuck, B., Poy, G., Sackett, D., Nicolauo, K. C., Fojo, T. A common pharmacophore for epithilone and taxanes: molecular basis for drug resistance conferred by tubulin mutations in human cancer cells, Proc. Natl. Acad. Sci. USA. 97: 2904-2909, 2000.) The clinical significance is being evaluated. One report found that 33% of patients with non-small cell carcinomas had tumors with tubulin mutations and such mutations are correlated with poor response to paclitaxel therapy (Monzo, M., Rosell, R., Sanchez, J. J., Lee, J. S., O'Brate, A., Gonzalez-Larriba, J. L., Alberola, V., Lorenzo, J. C., Nunez, L., Ro, J. Y., Martin, C. Paclitaxel resistance in son-small cell lung cancer associated with beta-tubulin gene mutations, J. Clin. Oncol. 17: 1786--179, 1999.) although others have not observed mutations in clinical samples of lung or ovarian tumors (Kelley M. J., Li S., Harpole D. H. Genetic analysis of the β-tubulin gene, TUBB, in non-small-cell lung cancer. J. Natl. Cancer Institute., 93: 1886-1888, 2001; Sale S., Sung R., Shen P., Yu K., Wang Y., Duran G. E., Kim J.-H., Fojo T., Oefner P. J., Sikic B. I. Conservation of the class I β-tubulin gene in human populations and lack of mutations in lung cancers and paclitaxel-resistant ovarian cancers. Mol. Cancer. Thera., 1: 215-225, 2002).

Differential expression of tubulin isoforms has been found in some cell lines selected for paclitaxel or vinca alkaloid resistance. (Burkart, C. A., Kavallaris, M., Horwitz, S. B. The role of b-tubulin isotypes in resistance to antimitotic drugs, Biochim. Biophys. Acta. 1471: 01-09, 2001.) The clinical association with isotype alterations has not been fully studied, but alterations in isotype expression in patients resistant to paclitaxel have been found. (Kavallaris, M., Kuo, D. Y-.S., Burkhart, C. A., Regf, D. L., Norris, M. D., Haber, M., Horwitz, S. B. Taxol-resistant epithelial ovarian tumors are associated with altered expression of specific b-tubulin isotypes, J. Clin. Invest. 100: 1282-1293, 1997; Nicoletti M. I., Valoti G., Giannakakou P., Zhan Z., Kim J.-H., Luccini V., Landoni F., Mayo J. G., Giavazzi R., Fojo T. Expression of β-tubulin isotypes in human ovarian carcinoma xenografts and in a sub-panel of human cancer cell lines from the NCI-anticancer drug screen: Correlation with sensitivity to microtubule active agents. Clinical Cancer Research. 7: 2912-2922, 2001.)

Accordingly, it is one of the purposes of this invention to overcome the above described limitations in cancer treatment by providing a method for treating tumors that are resistant to currently marketed antimitotic agents.

Additionally, it would be advantageous to provide a method for treating or inhibiting multiple drug resistance.

### BRIEF DESCRIPTION OF THE DRAWINGS

FIG. 1. Relative level of expression of MDR-1 protein in tumor cell lines.

### BRIEF SUMMARY OF THE INVENTION

It has now been discovered that the present invention provides a method of treating, inhibiting the growth of, or eradicating a tumor in a mammal in need thereof wherein said tumor is resistant to at least one chemotherapeutic agent which method comprises providing to said mammal an effective amount of a hemiasterlin compound of Formula (I):

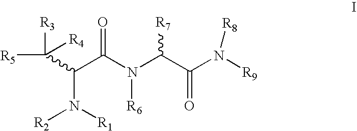

wherein:

R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀—NO₂, —SO₃H, —SOR₁₀, or —SO₂R₁₀, wherein R₁₀ is a linear, branched or cyclic, one to ten carbon saturated or unsaturated alkyl group; and aryl-R-;

R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁OH, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

or R₁ and R₂ taken together with the nitrogen atom to which they are attached is a three to seven membered ring;

R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

or R₃ and R₄ taken together with the carbon to which they are attached form a three to seven membered ring;

R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; aryl-R- and aryl;

R₆ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₇ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CH—O, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₉ is:

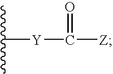

and wherein,

R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, -CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group;

X is a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;

Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;

Y is a moiety selected from the group consisting of: a linear, saturated or unsaturated, one to six carbon alkyl group, optionally substituted with R, ArylR-, or X; and,

Z is a moiety selected from the group consisting of: —OH, —OR; —SH; —SR; —NH₂; —NHR; —N(R)₂; —NHCH(R₁₁)COOH; and —NRCH(R₁₁)COOH, wherein R₁₁ is a moiety having the formula: R, or —(CH₂)ₙNR₁₂R₁₃, wherein n=1-4 and R₁₂ and R₁₃ are independently selected from the group consisting of: H; R; and —C(NH) (NH₂); or pharmaceutically acceptable salts thereof.

A further object of the present invention provides a method of treating, inhibiting the growth of, or eradicating a tumor in a mammal in need thereof wherein said tumor is resistant to at least one chemotherapeutic agent which method comprises providing to said mammal an effective amount of a compound of Formula (II):

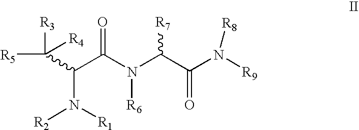

wherein:

R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

or R₁ and R₂ taken together with the nitrogen atom to which they are attached is a three to seven membered ring;

R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

or R₃ and R₄ taken together with the carbon to which they are attached form a three to seven membered ring;

R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group, aryl-R- and aryl and provided that when R₅ is an indolyl moiety of the formula

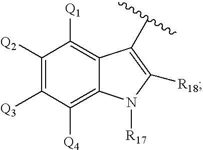

R₁₇ is H or an optionally substituted alkyl or acyl group; and

R₁₈, Q₁, Q₂, Q₃ and Q₄ are independently selected from H, halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;

R₆ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₇ is selected from the group consisting of a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₉ is:

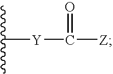

and wherein,

R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group;

X is a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;

Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;

Y is a moiety selected from the group consisting of: a linear, saturated or unsaturated, one to six carbon alkyl group, optionally substituted with R, ArylR-, or X; and,

Z is a moiety selected from the group consisting of: —OH, —OR; —SH; —SR; —NH₂; —NHR; —N(R)₂; —NHCH(R₁₁)COOH; and —NRCH(R₁₁)COOH, wherein R₁₁ is a moiety having the formula: R, or —(CH₂)ₙNR₁₂R₁₃, wherein n=1-4 and R₁₂ and R₁₃ are independently selected from the group consisting of: H; R; and —C(NH) (NH₂);

with the provisos that:

(1) when R₁ is H and R₂ is CH₃ of the moiety

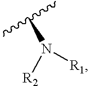

R₃ is CH₃, R₄ is CH₃, R₅ is phenyl, R₆ is H, R₈ is CH₃, and

a) when R₆ is

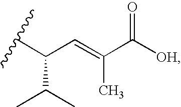

then R₇ is not

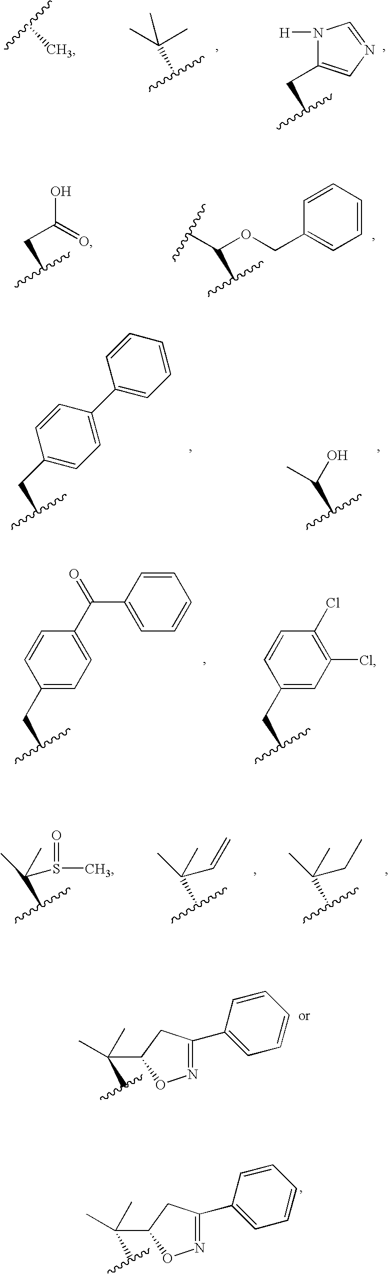

b) when R₉ is

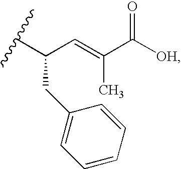

then R₇ is not

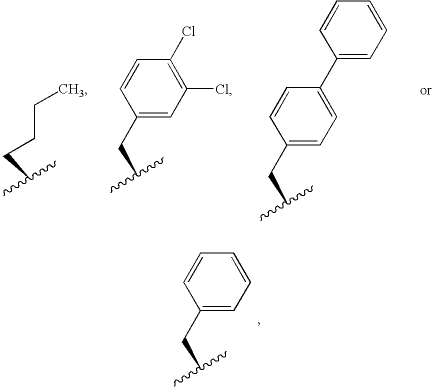

(2) when R₁ is H and R₂ is CH₃, of the moiety

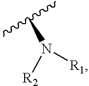

R₃ is CH₃, R₄ is CH₃, R₅ is phenyl, R₅ is H, R₈ is H,

a) R₉ is

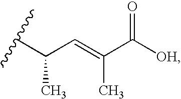

then R₇ is not

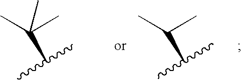

b) when R₉ is

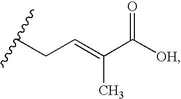

then R₇ is not

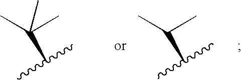

c) when R₉ is

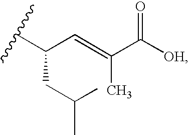

then R₇ is not

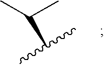

(3) when R₁ is H and R₂ is CH₃, of the moiety

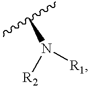

R₃ is CH₃, R₄ is CH₃, R₅ is phenyl, R₆ is H, R₇ is

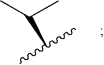

R₈ is

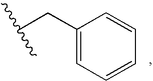

then R₉ is not

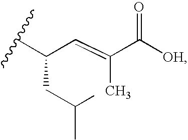

(4) when R₁ is H, R₂ is H, of the moiety

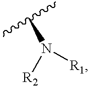

R₃ is CH₃, R₄ is CH₃, R₅ is phenyl, R₆ is H, R₇ is

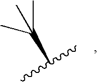

and

R₈ is CH₃,

then R₉ is not

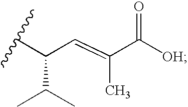

(5) when R₁ is H and R₂ is CH₃ of the moiety

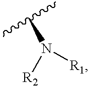

R₃ is CH₃, R₄ is CH₃, R₅ is phenyl, R₆ is H,

R₇ is

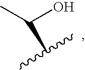

and

R₈ is CH₃,

then R₉ is not

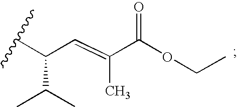

(6) when R₁ is H and R₂ is CH₃ of the moiety

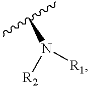

R₃ is CH₃, R₄ is CH₃, R₅ is phenyl, R₆ is H,

R₇ is

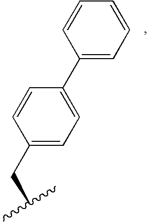

and

R₈ is CH₃,

then R₉ is not

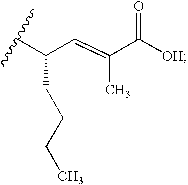

(7) when R₁ is H, R₂ is H, R₃ is CH₃, R₄ is CH₃, R₅ is

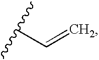

R₆ is H,

R₇ is

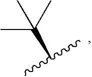

and

R₈ is CH₃,

then R₉ is not

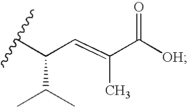

(8) when R₁ is H and R₂ is CH₃, of the moiety

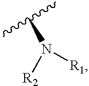

R₃ is CH₃, R₄ is CH₃, R₅ is phenyl, R₆ is H,

R₇ is

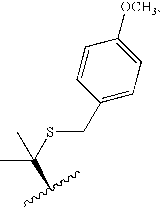

and

R₈ is CH₃,

then R₉ is not

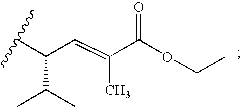

(9) when R₁ is H and R₂ is CH₃ of the moiety

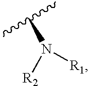

R₃ is CH₃, R₄ is CH₃, R₅ is phenyl,

R₆ is H,

R₇ is

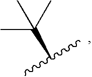

and

R₈ is H,

then R₉ is not

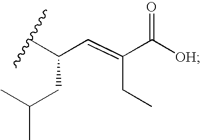

(10) when R₁ is H, R₂ is CH₃,

R₃ is H, R₄ is phenyl, R₅ is phenyl,

R₆ is H,

R₈ is CH₃,

and

R₉ is

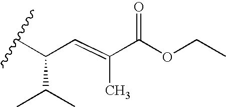

then R₇ is not

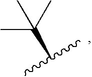

(11) when R₁ is H and R₂ is CH₃ of the moiety

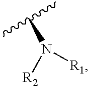

R₃ is CH₃, R₄ is CH₃, R₅ is phenyl,

R₆ is H,

R₈ is CH₃,

and

R₇ is

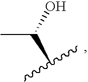

then R₉ is not

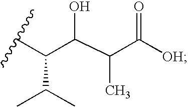

(12) when R₁ is H and R₂ is CH₃ of the moiety

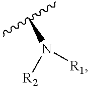

R₃ is CH₃, R₄ is CH₃, R₅ is phenyl,

R₆ is H,

R₇ is

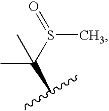

and

R₈ is CH₃,

then R₉ is not

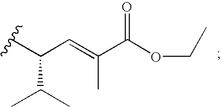

(13) when R₁ is H and R₂ is CH₃ of the moiety

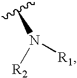

R₃ is CH₃, R₄ is CH₃, R₅ is phenyl,

R₆ is H,

R₇ is

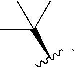

and

R₈ is CH₃,

then R₉ is not

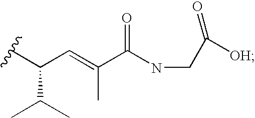

(14) when R₁ is H and R₂ is CH₃ of the moiety

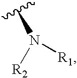

R₃ is CH₃, R₄ is CH₃, R₅ is phenyl,

R₆ is H,

R₇ is

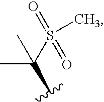

and

R₈ is CH₃,

then R₉ is not

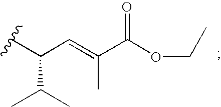

(15) when R₁ is CH₃, R₂ is H,

R₃ is H, R₄ is phenyl, R₅ is phenyl,

R₆ is H,

R₇ is

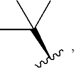

and

R₈ is CH₃,

then R₉ is not

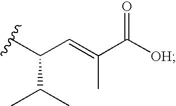

(16) when R₁ is CH₃ and R₂ is H of the moiety

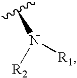

R₃ is CH₃, R₄ is methyl, R₅ is phenyl,

R₆ is H,

R₇ is

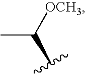

and

R₈ is CH₃,

then R₉ is not

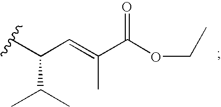

(17) when R₁ is CH₃ and R₂ is H of the moiety

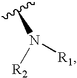

R₃ is CH₃, R₄ is methyl, R₅ is 4-methoxyphenyl,

R₆ is H,

R₇ is

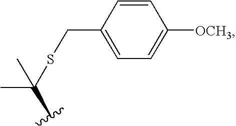

and

R₈ is CH₃,

then R₉ is not

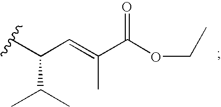

(18) when R₁ is CH₃ and R₂ is H of the moiety

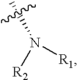

R₃ is CH₃, R₄ is CH₃, R₅ is 3-chlorophenyl,

R₆ is H,

R₇ is

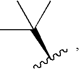

and

R₈ is CH₃,

then R₉ is not

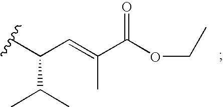

(19) when R₁ is CH₃ and R₂ is H of the moiety

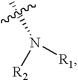

R₃ is CH₃, R₄ is CH₃, R₅ is phenyl,

R₆ is H,

R₇ is

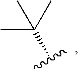

and

R₈ is CH₃,

then R₉ is not

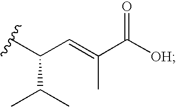

(20) when R₁ is CH₃ and R₂ is CH₃ of the moiety

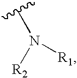

R₃ is H, R₄ is H, R₅ is 3-pyridyl,

R₆ is H,

R₇ is

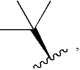

and

R₈ is CH₃,

then R₉ is not

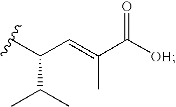

(21) when R₁ is CH₃ and R₂ is H, R₃ is

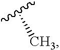

R₄ is H, R₅ is —O—CH₂-phenyl,

R₆ is H,

R₇ is

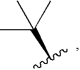

and

R₈ is CH₃,

then R₉ is not

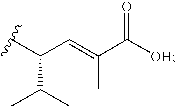

(22) when R₁ is H and R₂ is CH₃ of the moiety

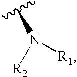

R₃ is CH₃, R₄ is CH₃, R₅ is phenyl, R₆ is CH₃, R is CH₃, and

R₉ is

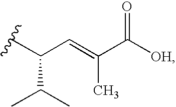

then R₇ is not

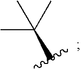

(23) when R₁ is H;

R₃ and R₄ are CH₃;

R₅ is phenyl;

R₆ is H;

R₇ is

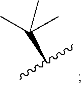

R₈ is CH₃;

R₉ is

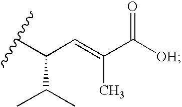

then R₂ of the moiety

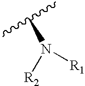

is not

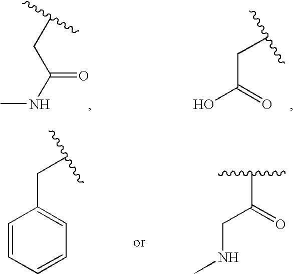

or pharmaceutically acceptable salts thereof.

Except where otherwise stated, any moiety referred to herein which is described as alkyl, will preferably be straight chain or, branched when possible, and will preferably have up to eight, more preferably up to six and even more preferably up to four carbon atoms. Except where otherwise stated optionally substituted alkyl groups are preferably unsubstituted. Methyl, isopropyl and t-butyl are the most preferred alkyl group.

In this specification, reference is made to alkyl moieties being saturated or unsaturated, thereby including within the definition of the moiety, alkene and alkyne groups (whether internal, terminal or part of a ring).

Halogen as used herein means chloro, fluoro, bromo and iodo.

Preferably the recitation of a compound of Formula (II) herein covers all possible salts of the compound, and denotes all possible isomers possible within the structural formula given for such compound, including geometrical and optical isomers. Unless otherwise stated, materials described herein comprising a compound for which isomers exist, are to be regarded as covering individual isomers, and, mixtures of isomers including racemic mixtures.

In a compound of Formula (II), the following substituents alone, or in combination, are preferred:

(a) R₁ is H, methyl, ethyl, propyl, n-butyl, or acetyl and R₂ is methyl, ethyl, propyl, n-butyl, or acetyl; or, where R₁ and R₂ taken together with the nitrogen atom to which they are attached form a three to six membered ring; more preferably R₁ is H and R₂ is CH₃;

(b) preferably no more than one of R₃ and R₄ is H; more preferably, R₃ and R₄are independently:

methyl, ethyl, n-propyl or n-butyl, or, where R₃ and R₄ are joined together to form a β-cyclopropyl, β-cyclobutyl, β-cyclopentyl or β-cyclohexyl ring; most preferably R₃ and R₄ are each methyl;

(c) R₅: is cyclohexyl and Aryl in the definition of R₅ is preferably phenyl, naphthyl, thienyl, anthracyl, pyrrolyl or indolyl; preferably R₅ is phenyl, or indolyl; most preferably R₅ is phenyl;

(d) R₆ and R₈ independently: H or methyl, more preferably R₆ is H and R₈ is methyl;

(e) R₇: a three to six carbon, branched alkyl group; more preferably R₇ is —C(CH₃)₃; and

(f) in R₉, Z is preferably OH, —OR₁₄ (wherein R₁₄, is a linear or branched one to six carbon alkyl group,

—NHCH(R)₁₁)COOH or —NCH₃CH(R₁₁)COOH wherein R₁₁ is R, or

—(CH₂)ₙ NHC (NH) (NH₂) ); or

R₉ is preferably —C(R₁₅)—C═C(R₁₆)C(O)—OH wherein R₁₅ is methyl, ethyl, n-propyl, isopropyl, tert-butyl, iso-butyl, or sec-butyl and R₁₆. is H, methyl, ethyl, propyl, iso-propyl, n-butyl, iso-butyl or sec-butyl; more preferably Z is OH and R₉ is:

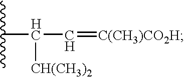

R₉ is more preferably;

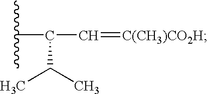

R₉ is most preferably;

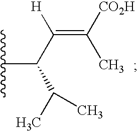

Preferably, compounds of Formula (II);

R₁ is selected from the group consisting of H; a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-—;

R₂ is selected from the group consisting of a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

or R₁ and R₂ taken together with the nitrogen atom to which they are attached is a three to seven membered ring;

R₃ is selected from the group consisting of a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₄ is selected from the group consisting of H; a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀OCOR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

or R₃ and R₄ taken together with the carbon to which they are attached form a three to seven membered ring;

R₅ is selected from the group consisting of H; a saturated or unsaturated moiety having a linear, branched, or cyclic skeleton containing one to ten carbon atoms, said carbon atoms optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀OCOR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; aryl-R- and aryl;

R₆ is selected from the group consisting of H; a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₇ is selected from the group consisting of a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₈ is selected from the group consisting of H; a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀OCOR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₉ is:

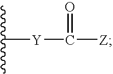

and wherein,

R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group;

X is a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;

Aryl is an aromatic ring selected from the group consisting of: phenyl, naphthyl, anthracyl, phenanthryl, furyl, indolyl, thienyl, pyrrolyl, thiophenyl, benzofuryl, benzothiophenyl, quinolyl, isoquinolyl, imidazolyl, thiazolyl, oxazolyl, and pyridyl, optionally substituted with R or X;

Y is a moiety selected from the group consisting of: a linear, saturated or unsaturated, one to six carbon alkyl group, optionally substituted with R, ArylR-, or X; and,

Z is a moiety selected from the group consisting of: —OH, —OR; —SH; —SR; —NH₂; —NHR; —N(R)₂; —NHCH(R₁₁)COOH; and —NRCH(R₁₁)COOH, wherein R₁₁ is a moiety having the formula: R, or —(CH₂)ₙNR₁₂R₁₃, wherein n=1-4 and R₁₂ and R₁₃ are independently selected from the group consisting of: H; R; and —C(NH) (NH₂);

or pharmaceutically acceptable salts thereof.

Additionally preferred compounds of the invention are those of Formula (II) wherein:

R₁ is methyl;

R₂ is H;

R₃ and R₄ are methyl;

R₅ is selected from the group consisting of H; a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SOCR₁₀, —NH₂, —NR₁₀OH, —N(R₁₀)₂, —NHCOR₁₀, —NR₁₀OCOR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group, aryl-R-; and aryl;

provided that when R₅ is an indolyl moiety of the formula

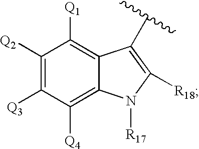

R₁₇ is H or an optionally substituted alkyl or acyl group; and

R₁₈, Q₁, Q₂, Q₃, and Q₄ are independently selected from H, halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;

R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group;

X is a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;

Aryl is an aromatic ring selected from the group consisting of: phenyl, naphthyl, anthracyl, phenanthryl, furyl, pyrrolyl, thienyl, thiophenyl, benzofuryl, benzothiophenyl, indolyl, quinolyl, isoquinolyl, imidazolyl, thiazolyl, oxazolyl, and pyridyl, optionally substituted with R or X;

R₆ is H;

R₈ is methyl; and

R₇ is selected from the group consisting of a saturated or unsaturated moiety having a linear, branched, or cyclic skeleton containing one to ten carbon atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀OCOR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₇ is preferably t-butyl;

R₉ is preferably —C(R₁₅)—C═C(R₁₆)C(O)—OH wherein R₁₅ is methyl, ethyl, n-propyl, isopropyl, tert-butyl, iso-butyl, or sec-butyl and R₁₆. is H, methyl, ethyl, propyl, iso-propyl, n-butyl, iso-butyl or sec-butyl; R₉ is:

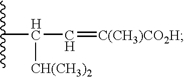

R₉ is more preferably

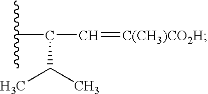

R₉ is most preferably

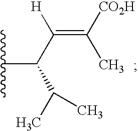

The most preferred absolute configurations of compounds of Formula (II) wherein the absolute configurations of moieties a, b and c of Formula (II)

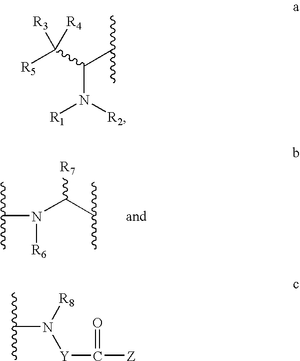

are selected from:

| | a | b | c |
|---|---|---|---|
| | S | S | S |
| | R | S | S |
| and | S | S | R. |

Among the specifically preferred compounds of Formula (II) of this invention for a method of treating, inhibiting the growth of, or eradicating a tumor in a mammal in need thereof wherein said tumor is resistant to at least one chemotherapeutic agent which method comprises providing to said mammal an effective amount of a compound of Formula (II) and pharmaceutically acceptable salts thereof are those set forth below:

3-Chloro-N,β,β-trimethyl-L-phenylalanyl-N¹-[(1S,2E)-4-ethoxy-1-isopropyl-3-methyl-4-oxo-2-butenyl]-N¹⁻,3-dimethyl-L-valinamide,

4-Chloro-N,β,β-trimethyl-L-phenylalanyl-N¹-[(1S,2E)-4-ethoxy-1-isopropyl-3-methyl4-oxo-2-butenyl]-N¹⁻,3-dimethyl-L-valinamide,

4-chloro-N,β,β-trimethyl-D-phenylalanyl-N¹-[(1S,2E)-4-ethoxy-1-isopropyl-3-methyl4-oxo-2-butenyl]-N¹⁻,3-dimethyl-L-valinamide,

4-Chloro-N,β,β-triethyl-L-phenylalanyl-N¹-[(1S,2E)-3-carboxy-1-isopropyl-2-butenyl]-N¹,3-dimethyl-L-valinamide,

4-Chloro-N,β,β-trimethyl-D-phenylalanyl-N¹-[(1S,2E)-3-carboxy-1-isopropyl-2-butenyl]-N¹,3-dimethyl-L-valinamide,

N,β,β,3-Tetramethyl-L-phenylalanyl-N¹-[(1S,2E)-4-ethoxy-1-isopropyl-3-methyl4-oxo-2-butenyl]-N¹,3-dimethyl-L-valinamide,

N,β,β,3-tetramethyl-D-phenylalanyl-N¹-[(1S,2E)4-ethoxy-1-isopropyl-3-methyl-4-oxo-2-butenyl]-N¹,3-dimethyl-L-valinamide,

N,β,β,3-Tetramethyl-L-phenylalanyl-N¹-[(1S,2E)-3-carboxy-1-isopropyl-2-butenyl]-N¹,3-dimethyl-L-valinamide,

N,β,β,3-Tetramethyl-D-phenylalanyl-N¹-[(1S,2E)-3-carboxy-1-isopropyl-2-butenyl]-N¹,3-dimethyl-L-valinamide,

N,β,β,3-Tetramethyl-L-phenylalanyl-N¹-[(1S,2E)4-ethoxy-1-isopropyl-3-methyl-4-oxo-2-butenyl]-N¹, 3-dimethyl-L-valinamide,

N,β,β,4-tetramethyl-D-phenylalanyl-N¹-[(1S,2E)-4-ethoxy-1-isopropyl-3-methyl-4-oxo-2-butenyl]-N¹,3-dimethyl-L-valinamide,

N,β,β,4-Tetramethyl-L-phenylalanyl-N¹-[(1S,2E)-3-carboxy-1-isopropyl-2-butenyl]-N¹,3-dimethyl-L-valinamide,

N,β,β,4-Tetramethyl-D-phenylalanyl-N¹-[(1S,2E)-3-carboxy-1-isopropyl-2-butenyl]-N¹,3-dimethyl-L-valinamide,

N,β,β-3,4-Pentamethyl-L-phenylalanyl-N¹-[(1S,2E)4-ethoxy-1-isopropyl-3-methyl-4-oxo-2-butenyl]-N¹, 3-dimethyl-L-valinamide,

N,β,β-3,4-pentamethyl-D-phenylalanyl-N¹-[(1S,2E)4-ethoxy-1-isopropyl-3-methyl4-oxo-2-butenyl]-N¹, 3-dimethyl-L-valinamide,

N,β,β,3,4-Pentamethyl-L-phenylalanyl-N¹-[(1S,2E)-3-carboxy-1-isopropyl-2-butenyl]-N¹,3-dimethyl-L-valinamide,

N,β,β,3,4-Pentamethyl-D-phenylalanyl-N¹,[(1S,2E)-3-carboxy-1-isopropyl-2-butenyl]-N¹,3-dimethyl-L-valinamide,

N,β,β,3,5-Pentamethyl-L-phenylalanyl-N¹-[(1S,2E)4-ethoxy-1-isopropyl-3-methyl-4-oxo-2-butenyl]-N¹, 3-dimethyl-L-valinamide,

N,β,β,3,5-pentamethyl-D-phenylalanyl-N¹-[(1S,2E)-4-ethoxy-1-isopropyl-3-methyl-4-oxo-2-butenyl]-N¹, 3-dimethyl-L-valinamide,

N,β,β,3,5-Pentamethyl-L-phenylalanyl-N¹-[(1S,2E)-3-carboxy-1-isopropyl-2-butenyl]-N¹,3-dimethyl-L-valinamide,

N,β,β,3,5-Pentamethyl-D-phenylalanyl-N¹-[(1S,2E)-3-carboxy-1-isopropyl-2-butenyl]-N¹, 3-dimethyl-L-valinamide,

N-Methyl-3-(2-thienyl)-L-valyl-N¹-[(1S,2E)-4-ethoxy-1-isopropyl-3-methyl-4-oxo-2-butenyl]-N¹,3-dimethyl-L-valinamide,

N-methyl-3-(2-thienyl)-D-valyl-N¹-[(1S,2E)-4-ethoxy-1-isopropyl-3-methyl-4-oxo-2-butenyl]-N¹, 3-dimethyl-L-valinamide,

N-Methyl-3-(2-thienyl)-L-valyl-N¹-[(1S,2E)-3-carboxy-1-isopropyl-2-butenyl]-N¹,3-dimethyl-L-valinamide,

N-Methyl-3-(2-thienyl)-D-valyl-N¹-[(1S,2E)-3-carboxy-1-isopropyl-2-butenyl]-N¹,3-dimethyl-L-valinamide,

N-Methyl-3-thien-3-yl-L-valyl-N¹-[(1S,2E)-4-ethoxy-1-isopropyl-3-methyl4-oxobut-2-enyl]-N¹,3-dimethyl-L-valinamide,

N-methyl-3-thien-3-yl-D-valyl-N¹-[(1S,2E)4-ethoxy-1-isopropyl-3-methyl4-oxobut-2-enyl]-N¹, 3-dimethyl-L-valinamide,

N-Methyl-3-thien-3-yl-L-valyl-N¹-[(1S,2E)-3-carboxy-1-isopropylbut-2-enyl]-N¹,3-dimethyl-L-valinamide,

N-Methyl-3-thien-3-yl-D-valyl-N¹-[(1S,2E)-3-carboxy-1-isopropylbut-2-enyl]-N¹,3-dimethyl-L-valinamide,

3-(1-Benzothien-3-yl)-N-methylvalyl-N¹-[(1S,2E)4-ethoxy-1-isopropyl-3-methyl-4-oxo-2-butenyl]-N¹,3-dimethyl-L-valinamide,

3-(1-Benzothien-3-yl)-N-methylvalyl-N¹-[(1S,2E)-3-carboxy-1-isopropyl-2-butenyl]-N¹,3-dimethyl-L-valinamide,

3-(1-Benzothien-2-yl)-N-methylvalyl-N¹-[(1S,2E)-4-ethoxy-1-isopropyl-3-methyl4-oxo-2-butenyl]-N¹,3-dimethyl-L-valinamide,

3-(1-Benzothien-2-yl)-N-methylvalyl-N¹-[(1S,2E)-3-carboxy-1-isopropyl-2-butenyl]-N¹,3-dimethyl-L-valinamide,

4-tert-Butyl-N,β,β-trimethylphenylalanyl-N¹-[(1S,2E)-4-ethoxy-1-isopropyl-3-methyl-4-oxo-2-butenyl]-N¹,3-dimethyl-L-valinamide,

4-tert-Butyl-N,β,β-trimethylphenylalanyl-N¹-[(1S,2E)-3-carboxy-1-isopropyl-2-butenyl]-N¹,3-dimethyl-L-valinamide,

N-Ethyl-β,β-dimethylphenylalanyl-N¹-[(1S,2E)4-ethoxy-1-isopropyl-3-methyl4-oxo-2-butenyl]-N¹,3-dimethyl-L-valinamide,

N-Ethyl-β,β-dimethylphenylalanyl-N¹-[(1S,2E)-3-carboxy-1-isopropyl-2-butenyl]-N¹,3-dimethyl-L-valinamide,

N-(tert-Butoxycarbonyl)-N-β,β,2-tetramethylphenylalanyl-N¹-[(1S,2E)-4-ethoxy-1-isopropyl-3-methyl-4-oxo-2-butenyl]-N¹,3-dimethyl-L-valinamide,

N,β,β,2-tetramethyl-L-phenylalanyl-N¹-[(1S,2E)-4-ethoxy-1-isopropyl-3-methyl4-oxo-2-butenyl]-N¹,3-dimethyl-L-valinamide,

N,β,β,2-Tetramethyl-D-phenylalanyl-N¹-[(1S,2E)4-ethoxy-1-isopropyl-3-methyl-4-oxo-2-butenyl]-N¹,3-dimethyl-L-valinamide,

N,β,β,2-Tetramethyl-L-phenylalanyl-N¹-[(1S,2E)-3-carboxy-1-isopropyl-2-butenyl]-N¹,3-dimethyl-L-valinamide,

N,β,β,2-Tetramethyl-D-phenylalanyl-N¹-[(1S,2E)-3-carboxy-1-isopropyl-2-butenyl]-N¹,3-dimethyl-L-valinamide,

3-bromo-N,β,β-trimethyl-L-phenylalanyl-N¹-[(1S,2E)-3-carboxy-1-isopropylbut-2-enyl]-N¹,3-dimethyl-L-valinamide,

3-bromo-N,β,β-trimethyl-D-phenylalanyl-N¹-[(1S,2E)-3-carboxy-1-isopropylbut-2-enyl]-N¹,3-dimethyl-L-valinamide,

3-phenyl-N,β,β-trimethyl-L-phenylalanyl-N¹-[(1S,2E)-3-carboxy-1-isopropylbut-2-enyl]-N¹,3-dimethyl-L-valinamide,

3-phenyl-N,β,β-trimethyl-D-phenylalanyl-N¹-[(1S,2E)-3-carboxy-1-isopropylbut-2-enyl]-N¹,3-dimethyl-L-valinamide,

N,β,β-trimethyl-3-vinyl-L-phenylalanyl-N¹-[(1S,2E)-3-carboxy-1-isopropylbut-2-enyl]-N¹,3-dimethyl-L-valinamide,

3-ethyl- N,β,β-trimethyl-L-phenylalanyl-N¹-[(1S,2E)-3-carboxy-1-isopropylbut-2-enyl]-N¹,3-dimethyl-L-valinamide,

4-bromo-N,β,β-trimethylphenylalanyl-N¹-[(1S,2E)-3-carboxy-1-isopropyl-2-butenyl]-N¹,3-dimethyl-L-valinamide,

4-phenyl-N,β,β--trimethyl-L-phenylalanyl-N¹-[(1S,2E)-3-carboxy-1-isopropyl-2-butenyl]-N¹,3-dimethyl-L-valinamide,

4-carboxy-N,β,β--trimethylphenylalanyl-N¹-[(1S,2E)-3-carboxy-1-isopropylbut-2-enyl]-N¹,3-dimethyl-L-valinamide,

3-Methoxy-N,β,β-trimethylphenylalanyl-N¹-[(1S,2E)-3-carboxy-1-isopropyl-2-butenyl]-N¹,3-dimethyl-L-valinamide,

3-Hydroxy-N,β,β-trimethylphenylalanyl-N¹-[(1S,2E)-3-carboxy-1-isopropyl-2-butenyl]-N¹,3-dimethyl-L-valinamide,

N,3-Dimethyl-4-phenyl-L-valyl-N¹-[(1S,2E)-3-carboxy-1-isopropyl-2-butenyl]-N¹,3-dimethyl-L-valinamide,

N,3-dimethyl-4-phenyl-D-valyl-N¹-[(1S,2E)-3-carboxy-1-isopropyl-2-butenyl]-N¹,3-dimethyl-L-valinamide,

(2E,4S)-4-[((2S)-2-{[(2S)-3,3-dimethyl-2-(methylamino)octanoyl]amino}-3,3-dimethylbutanoyl)(methyl)amino]-2,5-dimethyl-2-hexenoic acid,

(2E,4S)-4-[((2S)-2-{[(2R)-3,3-dimethyl-2-(methylamino)octanoyl]amino}-3,3-dimethylbutanoyl)(methyl)amino]-2,5-dimethyl-2-hexenoic acid,

N,N,β,β-Tetramethyl-L-phenylalanyl-N¹-[(1S,2E)-3-carboxy-1-isopropyl-2-butenyl]-N¹,3-dimethyl-L-valinamide,

N-(2-hydroxyethyl)-N,β,β-trimethyl-L-phenylalanyl-N¹-[(1S,2E)-3-carboxy-1-isopropylbut-2-enyl]-N¹,3-dimethyl-L-valinamide,

2-Methoxy-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide,

2-Methoxy-N,β,β-trimethyl-L-phenylalanyl-N¹-[(1S,2E)-3-carboxy-1-isopropyl-2-butenyl]-N¹,3-dimethyl-L-valinamide,

N,O,β,β-tetramethyl-L-tyrosyl-N¹-[(1S,2E)4-ethoxy-1-isopropyl-3-methyl-4-oxo-2-butenyl]-N¹,3-dimethyl-L-valinamide,

N,O,β,β-tetramethyl-L-tyrosyl-N¹-[(1S,2E)-3-carboxy-1-isopropyl-2-butenyl]-N¹,3-dimethyl-L-valinamide,

2-Methoxy-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide,

2-Methoxy-N,O,β,β-tetramethyl-L-tyrosyl-N¹-[(1S,2E)-3-carboxy-1-isopropyl-2-butenyl]-N¹,3-dimethyl-L-valinamide,

3—Fluoro-N,β,β-trimethyl-L-phenylalanyl-N¹-[(1S,2E)-3-carboxy-1-isopropyl-2-butenyl]-N¹,3-dimethyl-L-valinamide,

3—Fluoro- N,β,β-trimethyl-D-phenylalanyl-N¹-[(1S,2E)-3-carboxy-1-isopropyl-2-butenyl]-N¹,3-dimethyl-L-valinamide,

N,β,β-Trimethyl-3-(trifluoromethyl)-L-phenylalanyl-N¹-[(1S,2E)-3-carboxy-1-isopropyl-2-butenyl]-N¹,3-dimethyl-L-valinamide,

N,β,β-Trimethyl-3-(trifluoromethyl)-D-phenylalanyl-N¹-[(1S,2E)-3-carboxy-1-isopropyl-2-butenyl]-N¹,3-dimethyl-L-valinamide,

3,5-Difluoro-N,β,β-trimethyl-L-phenylalanyl-N¹-[(1S,2E)-3-carboxy-1-isopropylbut-2-enyl]-N¹,3-dimethyl-L-valinamide,

3,5-Difluoro-N,β,β-trimethyl-D-phenylalanyl-N¹-[(1S,2E)-3-carboxy-1-isopropylbut-2-enyl]-N¹,3-dimethyl-L-valinamide,

N,β,β-trimethyl-3,5-bis(trifluoromethyl)-L-phenylalanyl-N¹-[(1S,2E)-3-carboxy-1-isopropylbut-2-enyl]-N¹,3-dimethyl-L-valinamide,

N,β,β-trimethyl-3,5-bis(trifluoromethyl)-D-phenylalanyl-N¹-[(1S,2E)-3-carboxy-1-isopropylbut-2-enyl]-N¹,3-dimethyl-L-valinamide,

O-isopropyl-N,β,β-trimethyl-L-tyrosyl-N¹-[(1S,2E)-3-carboxy-1-isopropylbut-2-enyl]-N¹,3-dimethyl-L-valinamide,

O-isopropyl- N,β,β-trimethyl-D-tyrosyl-N¹-[(1S,2E)-3-carboxy-1-isopropylbut-2-enyl]-N¹,3-dimethyl-L-valinamide,

3-Cyclohexyl-N-methyl-L-valyl-N¹-[(1S,2E)-3-carboxy-1-isopropyl-2-butenyl]-N¹,3-dimethyl-L-valinamide,

(2E,4S)-2,5-dimethyl4-(methyl{3-methyl-N-[(2S)-2-(methylamino)-2-(1-phenylcyclopentyl)ethanoyl]-L-valyl}amino)-2-hexenoic acid,

(2E,4S)-2,5-dimethyl4-(methyl{3-methyl-N-[(2R)-2-(methylamino)-2-(1-phenylcyclopentyl)ethanoyl]-L-valyl}amino)-2-hexenoic acid,

(2E,4R)-2,5-dimethyl4-(methyl{3-methyl-N-[(methylamino)(1-phenylcyclohexyl)acetyl]-L-valyl}amino)-2-hexenoic acid,

(E,4S)-2,5-Dimethyl4-[methyl((2S)-2-{[(2S)-3-methyl-2-(methylamino)-3-phenylbutanoyl]amino}-3-phenylpropanoyl)amino]-2-hexenoic acid,

N,β,β-Trimethyl-L-phenylalanyl-N¹-[(1S,2E)-1-butyl-3-carboxybut-2-enyl]-N¹,3-dimethyl-L-valinamide,

N,β,β-Trimethyl-L-phenylalanyl-N¹-[(1S,2E)-3-carboxy-1-isobutyl-2-pentenyl]-N¹,3-dimethyl-L-valinamide,

(E ,4S)-2-Butyl-4-[((2S)-3,3-dimethyl-2-{[(2S)-3-methyl-2-(methylamino)-3-phenylbutanoyl]amino}butanoyl)amino]-5-methyl-2-hexenoic acid,

N,β,β-Trimethyl-L-phenylalanyl-N¹-[(1S,2E)-3-carboxy-1-isopropyl-2-pentenyl]-N¹,3-dimethyl-L-valinamide,

Ethyl (E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoate,

(E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoic acid,

N,β,β-trimethyl-L-phenylalanyl-N¹-[(1S,2E)-3-carboxy-1-isopropyl-2-butenyl]-N¹-methyl-3-(methylsulfonyl)-L-valinamide,

N,β,β-trimethyl-L-phenylalanyl-N¹-[(1S,2E)-3-carboxy-1-isopropyl-2-butenyl]-3-[(4-methoxybenzyl)sulfanyl]-N¹-methyl-L-valinamide,

N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide,

N,O,β,β-tetramethyl-L-tyrosyl-N¹-[(1S,2E)-3-carboxy-1-isopropyl-2-butenyl]-N¹-methyl-3-(methylsulfanyl)-L-valinamide,

N,β,β-trimethyl-L-phenylalanyl-N¹-[(1R,2E)-4-ethoxy-1-isopropyl-3-methyl-4-oxo-2-butenyl]-N¹-methyl-L-allothreoninamide,

N,β,β-trimethyl-L-phenylalanyl-N¹-[(1S,2E)-3-carboxy-1-isopropyl-2-butenyl]-N¹-methyl-L-allothreoninamide,

N,β,β-trimethyl-L-phenylalanyl-N¹-[(1S,2E)-3-carboxy-1-isopropyl-2-butenyl]-N,O,β,β-tetramethyl-L-tyrosinamide,

N,β,β-trimethyl-L-phenylalanyl-N¹-[(1S,2E)-3-carboxy-1-isopropyl-2-butenyl]-N¹,O-dimethyl-L-allothreoninamide,

(E ,4S)-2,5-Dimethyl-4-[methyl((2S)-2-{[(2S)-3-methyl-2-(methylamino)-3-phenylbutanoyl]amino}-4-phenylbutanoyl)amino]-2-hexenoic acid,

N,β,β-trimethyl-L-phenylalanyl-4-benzoyl-N¹-[(1S,2E)-3-carboxy-1-isopropyl-2-butenyl]- N,β,β-trimethyl-L-phenylalaninamide,

4-benzoyl-N,β,β-trimethyl-L-phenylalanyl-N¹-[(1S,2E)-3-carboxy-1-isopropylbut-2-enyl]-N¹,3-dimethyl-L-valinamide,

N,β,β-trimethyl-L-phenylalanyl-N¹-[(1S,2E)-3-carboxy-1-isobutylbut-2-enyl]-N¹-methyl-L-valinamide,

N,β,β-trimethyl-L-phenylalanyl-N¹-[(1S,2E)-3-carboxy-1-isobutylbut-2-enyl]-3-methyl-L-valinamide,

N,β,β-trimethyl-L-phenylalanyl-N¹-[(1S,2E)-3-carboxy-1-isopropylbut-2-enyl]-N¹-ethyl-3-methyl-L-valinamide,

N,β,β-trimethyl-L-phenylalanyl-N¹-[(1S,2E)-3-carboxy-1-isopropylbut-2-enyl]-N¹-ethyl-L-valinamide,

N,β,β-trimethyl-L-phenylalanyl-N¹-[(1S,2E)-3-carboxy-1-isopropylbut-2-enyl]-N¹-methyl-L-leucinamide,

N,β,β-trimethyl-L-phenylalanyl-N¹-[(1S,2E)-3-carboxy-1-isopropylbut-2-enyl]-N¹-methyl-L-norvalinamide,

(2E,4S)4-[{(2R)-2-cyclohexyl-2-[(N,β,β-trimethyl-L-phenylalanyl)amino]ethanoyl}(methyl)amino]-2,5-dimethylhex-2-enoic acid,

(2E,4S)-2,5-dimethyl4-(methyl{(2S)-2-[(N,β,β-trimethyl-L-phenylalanyl)amino]butanoyl}amino)hex-2-enoic acid,

4-{[3,3-Dimethyl-2-(2-methylamino-3-phenyl-butyrylamino)-butyryl]-methyl-amino}-2,5-dimethyl-hex-2-enoic acid,

N,β,β-trimethyl-L-phenylalanyl-N¹-[(1S,2E)-3-carboxy-1-isopropylbut-2-enyl]-3-methyl-L-valinamide,

N,β,β-trimethyl-L-phenylalanyl-N¹-[(1S,2E)-3-carboxy-1-isopropylbut-2-enyl]-L-valinamide,

2,5-dimethyl-4-{methyl-[2-(3-methyl-2-methylamino-3-phenyl-butyrylamino)-propionyl]-amino}-hex-2-enoic acid,

4-{[3,3-Dimethyl-2-(3-methyl-2-methylamino-3-phenyl-butyrylamino)-butyryl]-methyl-amino}-2,6-dimethyl-hept-2-enoic acid,

N,β,β-trimethyl-L-phenylalanyl-N¹-[(1S,2E)-3-carboxy-1-isopropyl-2-butenyl]-N¹-methyl-L-valinamide,

N,β,β-trimethyl-L-phenylalanyl-N¹-[(1S,2E)-3-carboxy-1-isopropyl-2-butenyl]-N¹-methyl-L-isoleucinamide,

(E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenamide,

(E,4S)-4-[((2S)-3,3-dimethyl-2-{[(2S)-3-methyl-2-(methylamino)-3-phenylbutanoyl]amino}butanoyl)(methyl)amino]-N,2,5-trimethyl-2-hexenamide,

N,β,β-trimethyl-L-phenylalanyl-N¹-{(1S,2E)4-[(2-cyanoethyl)amino]-1-isopropyl-3-methyl-4-oxo-2-butenyl}-N¹,3-dimethyl-L-valinamide,

N,β,β-trimethyl-L-phenylalanyl-N¹-{(1S,2E)4-[(carboxymethyl)amino]-1-isopropyl-3-methyl-4-oxo-2-butenyl}-N¹,3-dimethyl-L-valinamide,

N,β,β-trimethyl-L-phenylalanyl-N¹-{(1S,2E)-4-[(4-azidophenyl)amino]-1-isopropyl-3-methyl4-oxo-2-butenyl}-N¹,3-dimethyl-L-valinamide,

N,β,β-trimethyl-L-phenylalanyl-N¹-{(1S,2E)-1-isopropyl-3-methyl-4-oxo-4-[(2-phenylethyl)amino]but-2-enyl}-N¹-,3-dimethyl-L-valinamide,

N,β,β-trimethyl-L-phenylalanyl-N¹-{(1S,2E)4-[[(1S,2E)4-ethoxy-1-isopropyl-3-methyl4-oxobut-2-enyl](methyl)amino]-1-isopropyl-3-methyl-4-oxobut-2-enyl}-N¹-,3-dimethyl-L-valinamide,

N,β,β-trimethyl-L-phenylalanyl-N¹-{(1S,2E)4-[[(1S,2E)-3-carboxy-1-isopropylbut-2-enyl](methyl)amino]-1-isopropyl-3-methyl-4-oxobut-2-enyl}-N-1-,3-dimethyl-L-valinamide,

N,β,β-trimethyl-L-phenylalanyl-N¹-[(1S,2E)-1-isopropyl-3-methyl4-oxo-4-(thien-2-ylmethoxy)but-2-enyl]-N¹-,3-dimethyl-L-valinamide,

N,β,β-trimethyl-L-phenylalanyl-N¹-[(1S,2E)-1-isopropyl-3-methyl-4-(octyloxy)-4-oxobut-2-enyl]-N¹-,3-dimethyl-L-valinamide,

N,β,β-trimethyl-L-phenylalanyl-N¹-[(1S,2Z)-3-carboxy-1-isopropyl-2-butenyl]-N¹,3-dimethyl-L-valinamide,

N,β,β-trimethyl-L-phenylalanyl-N¹-[(1S,2E)-3-carboxy-1-isopropylprop-2-enyl]-N¹,3-dimethyl-L-valinamide,

N,β,β-trimethyl-L-phenylalanyl-N¹-[(1S,2E)-1-allyl-3-carboxybut-2-enyl]-N¹,3-dimethyl-L-valinamide,

(2E,4S)-4-[{(2S)-3,3-dimethyl-2-[(N,β,β-trimethyl-L-phenylalanyl)amino]4-pentenoyl}(methyl)amino]-2,5-dimethyl-2-hexenoic acid,

(2E, 4S)-4-[((2S)-2-{[3,3-dimethyl-2-(methylamino)-4-pentenoyl]amino}-3,3-dimethylbutanoyl)(methyl)amino]-2,5-dimethyl-2-hexenoic acid,

N,β,β-trimethyl-L-phenylalanyl-N¹-[(1S,2E)-3-carboxy-1-isopropylbut-2-enyl]-N¹,3-dimethyl-L-isoleucinamide,

N,β,β-trimethyl-L-phenylalanyl-N¹-[(1R,3S)-3-carboxy-1-isopropylbutyl]-N¹,3-dimethyl-L-valinamide,

N,β,β-trimethyl-L-phenylalanyl-N¹-[(1R,3R)-3-carboxy-1-isopropylbutyl]-N¹,3-dimethyl-L-valinamide,

β,β-diethyl-N-methyl-L-phenylalanyl-N¹-[(1S,2E)-3-carboxy-1-isopropylbut-2-enyl]-N¹,3-dimethyl-L-valinamide,

β,β-diethyl-N-methyl-D-phenylalanyl-N¹-[(1S,2E)-3-carboxy-1-isopropylbut-2-enyl]-N¹,3-dimethyl-L-valinamide,

β,β-dimethyl-L-phenylalanyl-N¹-[(1S,2E)-3-carboxy-1-isopropylbut-2-enyl]-N¹,3-dimethyl-L-valinamide,

O-benzyl-N-methyl-L-threonyl-N¹-[(1S,2E)-3-carboxy-1-isopropylbut-2-enyl]-N¹,3-dimethyl-L-valinamide,

N,β,β-trimethyl-L-phenylalanyl-N¹-[(1S,2E)-3-carboxy-1-isopropylbut-2-enyl]-N¹,3-dimethyl-L-valinamide,

(2E,4S)-4-[((2S)-2-{[(2S)-2-Amino-3-(1-naphthyl)propanoyl]amino}-3,3-dimethylbutanoyl)(methyl)amino]-2,5-dimethyl-2-hexenoic acid,

N,β,β-trimethyl-L-phenylalanyl-N¹-[(1S,2E)-3-carboxy-1-isopropylbut-2-enyl]-N¹-methyl-D-valinamide,

(E,4S)-4-[((2S)-3,3-dimethyl-2-{[(2S)-3-methyl-2-(methylamino)-3-(1-methyl-1H-ethyl-1H-indol-3-yl)butanoyl]amino}butanoyl)amino]-2,5-dimethyl-2-hexenoic acid,

ethyl (E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenoate,

(E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenoic acid,

Ethyl (E,4S)-4-[((2S)-3,3-dimethyl-2-{[(2R)-3-methyl-2-(methylamino)-3-phenylbutanoyl]amino}butanoyl)(methyl)amino]-2,5-dimethyl-2-hexenoate,

(E,4S)-4-[((2S)-3,3-dimethyl-2-{[(2S)-3-methyl-2-(methylamino)-3-phenylbutanoyl]amino}butanoyl)(methyl)amino]-2-methyl-5-phenyl-2-pentenoic acid,

(E,4S)-2,5-dimethyl-4-[methyl((2S)-2-{[(2S)-3-methyl-2-(methylamino)-3-phenylbutanoyl]amino}-3-phenylpropanoyl)amino]-2-hexenoic acid,

(4R)-4-[((2S)-2-{[(2S)-2-amino-4-methylpentanoyl]amino}-3,3-dimethylbutanoyl)amino]-2,5-dimethylhexanoic acid,

N,β,β-trimethyl-L-phenylalanyl-N¹-[(1S,2E)-3-carboxy-1-isopropyl-2-butenyl]-N¹-methyl-L-alpha-glutamine,

N,3-dimethyl-L-valyl-N¹-[(1S,2E)-3-carboxy-1-isopropyl-2-butenyl]-N¹,3-dimethyl-L-valinamide,

N,β,β-trimethyl-L-tryptophyl-N¹-[(1S,2E)-3-carboxy-1-isopropyl-2-butenyl]-N¹,3-dimethyl-L-valinamide,

3-cyclohexyl-N-methyl-L-valyl-N¹-[(1S,2E)-4-ethoxy-1-isopropyl-3-methyl-4-oxo-2-butenyl]-N¹,3-dimethyl-L-valinamide,

(2E,4S)-2,5-dimethyl-4-(methyl{3-methyl-N-[(2S)-2-(methylamino)-2-(1-phenylcyclopropyl)acetyl]-L-valyl}amino)hex-2-enoic acid,

(2E,4S)-2,5-dimethyl-4-(methyl{3-methyl-N-[(2R)-2-(methylamino)-2-(1-phenylcyclopropyl)acetyl]-L-valyl}amino)hex-2-enoic acid,

2-(4-{[3,3-Dimethyl-2-(3-methyl-2-methylamino-3-phenyl-butyrylamino)-butyryl]-methyl-amino}-2,5-dimethyl-hex-2-enoylamino)-4-methylsulfanyl-butyric acid methyl ester,

N,β,β-trimethyl-L-phenylalanyl-N1-((1S,2E)-4-{[(1S)-1-carboxy-3-(methylthio)propyl]amino}-1-isopropyl-3-methyl-4-oxobut-2-enyl)-N 1 ,3-dimethyl-L-valinamide,

N,β,β-trimethyl4-[(E)-2-phenylvinyl]-L-phenylalanyl-N¹-[(1S,2E)-3-carboxy-1-isopropylbut-2-enyl]-N¹,3-dimethyl-L-valinamide,

N,β,β,trimethyl-4-[(E)-2-phenylvinyl]-D-phenylalanyl-N¹-[(1S,2E)-3-carboxy-1-isopropylbut-2-enyl]-N¹,3-dimethyl-L-valinamide,

N,β,β-timethyl-L-phenylalanyl-N¹-[(1S,2E)-3-carboxy-1-isopropylbut-2′enyl]-3-fluoro-N¹-methyl-D-valinamide,

N,β,β-trimethyl-L-phenylalanyl-N¹-[(1S,2E)-3-carboxy-1-isopropylbut-2-enyl]-3-fluoro-N¹-methyl-L-valinamide,

3-[(4-methoxybenzyl)thio]-N-methyl-L-valyl-N¹-[(1S,2E)-3-carboxy-1-isopropylbut-2-enyl]-N¹,3-dimethyl-L-valinamide,

N-ethyl-β,β-dimethyl-L-phenylalanyl-N¹-[(1S,2E)-3-carboxy-1-isopropylbut-2-enyl]-N¹,3-dimethyl-L-valinamide,

(2E,4S)-2,5-dimethyl-4-(methyl{3-methyl-N-[(2S)-3-methyl-3-phenyl-2-pyrrolidin-1-ylbutanoyl]-L-valyl}amino)hex-2-enoic acid,

N-(2-hydroxyethyl)-β,β-dimethyl-L-phenylalanyl-N¹-[(1S,2E)-3-carboxy-1-isopropylbut-2-enyl]-N¹,3-dimethyl-L-valinamide,

(βR)-N,β-dimethyl-L-phenylalanyl-N¹-[(1S,2E)-3-carboxy-1-isopropylbut-2- enyl]-N¹,3-dimethyl-L-valinamide,

3-acetyl-N,β,β-trimethyl-L-phenylalanyl-N¹-[(1S,2E)-3-carboxy-1-isopropylbut-2-enyl]-N¹,3-dimethyl-L-valinamide,

N,β,β-trimethyl-L-phenylalanyl-N¹-[(1S,2E)-3-carboxy-1-isopropylbut-2-enyl]-3-hydroxy-N¹-methyl-L-valinamide, and

N,β,β-trimethyl-L-phenylalanyl-N¹-[(1R,2E)-3-carboxy-1-isopropylbut-2- enyl]-N¹,3-dimethyl-L-valinamide.

Among the specifically preferred compounds of Formula (II) of this invention for a method of treating, inhibiting the growth of, or eradicating a tumor in a mammal in need thereof wherein said tumor is resistant to at least one chemotherapeutic agent which method comprises providing to said mammal an effective amount of a compound of Formula (II) and pharmaceutically acceptable salts thereof are those set forth below as groups A-T below:

A)

3-Chloro-N,β,β-trimethyl-L-phenylalanyl-N¹-[(1S,2E)-4-ethoxy-1-isopropyl-3-methyl4-oxo-2-butenyl]-N¹,3-dimethyl-L-valinamide,

4-Chloro- N,β,β-trimethyl-L-phenylalanyl-N¹-[(1S,2E)4-ethoxy-1-isopropyl-3-methyl-4-oxo-2-butenyl]-N¹,3-dimethyl-L-valinamide,

4-chloro- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide,

4-Chloro- N,β,β-triethyl-L-phenylalanyl-N¹-[(1S,2E)-3-carboxy-1-isopropyl-2-butenyl]-N¹,3-dimethyl-L-valinamide,

4-Chloro-N,β,β-trimethyl-D-phenylalanyl-N¹-[(1S,2E)-3-carboxy-1-isopropyl-2-butenyl]-N¹,3-dimethyl-L-valinamide and

3-ethyl- N,β,β-trimethyl-L-phenylalanyl-N¹-[(1S,2E)-3-carboxy-1-isopropylbut-2-enyl]-N¹,3-dimethyl-L-valinamide.

B)

N,β,β,3-Tetramethyl-L-phenylalanyl-N¹-[(1S,2E)-4-ethoxy-1-isopropyl-3-methyl-4-oxo-2-butenyl]-N¹,3-dimethyl-L-valinamide,

N,β,β,3-tetramethyl-D-phenylalanyl-N¹-[(1S,2E)-4-ethoxy-1-isopropyl-3-methyl-4-oxo-2-butenyl]-N¹,3-dimethyl-L-valinamide,

N,β,β,3-Tetramethyl-L-phenylalanyl-N¹-[(1S,2E)-3-carboxy-1-isopropyl-2-butenyl]-N¹,3-dimethyl-L-valinamide,

N,β,β,3-Tetramethyl-D-phenylalanyl-N¹-[(1S,2E)-3-carboxy-1-isopropyl-2-butenyl]-N¹,3-dimethyl-L-valinamide,

N,β,β,4-Tetramethyl-L-phenylalanyl-N¹-[(1S,2E)4-ethoxy-1-isopropyl-3-methyl4-oxo-2-butenyl]-N¹,3-dimethyl-L-valinamide,

N,β,β,4-tetramethyl-D-phenylalanyl-N¹-[(1S,2E)4-ethoxy-1-isopropyl-3-methyl-4-oxo-2-butenyl]-N¹,3-dimethyl-L-valinamide,

N,β,β,4-Tetramethyl-L-phenylalanyl-N¹-[(1S,2E)-3-carboxy-1-isopropyl-2-butenyl]-N¹,3-dimethyl-L-valinamide,

N,β,β,4-Tetramethyl-D-phenylalanyl-N¹-[(1S,2E)-3-carboxy-1-isopropyl-2-butenyl]-N¹,3-dimethyl-L-valinamide,

N,β,β4-Pentamethyl-L-phenylalanyl-N¹-[(1S,2E)-4-ethoxy-1-isopropyl-3-methyl-4-oxo-2-butenyl]-N¹,3-dimethyl-L-valinamide,

N,β,β-3,4-pentamethyl-D-phenylalanyl-N¹-[(1S,2E)-4-ethoxy-1-isopropyl-3-methyl4-oxo-2-butenyl]-N¹,3-dimethyl-L-valinamide,

N,β,β,3,4-Pentamethyl-L-phenylalanyl-N¹-[(1S,2E)-3-carboxy-1-isopropyl-2-butenyl]-N¹,3-dimethyl-L-valinamide,

N,β,β,3,4-Pentamethyl-D-phenylalanyl-N¹-[(1S,2E)-3-carboxy-1-isopropyl-2-butenyl]-N¹,3-dimethyl-L-valinamide,

N,β,β,3,5-Pentamethyl-L-phenylalanyl-N¹-[(1S,2E)4-ethoxy-1-isopropyl-3-methyl4-oxo-2-butenyl]-N¹,3-dimethyl-L-valinamide,

N,β,β,3,5-pentamethyl-D-phenylalanyl-N¹-[(1S,2E)-4-ethoxy-1-isopropyl-3-methyl-4-oxo-2-butenyl]-N¹,3-dimethyl-L-valinamide,

N,β,β,3,5-Pentamethyl-L-phenylalanyl-N¹-[(1S,2E)-3-carboxy-1-isopropyl-2-butenyl]-N¹,3-dimethyl-L-valinamide and

N,β,β,3,5-Pentamethyl-D-phenylalanyl-N¹-[(1S,2E)-3-carboxy-1-isopropyl-2-butenyl]-N¹,3-dimethyl-L-valinamide.

C)

N-Methyl-3-(2-thienyl)-L-valyl-N¹-[(1S,2E)-4-ethoxy-1-isopropyl-3-methyl-4-oxo-2-butenyl]-N¹,3-dimethyl-L-valinamide,

N-methyl-3-(2-thienyl)-D-valyl-N¹-[(1S,2E)4-ethoxy-1-isopropyl-3-methyl-4-oxo-2-butenyl]-N¹,3-dimethyl-L-valinamide,

N-Methyl-3-(2-thienyl)-L-valyl-N¹-[(1S,2E)-3-carboxy-1-isopropyl-2-butenyl]-N¹,3-dimethyl-L-valinamide,

N-Methyl-3-(2-thienyl)-D-valyl-N¹-[(1S,2E)-3-carboxy-1-isopropyl-2-butenyl]-N¹,3-dimethyl-L-valinamide,

N-Methyl-3-thien-3-yl-L-valyl-N¹-[(1S,2E)4-ethoxy-1-isopropyl-3-methyl4-oxobut-2-enyl]-N¹,3-dimethyl-L-valinamide,

N-methyl-3-thien-3-yl-D-valyl-N¹-[(1S,2E)4-ethoxy-1-isopropyl-3-methyl-4-oxobut-2-enyl]-N¹,3-dimethyl-L-valinamide,

N-Methyl-3-thien-3-yl-L-valyl-N¹-[(1S,2E)-3-carboxy-1-isopropylbut-2-enyl]-N¹,3-dimethyl-L-valinamide and

N-Methyl-3-thien-3-yl-D-valyl-N¹-[(1S,2E)-3-carboxy-1-isopropylbut-2-enyl]-N¹,3-dimethyl-L-valinamide.

D)

3-(1-Benzothien-3-yl)-N-methylvalyl-N¹-[(1S,2E)4-ethoxy-1-isopropyl-3-methyl4-oxo-2-butenyl]-N¹,3-dimethyl-L-valinamide,

3-(1-Benzothien-3-yl)-N-methylvalyl-N¹-[(1S,2E)-3-carboxy-1-isopropyl-2-butenyl]-N¹,3-dimethyl-L-valinamide,

3-(1-Benzothien-2-yl)-N-methylvalyl-N¹-[(1S,2E)4-ethoxy-1-isopropyl-3-methyl4-oxo-2-butenyl]-N¹,3-dimethyl-L-valinamide and

3-(1-Benzothien-2-yl)-N-methylvalyl-N¹-[(1S,2E)-3-carboxy-1-isopropyl-2-butenyl]-N¹,3-dimethyl-L-valinamide.

E)

4-tert-Butyl-N,β,β-trimethylphenylalanyl-N¹-[(1S,2E)-4-ethoxy-1-isopropyl-3-methyl-4-oxo-2-butenyl]-N¹,3-dimethyl-L-valinamide,

4-tert-Butyl-N,β,β-trimethylphenylalanyl-N¹-[(1S,2E)-3-carboxy-1-isopropyl-2-butenyl]-N¹,3-dimethyl-L-valinamide,

N-Ethyl-β,β-dimethylphenylalanyl-N¹-[(1S,2E)-4-ethoxy-1-isopropyl-3-methyl4-oxo-2-butenyl]-¹,3-dimethyl-L-valinamide and

N-Ethyl-β,β-dimethylphenylalanyl-N¹-[(1S,2E)-3-carboxy-1-isopropyl-2-butenyl]-N¹,3-dimethyl-L-valinamide.

F)

N-(tert-Butoxycarbonyl)-N-β,β,2-tetramethylphenylalanyl-N¹-[(1S,2E)-4-ethoxy-1-isopropyl-3-methyl4-oxo-2-butenyl]-N¹,3-dimethyl-L-valinamide,

N,β,β,2-tetramethyl-L-phenylalanyl-N¹-[(1S,2E)4-ethoxy-1-isopropyl-3-methyl4-oxo-2-butenyl]-N¹,3-dimethyl-L-valinamide,

N,β,β,2-Tetramethyl-D-phenylalanyl-N¹-[(1S,2E)4-ethoxy-1-isopropyl-3-methyl4-oxo-2-butenyl]-N¹,3-dimethyl-L-valinamide,

N,β,β,2-Tetramethyl-L-phenylalanyl-N¹-[(1S,2E)-3-carboxy-1-isopropyl-2-butenyl]-N¹,3-dimethyl-L-valinamide and

N,β,β,2-Tetramethyl-D-phenylalanyl-N¹-[(1S,2E)-3-carboxy-1-isopropyl-2-butenyl]-N¹,3-dimethyl-L-valinamide.

G)

3-bromo-N,β,β-trimethyl-L-phenylalanyl-N¹-[(1S,2E)-3-carboxy-1-isopropylbut-2-enyl]-N¹,3-dimethyl-L-valinamide,

3-bromo-N,β,β-trimethyl-D-phenylalanyl-N¹-[(1S,2E)-3-carboxy-1-isopropylbut-2-enyl]-N¹,3-dimethyl-L-valinamide and

4-bromo-N,β,β-trimethylphenylalanyl-N¹-[(1S,2E)-3-carboxy-1-isopropyl-2-butenyl]-N¹,3-dimethyl-L-valinamide.

H)

3-phenyl-N,β,β-trimethyl-L-phenylalanyl-N¹-[(1S,2E)-3-carboxy-1-isopropylbut-2-enyl]-N¹,3-dimethyl-L-valinamide,

3-phenyl-N,β,β-trimethyl-D-phenylalanyl-N¹-[(1S,2E)-3-carboxy-1-isopropylbut-2-enyl]-N¹,3-dimethyl-L-valinamide and

4-phenyl-N,β,β--trimethyl-L-phenylalanyl-N¹-[(1S,2E)-3-carboxy-1-isopropyl-2-butenyl]-N¹,3-dimethyl-L-valinamide.

I)

4-carboxy- N,β,β-trimethylphenylalanyl-N¹-[(1S,2E)-3-carboxy-1-isopropylbut-2-enyl]-N¹,3-dimethyl-L-valinamide,

3-Methoxy- N,β,β-trimethylphenylalanyl-N¹-[(1S,2E)-3-carboxy-1-isopropyl-2-butenyl]-N¹,3-dimethyl-L-valinamide and

3-Hydroxy- N,β,β-trimethylphenylalanyl-N¹-[(1S,2E)-3-carboxy-1-isopropyl-2-butenyl]-N¹,3-dimethyl-L-valinamide.

J)

N,β,β-trimethyl-3-vinyl-L-phenylalanyl-N¹-[(1S,2E)-3-carboxy-1-isopropylbut-2-enyl]-N¹,3-dimethyl-L-valinamide,

N,3-Dimethyl-4-phenyl-L-valyl-N¹-[(1S,2E)-3-carboxy-1-isopropyl-2-butenyl]-N¹,3-dimethyl-L-valinamide,

N,3-dimethyl-4-phenyl-D-valyl-N¹-[(1S,2E)-3-carboxy-1-isopropyl-2-butenyl]-N¹,3-dimethyl-L-valinamide,

(2E ,4S)-4-[((2S)-2-{[(2S)-3 ,3-dimethyl-2-(methylamino)octanoyl]amino}-3,3-dimethylbutanoyl)(methyl)amino]-2,5-dimethyl-2-hexenoic acid,

(2E,4S)-4-[((2S)-2-{[(2R)-3,3-dimethyl-2-(methylamino)octanoyl]amino}-3,3-dimethylbutanoyl)(methyl)amino]-2,5-dimethyl-2-hexenoic acid,

N,N,β,β-Tetramethyl-L-phenylalanyl-N¹-[(S,2E)-3-carboxy-1-isopropyl-2-butenyl]-N¹,3-dimethyl-L-valinamide and

N-(2-hydroxyethyl)-N,β,β-trimethyl-L-phenylalanyl-N¹-[(S,2E)-3-carboxy-1-isopropylbut-2-enyl]-N¹,3-dimethyl-L-valinamide.

K)

2-Methoxy-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide,

2-Methoxy-N,β,β-trimethyl-L-phenylalanyl-N¹-[(1S,2E)-3-carboxy-1-isopropyl-2-butenyl]-N¹,3-dimethyl-L-valinamide,

N,O,β,β-tetramethyl-L-tyrosyl-N¹-[(1S,2E)4-ethoxy-1-isopropyl-3-methyl-4-oxo-2-butenyl]-N¹,3-dimethyl-L-valinamide,

N,O,β,β-tetramethyl-L-tyrosyl-N¹-[(1S,2E)-3-carboxy-1-isopropyl-2-butenyl]-N¹,3-dimethyl-L-valinamide,

2-Methoxy-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide,

2-Methoxy-N,O,β,β-tetramethyl-L-tyrosyl-N¹-[(1S,2E)-3-carboxy-1-isopropyl-2-butenyl]-N¹,3-dimethyl-L-valinamide,

O-isopropyl-N,β,β-trimethyl-L-tyrosyl-N¹-[(1S,2E)-3-carboxy-1-isopropylbut-2-enyl]-N¹,3-dimethyl-L-valinamide and

O-isopropyl-N,β,β-trimethyl-D-tyrosyl-N¹-[(1S,2E)-3-carboxy-1-isopropylbut-2-enyl]-N¹,3-dimethyl-L-valinamide.

L)

3-Fluoro-N,β,β-trimethyl-L-phenylalanyl-N¹-[(1S,2E)-3-carboxy-1-isopropyl-2-butenyl]-N¹,3-dimethyl-L-valinamide,

3-Fluoro-N,β,β-trimethyl-D-phenylalanyl-N¹-[(1S,2E)-3-carboxy-1-isopropyl-2-butenyl]-N¹,3-dimethyl-L-valinamide,

N,β,β-Trimethyl-3-(trifluoromethyl)-L-phenylalanyl-N¹-[(1S,2E)-3-carboxy-1-isopropyl-2-butenyl]-N¹,3-dimethyl-L-valinamide,

N,β,β-Trimethyl-3-(trifluoromethyl)-D-phenylalanyl-N¹-[(1S,2E)-3-carboxy-1-isopropyl-2-butenyl]-N¹,3-dimethyl-L-valinamide,

3,5-Difluoro-N,β,β-trimethyl-L-phenylalanyl-N¹-[(1S,2E)-3-carboxy-1-isopropylbut-2-enyl]-N¹,3-dimethyl-L-valinamide,

3,5-Difluoro-N,β,β-trimethyl-D-phenylalanyl-N¹-[(1S,2E)-3-carboxy-1-isopropylbut-2-enyl]-N¹,3-dimethyl-L-valinamide,

N,β,β-trimethyl-3,5-bis(trifluoromethyl)-L-phenylalanyl-N¹-[(1S,2E)-3-carboxy-1-isopropylbut-2-enyl]-N¹,3-dimethyl-L-valinamide and

N,β,β-trimethyl-3,5-bis(trifluoromethyl)-D-phenylalanyl-N¹-((1S,2E)-3-carboxy-1-isopropylbut-2-enyl]-N¹,3-dimethyl-L-valinamide.

M)

(2E,4S)-2,5-dimethyl-4-(methyl{3-methyl-N-[(2S)-2-(methylamino)-2-(1-phenylcyclopentyl)ethanoyl]-L-valyl}amino)-2-hexenoic acid,

(2E,4S)-2,5-dimethyl-4-(methyl{3-methyl-N-[(2R)-2-(methylamino)-2-(1-phenylcyclopentyl)ethanoyl]-L-valyl}amino)-2-hexenoic acid and

(2E,4R)-2,5-dimethyl4-(methyl{3-methyl-N-[(methylamino)(1-phenylcyclohexyl)acetyl]-L-valyl}amino)-2-hexenoic acid.

N)

(E,4S)-2,5-Dimethyl-4-[methyl((2S)-2-{[(2S)-3-methyl-2-(methylamino)-3-phenylbutanoyl]amino}-3-phenylpropanoyl)amino]-2-hexenoic acid,

N,β,β-Trimethyl-L-phenylalanyl-N¹-[(1S,2E)-1-butyl-3-carboxybut-2-enyl]-N¹,3-dimethyl-L-valinamide,

N,β,β-Trimethyl-L-phenylalanyl-N¹-[(1S,2E)-3-carboxy-1-isobutyl-2-pentenyl]-N¹,3-dimethyl-L-valinamide,

(E,4S)-2-Butyl-4-[((2S)-3,3-dimethyl-2-{[(2S)-3-methyl-2-(methylamino)-3-phenylbutanoyl]amino}butanoyl)amino]-5-methyl-2-hexenoic acid,

N,β,β-Trimethyl-L-phenylalanyl-N¹-[(1S,2E)-3-carboxy-1-isopropyl-2-pentenyl]-N¹,3-dimethyl-L-valinamide,

Ethyl (E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoate,

(E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoic acid,

N,β,β-trimethyl-L-phenylalanyl-N¹-[(1S,2E)-3-carboxy-1-isopropyl-2-butenyl]-N¹-methyl-3-(methylsulfonyl)-L-valinamide,

N,β,β-trimethyl-L-phenylalanyl-N¹-[(1S,2E)-3-carboxy-1-isopropyl-2-butenyl]-3-[(4-methoxybenzyl)sulfanyl]-N¹-methyl-L-valinamide,

N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide and

N,O,β,β-tetramethyl-L-tyrosyl-N¹-[(1S,2E)-3-carboxy-1-isopropyl-2-butenyl]-N¹-methyl-3-(methylsulfanyl)-L-valinamide.

O)

N,β,β-trimethyl-L-phenylalanyl-N¹-[(1R,2E)-4-ethoxy-1-isopropyl-3-methyl4-oxo-2-butenyl]-N¹-methyl-L-allothreoninamide,

N,β,β-trimethyl-L-phenylalanyl-N¹-[(1S,2E)-3-carboxy-1-isopropyl-2-butenyl]-N¹-methyl-L-allothreoninamide,

N,β,β-trimethyl-L-phenylalanyl-N-[(1S,2E)-3-carboxy-1-isopropyl-2-butenyl]-N,O,β,β-tetramethyl-L-tyrosinamide,

N,β,β-trimethyl-L-phenylalanyl-N¹-[(1S,2E)-3-carboxy-1-isopropyl-2-butenyl]-N¹,O-dimethyl-L-allothreoninamide,

(E,4S)-2,5-Dimethyl-4-[methyl((2S)-2-{[(2S)-3-methyl-2-(methylamino)-3-phenylbutanoyl]amino}4-phenylbutanoyl)amino]-2-hexenoic acid,

N,β,β-trimethyl-L-phenylalanyl-4-benzoyl-N-[(1S,2E)-3-carboxy-1-isopropyl-2-butenyl]- N,β,β-trimethyl-L-phenylalaninamide and

4-benzoyl-N,β,β-trimethyl-L-phenylalanyl-N¹-[(1S,2E)-3-carboxy-1-isopropylbut-2-enyl]-N¹,3-dimethyl-L-valinamide.

P)

N,β,β-trimethyl-L-phenylalanyl-N¹-[(1S,2E)-3-carboxy-1-isobutylbut-2-enyl]-N¹-methyl-L-valinamide,

N,β,β-trimethyl-L-phenylalanyl-N¹-[(1S,2E)-3-carboxy-1-isobutylbut-2-enyl]-3-methyl-L-valinamide,

N,β,β-trimethyl-L-phenylalanyl-N¹-[(1S,2E)-3-carboxy-1-isopropylbut-2-enyl]-N¹-ethyl-3-methyl-L-valinamide,

N,β,β-trimethyl-L-phenylalanyl-N¹-[(1S,2E)-3-carboxy-1-isopropylbut-2-enyl]-N¹-ethyl-L-valinamide,

N,β,β-trimethyl-L-phenylalanyl-N¹-[(1S,2E)-3-carboxy-1-isopropylbut-2-enyl]-N¹-methyl-L-leucinamide,

N,β,β-trimethyl-L-phenylalanyl-N¹-[(1S,2E)-3-carboxy-1-isopropylbut-2-enyl]-N¹-methyl-L-norvalinamide,

(2E,4S)-4-[{(2R)-2-cyclohexyl-2-[(N,β,β-trimethyl-L-phenylalanyl)amino]ethanoyl}(methyl)amino]-2,5-dimethylhex-2-enoic acid,

(2E,4S)-2,5-dimethyl-4-(methyl{(2S)-2-[(N,β,β-trimethyl-L-phenylalanyl)amino]butanoyl}amino)hex-2-enoic acid,

4-{[3,3-Dimethyl-2-(2-methylamino-3-phenyl-butyrylamino)-butyryl]-methyl-amino}-2,5-dimethyl-hex-2-enoic acid,

N,β,β-trimethyl-L-phenylalanyl-N¹-[(1S,2E)-3-carboxy-1-isopropylbut-2-enyl]-3-methyl-L-valinamide,

N,β,β-trimethyl-L-phenylalanyl-N¹-[(1S,2E)-3-carboxy-1-isopropylbut-2-enyl]-L-valinamide,

2,5-dimethyl-4-{methyl-[2-(3-methyl-2-methylamino-3-phenyl-butyrylamino)-propionyl]-amino}-hex-2-enoic acid,

4-{[3,3-Dimethyl-2-(3-methyl-2-methylamino-3-phenyl-butyrylamino)-butyryl]-methyl-amino}-2,6-dimethyl-hept-2-enoic acid,

N,β,β-trimethyl-L-phenylalanyl-N¹-[(1S,2E)-3-carboxy-1-isopropyl-2-butenyl]-N¹-methyl-L-valinamide and

N,β,β-trimethyl-L-phenylalanyl-N¹-[(1S,2E)-3-carboxy-1-isopropyl-2-butenyl]-N¹-methyl-L-isoleucinamide.

Q)

(E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenamide,

(E,4S)-4-[((2S)-3,3-dimethyl-2-{[(2S)-3-methyl-2-(methylamino)-3-phenylbutanoyl]amino}butanoyl)(methyl)amino]-N,2,5-trimethyl-2-hexenamide,

N,β,β-trimethyl-L-phenylalanyl-N¹-{(1S,2E)-4-[(2-cyanoethyl)amino]-1-isopropyl-3-methyl-4-oxo-2-butenyl}-N¹,3-dimethyl-L-valinamide,

N,β,β-trimethyl-L-phenylalanyl-N¹-{(1S,2E)-4-[(carboxymethyl)amino]-1-isopropyl-3-methyl-4-oxo-2-butenyl}-N¹,3-dimethyl-L-valinamide,

N,β,β-trimethyl-L-phenylalanyl-N¹-{(1S,2E)-4-[(4-azidophenyl)amino]-1-isopropyl-3-methyl-4-oxo-2-butenyl}-N¹,3-dimethyl-L-valinamide,

N,β,β-trimethyl-L-phenylalanyl-N¹-{(1S,2E)-1-isopropyl-3-methyl-4-oxo-4-[(2-phenylethyl)amino]but-2-enyl}-N¹-,3-dimethyl-L-valinamide,

N,β,β-trimethyl-L-phenylalanyl-N¹-{(1S,2E)-4-[[(1S,2E)-4-ethoxy-1-isopropyl-3-methyl-4-oxobut-2-enyl](methyl)amino]-1-isopropyl-3-methyl-4-oxobut-2-enyl}-N¹-,3-dimethyl-L-valinamide,

N,β,β-trimethyl-L-phenylalanyl-N¹-{(1S,2E)-4-[[(1S,2E)-3-carboxy-1-isopropylbut-2-enyl](methyl)amino]-1-isopropyl-3-methyl-4-oxobut-2-enyl}-N¹-,3-dimethyl-L-valinamide,

N,β,β-trimethyl-L-phenylalanyl-N¹-[(1S,2E)-1-isopropyl-3-methyl4-oxo-4-(thien-2-ylmethoxy)but-2-enyl]-N¹-,3-dimethyl-L-valinamide,

N,β,β-trimethyl-L-phenylalanyl-N¹-[(1S,2E)-1-isopropyl-3-methyl-4-(octyloxy)-4-oxobut-2-enyl]-N¹-,3-dimethyl-L-valinamide,

N,β,β-trimethyl-L-phenylalanyl-N¹-[(1S,2Z)-3-carboxy-1-isopropyl-2-butenyl]-N¹,3-dimethyl-L-valinamide and

N,β,β-trimethyl-L-phenylalanyl-N¹-[(1S,2E)-3-carboxy-1-isopropylprop-2-enyl]-N¹,3-dimethyl-L-valinamide.

R)

N,β,β-trimethyl-L-phenylalanyl-N¹-[(1S,2E)-1-allyl-3-carboxybut-2-enyl]-N¹,3-dimethyl-L-valinamide,

(2E,4S)-4-[{(2S)-3,3-dimethyl-2-[(N,β,β-trimethyl-L-phenylalanyl)amino]-4-pentenoyl}(methyl)amino]-2,5-dimethyl-2-hexenoic acid,

N,β,β-trimethyl-L-phenylalanyl-N¹-[(1S,2E)-3-carboxy-1-isopropylbut-2-enyl]-N¹,3-dimethyl-L-isoleucinamide,

N,β,β-trimethyl-L-phenylalanyl-N¹-[(1R,3S)-3-carboxy-1-isopropylbutyl]-N¹,3-dimethyl-L-valinamide,

N,β,β-trimethyl-L-phenylalanyl-N¹-[(1R,3R)-3-carboxy-1-isopropylbutyl]-N¹,3-dimethyl-L-valinamide,

β,β-diethyl-N-methyl-L-phenylalanyl-N¹-[(1S,2E)-3-carboxy-1-isopropylbut-2-enyl]-N¹,3-dimethyl-L-valinamide,

β,β-diethyl-N-methyl-D-phenylalanyl-N¹-[(1S,2E)-3-carboxy-1-isopropylbut-2-enyl]-N¹,3-dimethyl-L-valinamide,

N,β,β-dimethyl-L-phenylalanyl-N¹-[(1S,2E)-3-carboxy-1-isopropylbut-2-enyl]-N¹,3-dimethyl-L-valinamide and

O-benzyl-N-methyl-L-threonyl-N¹-[(1S,2E)-3-carboxy-1-isopropylbut-2-enyl]-N¹,3-dimethyl-L-valinamide.

S)

3-Cyclohexyl-N-methyl-L-valyl-N¹-[(1S,2E)-3-carboxy-1-isopropyl-2-butenyl]-N¹,3-dimethyl-L-valinamide and

3-cyclohexyl-N-methyl-L-valyl-N¹-[(1S,2E)-4-ethoxy-1-isopropyl-3-methyl-4-oxo-2-butenyl]-N¹,3-dimethyl-L-valinamide.

T)

(2E,4S)-2,5-dimethyl-4-(methyl{3-methyl-N-[(2S)-2-(methylamino)-2-(1-phenylcyclopropyl)acetyl]-L-valyl}amino)hex-2-enoic acid,

(2E,4S)-2,5-dimethyl-4-(methyl{3-methyl-N-[(2R)-2-(methylamino)-2-(1-phenylcyclopropyl)acetyl]-L-valyl}amino)hex-2-enoic acid,

2-(4-{[3,3-Dimethyl-2-(3-methyl-2-methylamino-3-phenyl-butyrylamino)-butyryl]-methyl-amino}-2,5-dimethyl-hex-2-enoylamino)-4-methylsulfanyl-butyric acid methyl ester,

N,β,β-trimethyl-L-phenylalanyl-N1-((1S,2E)-4-{[(1S)-1-carboxy-3-(methylthio)propyl]amino}-1-isopropyl-3-methyl-4-oxobut-2-enyl)-N-1,3-dimethyl-L-valinamide,

N,β,β-trimethyl4-[(E)-2-phenylvinyl]-L-phenylalanyl-N¹-[(1S,2E)-3-carboxy-1-isopropylbut-2-enyl]-N¹,3-dimethyl-L-valinamide,

N,β,β-trimethyl4-[(E)-2-phenylvinyl]-D-phenylalanyl-N¹-[(1S,2E)-3-carboxy-1-isopropylbut-2-enyl]-N¹,3-dimethyl-L-valinamide,

N,β,β-trimethyl-L-phenylalanyl-N¹-[(1S,2E)-3-carboxy-1-isopropylbut-2′enyl]-3-fluoro-N¹-methyl-D-valinamide,

N,β,β-trimethyl-L-phenylalanyl-N¹-[(1S,2E)-3-carboxy-1-isopropylbut-2-enyl]-3-fluoro-N¹-methyl-L-valinamide,

3-[(4-methoxybenzyl)thio]-N-methyl-L-valyl-N¹-[(1S,2E)-3-carboxy-1-isopropylbut-2-enyl]-N¹,3-dimethyl-L-valinamide,

N-ethyl-β,β-dimethyl-L-phenylalanyl-N¹-[(1S,2E)-3-carboxy-1-isopropylbut-2-enyl]-N¹,3-dimethyl-L-valinamide,

(2E,4S)-2,5-dimethyl-4-(methyl{3-methyl-N-[(2S)-3-methyl-3-phenyl-2-pyrrolidin-1-ylbutanoyl]-L-valyl}amino)hex-2-enoic acid,

N-(2-hydroxyethyl)-β,β-dimethyl-L-phenylalanyl-N¹-[(1S,2E)-3-carboxy-1-isopropylbut-2-enyl]-N¹,3-dimethyl-L-valinamide,

(βR)-N,β-dimethyl-L-phenylalanyl-N¹-[(1S,2E)-3-carboxy-1-isopropylbut-2-enyl]-N¹,3-dimethyl-L-valinamide,

3-acetyl-N,β,β-trimethyl-L-phenylalanyl-N¹-[(1S,2E)-3-carboxy-1-isopropylbut-2-enyl]-N¹,3-dimethyl-L-valinamide,

N,β,β-trimethyl-L-phenylalanyl-N¹-[(1S,2E)-3-carboxy-1-isopropylbut-2-enyl]-3-hydroxy-N¹-methyl-L-valinamide and

N,β,β-trimethyl-L-phenylalanyl-N¹-[(1R,2E)-3-carboxy-1-isopropylbut-2- enyl]-N¹,3-dimethyl-L-valinamide.

Among the more specifically preferred compounds of Formula (II) of this invention for a method of treating, inhibiting the growth of, or eradicating a tumor in a mammal in need thereof wherein said tumor is resistant to at least one chemotherapeutic agent which method comprises providing to said mammal an effective amount of a compound of Formula (II) and pharmaceutically acceptable salts thereof are those set forth below:

3-Chloro-N,β,β-trimethyl-L-phenylalanyl-N¹-[(1S,2E)-3-carboxy-1-isopropyl-2-butenyl]-N¹,3-dimethyl-L-valinamide,

3-bromo-N,β,β-trimethyl-L-phenylalanyl-N¹-[(1S,2E)-3-carboxy-1-isopropylbut-2-enyl]-N¹,3-dimethyl-L-valinamide,

N,β,β,3-Tetramethyl-L-phenylalanyl-N¹-[(1S,2E)-3-carboxy-1-isopropyl-2-butenyl]-N¹,3-dimethyl-L-valinamide,

3-Cyclohexyl-N-methyl-L-valyl-N¹-[(1S,2E)-3-carboxy-1-isopropyl-2-butenyl]-N¹,3-dimethyl-L-valinamide,

N,O,β,β-tetramethyl-L-tyrosyl-N¹-[(1S,2E)-3-carboxy-1-isopropyl-2-butenyl]-N¹,3-dimethyl-L-valinamide,

N,O,β,β-tetramethyl-L-tyrosyl-N¹-[(1S,2E)-3-carboxy-1-isopropyl-2-butenyl]-N¹-methyl-3-(methylsulfanyl)-L-valinamide, and

N,β,β-3,4-Pentamethyl-L-phenylalanyl-N¹-[(1S,2E)-4-ethoxy-1-isopropyl-3-methyl-4-oxo-2-butenyl]-N¹,3-dimethyl-L-valinamide.

A more specifically preferred compound of Formula (II) of this invention for a method of treating, inhibiting the growth of, or eradicating a tumor in a mammal in need thereof wherein said tumor is resistant to at least one chemotherapeutic agent which method comprises providing to said mammal an effective amount of a compound of Formula (II) and pharmaceutically acceptable salts thereof is the compound set forth below:

N,β,β-trimethyl-L-phenylalanyl-N¹-[(1S,2E)-3-carboxy-1-isopropylbut-2-enyl]-N¹,3-dimethyl-L-valinamide.

An additional more specifically preferred compound of Formula (II) is N,O,β,β-tetramethyl-L-tyrosyl-N¹-[(1S,2E)-3-carboxy-1-isopropyl-2-butenyl]-N¹,3-dimethyl-L-valinamide.

In particular, the method of this invention is useful in tumor cells that overexpress MDR-1, MXR, or MRP. Consistent with this in vitro data, and in particular N,β,β-trimethyl-L-phenylalanyl-N¹-[(1S,2E)-3-carboxy-1-isopropylbut-2-enyl]-N¹,3-dimethyl-L-valinamide retains antitumor activity in xenograft mouse models where tumor cells are derived from cells selected for overexpression of MDR-1 or have inherent resistance to paclitaxel or vinblastine mediated by mechanisms including, but not limited to, MDR-1. Further, two tumor cell lines of distinct origin when selected for low-level resistance to N,β,β-trimethyl-L-phenylalanyl-N¹-[(1S,2E)-3-carboxy-1-isopropylbut-2-enyl]-N¹,3-dimethyl-L-valinamide do not overexpress MDR-1 and retain, or have increase sensitivity, to agents that polymerize tubulin.

As used herein an effective amount refers to the quantity of a compound of the invention which is sufficient to yield a desired therapeutic response without undue adverse side effects (such as toxicity) commensurate with a reasonable benefit/risk ration when used in the method of this invention.

A further object of the invention is a process for the preparation of a carboxylic acid of the formula

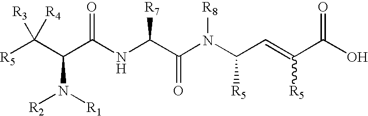

wherein:

R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, NO₂, —SO₃H, —SOR₁₀, or —SO₂R₁O, wherein R₁₀ is a linear, branched or cyclic, one to ten carbon saturated or unsaturated alkyl group; and aryl-R-; or R₁ and R₂ taken together with the nitrogen atom to which they are attached is a three to seven membered ring;

R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

or R₃ and R₄ taken together with the carbon to which they are attached form a three to seven membered ring;

R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, -COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R- and aryl and provided that when R₅ is an indolyl moiety of the formula

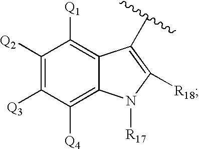

R₁₇ is H or an optionally substituted alkyl or acyl group; and

R₁₈ Q₁, Q₂, Q₃ and Q₄ are independently selected from H, halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;

R₇ is selected from the group consisting of a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

and wherein,

R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group;

X is a moiety selected from the group consisting of —OH, —OR, ═O, ═S, —O₂CR, —SH, —SR, —SOCR, —NH₂, —NHR, —N(R)₂, —NHCOR, NRCOR, —I, Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;

Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;

comprising the steps of:

a) treating a carboxylic acid of the formula

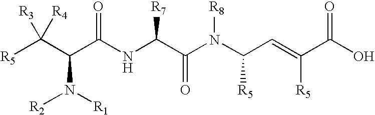

with ozone in methanol followed by further treating with dimethylsulfide to obtain an aldehyde of the formula

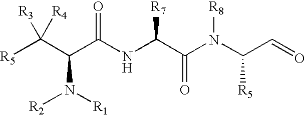

b) reacting said aldehyde with a phosphonate of the formula

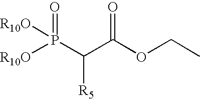

where R₁₀ is optionally fluoro substituted alkyl of 1 to 10 carbon atoms, in the presence of potassium hexamethyldisilazide and 1 8-crown-6 and hydrolyzing with base to obtain a carboxylic acid of the formula

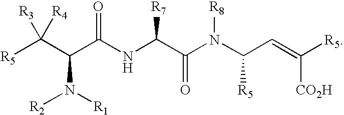

A further object of the invention is a process for the preparation of a carboxylic acid of the formula

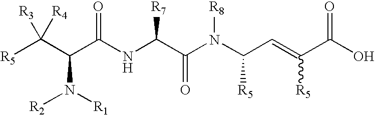

wherein:

R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;

R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, NO₂, —SO₃H, —SOR₁₀ or —SO₂R₁₀, wherein R₁₀ is a linear, branched or cyclic, one to ten carbon saturated or unsaturated alkyl group; and aryl-R-; or R₁ and R₂ taken together with the nitrogen atom to which they are attached is a three to seven membered ring;

R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁O is a linear, branched or cyclic, one to ten carbon saturated or unsaturated alkyl group; and aryl-R-;

R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

or R₃ and R₄ taken together with the carbon to which they are attached form a three to seven membered ring;

R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R- and aryl and provided that when R₅ is an indolyl moiety of the formula

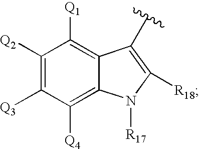

R₁₇ is H or an optionally substituted alkyl or acyl group; and

R₁₈ Q₁, Q₂, Q₃ and Q₄ are independently selected from H, halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;

R₇ is selected from the group consisting of a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;

and wherein,

R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁OH, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group;

X is a moiety selected from the group consisting of —OH, —OR, ═O, ═S, —O₂CR, —SH, —SR, —SOCR, —NH₂, —NHR, —N(R)₂, —NHCOR, NRCOR, —I, Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;

Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;

comprising the steps of:

a) treating a carboxylic acid of the formula

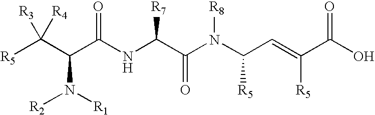

with ozone in methanol followed by further treating with dimethylsulfide to obtain an aldehyde of the formula

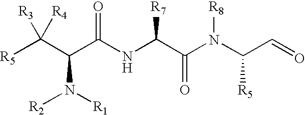

b) reacting said aldehyde of step a) with triphenylphosphorane of the formula

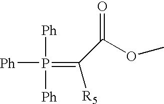

and hydrolyzing with base to obtain said carboxylic acid having the formula

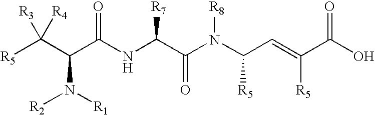

### DETAILED DESCRIPTION OF THE INVENTION

Methods and procedures to prepare compounds of Formula (II) where R₅ is indolyl is described in U.S. Pat. No. 6,153,590, the entire disclosure of which is hereby incorporated by reference. Further synthetic methods are described in WO 99/32509 and WO 96/33211. In addition compounds of this invention may be prepared as outlined in the following schemes.

As described in Scheme I, acetyl glycine 1 is reacted with aldehyde 2 in the presence of sodium acetate and acetic anhydride to afford 1,3-oxazole 3 which is further treated with base and following acidification gives 2-oxopropanoic acid 4. Alternatively, reacting aldehyde 2 with hydantoin 5 in the presence of ammonium bicarbonate affords 2,4-imidazolidinedione 6 which is further treated with base and following acidification gives 2-oxopropanoic acid 4. Reaction of 2-oxopropanoic acid 4 with R₃X or XR₃)(optionally followed by R₄X or with XR₃R₄X gives pyruvic acid 7 which is further reacted with amine R,NH₂ 8 in the presence of BH₃-pyridine complex to afford carboxylic acid 9. Carboxylic acid 9 is coupled with amine 10 in the presence of benzotriazole-1-yl-oxy-tris-pyrrolidinophosphonium hexafluorophosphate (PyBOP), N,N-diisopropylethylamine or 1-hydroxybenzotriazole hydrate(HOBT), coupling reagent 1-(3-dimethylaminopropyl)-3-ethylcarbondiimine hydrochloride(EDCI) and N-methylmorpholine followed by treatment with lithium hydroxide in aqueous methanol to afford acid 11 and acid 12.

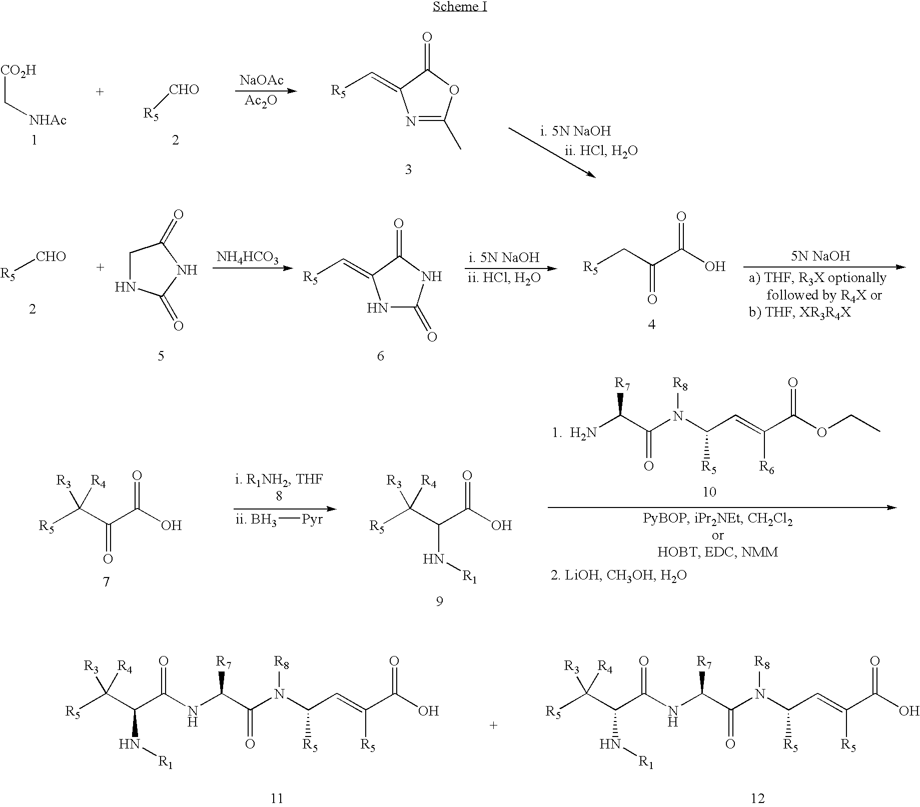

As further described in Scheme II, reacting diphenylsulfide 13 with silverborate affords borate salt 14 which is further reacted with lithium diisopropylamine followed by the addition of aldehyde 15 to afford oxirane 16 which is rearranged with tris(pentafluorophenyl)borane to give aldehyde 17. Treating aldehyde 17 with methylamine in the presence of potassium cyanide affords nitrile 18 which after further treatment with lithium hydroxide gives amide 19. Reacting amide 19 with di-t-butyl dicarbonate in the presence of dimethylaminopyridine gives protected 2,4-imidazolidinedione 20 or alternatively reaction with di-t-butyl dicarbonate over 4 to 5 days gives unisolated intermediate 21 which is then treated with dimethylaminopyridine in the presence of N,N-diisopropylethylamine to afford tri-substituted amide 22 which after treating with sodium hydroxide gives carboxylic acid 23.

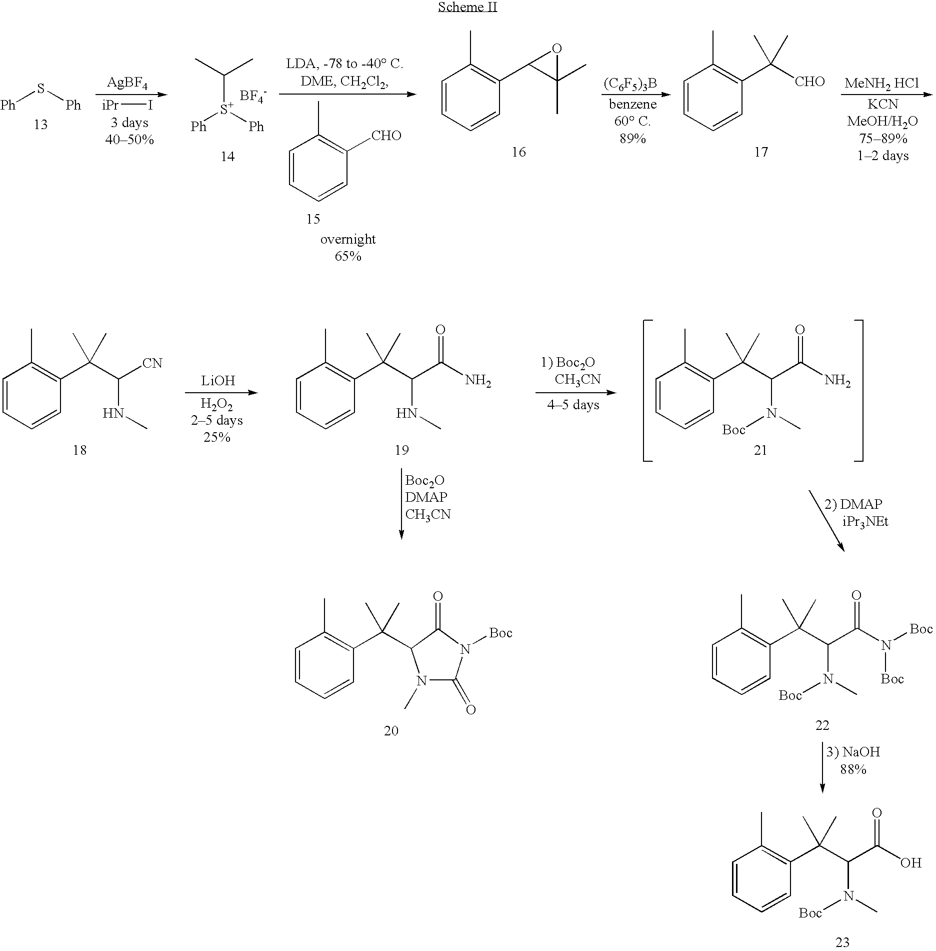

Described in Scheme III, alkylation of ester 24 with R₂X followed by hydrolysis affords carboxylic acid 25.

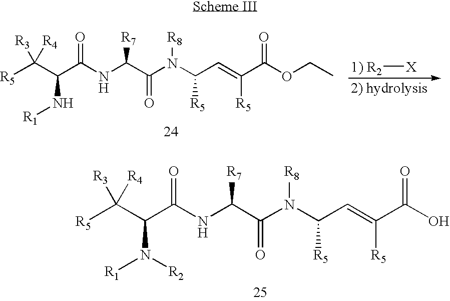

As described in Scheme IV, oxidation of sulfanyl 26 with metachloroperoxybenzoic acid affords sulfonyl 27 which is deblocked with acid to afford carboxylic acid 28.

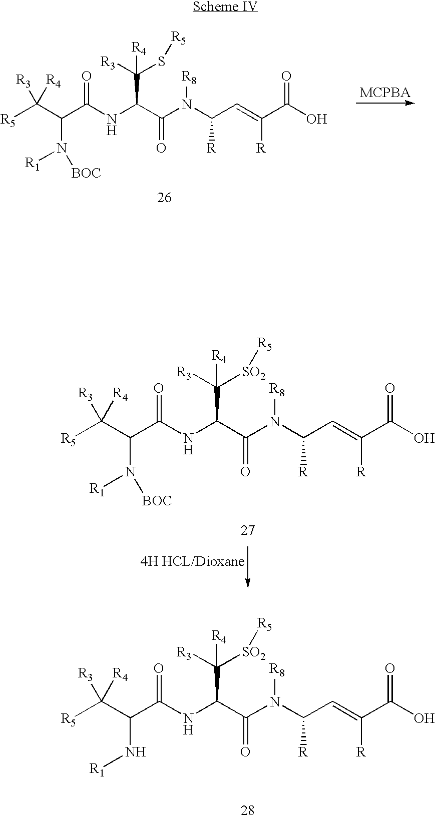

Described in Scheme V is the bromination of pyruvic acid 29 with bromine in the presence of iron in carbon tetrachloride to afford bromophenyl pyruvic acid 30 which undergoes reductive amination in the presence of amine 31 to afford substituted amine 32 which is coupled with amine 10 to afford ester 33 which is then hydrolyzed to afford carboxylic acid 35. As further described, using a Suzuki coupling ester 33 is reacted with phenylboronic acid and Pd(O) followed by hydrolysis to give acid 34.

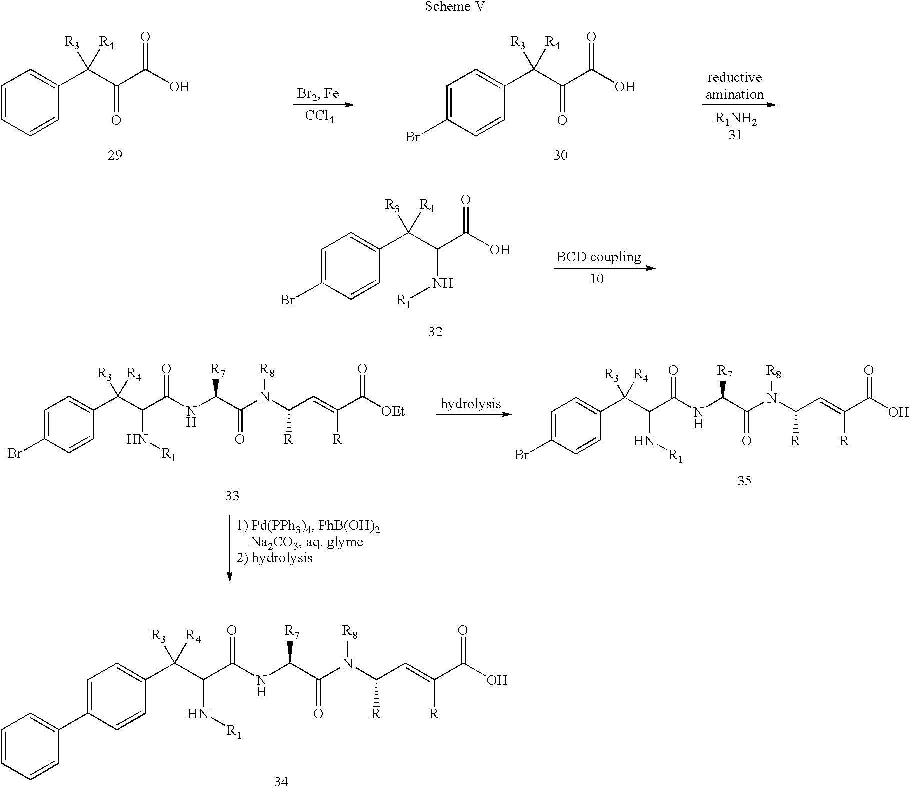

Referring to Scheme VI, bromopyruvic acid 30 is treated with ammonia followed by further treatment with borane-pyridine complex, followed by reaction with di-t-butyl dicarbonate in the presence of potassium carbonate followed by alkylation (R₁₁l) in the presence of sodium hydride to afford ester 36 . Ester 36 is further reacted with tetrakis(triphenylphosphine)palladium with the further addition of tri-n-butyl(vinyl)tin to afford vinyl intermediate 37 which is reacted with ozone with the further addition of dimethylsulfide to give aldehyde 38. Reacting aldehyde 38 with sulfamic acid followed by adding sodium chlorite gives carboxylic acid 39 which is then reacted with dimethylformamide di-t-butylacetal to afford t-butyl ester 40 following hydrolysis. Coupling t-butyl ester 40 with amine 10 with further deprotections as described in Scheme I affords carboxylic acid 41.

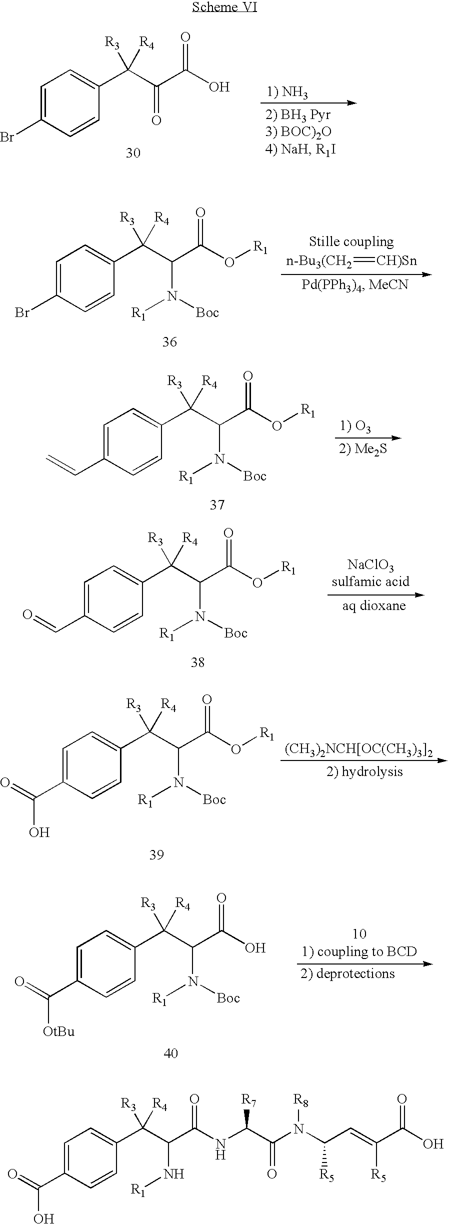

As described in Scheme VII, ester 42 is reacted with benzoyl chloride 43 to afford butanoate 44 which on further reaction with ethylene glycol in the presence of p-toluenesulfonic acid in toluene affords ketal 45. Ketal 45 is hydrolyzed with lithium hydroxide followed by reaction with pivaloyl chloride and coupling with chiral oxazolidinone 46 to give oxazolidin-2-one 47 which is further reacted with potassium hexamethylsilazide followed by trisyl azide with a final acetic acid quench to give azide 48. Reaction of azide 48 with di-t-butyl dicarbonate in the presence of hydrogen and Pd/C followed by hydrolysis with lithium hydroxide and hydrogen peroxide gives carboxylic acid 49.

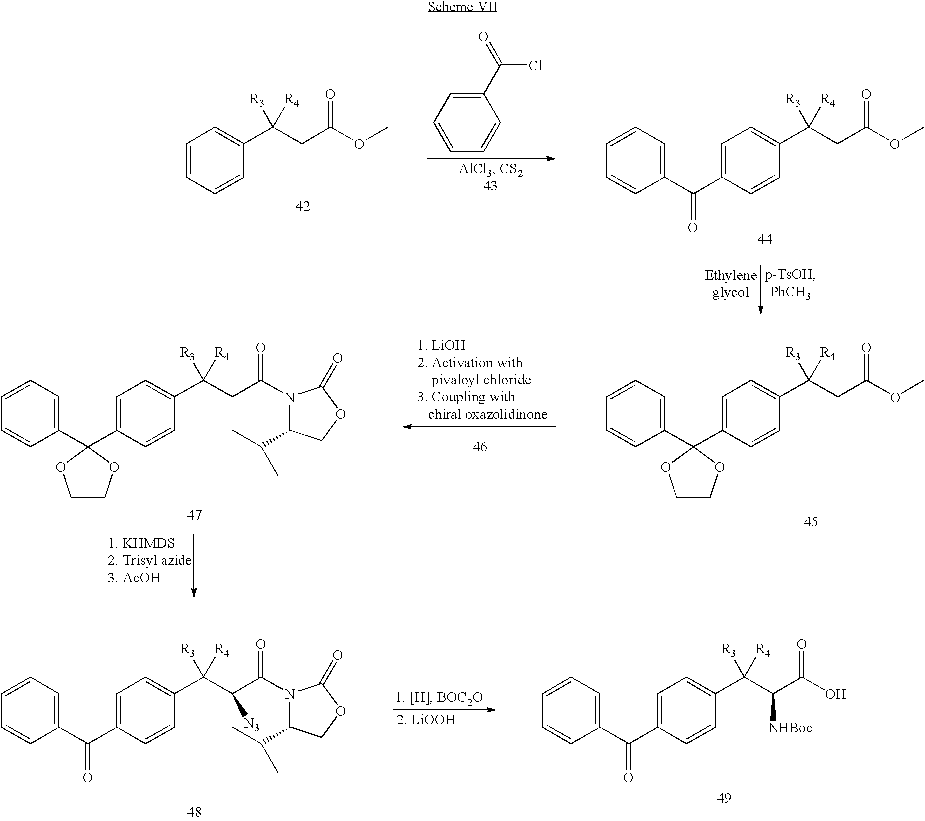

Referring to Scheme VIII, pyruvic acid 50 is reacted with borontribromide in methylene chloride to afford phenol 51 which is further reacted with amine 52 in the presence of borane-pyridine to afford carboxylic acid 53 which is coupled with ester 10 to give phenol 54 which is hydrolyzed with lithium hydroxide to afford carboxylic acid 55.

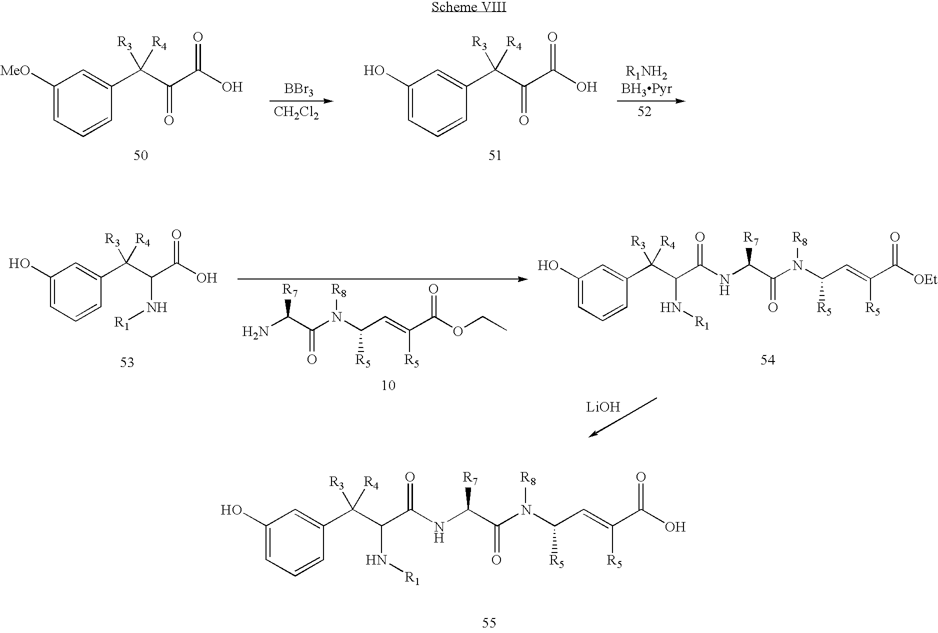

As shown in Scheme IX, carboxylic acid 49 is coupled to amine 56 using N,N-diisopropylethylamine, dimethylaminopyridine and benzotriazol-1-yloxy-tris(dimethylamino)phosphonium hexafluorophosphate(BOP coupling reagent) to afford ester 57 which is deprotected with acid then coupled with acid 58 followed by hydrolysis with lithium hydroxide to give amine 59.

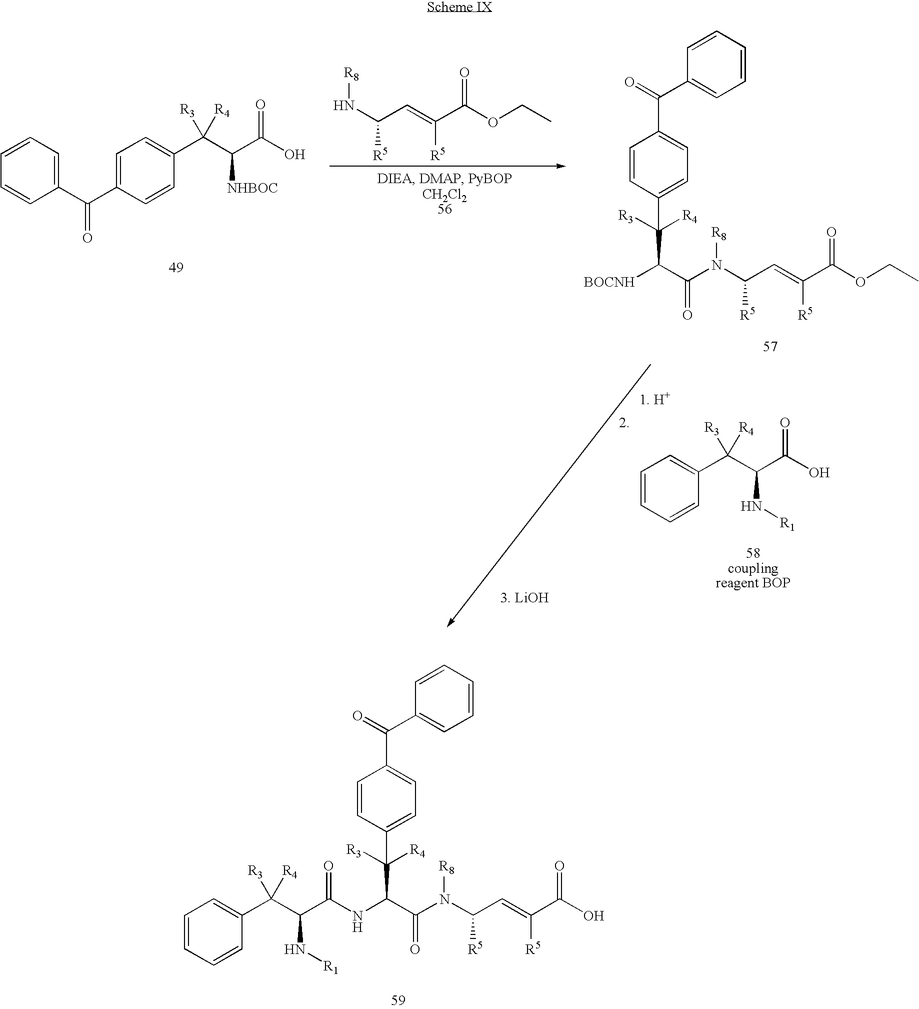

Referring to Scheme X, carboxylic acid 60 is alkylated in the presence of sodium hydride and R₁X in dimethylformamide followed by hydrolysis with lithium hydroxide in water to give ketal 61 which is further coupled with ester 10 in the presence of (EDCI), HOBT, and N-methylmorpholine in dimethylformamide to afford ester 62. Ester 62 is deblocked with aqueous hydrochloric acid followed by trifluoroacetic acid in methylene chloride and hydrolyzed with lithium hydroxide in aqueous methanol to give ketone 63.

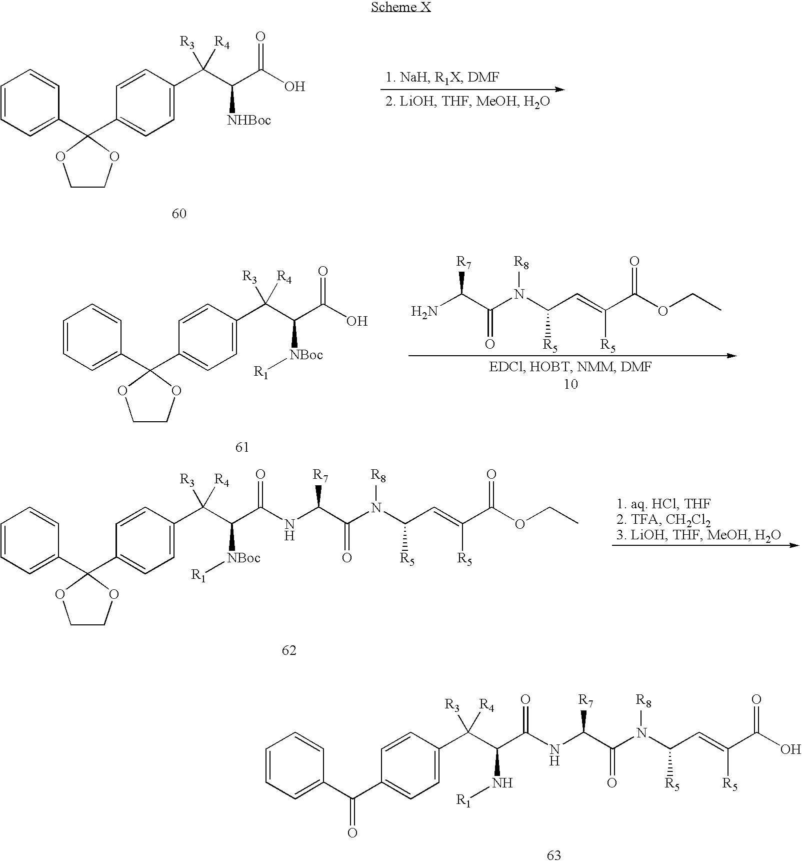

As shown in Scheme XI, ester 64 is reacted with tri-n-butyl(vinyl)tin and tetrakis(triphenylphosphine)palladium to afford olefin 65 which is hydrolyzed with lithium hydroxide to give carboxylic acid 66. Carboxylic acid 66 is reduced in the presence of palladium/carbon and hydrogen to give amine 67. Ester 64 is reacted with phenyl boronic acid in the presence of sodium carbonate and Pd(O) to afford biphenyl 68 which is further reacted with lithium hydroxide in aqueous methanol to give carboxylic acid 69.

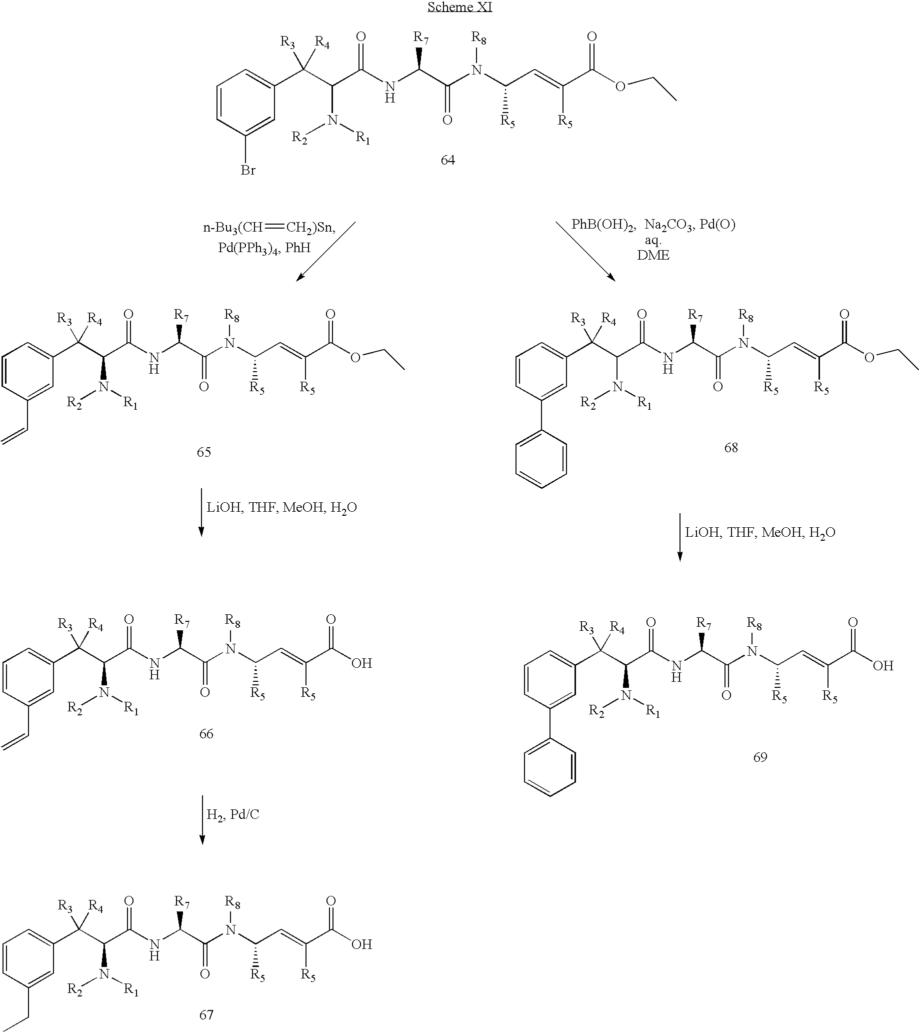

Referring to Scheme XII, reaction of carboxylic acid 70 with amine 71 in the presence of HOBT, EDCI and N,N-diisopropylethylamine in dimethylformamide affords amide 72. Also, reaction of carboxylic acid 70 with alcohol 73 in the presence of EDCI and dimethylaminopyridine in dichloromethane affords ester 74.

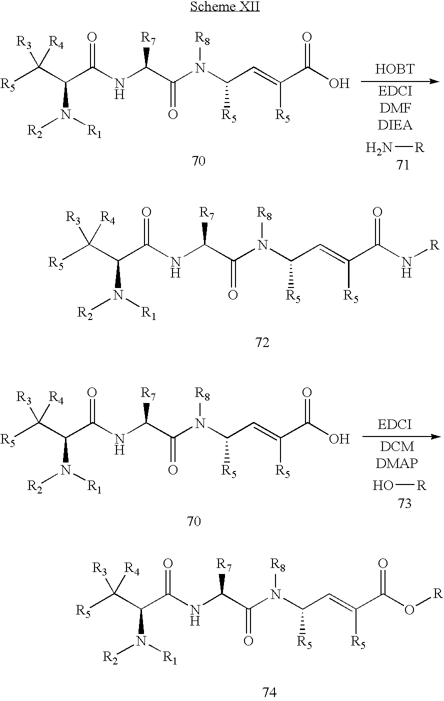

Referring to Scheme XIII, sodium salt of pyruvic acid 75 is reacted with diiodide 76 in the presence of sodium hydroxide to afford spiro 77 which is further reacted with methylamine in the presence of borane-pyridine complex to afford amino acid 78.

Described in Scheme XIV is the reduction of carboxylic acid 79 in the presence of platinum oxide in acetic acid to afford cyclohexyl aminoacid 80.

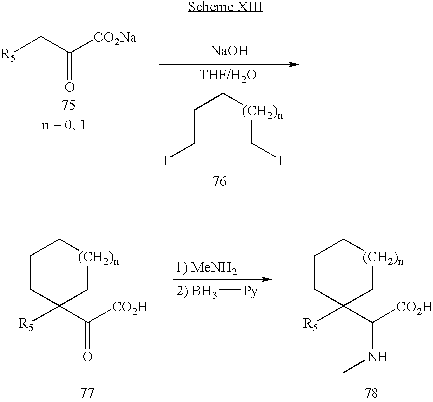

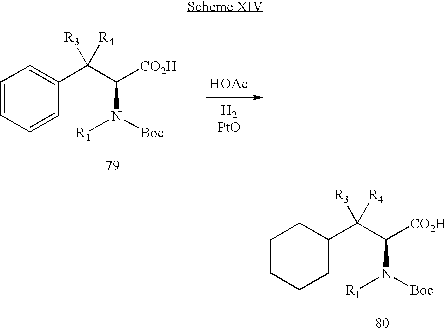

As described in Scheme XV, carboxylic acid 81 is treated with ozone in methanol followed by reaction with dimethylsulfide to afford aldehyde 82 which is further treated with phosphonate 83 where R₁₀ is an alkyl of 1 to 10 carbon atoms optionally substituted with fluorine of potassium hexamethyldisilazide (KHMDS) and 1 8-crown-6 followed by hydrolysis with lithium hydroxide to give carboxylic acid 84. Reaction of aldehyde 82 with (carboethoxyetylidene)triphenylphosphorane 86 followed by hydrolysis with lithium hydroxide gives carboxylic acid 85.

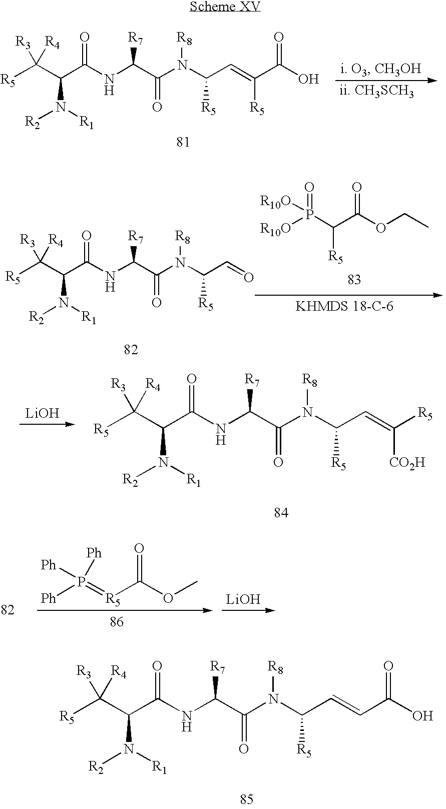

Cytotoxicity Standard Pharmacological Test Procedures

2. Methods

2.1 Cells.

Cells lines with previously known mechanisms of resistance. Drug sensitive (parental) cells and their resistant counterparts are obtained from the originator of the cell lines and maintained in media specified in the indicated reports: S1 (parental derived from a subclone of human colon carcinoma cell, LS174T) and S1-M1-3.2 cells (Greenberger, L. M., Collins, K. I., Annable, T., Boni, J. P., May, M. K., Lai, F. M., Kramer, R., Citeralla, R. V., Hallett, W. A., Powell, D. α-(3,4-dimethyoxyphenyl)-3,4-dihydro-6,7-dimethoxy-alpha-[(4-methylphenyl)thio]-2(1 H)-isoquinolineheptanenitrile (CL 329,753): a novel chemosensitizing agent for P-glycoprotein-mediated resistance with improved biological properties compared with verapamil and cyclosporine A. Oncol. Res. 8: 207-218,1996); KB-3-1 (parental derived from an epidermoid carcinoma), KB-8.5, KB-V1 (Akiyama S-I, Fojo A, Hanover J A, Pastan I, Gottesman M M. Isolation and genetic characterization of human KB cell lines resistant to multiple drugs. Somatic Cell Mol. Gen., 11: 117-126,1985; Shen D-W, Cardarelli C, Hwang J, Cornwell M, Richert N, Ishii S, Pastan I, Gottesman MM. Multiple drug-resistant human KB carcinoma cells independently selected for high-level resistance to colchicine, adriamycin or vinblastine show changes in expression of specific proteins. J. Biol. Chem., 261: 7762-7770, 1986); and HL-60 (parental derived from human leukemia) and HL60/AR (McGrath T., Center M. S. Adriamycin resistance in HL60 cells in the absence of detectable P-glycoprotein. Biochem. Res. Commun., 145:1171-1176,1987); A549 and A549.EpoB40 (He L., Yang C.-P.H., Horwitz S. B. Mutations in p-tubulin map to domains involved in regulation of microtubule stability in epothilone-resistant cell lines. Molecular Cancer Therapeutics. 1: 3-10, 2001). HCT-15 human colon carcinoma and DLD-1 human colon carcinoma are obtained from American Tissue Culture Collection. The MX-1W carcinoma is a Wyeth variant orginally obtained as MX-1 breast carcinoma from the National Cancer Institute. Cell lines selected for resistance to Representative Example 129. KB-3-1 cells are selected for resistance to Example 129 by chronically exposing cells to increasing concentrations of Example 129. In general, selection is begun at or near the IC₅₀ (approximately 1 nM), and the concentration of Example 129 is increased up to approximately 6 nM over the course of 6-12 months or at least until a level of resistance of greater than 10-fold is observed.

2.2. Cell Survival Assay: Standard Pharmacological Test Procedure. The concentration of candidate inhibitor required to inhibit 50% of cell growth (IC₅₀) is done according to previously reported methods. (Discafani, C. M., Carroll, M. L., Floyd Jr., M. B. F., Hollander, I. J., Husain, Z., Johnson, B. D., Kitchen, D., May, M. K., Malo, M. S., Minnick Jr., A. A., Nilakantan, R., Shen, R., Wang Y-F., Wissner, A., Greenberger, L. M. Irreversible inhibition of epidermal growth factor receptor tyrosine kinase with in vivo activity by N-[4-[3-bromophenyl)amino]-6-quinaxolinyl]-2-butynamide (CL-387,785), Biochem. Pharmacol. 57: 917-925, 1999) Briefly, cells are plated in 100 μl of media in the morning of day 1 and allowed to adhere to the plates for 2-6 hr. Compounds are serially diluted into media as 2× stocks and 100 μl added to cells in duplicate. Compounds are incubated with cells for 3 days. At the end of the incubation period the sulforhodamine B (SRB) assay, which measures protein content as an assessment of cell survival, is performed as described previously (Skehan P, Storeng R, Scudiero D, Monks A, McMahon J, Vistica D, Warren J T, Bokesch H, Kenney S, Boyd MR. New calorimetric cytotoxicity assay for anticancer-drug screening. J Natl Cancer Inst 1990 Jul 4;82(13):1107-12) with some modification as follows. For KB-3-1 cells with 20% FCS, media is gently decanted and replaced with 200 μl of serum-free media mixed with cold 50% TCA for a final concentration of 10% TCA. The plates are incubated for 1 hr at 4° C., followed by washing 5 times in cold distilled water, then air dried overnight. The fixed cells are stained for 10 min with 80 μl of 0.04% SRB solution prepared in 1% glacial acetic acid. Stain is discarded and plates washed 5 times in 1% glacial acetic acid, then air-dried until completely dry. Stained cell product is dissolved in 150 μl of 10 mM Trizma Base and placed on a shaker for 20 minutes until fully dissolved. Absorbance is read on a Victor V multi-label plate reader (Perkin Elmer, Gaithersburg, Md.).

2.3 Immunoblot analysis The level of P-glycoprotein (MDR₁) in tumor cell lines is determined by isolating membranes from cells, followed by immunoblot analyses. Briefly, nearly confluent cells in a T225 flask are trypsinized, collected in serum-containing media and centrifuged at 1500 rpm for 5 min. Cell pellets are resuspended in 5 ml of PBS, centrifuged again and lysed in 2 ml of lysis solution (10 mM Tris, pH 8.0, 10 mM NaCl, 1 mM MgCl₂, 100 units/ml aprotinin, 30 μM leupeptin, 1 μg/ml pepstatin, 1 mM PMSF) at 4° C. for 20 min. Samples are sonicated 3 cycles for 10 sec each and centrifuged at 2000 rpm for 10 min at 4° C. Supernatants are transferred to fresh tubes, normalized to 3 ml with 10 mM Tris, pH 7.4 with 100 units/ml aprotinin, and centrifuged at 100,000×g in a SW 28.1 rotor (Beckman, Palo Alto, Calif.) for 2 hr at 4° C. Pellets are completely resuspended in 100 μl of 10 mM Tris, 7.4 with aprotinin, then frozen at −70° C. until use. Equal amounts of total protein (20 μg) are resolved in 12% polyacrylamide gels containing SDS (SDS-PAGE) in 1×SDS/Tris/glycine running buffer and transferred to PVDF membranes for 2-3 hr at 90 V in 1× Tris/glycine transfer buffer. Western blots are first blocked at room temperature in 5% fat-free milk in TBST (Tris-buffered saline with 0.1% Tween-20) for 1 hr. Blots are then incubated with anti-P-glycoprotein antibody (Calbiochem) at 1:500 final dilution overnight at 4° C. with shaking in 5% milk/TBST. Blots are washed 3X in TBST for 10 min each then incubated with 1:2000 dilution of goat anti-rabbit IgG-HRP as secondary antibody (HRP-conjugated sheep-anti-mouse IgG; Amersham, Arlington Heights, Ill.) for 1 hr at room temperature in 5% milk/TBST and washed again in TBST. Protein signals are detected by exposure for 1 min to ECL reagents (Enhanced Chemiluminescence kit, Amersham), followed by exposure to autoradiographic film (Amersham) for various times.

2.4. Growth of Tumors in Nude Mice. Drug efficacy studies in mice are performed similar to previously reported studies. (Discafani, C. M., Carroll, M. L., Floyd Jr., M. B. F., Hollander, I. J., Husain, Z., Johnson, B. D., Kitchen, D., May, M. K., Malo, M. S., Minnick Jr., A. A., Nilakantan, R., Shen, R., Wang Y-F., Wissner, A., Greenberger, L. M. Irreversible inhibition of epidermal growth factor receptor tyrosine kinase with in vivo activity by N-[4-[3-bromophenyl)amino]-6-quinaxolinyl]-2-butynamide (CL-387,785), Biochem. Pharmacol. 57: 917-925, 1999) Briefly, athymic nu/nu female mice (Charles River Laboratories) are implanted SC (subcutaneously) with 1.5×10⁶ LOX melanoma cells, 2.5×10⁶ KB-3-1 cells, 7.0×10⁶ KB-8.5 cells, 5×10⁶ HCT15 cells, 6×10⁶ DLD1 cells, or with approximately 5 tumor fragments of MX1W. When tumors attain a mass of between 80 and 120 mg (day 0), animals are randomized into treatment groups each containing either 5 or 10 animals (dependent upon the experiment). In some experiments, tumors are allowed to grow up to 2.5 grams in size before drug treatment is initiated. After staging, animals are treated intravenously (IV) with one or more doses of Example 129 formulated in saline, 60 mg/kg/dose IV paclitaxel formulated in 6% ethanol/6% Cremophor EL/saline, 1.0 mg/kg/dose IP (intraperitoneal) vincristine formulated in saline or vehicle control. The doses chosen for both paclitaxel and vincristine are between 80 and 90% of the maximum tolerated dose for each drug. Tumor mass ([Length×Width 21/2) is determined once a week for up to 35 days. The relative tumor growth (mean tumor mass on day measured divided by the mean tumor mass on day zero) and the percent Tumor/Control (% T/C) are then calculated for each treatment group for the duration of each experiment. The % T/C is defined as the Mean Relative Tumor Growth of the Treated Group divided by the Mean Relative Tumor Growth of Vehicle Control Group multiplied by 100. The data are analyzed via a one-sided Student's t-test. A p-value ≦0.05 indicates a statistically significant reduction in relative tumor growth of treated group compared to that of the vehicle control group. A drug dose is considered toxic if there is greater than 20% lethality or if animals have lost ≧20% of their initial body weight.

In vitro Analysis

3.1.1.Cell Lines With Inherent Resistance to Taxanes

Hemiasterlin, a natural product analog of Example 129, is a potent inhibitor of growth that causes arrest at the G₂/M phase of the cell cycle. (Anderson, H. J., Coleman, J. E., Andersen, R. J., Roberge, M. Cytotoxic peptides hemiasterlin, hemiasterlin A and hemiasterlin B induce mitotic arrest and abnormal spindle formation, Cancer Chemother. Pharmacol. 39: 223-226, 1997).

Resistance to antimicrotubule agents (e.g. paclitaxel or Vinca alkaloids) frequently occurs in the clinic, and Example 129 and analogs thereof overcome resistance in tumor cell lines in culture and in animal models.

Example 129 is a potent inhibitor of cell growth in 34 tumor cell lines (mean IC₅₀=2.1∓1.7 nM, median 1.7 nM, range 0.2-7.3 nM; Table 1) and is distinct from paclitaxel which has an usually large range of activity (N=31 cell lines; mean IC₅₀=80.2±292 nM, median 9.0 nM, range 1.8-1594 nM). The activity of Example 129 is 10 independent of tumor origin and in many cases is considerably more potent than paclitaxel (Table 1). The latter phenomena is related to both the chemical properties of Example 129 and its ability to overcome various mechanisms of taxane resistance.

TABLE 1
Example 129 Compared Against a Panel of Tumor Cell Lines^{a}

| | | IC₅₀ (nM) | |
|---|---|---|---|
| cell line | tumor origin | HTI-286 | paclitaxel |
| BT474 | breast | 2.5 ± 1.6 (2) | 10 (1) |
| MCF7 | breast | 7.3 ± 2.3 (3) | 8.1 ± 1.9 (3) |
| MDA-MB-435 | breast | 0.5 (1) | |
| MX-1W | breast | 1.8 ± 0.6 (2) | 15.4 ± 2.9 (4) |
| SKBR3 | breast | 2.3 (1) | |
| Colo205 | colon | 1.5 ± 0.6 (8) | 6.9 ± 2.3 (8) |
| CX-1 | colon | 2.3 (1) | |
| DLD-1 | colon | 1.1 ± 0.4 (7) | 30.8 ± 12.7 (2) |
| HCT-15 | colon | 4.2 ± 2.5 (9) | 438 ± 248 (6) |
| HCT-116 | colon | 0.7 ± 0.2 (6) | 4.5 (1) |
| HT-29 | colon | 1.6 (1) | 6.1 (1) |
| KM20 | colon | 1.8 ± 0.6 (5) | 8.9 ± 1.9 (3) |
| Moser | colon | 5.3 ± 4.1 (3) | 1594 ± 1989 (2) |
| S1 | colon | 3.7 ± 2.0 (6) | 92 ± 100 (6) |
| SW620 | colon | 3.6 ± 0.8 (2) | 18 (1) |
| KB-3-1 | epidermoid | 0.96 ± 0.5 (79) | 3.9 ± 1.8 (74) |
| U87 | glioblastoma | 2.4 ± 0.3 (2) | 25.1 (1) |
| CCRF-CEM | leukemia | 0.2 ± 0.03 (2) | 3.3 ± 1.3 (2) |
| HL60 | leukemia | 0.21 ± 0.05 (5) | 1.8 (1) |
| A375 | melanoma | 1.1 ± 0.8 (5) | 14.1 ± 7.3 (3) |
| Lox | melanoma | 1.4 ± 0.6 (6) | 17.3 ± 5.6 (3) |
| SK-Mel-2 | melanoma | 1.7 ± 0.5 (3) | 3.2 ± 1.8 (3) |
| A549 | NSCLC | 1.1 ± 0.5 (3) | 8.8 ± 3.6 (3) |
| NCI-H1299 | NSCLC | 6.8 ± 6.1 (10) | 37.7 ± 12.6 (8) |
| NCI-H838 | NSCLC | | 6.3 ± 0.6 (8) |
| SK-MES-1 | NSCLC | 2.6 (1) | 29 (1) |
| 1A9 | ovarian | 0.56 ± 0.09 (8) | 5.1 ± 0.9 (7) |
| SKOV3 | ovarian | 1.7 (1) | 9 (1) |
| BxPC3 | pancreatic | 0.7 (1) | 10.8 (1) |
| Capan-1 | pancreatic | 1.4 (1) | 4 (1) |
| Panc1 | pancreatic | 2.9 ± 0.1 (2) | 28.9 (1) |
| DU145 | prostate | 2.0 (1) | nd |
| PC3MM2 | prostate | 1.9 ± 0.1 (2) | 8.7 (1) |
| A498 | renal | 0.8 (1) | 27.5 (1) |
| A431 | squamous | 2.4 ± 0.4 (2) | 7.6 (1) |
| AVERAGE | | 2.1 ± 1.7 (34) | 80.2 ± 292 (31) |

^{a}Cells were grown in the presence of HTI-286 or paclitaxel for 3 days and growth assessed by SRB staining or, for leukemia lines, MTS assay. Data are mean IC₅₀ (nM) ± standard deviation and number of experimental determinations (N).

3.1.2. Cell Lines With Acquired Resistance to Taxane or Vinca Alkaloids.

3.1.2.1. Resistance Mediated by Drug Efflux Pumps

Over-expression of drug efflux pumps is known to mediate resistance to various chemotherapeutics, including anti-microtubule drugs. These protein pumps prevent drug levels from reaching a cytotoxic threshold by actively transporting the agents out of the cell (Gottesman M M, Pastan I. Biochemistry of multidrug resistance mediated by the multidrug transporter. Ann Rev Biochem., 62: 385-427, 1993). Since Example 129 is an anti-microtubule drug, and resistance to many such drugs can be due in part to MDR₁ (ABCB1, P-glycoprotein), it was determined whether or not Example 129 would be effective in MDR₁-expressing cell models.

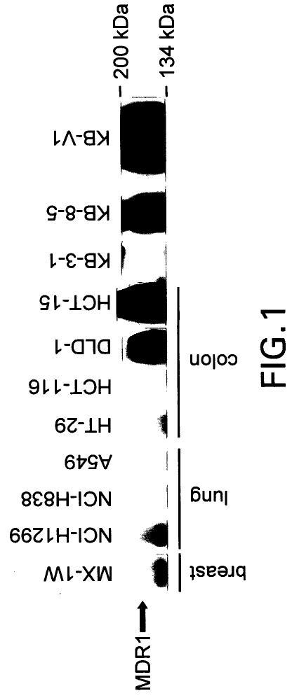

The expression of MDR₁ protein is assessed in a panel of tumor cell lines by immunoblot analyses. Undetectable levels of MDR₁ protein are observed in many cell lines (FIG. 1, NCl-H838, A549, HT-29, HCT-1 16, KB-3-1). In contrast, moderate levels of MDR₁ protein are detected in MX-1W breast carcinoma cells and NCl-H1299 lung carcinoma cells. Very high levels of MDR₁ protein are found in DLD-1 and HCT-1 5 colon carcinoma cells. Levels of MDR₁ mRNA are also examined in these cells using a real-time polymerase chain reaction assay and are found to correlate with protein levels. Consistent with these observations, higher concentrations of paclitaxel are required to inhibit in vitro growth of the latter four cell lines (IC₅₀=15-438 nM) compared with the 5 cell lines that express low levels of MDR₁ (IC₅₀=3.9-8.8 nM). In contrast, Example 129 potently inhibits the growth of cells that express undetectable or low level MDR₁ (IC₅₀=0.7-1.6 nM) or high level MDR₁ (IC₅₀=1.1-6.8 nM).

As shown in FIG. 1. Relative level of expression of MDR-1 protein in tumor cell lines. Membrane preparations of each cell line is prepared. Fifty micrograms of membrane protein is resolved on SDS-PAGE, transferred to PVDF membrane, and the membrane probed for MDR-1 (P-glycoprotein) expression.

Furthermore, an MDR₁-specific inhibitor has been shown previously to resensitize cells to MDR₁-substrates (Greenberger, L. M., Collins, K. I., Annable, K. I., Boni, J. P., May, M. K., Lai, F. M., Kramer, R., Citarella, R. V., Hallett, W. A., and Powell, D. α-(3,4-dimethyoxyphenyl)-3,4-dihydro-6,7-dimethoxy-α-[(4-methylphenyl)thio]-2(1H)-isoquinolineheptane-nitrile (CL 329,753): a novel chemosensitizing agent for P-glycoprotein-mediated resistance with improved biological properties compared to verapamil and cyclosporine A., Oncol. Res. 8: 207-218,1996). The cell lines MX-1W, HCT-15 and DLD-1 can be re-sensitized to paclitaxel with this MDR₁-specific inhibitor, supporting the role of MDR₁ in poor responsiveness to paclitaxel (Table 2). Therefore, cells that over-express MDR₁ are inherently resistant to paclitaxel in the absence of drug-selection. The MDR₁-specific inhibitor does not significantly alter the sensitivity to Example 129. This further supports the conclusion that Example 129 does not interact with MDR₁ in inherently resistant cells.

TABLE 2
Reversal of resistance to paclitaxel by an MDR-1 inhibitor, 7-(6,7-dimethoxy-
3,4-dihydro-1H-isoquinolin-2-yl)-2(3,4-dimethoxy-phenyl)-2-p-tolysulfanyl-
heptanenitrile¹

| | | | | IC₅₀ Example 129 + |
|---|---|---|---|---|
| | | IC₅₀ paclitaxel + | | 5.0 uM 7-(6,7- |
| | | 5.0 uM 7-(6,7- | | dimethoxy-3,4- |
| | | dimethoxy-3,4- | | dihydro-1H- |
| | | dihydro-1H- | | isoquinolin-2-yl)- |
| | | isoquinolin-2-yl)-2(3,4- | | 2(3,4-dimethoxy- |
| | IC₅₀ | dimethoxy-phenyl)-2- | IC₅₀ | phenyl)-2-p- |
| Cell | paclitaxel | p-tolysulfanyl- | Example | tolysulfanyl- |
| Line | (nM) | heptanenitrile | 129 | heptanenitrile |
| MX-1W | 138 | 8.3 | 1.4 | 1.3 |
| DLD-1 | 21.8 | 3.8 | 1.2 | 1.1 |
| HCT-15 | 479 | 5.0 | 2.2 | 0.5 |
| ¹Cells are grown for 3 days in media in the presence of an increasing concentration of paclitaxel or Example 129. Simultaneously, cells are exposed to 7-(6,7-dimethoxy-3,4-dihydro-1H-isoquinolin-2-yl)-2(3,4-dimethoxy-phenyl)-2-p-tolysulfanyl-heptanenitrile or a vehicle control (DMSO). Cell growth is determined by the SRB method. | | | | |
| # Five micromolar 7-(6,7-dimethoxy-3,4-dihydro-1H-isoquinolin-2-yl)-2(3,4-dimethoxy-phenyl)-2-p-tolysulfanyl-heptanenitrile has no inhibitory effect on cell growth when given alone. | | | | |

Cell lines selected for expression of drug efflux pumps by chronic drug exposure were also utilized to compare the activity of Example 129 with other chemotherapeutics (Table 3). KB-8-5 cells, which were selected for low-level resistance to colchicine and over-express moderate levels of MDR₁ approximately equivalent to levels observed in DLD-1 (see FIG. 1), were moderately resistant to paclitaxel (19-fold), docetaxel (18-fold), vinblastine (37-fold), vinorelbine (38-fold), colchicine (9.8-fold), and adriamycin (15-fold). In contrast, KB-8-5 cells had only 2.4-5 fold resistance to HTI-286.

Cells resistant to paclitaxel or vinblastine (eg, KB-85 at 19-fold or 37-fold, respectively) are not responsive to these drug in vivo, suggesting that this level of resistance in vitro translates to resistance in animals.

TABLE 3
Example 129 Overcomes Cellular Resistance to Standard
Chemotherapeutics Mediated by MDR1

| | KB-3-1 | KB-8-5 (MDR1+) | | KB-V1 (MDR1++) | |
|---|---|---|---|---|---|
| Compound | IC50, nM (N) | IC50, nM (N) | RR | IC50, nM (N) | RR |
| Example 129 | 0.96 ± 0.5 (79) | 2.3 ± 1.2 (69) | 2.4 | 77.4 ± 44 (67) | 81 |
| Hemiasterlin | 0.319 ± 0.095 (3) | 1.0 ± 0.5 (3) | 3.2 | 76.1 ± 13.8 (3) | 239 |
| Paclitaxel | 3.9 ± 1.8 (74) | 63.3 ± 29 (56) | 19 | 5484 ± 2780 (37) | 1406 |
| Docetaxel | 0.55 ± 0.45 (6) | 9.7 ± 6.6 (2) | 18 | 368 ± 257 (2) | 669 |
| Vinblastine | 0.79 ± 0.5 (9) | 29.2 ± 22 (2) | 37 | 1464 ± 1022 (2) | 1848 |
| Vinorelbine | 3.5 (1) | 131.9 (1) | 38 | >3000 | >857 |
| Colchicine | 6.4 ± 1.3 (9) | 62.7 ± 9.2 (2) | 9.8 | 2442 ± 2176 (2) | 382 |
| Dolastatin-10 | 0.037 ± 0.02 (13) | 0.263 ± 0.05 (4) | 7.1 | 21.2 ± 1.4 (4) | 573 |
| Adriamycin | 43.3 ± 32 (9) | 658 ± 483 (2) | 15 | 11489 ± 9672 (2) | 265 |
| Mitoxantrone | 10.3 ± 6.9 (10) | 19.1 (1) | 1.9 | >3000 (1) | >291 |
| ^{a}Data are mean IC₅₀ (nM) ± standard deviation for the indicated agents from multiple or single experiments (N = number of determinations, in parentheses). RR, relative resistance = ratio of IC₅₀ of the resistant cell model to IC₅₀ of the corresponding sensitive cell line. Lower relative resistance values indicate greater sensitivity of cells to the drug, values close to 1 indicate no resistance, | | | | | |
| # while values greater than 3 indicate resistance. Drug transporter is MDR1 (ABCB1, P-glycoprotein, multidrug resistance protein-1); MDR+ = moderate level expression; MDR1++ = high level expression. | | | | | |

Resistance to Example 129 can be mediated by MDR₁ in extreme circumstances, since 81-fold resistance to Example 129 is found in a cell line expressing very high levels of MDR₁ (KB-V1) and resistant to paclitaxel or vinblastine (>1000-fold). This is likely to be due to MDR₁ itself and not a co-selected factor since resistance to Example 129 paclitaxel or vinblastine is partially reversible with an MDR₁-specific inhibitor, 7-(6,7-Dimethoxy-3,4-dihydro-1H-isoquinolin-2-yl)-2-(3,4-dimethoxy-phenyl)-2-p-tolylsulfanyl-heptanenitrile (Table 4). However, the expression of MDR₁ in these cells is higher than what is typically found in clinical samples from a variety of tumors, including colon carcinomas that have the highest levels of the protein compared to most other tumor types (Goldstein L. J. MDR₁ gene expression in sold tumors. European J Cancer. 32A: 1039-1050,1996;). While these levels of MDR₁ may not be physiologically relevant, the upper range of MDR₁ expression in some human tumors may exist somewhere between that measured in the KB-8-5 and KB-V1 cell models (Alvarez, M., Paull, K., Monks, A., Hose, C., Lee, J.-S., Weinstein, J., Grever, M., Bates, S., Fojo, T. Generation of a drug resistance profile by quantitation of mdr-1/P-glycoprotein in the cell lines of the National Cancer Institute Anticancer Drug Screen. J. Clin. Invest. 95: 2205-2214, 1995) These data suggest that Example 129 overcomes clinically-relevant MDR₁-mediated resistance similar to the type that is found in cell lines that are inherently resistant to paclitaxel (e.g. HCT-1 5, DLD-1, MX-1W cells).

TABLE 4
Reversal of resistance to paclitaxel by an MDR-1 inhibitor, 7-(6,7-
dimethoxy-3,4-dihydro-1H-isoquinolin-2-yl)-2(3,4-dimethoxy-phenyl)-2-p-tolysulfanyl-
heptanenitrile in cells that acquire MDR-1 mediated drug resistance¹

| | | | | IC₅₀ Example 129 + |
|---|---|---|---|---|
| | | IC₅₀ paclitaxel + | | 5.0 uM 7-(6,7- |
| | | 5.0 uM 7-(6,7- | | dimethoxy-3,4- |
| | | dimethoxy-3,4- | | dihydro-1H- |
| | | dihydro-1H- | | isoquinolin-2-yl)- |
| | | isoquinolin-2-yl)-2(3,4- | | 2(3,4-dimethoxy- |
| | IC₅₀ | dimethoxy-phenyl)-2- | IC₅₀ | phenyl)-2-p- |
| Cell | paclitaxel | p-tolysulfanyl- | Example | tolysulfanyl- |
| Line | (nM) | heptanenitrile | 129 | heptanenitrile |
| KB-3-1 | 2.2 | 2.1 | 1.2 | 0.9 |
| KB-85 | 41.9 | 4.4 | 1.9 | 1.4 |
| KB-V1 | >3000 | 15 | 57 | 1.3 |
| ¹Cells are grown for 3 days in media in the presence of an increasing concentration of paclitaxel or Example 129. Simultaneously, cells are exposed to 7-(6,7-dimethoxy-3,4-dihydro-1H-isoquinolin-2-yl)-2(3,4-dimethoxy-phenyl)-2-p-tolysulfanyl-heptanenitrile or a vehicle control (DMSO). Cell growth is determined by the SRB method. Five micromolar 7-(6,7-dimethoxy-3,4-dihydro-1H-isoquinolin-2-yl)-2(3,4-dimethoxy-phenyl)-2-p-tolysulfanyl-heptanenitrile | | | | |
| # has no inhibitory effect on cell growth when given alone. Data are the mean of two experiments. | | | | |

The ABC-transporter MRP1 (multidrug resistance-associated protein-1, ABCC1) and the ABC-half-transporter MXR (ABC half-transporter, ABCG2, BCRP) are members of two other classes of drug transporters known to mediate resistance to chemotherapeutics, although they do not effectively handle anti-microtubule drugs (Loe D W, Deeley R G, Cole S P C. Biology of the multidrug resistance-associated protein, MRP. European J Cancer,32A: 945-957,1996; Litman T, Brangi M, Hudson E, Fetsch P, Abati A, Ross D D, Miyake K, Resau J H, Bates S E. The multidrug-resistant phenotype associated with overexpression of the new ABC half-transporter, MXR (ABCG2). J. Cell Science,113: 2011-2021, 2000). It was unknown whether the hemiasterlin family of peptides interact with these drug pumps. As observed for paclitaxel and vinblastine, no resistance to Example 129 is detected in cells selected for over-expression of MRP1 (HL60/AR) or MXR (S1-M1-3.2), although these cell lines are highly resistant to both doxorubucin (52- to >124-fold) and mitoxantrone (32- to 535-fold) (Table 5).

TABLE 5
Example 129 Overcomes Cellular Resistance to Standard
Chemotherapeutics Mediated by MRP1 and MXR

| | | HL60/AR | | S1 | S1-M1-3.2 | |
|---|---|---|---|---|---|---|
| | HL60 | (MRP1) | | IC50, nM | (MXR) | |
| Cell line | IC50, nM (N) | IC50, nM (N) | RR | (N) | IC50, nM (N) | RR |
| Example 129 | 0.212 ± 0.048 | 0.245 ± 0.45 (5) | 1.2 | 3.7 ± 2.0 (6) | 4.8 ± 2.1 (6) | 1.3 |
| | (5) | | | | | |
| Paclitaxel | 1.8 | 5.2 | 2.9 | 92 ± 100 (6) | 108 ± 88 (6) | 1.1 |
| Vinblastine | 0.5 | 2.0 | 3.7 | 6.5 | 12.7 | 1.9 |
| Adriamycin | 24.2 ± 6.8 (5) | >3000 (5) | >124 | 221 ± 113 | 11480 ± 367 | 52 |
| | | | | (2) | (2) | |
| Mitoxantrone | 5.9 | 190.5 | 32.4 | 31 | 16590 | 535 |
| ^{a}Data are mean IC₅₀ (nM) ± standard deviation for the indicated agents from multiple experiments (N = number of determinations, in parentheses) or single experiments. RR, relative resistance = ratio of IC₅₀ of the resistant cell model to IC₅₀ of the corresponding sensitive cell line. Lower relative resistance values indicate greater sensitivity of cells to the drug, values close to 1 indicate no resistance, | | | | | | |
| # while values greater than 3 indicate resistance. Drug transporters are MRP1 (ABCC1, multidrug resistance-associated protein) and MXR (ABCG2, BCRP, ABC half-transporter). | | | | | | |

3.1.2.1. Paclitaxel Resistance Mediated by Mechanisms Other Than MDR₁

In addition to over-expression of drug efflux pumps, other mechanisms of resistance to paclitaxel likely operate in the clinic. In order to identify alternative modes of resistance to paclitaxel that are not mediated by MDR_{1,} tumor cells grown in vitro are made resistant to paclitaxel in the presence of an MDR-specific inhibitor. This method of using a drug efflux pump inhibitor has been used successfully to prevent the expression of MDR₁ protein, allowing the acquisition of other mechanisms of resistance to paclitaxel (Giannakakou, P., Sackett, D. L., Kang, Y.-K., Zhan, A., Buters, J. T. M., Fojo, T., Poruchynsky, M. S. Paclitaxel-resistant human ovarian cancer cells have mutant beta-tubulins that exhibit impaired paclitaxel-driven polymerization. J. Biol. Chem., 272:17118-17125,1997).

KB-3-1 epidermoid tumor cells are selected by chronic exposure to low concentrations of paclitaxel (stepwise, up to 15 nM) in the presence of 5.0 uM of the MDR₁-specific inhibitor 7-(6,7-Dimethoxy-3,4-dihydro-1 H-isoquinolin-2-yl)-2-(3,4-dimethoxy-phenyl)-2-p-tolylsulfanyl-heptanenitrile. After approximately 8 months of exposure to both agents, a population of KB-3-1 cells (designated KB-PTX-15+099) remained viable and ultimately became 21-fold resistant to paclitaxel. These cells are 7.6-fold cross-resistant to the structurally-related taxane, docetaxel. In contrast, these cells have markedly increased sensitivity to Example 129 (3.4-fold), as well as to other tubulin depolymerizing agents such as vinblastine (3.7-fold) and dolastatin-10 (3.4-fold). (Table 6). Resistance to paclitaxel in these cells is likely mediated by mechanisms other than over-expression of MDR₁ since no MDR₁ RNA is detected by polymerase chain reaction or immunoblot analyses (not shown). In addition, no RNA for the drug pump MXR are detected in these cells nor is there altered accumulation of paclitaxel as assessed by radiolabeled drug accumulation studies (not shown). These data suggest that any drug transporters that might efflux paclitaxel are not expressed in these cells. The cDNA of the predominate isotypes of α- and β-tubulin (K-α-1 and M40, respectively) from this cell line were sequenced. A single mutation in the P-tubulin cDNA was found that converts Asp26 to Glu. Therefore, resistance in this line may be associated with a tubulin mutation, however other mechanisms may operate.

In vivo studies demonstrate that KB-PTX-15+099 tumor xenografts retain resistance to paclitaxel and sensitivity to Example 129. KB-PTX-15+099 cells grown in athymic mice are not inhibited by paclitaxel (60 mg/kg IV) but are highly responsive to Example 129 (1.25 mg/kg IV) (Table 7). Therefore, Example 129 overcomes in vitro and in vivo resistance to paclitaxel by mechanisms other than over-expression of drug efflux pumps.

TABLE 6
Example 129 Overcomes Cellular Resistance to Paclitaxel Mediated by
Non-MDR mechanisms.

| | KB-3-1 | KB-PTX-15+099 | Average Relative |
|---|---|---|---|
| Compound | IC50 (nM) | IC50 (nM) | Resistance (RR) |
| Example 129 | 0.612 ± 0.023 | 0.182 ± 0.028 | (0.30) |
| Vinblastine | 0.703 ± 0.097 | 0.192 ± 0.022 | (0.27) |
| Dolastatin 10 | 0.041 ± 0.010 | 0.012 ± 0.004 | (0.30) |
| Paclitaxel | 2.74 ± 0.39 | 57.8 ± 9.6 | (21.1) |
| Docetaxel | 0.594 ± 0.098 | 4.49 ± 1.91 | (7.6) |

Average relative resistance is defined as the ratio of the IC₅₀ of the resistant cell model to that of the respective sensitive cell counterpart. A ratio of approximately 1 indicates no resistance; a ratio less than 1 indicates increased sensitivity.

TABLE 7
Example 129 Inhibits Growth of Tumors, with Resistance Mediated by Non-
MDR mechanisms, in Animal Models.

| | | Dose | Schedule | % T/C on day | | | | |
|---|---|---|---|---|---|---|---|---|
| Tumor | Drug | (mg/kg) | (day) | 6 | 9 | 13 | 16 | 20 |
| KB-PTX- | Example | 1.25 | 1,8,15 | 32** | 19** | 9** | 10** | 7** |
| 15 + 099 | 129 | | | | | | | |
| KB-PTX- | paclitaxel | 60 | 1,8,15 | 103 | 93 | 89 | 87 | 90 |
| 15 + 099 | | | | | | | | |

Groups of 10 female nu/nu mice are injected with 5 × 10⁶ KB-PTX-15 + 099 cells. Animals bearing staged tumors are treated IV with vehicle, Example 129, or paclitaxel at the doses indicated. Relative tumor growth is determined during the experiment and % T/C calculated. Statistical analyses are Student's t-test of treated time points vs. vehicle (*, P < 0.05; **, P < 0.01)

In further support of these observations, Example 129 also overcomes resistance to another tubulin polymerizing drug, epothilone B, which induces tubulin mutations in an in vitro model. A lung carcinoma cell line, A549, was chronically exposed to epothilone B to produce a sub-line that is highly resistant to the selecting agent (He L., Yang C.-P. H., Horwitz S. B. Mutations in β-tubulin map to domains involved in regulation of microtubule stability in epothilone-resistant cell lines. Molecular Cancer Therapeutics. 1: 3-10, 2001). This selection was done in the absence of an MDR₁-specific inhibitor since epothilones do not readily induce MDR₁ expression. The resulting cell line, A549.EpoB40, is 107-fold resistant to epothilone B and expresses β-tubulin with a point mutation at position 292 that converts glutamine to glutamate. This line also has low-level cross-resistance to paclitaxel (13-fold). However, this cell line is sensitive to depolymerizing agents including HTI-286 (0.6-fold) and vinblastine (0.2-fold; ). Therefore, HTI-286 effectively inhibits the growth of tumor cell lines with resistance to epothilone B associated with tubulin mutations.

3.1.3. Development and Characterization of Cell Lines Resistant to Example 129

Cells are selected for resistance to Example 129 to understand the frequency and mechanistic basis of resistance. Resistance to Example 129 has been established in epithelial carcinoma cell lines. The epidermoid (KB-3-1)-derived cell lines develop approximately 12-fold resistance to Example 129 (and highly related analogs), 9-fold resistance to vinblastine, and 3-fold resistance to colchicine (Table 8). Cross-resistance to the peptide-like antimicrotubule drugs is observed in KB-3-1-selected cells (28-fold for dolastatin). Resistance to paclitaxel is not detected in the KB-3-1 line resistant to Example 129. MDR₁ over-expression is unlikely since paclitaxel resistance is not detected. Neither MDR₁ nor MXR over-expression has been found using highly sensitive PCR methodology.

TABLE 8
Cross-Resistance Profile of KB-3-1 Cell Line
Made Resistant to Example 129¹

| | Cell Line IC50 | | |
|---|---|---|---|
| | KB-3-1 | KB-HTI-4.0 | |
| Compound | IC50, nM | IC50, nM (RR) | |
| Example 129 | 0.53 ± 0.05 | 6.50 ± 1.60 | (12) |
| Hemiasterlin A | 0.33 ± 0.05 | 4.38 ± 3.37 | (13) |
| Vinca-domain | | | |
| Vinblastine | 0.73 ± 0.03 | 6.19 ± 1.26 | (9) |
| Vinorelbine | 1.22 ± 0.33 | 8.64 ± 2.94 | (7) |
| Rhizoxin | 9.13 ± 2.17 | 98.37 ± 37.60 | (11) |
| Maytansine | 0.06 ± 0.00 | 0.84 ± 0.19 | (14) |
| Vinca-peptide domain | | | |
| Dolastatin −10 | 0.03 ± 0.02 | 0.83 ± 0.33 | (28) |
| Dolastatin −15 | 0.04 ± 0.05 | 0.49 ± 0.14 | (12) |
| Phomopsin A | 671 ± 55 | 3529 ± 2988 | (5) |
| Cryptophycin-1 | 0.0064 ± 0.0045 | | |
| Colchicine | | | |
| Colchicine | 6.64 ± 1.02 | 22.35 ± 0.07 | (3) |
| Polymerizing Agents | | | |
| Paclitaxel | 2.66 ± 0.92 | 3.18 ± 0.62 | (1) |
| Docetaxel | 0.44 ± 0.20 | 1.25 ± 0.76 | (3) |
| Epothilone B | 0.63 ± 0.08 | 2.37 ± 2.20 | (4) |
| Eleutherobin | 17.41 ± 1.63 | 21.33 ± 4.40 | (1) |
| DNA active drugs | | | |
| Camptothecin | 14.86 ± 6.11 | 335 ± 157 | (23) |
| Topotecan | 18.13 ± 2.98 | 1029 ± 541 | (57) |
| Mitoxantrone | 10.01 ± 6.50 | 430 ± 159 | (43) |
| Bisantrene | 119 ± 47 | 299 ± 91 | (3) |
| Adriamycin | 33.93 ± 21.19 | 552 ± 111 | (16) |

¹Values shown are mean IC₅₀ (nM) ± standard deviation (Value in parantheses indicate relative resistance vs. parental cells).

3.2. In vivo Analysis

3.2.1 Effect of Example 129 on Human Carcinoma Xenografts

Initial studies are performed on tumors known to be sensitive to paclitaxel or vincristine. In these experiments, xenografts are implanted subcutaneously into the flanks of nude mice. When tumors attain a mass of 80-120 mg (ie, staged), animals are treated IV with Example 129 (200-1600 μg/kg) or paclitaxel (60 mg/kg), IP with vincristine (1000 μg/kg), or IV with vehicle control (saline) on days 1, 5 and 9. Both paclitaxel and vincristine are given at approximately 80-90% of the maximum tolerated dose (MTD). (The compounds have a steep dose-response curve and are lethal at >1.5×MTD). Relative tumor mass compared to controls are assessed during the 21-day post-staging period.

Example 129 inhibits tumor growth as well as paclitaxel and vincristine in both the KB-3-1 (Table 9A) and LOX (Table 9B) xenograft models. The minimum and maximum effective dose in the LOX xenograft model is approximately 200 and 1600 μg/kg, respectively.

TABLE 9
Effect of Example 129 in Two Xenograft Models Sensitive to
Paclitaxel and Vincristine
A. The Effect of Example 129 on the Growth of the
Human Epidermoid Carcinoma KB-3-1

| | | Dose | | Schedule | % T/C on day | | |
|---|---|---|---|---|---|---|---|
| Tumor | Drug | (mg/kg) | Route | (day) | 7 | 14 | 21 |
| KB-3-1 | Example | 1.6 | IV | 1,5,9 | 10** | 1** | 23* |
| | 129 | | | | | | |
| KB-3-1 | vincristine | 1 | IP | 1,5,9 | 15** | 12** | 17** |
| KB-3-1 | paclitaxel | 60 | IV | 1,5,9 | 5** | 0** | 0** |

B. The Effect of Example 129 on the Growth of the
Human Melanoma Carcinoma Lox

| | | Dose | | Schedule | % T/C on day | |
|---|---|---|---|---|---|---|
| Tumor | Drug | (mg/kg) | Route | (day) | 7 | 12 |
| Lox | Example | 1.6 | IV | 1,5,9 | 12** | 2** |
| | 129 | | | | | |
| Lox | vincristine | 1 | IP | 1,5,9 | 13** | 3** |
| Lox | paclitaxel | 60 | IV | 1,5,9 | 16** | 4** |

Groups of 5 female nu/nu mice are injected with 1.5×10⁶ KB-3-1 (A) or Lox cells (B). Animals bearing staged tumors are treated IV or IP with vehicle, Example 129, paclitaxel or vincristine at the doses indicated. Relative tumor growth is determined during the experiment and % T/C calculated. Statistical analyses are Student's t-test of treated time points vs. vehicle (*, P<0.05; **, P<0.01)

3.2.2 Effect of Example 129 on Human Carcinoma Xenografts Resistant to Taxanes and Vinca Alkaloids

The activity of Example 129 is studied in tumors derived from cell lines that have either acquired or inherent resistance to paclitaxel or vincristine. KB-85 tumors, which have acquired resistance to paclitaxel or vincristine by MDR₁ induction, are resistant to these drugs but retained sensitivity to Example 129 compared to KB-3-1-derived tumors (Table 10A and B). The results with paclitaxel are particularly remarkable since KB-85 cells are only 14-fold resistant to paclitaxel, in tissue culture and therefore suggests that a small change in resistance to paclitaxel in vitro is associated with complete resistance to paclitaxel in animals. It is likely that this represents the clinical scenario since antimitotics are used at the maximum tolerated dose in patients.

Example 129 also inhibits the growth of tumors that are inherently resistant to taxane or vinblastine. The compound inhibits growth 60-95% in tumors derived from DLD-1 (Table 10B), MX-1W (Table 10C) or HCT-15 (Table 10D), while no response is obtained with paclitaxel or vincristine at or near the MTD. In general, higher doses of Example 129 are needed to achieve efficacy in paclitaxel-resistant tumors, although these doses are still at or below the MTD of Example 129. These data suggest that patients with paclitaxel- and vincristine-resistant tumors may respond to Example 129.

TABLE 10
Effect of Example 129 on Paclitaxel and Vincristine-Resistant
Tumors in Xenograft Models
A: KB-8-5 xenograft model

| | | Dose | | Schedule | % T/C on day | | |
|---|---|---|---|---|---|---|---|
| Tumor | Drug | (mg/kg) | Route | (day) | 6 | 14 | 21 |
| KB-8.5 | Example 129 | 1.6 | IV | 1,5,9 | 25** | 16 | 47** |
| KB-8.5 | vincristine | 1 | IP | 1,5,9 | 71 | 85 | 81 |
| KB-8.5 | paclitaxel | 60 | IV | 1,5,9 | 63** | 86 | 109 |

B: DLD-1 xenograft model

| | | Dose | | Schedule | % T/C on day | | |
|---|---|---|---|---|---|---|---|
| Tumor | Drug | (mg/kg) | Route | (day) | 7 | 14 | 21 |
| DLD-1 | Example 129 | 1.6 | IV | 1,5,9 | 22** | 20** | 44 |
| DLD-1 | vincristine | 1 | IP | 1,5,9 | 113 | 119 | 177 |
| DLD-1 | paclitaxel | 60 | IV | 1,5,9 | 71 | 73 | 101 |

C: MX-1W xenograft model

| | | Dose | | Schedule | % T/C on day | |
|---|---|---|---|---|---|---|
| Tumor | Drug | (mg/kg) | Route | (day) | 7 | 14 |
| MX-1W | Example 129 | 1.6 | IV | 1,5,9 | 14** | 3** |
| MX-1W | vincristine | 1 | IP | 1,5,9 | 65 | 61 |
| MX-1W | paclitaxel | 60 | IV | 1,5,9 | 76 | 77 |

D: HCT-15 xenograft model

| | | Dose | | Schedule | % T/C on day | | | |
|---|---|---|---|---|---|---|---|---|
| Tumor | Drug | (mg/kg) | Route | (day) | 9 | 13 | 17 | 21 |
| HCT-15 | Example 129 | 1.4 | IV | 1,5,9 | 60* | 36* | 39* | 49* |
| HCT-15 | vincristine | 1 | IP | 1,5,9 | 91 | 81 | 95 | 107 |
| HCT-15 | paclitaxel | 60 | IV | 1,5,9 | 114 | 106 | 123 | 110 |

Groups of 5 female nu/nu mice are injected with 7.0×10⁶ KB-8.5 cells, 5×10⁶ HCT-15 cells,, 6×10⁶ DLD-1 cells, or with approximately 5 tumor fragments of MX-1W Animals bearing staged tumors are treated IV or IP with vehicle, Example 129, paclitaxel or vincristine at the doses indicated. Relative tumor growth is determined during the experiment and % T/C calculated. Statistical analyses are Student's t-test of treated time points vs. vehicle(*, P<0.05; **, P<0.01).

3.2.3 In vivo Activity by Oral Administration

MDR₁ is expressed by gastrointestinal epithelial cells and significantly limits the oral absorption of drugs that interact with MDR_{1,} including paclitaxel (Sparreboom A, Van Asperen J, Mayer U, Schinkel A H, Smit J W, Meijer D K F, Borst P, Nooijen W J, Beijnen J H, Tellingen O V. Limited oral bioavailability and active epithelial excretion of paclitaxel (Taxol) caused by P-glyoprotein in the intestine. Proc Natl Acad Sci USA. 94: 2031-2035, 1997). Weak interaction of Example 129 with MDR₁ in cell-based studies suggested that the drug might be efficacious upon oral administration. Consistent with this hypothesis, 2 and 3 mg/kg of Example 129 administered by oral gavage on days 1,5, and 9 significantly (P<0.01) inhibited the growth of Lox melanoma xenografts by 63% and 97%, respectively, compared to vehicle-only controls on day 14. These doses were well-tolerated with weight loss of less than 10%. Orally administered Example 129 also inhibited the growth of the epidermoid KB-3-1 xenograft model and the MDR₁ expressing tumor, MX-1W.

3.3 Evaluation of Synthetic Examples

It is also been discovered that synthetic examples are potent inhibitors of cell growth in paclitaxel-sensitive cells, had weak interaction with MDR₁ similar to Example 129 (since little resistance is observed in cells that overexpress MDR₁), and are active in the Lox melanoma model in vivo (a paclitaxel-sensitive tumor) (Table 9). Example 8, inhibited the growth of the MX-1W (paclitaxel-resistant) tumor. Based on the data described for Example 129 and Example 8, having the described potency in KB-3-1 cells (<100 nM) and low MDR ratios in KB85 vs. KB-3-1 cells, show the compounds of the invention useful for the treatment of tumors that are resistant to taxanes (Table 11).

TABLE 11
MDR1 profile of Examples compared to paclitaxel, docetaxel,
or vinca alkaloids

| | | | | | Activity in nude | |
|---|---|---|---|---|---|---|
| | | | | | mice animal | |
| | | | Relative | | model (% T/C | |
| | | IC₅₀ in | resistance | | @ MTD on | |
| | | parental | in MDR1 | | days 11-15)³ | |
| Compound | EX. | cell line¹ | expressing cell² | | | MX- |
| Name | NO. | KB-3-1 | KB-8-5 | KB-V1 | LOX | 1W |
| | 129 | 1.0 | 2.4 | 81 | 2.5 | 6 |
| Paclitaxel | | 3.9 | 19 | 1406 | 4 | 73 |
| Docetaxel | | 0.6 | 18 | 669 | | |
| Vinblastine | | 0.8 | 37 | 1848 | 9 | 64 |
| | 133 | 34 | 15 | 55 | 1 | |
| | 80 | 2.7 | 3 | 112 | 8⁴ | |
| | 78 | 5.4 | 3 | 95 | 17 | |
| | 106 | 15.6 | 2 | 41 | <47⁴ | |
| | 105 | 5.2 | 2 | 55 | 8 | |
| | 57 | 1.4 | 3 | 102 | <1⁴ | |
| | 8 | 0.3 | 3 | 68 | 2 | 9 |
| | 120 | 3.5 | 2 | 62 | 20 | |
| | 70 | 3.7 | 2 | 41 | 4 | |
| | 13 | 9.1 | 24 | 191 | <11 | |
| | 52 | 1.6 | 2 | 75 | 1 | |
| | 2 | 1.1 | 3 | 80 | 9 | |
| | 14 | 0.4 | 3 | 96 | 2 | |
| | 20 | 2 | 4 | 81 | 3 | |
| | 62 | 0.7 | 3 | 66 | 2 | |
| | 124 | 8.6 | 2 | 153 | 62 | |
| | 65 | 2 | 2 | 39 | 8 | |

¹Amount of drug needed to kill 50% of cells after three days continuous exposure.
²Relative resistance is the IC₅₀ of KB85 or KBV1 cells divided by the IC50 for KB-3-1 cells.
³Done in a staged xenograft tumor model with the specified tumor. % T/C is the size of the tumor in the treated animal divided by that in the control-treated animal multiplied by 100. All values shown for Examples and analogs are statistically significantly different compared to untreated tumors (Student's t-test; P < 0.05) with the exception of Example 124. Paclitaxel and vinblastine effects are no different than untreated MX-1W xenografts.
⁴Where “<” symbol is shown, the maximum tolerated dose (MTD) is not achieved.

TABLE 12

| | In vitro IC50 (nM) | | | | Ratio | Ratio |
|---|---|---|---|---|---|---|
| Ex. No | KB-3-1 | KB85 | KBV1 | N | KB85/KB | KBV1/KB |
| 103 | 170 | 2262 | 2606 | 4 | 13.3 | 15.3 |
| 106 | 16 | 34 | 660 | 2 | 2.2 | 42.3 |
| 96 | 19 | 30 | 1445 | 1 | 1.6 | 76.1 |
| 97 | 15 | 19 | 575 | 1 | 1.3 | 38.3 |
| 105 | 5.25 | 11.05 | 288 | 2 | 2.1 | 54.9 |
| 131 | 6 | 12 | 3000 | 2 | 2.0 | 512.8 |
| 98 | 132.8 | 185.7 | 2942 | 2 | 1.4 | 22.2 |
| 99 | 60 | 87 | 1324 | 3 | 1.4 | 22.0 |
| 100 | 114 | 197 | 3000 | 4 | 1.7 | 26.2 |
| 101 | 43.1 | 138.55 | 3000 | 2 | 3.2 | 69.6 |
| 94 | 109.2 | 196.7 | 3000 | 1 | 1.8 | 27.5 |
| 93 | 85.2 | 339.2 | 3000 | 1 | 4.0 | 35.2 |
| 104 | 27.7 | 50.55 | 2196 | 2 | 1.8 | 79.3 |
| 102 | 371.1 | 617.5 | 3000 | 1 | 1.7 | 8.1 |
| 95 | 38.6 | 82.05 | 2547.5 | 2 | 2.1 | 66.0 |
| 92 | 125.6 | 263.7 | 3000 | 1 | 2.1 | 23.9 |
| 129 | 0.96 | 2.3 | 77.4 | | 2.4 | 80.6 |
| 132 | 0.51 | 6.4 | 890 | 5 | 12.7 | 1756.9 |
| 133 | 34 | 499 | 1870 | 6 | 14.5 | 54.5 |
| 134 | 28 | 39 | 879 | 3 | 1.4 | 31.3 |
| 135 | 909 | 2089 | 2643 | 3 | 2.3 | 2.9 |
| 136 | 478.6 | 1318 | 3000 | 1 | 2.8 | 6.3 |
| 137 | 933.5 | 1578 | 3000 | 2 | 1.7 | 3.2 |
| 138 | 692 | 1513 | 3000 | 1 | 2.2 | 4.3 |
| 77 | 575.4 | 1737 | 3000 | 1 | 3.0 | 5.2 |
| 130 | 60.2 | 87 | 3000 | 1 | 1.4 | 49.8 |
| 79 | 449 | 1417 | 2435 | 3 | 3.2 | 5.4 |
| 80 | 2.7 | 7.1 | 303 | 5 | 2.6 | 112.2 |
| 107 | 64.7 | 162 | 3000 | 2 | 2.5 | 46.4 |
| 108 | 68.3 | 129 | 1590 | 2 | 1.9 | 23.3 |
| 89 | 180.3 | 273.9 | 3000 | 2 | 1.5 | 16.6 |
| 75 | 36 | 78 | 2131 | 4 | 2.2 | 60.0 |
| 139 | 503 | 975 | 3000 | 1 | 1.9 | 6.0 |
| 109 | 129 | 435 | 3000 | 3 | 3.4 | 23.3 |
| 78 | 5.4 | 13.65 | 512 | 2 | 2.5 | 94.9 |
| 120 | 20.7 | 51.6 | 688 | 1 | 2.5 | 33.2 |
| 85 | 115 | 1326 | 3000 | 6 | 11.5 | 26.0 |
| 121 | 136.2 | 185.5 | 3000 | 1 | 1.4 | 22.0 |
| 87 | 27.2 | 56.3 | 1966 | 2 | 2.1 | 72.4 |
| 82 | 58.95 | 100.4 | 2365 | 2 | 1.7 | 40.1 |
| 140 | 113.5 | 181.3 | 3000 | 2 | 1.6 | 26.4 |
| 86 | 310.6 | 669.4 | 3000 | 3 | 2.2 | 9.7 |
| 47 | 7 | 37.5 | 2308 | 1 | 5.4 | 329.7 |
| 76 | 70.8 | 169 | 3000 | 1 | 2.4 | 42.4 |
| 46 | 3.5 | 5.9 | 224 | 1 | 1.7 | 64.0 |
| 56 | 26.3 | 478.6 | 1778 | 1 | 18.2 | 67.6 |
| 57 | 1.4 | 4.1 | 142.4 | 2 | 2.9 | 102.0 |
| 71 | 141.3 | 186.4 | 3000 | 2 | 1.3 | 21.2 |
| 72 | 59.3 | 63.1 | 2665 | 2 | 1.1 | 45.0 |
| 73 | 204.2 | 363 | 3000 | 1 | 1.8 | 14.7 |
| 48 | 2.7 | 6 | 218.7 | 1 | 2.2 | 81.0 |
| 49 | 20 | 55 | 1995 | 1 | 2.8 | 99.8 |
| 54 | 125.8 | 602.5 | 2089 | 1 | 4.8 | 16.6 |
| 55 | 1.7 | 4.4 | 190.5 | 1 | 2.6 | 112.1 |
| 7a | 21.9 | 525.3 | 2410 | 2 | 24.0 | 110.0 |
| 7b | 263 | 1698 | 3000 | 1 | 6.5 | 11.4 |
| 8 | 0.274 | 0.699 | 18.7 | 3 | 2.6 | 68.5 |
| 51 | 223.8 | 363 | 3000 | 1 | 1.6 | 13.4 |
| 50 | 8.7 | 19 | 512.8 | 1 | 2.2 | 58.9 |
| 9 | 7.9 | 19.9 | 446.6 | 1 | 2.5 | 56.5 |
| 81 | 177.8 | 588.8 | 3000 | 1 | 3.3 | 16.9 |
| 58 | 144.5 | 1695 | 3000 | 1 | 11.7 | 20.8 |
| 59 | 2.4 | 13.2 | 1072 | 1 | 5.5 | 446.7 |
| 43 | 1.5 | 4.4 | 104.7 | 1 | 2.9 | 69.8 |
| 25 | 478.6 | 1513 | 2951 | 1 | 3.2 | 6.2 |
| 27 | 331.1 | 1584 | 3000 | 1 | 4.8 | 9.1 |
| 26 | 4.5 | 9.1 | 416.8 | 1 | 2.0 | 92.6 |
| 10a | 33.9 | 524.8 | 1778 | 1 | 15.5 | 52.4 |
| 10b | 446.6 | 1737 | 3000 | 1 | 3.9 | 6.7 |
| 28 | 2.55 | 4.6 | 138.7 | 2 | 1.8 | 54.4 |
| 31 | 436.5 | 1862 | 3000 | 1 | 4.3 | 6.9 |
| 44 | 16.9 | 33.1 | 1023 | 1 | 2.0 | 60.5 |
| 44 | 3.2 | 6.75 | 176.3 | 2 | 2.1 | 55.1 |
| 88 | 870.9 | 1737 | 3000 | 1 | 2.0 | 3.4 |
| 12 | 17 | 27.5 | 1072 | 1 | 1.6 | 63.1 |
| 11 | 1.5 | 2.6 | 55 | 1 | 1.7 | 36.7 |
| 32 | 5.5 | 14.5 | 354.8 | 1 | 2.6 | 64.5 |
| 111 | 178 | 513 | 2238 | 2.9 | 12.6 | |
| 83 | 15.8 | 43.7 | 2089 | 1 | 2.8 | 132.2 |
| 120 | 3.45 | 6.1 | 215.2 | 2 | 1.8 | 62.4 |
| 29 | 35.4 | 380 | 3000 | 1 | 10.7 | 84.7 |
| * | 229 | 602.5 | 1949 | 1 | 2.6 | 8.5 |
| 70 | 3.7 | 5.6 | 151.3 | 1 | 1.5 | 40.9 |
| 84 | 1.6 | 5.1 | 301.9 | 1 | 3.2 | 188.7 |
| 30 | 3 | 5.1 | 107.65 | 2 | 1.7 | 35.9 |
| 90 | 22.4 | 96.9 | 3000 | 2 | 4.3 | 133.9 |
| 122 | 2.1 | 5.8 | 257 | 1 | 2.8 | 122.4 |
| 1a | 562.3 | 1737 | 3000 | 1 | 3.1 | 5.3 |
| 16a | 15.1 | 301.9 | 1819 | 1 | 20.0 | 120.5 |
| 16b | 257 | 1513 | 3000 | 1 | 5.9 | 11.7 |
| 13a | 9.1 | 218.7 | 1738 | 1 | 24.0 | 191.0 |
| 13b | 295.1 | 1413 | 3000 | 1 | 4.8 | 10.2 |
| 52 | 1.55 | 3.7 | 115.65 | 2 | 2.4 | 74.6 |
| 2 | 1.138 | 3.2 | 90.95 | 2 | 2.8 | 80.0 |
| 3 | 15.1 | 20 | 1148 | 1 | 1.3 | 76.0 |
| 14 | 0.408 | 1.131 | 38.95 | 2 | 2.8 | 95.6 |
| 15 | 13.8 | 21.4 | 1202 | 1 | 1.6 | 87.1 |
| 17 | 1 | 1 | 44 | 2 | 2.1 | 75.7 |
| 18 | 7.2 | 17.4 | 575.4 | 1 | 2.4 | 79.9 |
| 20 | 2 | 3.9 | 161.9 | 2 | 2.0 | 80.9 |
| 21 | 10.2 | 18.2 | 758.5 | 1 | 1.8 | 74.4 |
| 141 | 0.695 | 1.65 | 96.9 | 2 | 2.4 | 139.4 |
| 120 | 1.9 | 14.8 | 912 | 1 | 7.8 | 480.0 |
| 60 | 0.997 | 1.8 | 77 | 2 | 1.8 | 77.4 |
| 61 | 12.6 | 18.6 | 1000 | 1 | 1.5 | 79.4 |
| 62 | 0.647 | 1.7 | 42.7 | 2 | 2.6 | 66.0 |
| 63 | 19.1 | 45.7 | 1318 | 1 | 2.4 | 69.0 |
| 117 | 28 | 58 | 2630 | | 2.1 | 93.9 |
| 123 | 891 | 537 | >3 uM | | 0.6 | |
| 23 | 2.1 | 6.6 | 724.4 | 1 | 3.1 | 345.0 |
| 36 | 6 | 13.8 | 501.2 | 1 | 2.3 | 83.5 |
| 91 | 1.7 | 6.8 | 524.8 | 1 | 4.0 | 308.7 |
| 35 | 0.912 | 1.8 | 56.2 | 1 | 2.0 | 61.6 |
| 24 | 9.5 | 22.4 | 1738 | 1 | 2.4 | 182.9 |
| 124 | 8.6 | 18.9 | 1316 | 3 | 2.2 | 153.0 |
| 118 | 190.5 | 478.6 | 3000 | 1 | 2.5 | 15.7 |
| 64 | 6.2 | 16.2 | 457.1 | 1 | 2.6 | 73.7 |
| 65 | 2 | 2.9 | 77.6 | 1 | 1.5 | 38.8 |
| 53 | 44.7 | 61.7 | 3000 | 1 | 1.4 | 67.1 |
| 6 | 8.5 | 17.4 | 1380 | 1 | 2.0 | 162.4 |
| 5 | 0.562 | 1.7 | 52.5 | 1 | 3.0 | 93.4 |
| 68 | 1.5 | 4.3 | 70.8 | 1 | 2.9 | 47.2 |
| 69 | 55 | 151.4 | 2344 | 1 | 2.8 | 42.6 |
| 66 | 17 | 19 | 457 | | 1.1 | 26.9 |
| 67 | 170 | 186 | >3 uM | | 1.1 | |
| 45 | 680 | 1531 | >3 uM | | 2.3 | |
| 119 | 55 | 164 | >3 uM | | 3.0 | |
| 125 | 8.5 | 18 | 479 | | 2.1 | 56.4 |
| 126 | 18 | 23 | 1139 | | 1.3 | 63.3 |
| 13 | 14 | 49 | 1537 | | 3.5 | 109.8 |
| 112 | 163 | 226 | 1768 | | 1.4 | 10.8 |
| 115 | 5 | 231 | >3 uM | | 46.2 | |
| 113 | 18 | 34 | 582 | | 1.9 | 32.3 |
| 116 | 150 | 1634 | >3 uM | | 10.9 | |
| 114 | 55 | 192 | >3 uM | | 3.5 | |
| 37 | 1.8 | 4.1 | 148 | | 2.3 | 82.2 |
| 39 | 1.9 | 5.4 | 149 | | 2.8 | 78.4 |
| 142 | 227 | 513 | >3000 | | 2.2 | 13.2 |
| 143 | 27 | 57 | 1664 | | 2.1 | 61.6 |
| 144 | 3.7 | 55 | 1687 | | 14.9 | 45.6 |
| 145 | 131 | 1602 | >3000 | | 12.2 | 22.9 |
| 146 | 1.3 | 3.1 | 70 | | 2.4 | 53.8 |
| 147 | 11.6 | 20 | 620 | | 1.7 | 53.4 |
| 148 | 88 | 193 | >3000 | | 2.2 | 34.1 |
| 149 | 1.6 | 3.5 | 117 | | 2.2 | 13.1 |
| 150 | 5.2 | 11.3 | 190 | | 2.1 | 36.5 |
| 151 | 1.6 | 2.4 | 114 | | 1.5 | 11.3 |
| 152 | 75 | 158 | >3000 | | 2.1 | 40 |
| 153 | 183 | 616 | >3000 | | 3.4 | 16.4 |
| 154 | 438 | 584 | >3000 | | 1.3 | 6.8 |
| 155 | 1.8 | 5.8 | 370 | | 3.2 | 206 |
| 156 | 58 | 95 | >3000 | | 1.6 | 51.7 |
| 157 | 605 | 1654 | >3000 | | 2.7 | 5.0 |

*Reference Example 100

Based on the results of these standard pharmacological test procedures the compounds of this invention are useful as agents for treating, inhibiting or controlling the growth of tumors which are resistant to chemotherapeutic agents and in particular antimitotic agents which include paclitaxel.

In particular, Example 129, N,β,β-trimethyl-L-phenylalanyl-N¹-[(1S,2E)-3-carboxy-1-isopropylbut-2-enyl]-N¹,3-dimethyl-L-valinamide is:

a) an inhibitor of polymerization of MAP-associated tubulin in a cell-free spectrophotometric assay and depolymerizes cellular microtubules as assessed by immunofluorescence microscopy,

b) an inhibitor of proliferation in 34 tumor cell lines (mean IC₅₀=2.1±1.7 nM; range 0.2-7 nM) wherein cytotoxicity is independent of tumor origin and in all cases is more potent than paclitaxel (2- to 500-fold),

c) retains sensitivity to cell lines inherently-resistant to paclitaxel (HCT15, DLD1, MX1W),

d) induces mitotic arrest (3-10 nM at 24 hr) followed by apoptosis (48 hr),

e) retains potency in tumor cell lines transfected with or selected for over-expression of the drug transporters MDR₁ (P-glycoprotein), MRP1, or MXR,

f) overcomes resistance in epidermoid tumor cell line (KB-PTX-15+099) that is resistant to paclitaxel as a result of expressing β-tubulin with a site-specific amino acid mutation,

g) administered IV in saline in human tumor xenograft models on days 1, 5, 9 post-staging (0.5-1 mg/kg) produces >95% growth inhibition of melanoma Lox and epidermoid KB-3-1 tumors (1-2 gram),

The compounds of Formula (II) may be obtained as inorganic or organic salts using methods known to those skilled in the art (Richard C. Larock, Comprehensive Organic Transformations, VCH publishers, 411-415, 1989). It is well known to one skilled in the art that an appropriate salt form is chosen based on physical and chemical stability, flowability, hydroscopicity and solubility.

Pharmaceutically acceptable salts of the compounds of Formula (II) with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. The compounds can also be used in the form of esters, carbamates and other conventional prodrug forms, which when providing in such form, convert to the active moiety in vivo.

Providing means to make available and is intended to include direct administration as well as in vivo (e.g. pro-drugs.) of compounds used in the method of treating, inhibiting the growth of, or eradicating a tumor in a mammal in need thereof wherein said tumor is resistant to at least one chemotherapeutic agent which method comprises providing to said mammal an effective amount of a compound of Formula (II).

Based on the results of these standard pharmacological test procedures, the compounds of this invention are useful as agents for treating, inhibiting the growth of or eradicating tumors resistant to chemotherapeutic agents and in particular to antimitotic compounds, including paclitaxel. Allometric scaling of data from toxicology studies of Example 129 in mice, rats and dogs is performed to predict a potentially efficacious dose in humans. Based on these calculations, it is estimated that a 70 kg human will receive an exposure, or AUC, to drug that is nearly equivalent to the efficacious dose in mice upon a preferred effective intravenous dose of 0.6 to 1.3 mg of Example 129. Therefore, a more preferred effective regimen for optimum results would be from about 0.8 to 200 micrograms (ug)/kg of body weight per cycle and such dosage units are employed that a total of from about 0.05 mg to about 150 mg of the active compound for a subject of about 70 kg of body weight are administered intravenously in a cycle. A cycle may be once every 1, 2 or 3 weeks as typically used for other anti-microtubule drugs in the clinic.

The dosage regimen for treating mammals may be adjusted to provide the optimum therapeutic response. For example, several divided doses may be provided per cycle or the dose may be proportionally reduced as indicated by the exigencies of the therapeutic situation. A decidedly practical advantage is that these active compounds may be provided in any convenient manner such as by the oral, intravenous, intramuscular or subcutaneous routes. Preferably compounds of the invention are provided by intravenous routes.

The active compounds may be orally provided, for example, with an inert diluent or with an assimilable edible carrier, or they may be enclosed in hard or soft shell gelatin capsules, or they may be compressed into tablets or they may be incorporated directly with the food of the diet. For oral therapeutic administration, these active compounds may be incorporated with excipients and used in the form of ingestible tablets, buccal tablets, troches, capsules, elixirs, suspensions, syrups, wafers and the like. Such compositions and preparations should contain at least 0.1% of active compound. The percentage of the compositions and preparations may, of course, be varied and may conveniently be between about 2% to about 60% of the weight of the unit. The amount of active compound in such therapeutically useful compositions is such that a suitable dosage will be obtained. Preferred compositions or preparations according to the present invention are prepared so that an oral dosage unit form contains between 0.5 and 1500 mg of active compound. The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose, or saccharin may be added or a flavoring agent such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose, as a sweetening agent, methyl and propylparabens as preservatives, a dye and flavoring such as cherry or orange flavor. Of course, any material used in preparing any dosage unit form should be pharmaceutically pure and substantially non-toxic in the amounts used. In addition, these active compounds may be incorporated into sustained-release preparations and formulations.

The active compounds of the invention may also be provided parenterally or intraperitoneally. Solutions or suspensions of these active compounds as a free base or pharmacologically acceptable salt can be prepared in water suitably mixed with a surfactant such as hydroxypropylcellulose. Dispersions can also be prepared in glycerol, liquid polyethylene glycols, and mixtures therof in oils. Under ordinary conditions of storage and use, these preparations contain a preservative to prevent the growth or microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and starage and must be prepared against the contaminating action of microorganisms such as bacteria and fungi. The carrier can be a solvent or dispersion medium containing, for example, water, ethanol, polyol (e.g., glycerol, propylene glycol and liquid poly-ethylene glycol), suitable mixtures thereof, and vegetable oils. Preferably, compounds of this invention are provided by an IV route.

The following examples are representative compounds of this invention which are useful in treating tumors resistant to chemotherapeutic agents and in particular antimitotic agents. The compounds of this invention are prepared by the procedures of WO 99/32509, W096/33211 and U.S. Pat. No. 6,153,590 or according to the procedures of Examples described herein.

General Procedure I: Preparation of Pyruvic Acids (2-Oxopropanoic Acids) From Aldehydes

General Procedure Ia: Preparation of Aza-lactones (2-Methyl-1,3-oxazol-5(4H)-ones)

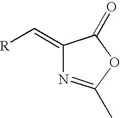

By using a procedure analogous to that described in the literature (Meiwes, J.; Schudok, M.; Kretzschmar, G., Tetrahedron, asymm., 1997, 8, 527-536), an aldehyde (1.0 mol eq), acetylglycine (1.0 mol eq) and sodium acetate (1.0 mol eq) in acetic anhydride (2 mmol) are heated at reflux for 2-5 hours. The mixture is cooled to room temperature, and the resulting precipitate is collected on the Buchner funnel. The solid is washed thoroughly with ice cold water, and dried under high vacuum.

General Procedure Ib: Preparation of 2-hydroxy-acrylic Acid (Pyruvic Acid) from Aza-lactone

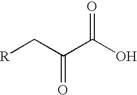

By using a procedure analogous to that described in the literature (Audia, J. E.; Evrard, D. A.; Murdoc, G. R.; Droste, J. J.; Nissen, J. S.; Schenck, K. W.; Fluzinski, R.; Lucaites, V. L.; Nelson, D. L.; Cohen, M. L., J. Med. Chem, 1996, 39(14), 2773-2780), an aza-lactone (0.125 mol, obtained from General Procedure Ia) is suspended in 1.0 N aqueous sodium hydroxide solution (200 mL), and the resulting mixture is heated at 85° C., until a clear reddish solution is obtained. The mixture is cooled and acidified to Congo red acidity with 5N hydrogen chloride. Concentrated hydrogen chloride (30 mL) is added, and water is added to a final volume of 500 mL. The resulting mixture is heated at reflux for 4-5 hours. The mixture is cooled with an ice-water bath, and the resulting precipitate is collected by filtration. The solid is washed with water, and dried under high vacuum, to provide the desired pyruvic acid.

General Procedure Ic: Preparation of Hydantoin

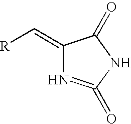

By using a procedure analogous to that described in the literature (Meiwes, J.; Schudok, M.; Kretzschmar, G., Tetrahedron, asymm., 1997, 8, 527-536), to a mixture of hydantoin (0.25 mol) and an aldehyde (0.275 mol) in water (125 mL) is added ammonium hydrogencarbonate portionwise over a period of 10-20 minutes. The resulting mixture is heated at reflux for 4-5 hours under a nitrogen atmosphere. During this process, the solid dissolved and a clear solution is obtained. After cooling to room temperature, a precipitate is collected by filtration. The solid is washed with cold water, cold ethanol and finally ether, and dried under high vacuum.

General Procedure Id: Preparation of Pyruvic Acid from Hydantoin

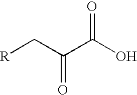

By using a procedure analogous to that described in the literature (Meiwes, J.; Schudok, M.; Kretzschmar, G., Tetrahedron, asymm., 1997, 8, 527-536), a solution of the hydantoin (obtained from General Procedure Ic) in aqueous 5N sodium hydroxide (3 mL/mmol) is heated at reflux for 1-2 hours under a nitrogen atmosphere. The mixture is cooled with an ice water bath, and acidified with concentrated hydrogen chloride to pH 1.0. Some of the product precipitated directly, and the solid is collected by filtration. The remaining product is extracted from the aqueous filtrate with ethyl acetate, and the combined extracts are washed with saturated aqueous sodium chloride, dried over anhydrous magnesium sulfate, and concentrated in vacuo, to give the product.

General Procedure II: Preparation of α,α-Dimethylpyruvic Acid

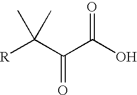

To a solution of a pyruvic acid (obtained from General Procedure Ib) in tetrahydrofuran (ca 100 mL/0.1 mol) is added methyl iodide (2.5-3.0 mol equivalent) and 5N aqueous sodium hydroxide solution (3-3.5 mol equivalent) under a nitrogen atmosphere with cooling with an ice-water bath. The cooling bath is removed, and the resulting mixture is stirred at room temperature for 15-48 hours. The volatiles are removed under reduced pressure (bath temperature is kept below 35° C.), and the remaining aqueous solution is extracted with ethyl acetate to remove non-acidic components. The residue is cooled and acidifed with 10% aqueous hydrogen chloride to pH 1.0, and the resulting aqueous layer is extracted with ethyl acetate (3 times). The combined extracts are washed with saturated aqueous sodium chloride, dried over anhydrous sodium sulfate or magnesium sulfate, filtered and concentrated in vacuo. The product thus obtained is recrystalized, or is used for the next step without further purification.

General Procedure III: Preparation of Racemic A-Segment (N,β,β,-Trimethylalanine Derivative)

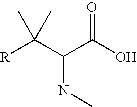

To a solution of α,α-Dimethylpyruvic acid (obtained from General Procedure II) in anhydrous tetrahydrofuran (100 mU0.1 mol, Aldrich) is added methylamine (2.0-2.5 mol eq., 2 M solution in anhydrous tetrahydrofuran, Aldrich) at room temperature under a nitrogen atmosphere. During this procedure, the formation of thick solid may be observed. The resulting mixture is stirred for 1-2 hours, and a solution of borane-pyridine complex (1.2 mol eq., 8 M solution, Aldrich) is introduced. The mixture is then heated at 45-55° C. (bath temperature) for 2.0-2.5 hours, and cooled to room temperature. Methanol (ca 30 mU0.1 mol) is added to the mixture with stirring, and the volatiles are removed in vacuo. The resulting syrupy residue is triturated with tetrahydrofuran. The product precipitated upon cooling with an ice water bath. The solid is collected by filtration, and dried under high vacuum.

General procedure IV: Coupling of the A-segment (N,β,β,-Trimethylalanine Derivative) with the B-C-D Segment (Ethyl (2E,4S)-2,5-dimethyl-4-[methyl(3-methyl-L-valyl)amino]hex-2-enoate): (As Described in Andersen, R, WO 96/33211)

Method IVa: Using Benzotriazole-1-yl-oxy-tris-pyrrolidinophosphonium Hexafluorophosphate as a Coupling Agent

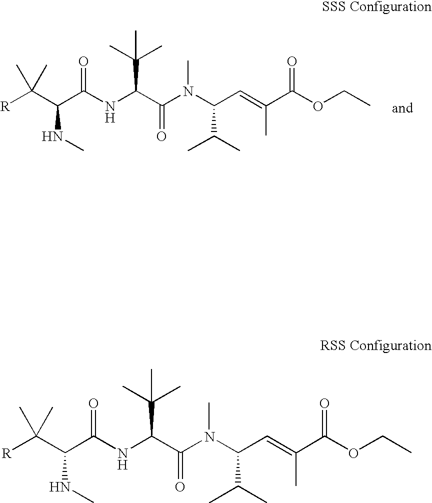

To a cooled (0° C., ice bath) solution of a N,β,β,-trimethylalanine derivative (1.1 mmol, obtained from General Procedure III) and benzotriazole-1-yl-oxy-tris-pyrrolidinophosphonium hexafluorophosphate (1.1 mmol) in anhydrous dimethylformamide (3-5 mL, Aldrich) is added diisopropylethylamine (1.0 mmol) under a nitrogen atmosphere. To the resulting solution is added a solution of ethyl (2E,4S)-2,5-dimethyl4-[methyl(3-methyl-L-valyl)amino]hex-2-enoate hydrochloride (1.0 mmol, Andersen, R, WO 96/33211) in anhydrous dimethylformamide (3 mL). After stirring at 0° C. for 5-10 minutes, the cooling bath is removed, and the resulting reaction mixture is stirred at room temperature for 15-20 hours. The mixture is diluted with water, and the aqueous layer is extracted with ethyl acetate (3 times). The combined extracts are washed with saturated aqueous sodium chloride, dried over anhydrous sodium sulfate, filtered and concentrated in vacuo. The residue is chromatographed (silica gel, flash column), to provide two diastereoisomers (SSS and RSS). The first elute is generally the diasteromer of SSS configuration, and the second is the diasteromer of RSS configuration.

Method IVb: Using Hydroxybenzotriazole and 1-(3-Dimethylaminopropyl)-3-ethylcarbodiimine as Coupling Agents.

To a cooled (0° C., ice bath) solution of a N,β,β,-trimethylalanine derivative (1.1 mmol, obtained from General Procedure III), hydroxybenzotriazole (1.1 mmol) and 1-(3-dimethylaminopropyl)-3-ethylcarbodiimine hydrochloride (1.2 mmol) in anhydrous dimethylformamide (3-5 mL) is added N-methylmorpholine (1.4 mmol) via syringe under a nitrogen atmosphere. After stirring for 15 minutes at 0° C., the cooling bath is removed, and the resulting mixture is stirred for 2-24 hours (the reaction time depends on the solubility of trimethylalanine derivative and the time for activation of the acid). The solution is cooled at 0° C. (ice water bath), and to this mixture is added a solution of ethyl (2E,4S)-2,5-dimethyl-4-[methyl(3-methyl-L-valyl)amino]hex-2-enoate hydrochloride (1.0 mmol, Andersen, R. WO 96/33211) in anhydrous dimethylformamide (3 mL). The cooling bath is removed, and the resulting mixture is stirred for 15-36 hours at room temperature under a nitrogen atmosphere. The mixture is diluted with water, and the aqueous layer is extracted with ethyl acetate (3 times). The combined extracts are washed with saturated aqueous sodium chloride, dried over anhydrous sodium sulfate, filtered and concentrated in vacuo. The residue is chromatographed (silica gel, flash column), to provide two diastereoisomers (SSS and RSS). The first elute is generally the diasteromer of the SSS configuration, and the second is the diasteromer of the RSS configuration.

General Procedure V: Hydrolysis of the Ester

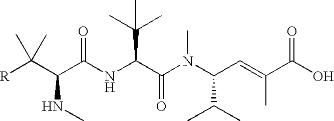

By using a procedure analogous to that described in the literature (Andersen, R. WO 96/33211) The tripeptide ester (obtained from General Procedure IV) is dissolved in methanol (24 mL/mmol), and cooled to 0° C. (ice-water bath). To this solution is added water (8 mL/mmol) and aqueous lithium hydroxide solution (8 mol equivalents). The cooling bath is removed, and the resulting mixture is stirred at room temperature for 15 hours. Methanol is removed in vacuo, and the residual aqueous mixture is cooled with an ice water bath, and acidified to pH 5.5-6.0 with aqueous 1 N citric acid solution. The precipitate is collected by filtration, and the solid is washed with cold water, and dried over high vacuum. Alternatively, the product can be purified by preparative reverse phase HPLC.

General Procedure VI: D-Piece Amide Formation

Method a: To N,β,β-trimethyl-L-phenylalanyl-N¹-[(1S,2E)-3-carboxy-1-isopropylbut-2-enyl]-N¹,3-dimethyl-L-valinamide (0.1 mmol, Andersen, R. WO 99/32509) in acetonitrile (5.0 mL) at 25° C. is added 1-hydroxybenzotriazole hydrate (0.12 mmol) and 1-(3-dimethylaminopropyl)-3-ethylcarbodiimide hydrochloride (1.33 mmol). After 2-18 hours an amine (0.4 mmol) is added. After 1-18 hours the reaction mixture is concentrated in vacuo, dissolved in dimethylformamide, and the product is purified by reverse phase HPLC (0.01% aqueous trifluoroacetic acid/acetonitrile gradient system).

Method b: A mixture of N,β,β-trimethyl-L-phenylalanyl-N¹-[(1S,2E)-3-carboxy-1-isopropylbut-2-enyl]-N¹,3-dimethyl-L-valinamide (1.0 equivalent, Andersen, R. WO 99/32509), hydroxybenzotriazole hydrate (1.2 equivalents), 1-(3-dimethylaminopropyl)-3-ethylcarbodiimine hydrochloride (1.2 equivalents), an amine (1.2 equivalents) and Hunig's base (2.0 equivalents) in anhydrous dimethylformamide (approx 6 mummol) are stirred under a nitrogen atmosphere overnight. The solvent is removed, the residue is taken up in methanol, and the product is purified by reverse phase HPLC (0.01% trifluoroacetic acid in water/acetonitrile).

General Procedure VII: Formation of D-Piece esters.

A mixture of N,β,β-trimethyl-L-phenylalanyl-N¹-[(1S,2E)-3-carboxy-1-isopropylbut-2-enyl]-N¹,3-dimethyl-L-valinamide (1.0 equivalent, Andersen, R. WO 99/32509), 1-(3-dimethylaminopropyl)-3-ethylcarbodiimine hydrochloride (1.2 equivalents), an alcohol (1.2 equivalents) and dimethylaminopyridine (0.2 equivalents) in anhydrous dichloromethane (20 mL/mmol) are stirred under a nitrogen atmosphere at room temperature for 20 hours. The solvent is removed, the residue is taken up in methanol, and the product is purified by reverse phase HPLC (0.01% trifluoroacetic acid in water/acetonitrile).

### REFERENCE EXAMPLE 1

### (4Z)-4-(3-Chlorobenzylidene)-2-methyl-1,3-oxazol-5(4H)-one

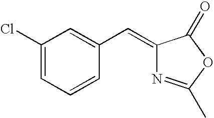

By using a procedure analogous to that described in General Procedure Ia, m-chlorobenzaldehyde (20 g, 142 mmol), acetylglycine (16.66 g, 142 mmol) and sodium acetate (11.67 g, 142 mmol) in acetic anhydride (40 mL) is heated at reflux for 5 hours, to provide (4Z)-4-(3-chlorobenzylidene)-2-methyl-1,3-oxazol-5(4H)-one (22.37 g, 71%) as a yellow solid. MS (ES): m/z 221.94 (M+H). IR cm⁻¹: 1787.38,1760.93, 1659.36. Analysis for C₁₁H₈ClNO₂: Calcd: C, 59.61; H, 3.64; N, 6.32. Found: C, 59.38; H, 3.86; N, 6.30.

### REFERENCE EXAMPLE 2

### 3-(3-Chlorophenyl)-2-oxopropanoic Acid

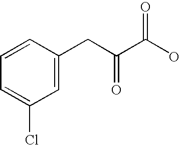

By using a procedure analogous to that described in General Procedure Ib, (4Z)-4-(3-chlorobenzylidene)-2-methyl-1,3-oxazol-5(4H)-one (22 g, 99 mmol, obtained from Reference Example 1) is converted to 3-(3-chlorophenyl)-2-oxopropanoic acid (19.25 g, 98%, a light tan solid). MS (ES): m/z 196.93 (M−H).

### REFERENCE EXAMPLE 3

### 3-(3-Chlorophenyl)-3-methyl-2-oxobutanoic Acid

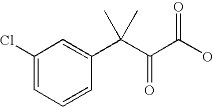

By using a procedure analogous to that described in General Procedure II, 3-(3-methylphenyl)-2-oxopropanoic acid (19.1 g, 96 mmol, obtained from Reference Example 2) is treated with methyl iodide (15.6 mL, 250 mmol) and 5N aqueous sodium hydroxide (69 mL, 346 mmol) in tetrahydrofuran (96 mL), to provide 3-(3-methylphenyl)-3-methyl-2-oxobutanoic acid (9.43 g, 43%) as a brown oil MS (ES): m/z 224.95 (M - H).

### REFERENCE EXAMPLE 4

### 3-Chloro-N,β,β-Trimethylphenylalanine

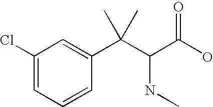

By using a procedure analogous to that described in General Procedure III, 3-(3-chlorophenyl)-3-methyl-2-oxobutanoic acid (9.31 g, 41 mmol, obtained from Reference Example 3) is heated at 50° C. with methylamine (45 mL, 90.4 mmol, 2 M solution in tetrahydrofuran, Aldrich) in anhydrous tetrahydrofuran (91 mL), followed by treatment with borane-pyridine complex (6.2 mL, 49 mmol, Aldrich). The product is purified by triturating with tetrahydrofuran, to give 3-chloro-N,β,β-trimethylphenylalanine (3.24 g, 33%) as a white solid. MS (ES): m/z 242.1 (M+H).

### REFERENCE EXAMPLE 5

### (4Z)-2-Methyl4-(4-chlorobenzylidene)-1,3-oxazol-5(4H)-one

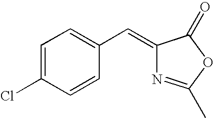

By using a procedure analogous to that described in General Procedure Ia, p-chlorobenzaldehyde (20 g, 142 mmol), acetylglycine (16.66 g, 142 mmol) and sodium acetate (11.67 g, 142 mmol) in acetic anhydride (50 mL) is heated at reflux for 5 hours, to provide (4Z)-4-(4-chlorobenzylidene)-2-methyl-1,3-oxazol-5(4H)-one (23.4 g, 74%) as a yellow solid. MS (ES): m/z 222.01 (M+H). IR cm⁻¹: 3436.01, 1799.29, 1772.53, 1660.38. Analysis for C₁₁H₈ClNO₂: Calcd: C, 59.61; H, 3.64; N, 6.32. Found: C, 59.37; H, 4.02; N, 6.10.

### REFERENCE EXAMPLE 6

### (2Z)-3-(4-Chlorophenyl)-2-hydroxy 2-propenoic Acid

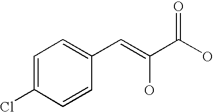

By using a procedure analogous to that described in General Procedure Ib, (4Z)-4-(4-chlorobenzylidene)-2-methyl-1,3-oxazol-5(4H)-one (23.3 g, 105 mmol, obtained from Reference Example 5) is converted to 3-(4-chlorophenyl)-2-oxopropanoic acid (19.65 g, 94%, a orange solid). MS (ES): m/z 196.90 (M−H). IR cm⁻¹: 3466.39, 3087.77, 3036.72, 1666.48, 1438.61, 1226.81. Analysis for C₉H₇ClO₃: Calcd: C, 54.43; H, 3.55; N, 0.00. Found: C, 54.44; H, 3.59; N, 0.00.

### REFERENCE EXAMPLE 7

### 3-(4-Chlorophenyl)-3-methyl-2-oxobutanoic Acid

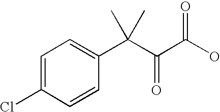

By using a procedure analogous to that described in General Procedure II, 3-(4-chlorophenyl)-2-oxopropanoic acid (19.53 g, 98.3 mmol, obtained from Reference Example 6) is treated with methyl iodide (15.9 mL, 256 mmol) and 5N aqueous sodium hydroxide solution (71 mL, 354 mmol) in tetrahydrofuran (100 mL), to give 3-(4-chlorophenyl)-3-methyl-2-oxobutanoic acid as a brown oil (22.7 g, 100%). MS (ES): m/z 225.16 (M−H). IR cm⁻¹: 3385.43, 2980.19, 1721.75.

### REFERENCE EXAMPLE 8

### 4-Chloro-N,β,β-trimethylphenylalanine

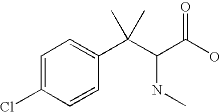

By using a procedure analogous to that described in General Procedure III, 3-(4-chlorophenyl)-3-methyl-2-oxobutanoic acid (22.47 g, 99 mmol, obtained from Reference Example 7) is heated at 50° C. with methylamine (109 mL, 218 mmol, 2 M solution in tetrahydrofuran, Aldrich) in anhydrous tetrahydrofuran (200 mL), followed by treatment with borane-pyridine complex (15 mL, 119 mmol, Aldrich). The product is purified by triturating with tetrahydrofuran, to give 4-chloro-N,β,β-trimethylphenylalanine (10.13 g, 42%) as a white solid. MS (ES): m/z 242.11 (M+H). IR cm⁻¹: 3038.48, 1609.36, 1584.22. Analysis for C₁₂H₁₆ClNO₂: Calcd: C, 59.63; H, 6.67; N, 5.79. Found: C, 59.44; H, 6.67; N, 5.74.

### REFERENCE EXAMPLE 9

### (4Z)-2-Methyl4-(3-methylbenzylidene)-1,3-oxazol-5(4H)-one

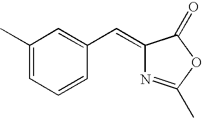

By using a procedure analogous to that described in General Procedure Ia, m-tolualdehyde (20 g, 166 mmol), acetylglycine (19.5 g, 166 mmol) and sodium acetate (13.66 g, 166 mmol) in acetic anhydride (40 mL) is heated at reflux for 4-5 hours, to provide (4Z)-2-methyl-4-(3-methylbenzylidene)-1,3-oxazol-5(4H)-one (19.29 g, 58%) as a yellow solid. MS (ES): m/z 202.1 (M+H). IR cm⁻¹: 3280.39, 1660.81

### REFERENCE EXAMPLE 10

### 3-(3-Methylphenyl)-2-oxopropanoic Acid

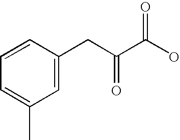

By using a procedure analogous to that described in General Procedure Ib, (4Z)-2-methyl-4-(3-methylbenzylidene)-1,3-oxazol-5(4H)-one (8.5 g, 42.3 mmol, obtained from Reference Example 9) is converted to 3-(3-methylphenyl)-2-oxopropanoic acid (5.56 g, 74%) as a tan solid. MS (ES): m/z 177.1 (M−H). IR cm⁻¹: 3274.92, 1686.84

### REFERENCE EXAMPLE 11

### 3-Methyl-3-(3-methylphenyl)-2-oxobutanoic Acid

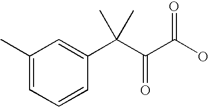

By using a procedure analogous to that described in General Procedure II, 3-(3-methylphenyl)-2-oxopropanoic acid (4.55 g, 25.5 mmol, obtained from Reference Example 10) is treated with methyl iodide (4.13 mL, 66.4 mmol) and 5N aqueous sodium hydroxide solution (18 mL, 92 mmol) in tetrahydrofuran (25 mL), to give 3-methyl-3-(3-methylphenyl)-2-oxobutanoic acid (4.64 g, 88%) as a amber oil. MS (ES): m/z 205.1 (M−H). IR cm⁻¹: 2977.78, 1717.04, 1606.27

### REFERENCE EXAMPLE 12

### N,β,β,3-Tetramethylphenylalanine

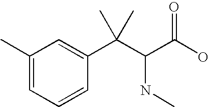

By using a procedure analogous to that described in General Procedure II, 3-methyl-3-(3-methylphenyl)-2-oxobutanoic acid (1 g, 4.85 mmol, obtained from Reference Example 11) is heated at 50° C. with methylamine (5.3 mL, 10.7 mmol, 2 M solution in tetrahydrofuran, Aldrich) in anhydrous tetrahydrofuran (10 mL), followed by treatment with borane-pyridine complex (0.73 mL, 5.8 mmol, Aldrich). The product is purified by triturating with tetrahydrofuran, to give N,β,β,3-tetramethylphenylalanine (0.485 g, 45%) as a white solid. MS (ES): m/z 222.2 (M+H). IR cm⁻¹: 2980.98, 1588.15, 1384.98

### REFERENCE EXAMPLE 13

### (4Z)-2-Methyl-4-(4-methylbenzylidene)-1,3-oxazol-5(4H)-one

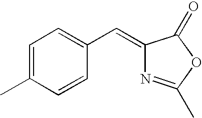

By using a procedure analogous to that described in General Procedure Ia, para-tolualdehyde (20 g, 166 mmol), acetylglycine (19.5 g, 166 mmol) and sodium acetate (13.66 g, 166 mmol) in acetic anhydride (90 mL) are heated at reflux for 4.5 hours, to provide (4Z)-2-methyl-4-(4-methylbenzylidene)-1,3-oxazol-5(4H)-one (14.1 g, 42%) as a dark yellow solid. MS (ES): m/z 202.06 (M+H). IR cm⁻¹: 1777.51, 1657.64. Analysis for C₁₂H₁₁NO₂: Calcd: C, 71.63; H, 5.51; N, 6.96. Found: C, 72.03; H, 5.41; N, 6.62.

### REFERENCE EXAMPLE 14

### (2Z)-2-Hydroxy-3-(4-methylphenyl)-2-propenoic Acid

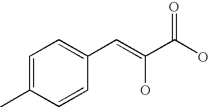

By using a procedure analogous to that described in General Procedure Ib, (4Z)-2-methyl-4-(4-methylbenzylidene)-1,3-oxazol-5(4H)-one (14.06 g, 69.9 mmol, obtained from Reference Example 13) is converted to 3-(4-methylphenyl)-2-oxopropanoic acid (11.1 g, 89%, tan solid). MS (ES): m/z 177.2 (M−H). IR cm⁻¹: 3467.96, 3022.84, 1672.02, 1458.72, 1240.38. Analysis for C₁₀H₁₀O₃: Calcd: C, 67.41; H, 5.66 Found: C, 67.02; H, 5.63

### REFERENCE EXAMPLE 15

### 3-Methyl-3-(4-methylphenyl)-2-oxobutanoic Acid

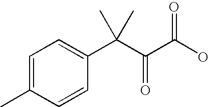

By using a procedure analogous to that described in General Procedure II, 3-(4-methylphenyl)-2-oxopropanoic acid (11 g, 61.7 mmol, obtained from Reference Example 14) is treated with methyl iodide (10 mL, 161 mmol) and 5N aqueous sodium hydroxide solution (45 mL, 222 mmol) in tetrahydrofuran (65 mL), to give 3-methyl-3-(4-methylphenyl)-2-oxobutanoic acid (10.23 g, 80%) as a brown oil. MS (ES): m/z 205.2 (M−H). IR cm⁻¹: 2976.68, 1710.10

### REFERENCE EXAMPLE 16

### N,β,β,4-Tetramethylphenylalanine

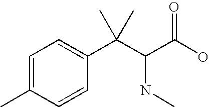

By using a procedure analogous to that described in General Procedure III, 3-methyl-3-(4-methylphenyl)-2-oxobutanoic acid (5 g, 24.2 mmol, obtained from Reference Example 15) is heated at 50° C. with methylamine (27 mL, 53.3 mmol, 2 M solution in tetrahydrofuran, Aldrich) in anhydrous tetrahydrofuran (50 mL), followed by treatment with borane-pyridine complex (10 mL, 80 mmol, Aldrich). The product is purified by triturating with tetrahydrofuran, to give N,β,β,4-tetramethylphenylalanine (0.98 g, 18%) as a pale yellow solid. MS (ES): m/z 222.3 (M+H). IR cm⁻¹: 3434.23, 2970.16, 1609.22, 1593.18. Analysis for C₁₃H₁₉NO₂: Calcd: C, 70.56; H, 8.65; N, 6.33. Found: C, 70.24; H, 8.35; N, 6.16.

### REFERENCE EXAMPLE 17

### (4Z)-4-(3,4-Dimethylbenzylidene)-2-methyl-1,3-oxazol-5(4H)-one

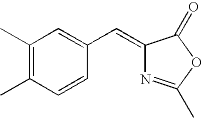

By using a procedure analogous to that described in general Procedure Ia, 3,4-dimethylbenzaldehyde (10 g, 74.5 mmol), acetylglycine (8.73 g, 74.5 mmol) and sodium acetate (6.11 g, 74.5 mmol) in acetic anhydride (50 mL) are heated at reflux for 4-5 hours, to provide (4Z)-4-(3,4-dimethylbenzylidene)-2-methyl-1,3-oxazol-5(4H)-one (7.46 g, 46.6%) as a brown solid. MS (ES): m/z 216 (M+H) IR cm⁻¹: 1806, 1795, 1659, 1599.

### REFERENCE EXAMPLE 18

### (2Z)-3-(3,4-Dimethylphenyl)-2-hydroxy-2-propenoic Acid

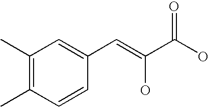

By using a procedure analogous described in General Procedure Ib, (4Z)-4-(3,4-dimethylbenzylidene)-2-methyl-1,3-oxazol-5(4H)-one (3.0 g, 13.94 mmol, obtained from Reference Example 17) is converted to (2Z)-3-(3,4-dimethylphenyl)-2-hydroxy-2-propenoic acid (2.3545 g, 87.9%, dark red solid). MS (ES): m/z 191 (M−H) IR cm⁻¹: 3470, 1673, 1632, 1440.

### REFERENCE EXAMPLE 19

### N-β,β,3,4-Pentamethylphenylalanine

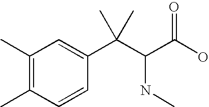

By using procedures analogous to those described in General Procedures II and III, (2Z)-3-(3,4-dimethylphenyl)-2-hydroxy-2-propenoic acid (2.3 g, 11.97 mmol, obtained from Reference Example 18) is treated with methyl iodide (1.94 mL, 31.11 mmol) and 5N aqueous sodium hydroxide solution (8.4 mL, 41.9 mmol) in tetrahydrofuran (30 mL), to provide 3-methyl-3-(3,4-dimethylphenyl)2-oxobuttanoic acid (2.742 g, 100%). This material (2.74 g, 12.44 mmol) is treated with methylamine (27.37 mmol), then with borane-pyridine compex (18.66 mmol), to provide N,β,β,3,4-pentamethylphenylalanine (1.0261 g, 35% overall for 2 steps) as a pale yellow solid. MS (ES): m/z 236 (M+H) IR cm⁻¹: 2974, 1611, 1582. Analysis for C₁₄H₂₁NO₂: Calcd: C, 71.46; H, 8.99; N, 5.95. Found: C, 71.74;H, 8.82; N, 5.75.

### REFERENCE EXAMPLE 20

### (4Z)-4-(3,5-Dimethylbenzylidene)-2-methyl-1,3-oxazol-5(4H)-one

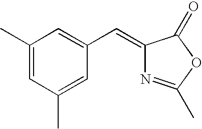

By using a procedure analogous to that described in General Procedure Ia, 3,5-dimethylbenzaldehyde (10 g, 74.5 mmol), acetylglycine (8.73 g, 74.5 mmol) and sodium acetate (6.11 g, 74.5 mmol) in acetic anhydride (50 mL) are heated at reflux for 4-5 hours, to provide (4Z)-4-(3,5-dimethylbenzylidene)-2-methyl-1,3-oxazol-5(4H)-one (7.46 g, 46.6%) as a yellow brown solid. MS (ES): m/z 216.2 (M+H) IR cm⁻¹: 1794, 1759, 1660.

### REFERENCE EXAMPLE 21

### (2Z)-3-(3,5-Dimethylphenyl)-2-hydroxy-2-propenoic Acid

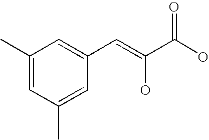

By using a procedure analogous described in General Procedure Ib, (4Z)-4-(3,5-dimethylbenzylidene)-2-methyl-1,3-oxazol-5(4H)-one (3.0 g, 13.94 mmol, obtained from Reference Example 20) is converted to (2Z)-3-(3,5-dimethylphenyl)-2-hydroxy-2-propenoic acid (2.057 g, 76.8, red solid). MS (ES): m/z 191.1 (M−H) IR cm⁻¹: 3439, 2917, 1677. Analysis for C₁₁H₁₂O₃: Calcd: C, 68.74; H, 6.29.. Found: C, 68.77;H, 6.53.

### REFERENCE EXAMPLE 22

### N,β,β,3,5-Pentamethylphenylalanine

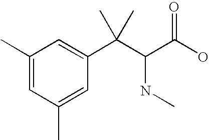

By using procedures analogous to those described in General Procedures II and III, (2Z)-3-(3,5-dimethylphenyl)-2-hydroxy-2-propenoic acid (2.05 g, 10.67 mmol, obtained from Reference Example 21) is treated with methyl iodide (1.73 mL, 27.73 mmol) and aqueous sodium hydroxide solution (7.5 mL, 37.35 mmol) in tetrahydrofuran (30 mL), to provide 3-methyl-3-(3,4-dimethylphenyl)2-oxobutanoic acid (2.277 g, 96.9%). This material (2.270 g, 10.31 mmol) is treated with methylamine (22.67 mmol), then with borane-pyridine compex (15.47 mmol), to provide N,β,β,3,5-pentamethylphenylalanine (763.3 mg, 31.4%) as a pale yellow solid. MS (ES): m/z 236.14 (M+H) IR cm⁻¹: 2974, 1589. Analysis for C₁₄H₂₁NO₂: Calcd: C, 71.46; H, 8.99; N, 5.95. Found: C, 71.69;H, 8.41; N, 5.87.

### REFERENCE EXAMPLE 23

### (4Z)-2-Methyl-4-(2-thienylmethylene)-1,3-oxazol-5(4H)-one

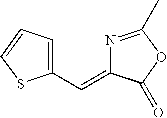

By using a procedure analogous to that described in General Procedure Ia, thiophene-2-aldehyde (10 g, 89.2 mmol), acetylglycine (10.5 g, 89.2 mmol) and sodium acetate (7.32 g, 89.2 mmol) in acetic anhydride are heated at reflux for 2-3 hours, to provide (4Z)-2-methyl-4-(2-thienylmethylene)-1,3-oxazol-5(4H)-one (5.53 g, 32.2%) as a yellowish tan solid. MS (ES):m/z 194 (M+H) IR cm⁻¹: 1796, 1772, 1655 ¹H NMR (δ, DMSO-d₆): 2.323 (s, 3H), 7.21-7.25 (2H, dd), 7.5868 (1H, s), 7.776 (1H, d, J=2.02 Hz), 7.991 (1H, d, J=6.35Hz)

### REFERENCE EXAMPLE 24

### 2-Oxo-3-(2-thienyl)propanoic Acid

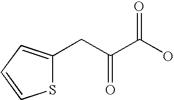

By using a procedure analogous to that described in General Procedure Ib, of (4Z)-2-methyl-4-(2-thienylmethylene)-1,3-oxazol-5(4H)-one (2.42 g, 12.5 mmol, obtained from Reference Example 23) is converted to 2-oxo-3-(2-thienyl)propanoic acid (1 g, 46.9%, reddish solid). MS (ES): m/z 168.9 (M−H) IR cm⁻¹: 3410, 1653, 1453, 1418. Analysis for C₇H₇NO2S: Calcd: C, 49.40; H, 3.55. Found: C, 49.98;H, 3.83.

### REFERENCE EXAMPLE 25

### N-Methyl-3-thien-2-ylvaline

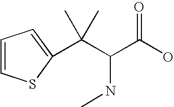

By using procedures analogous to those described in General Procedures II and III, 2-oxo-3-(2-thienyl)propanoic acid (3g, 17.63 mmol, obtained from Reference Example 24) is treated with methyl iodide (3.3 mL, 52.9 mmol) and 5N aqueous sodium hydroxide solution (12 mL, 61.7 mmol) in tetrahydrofuran (40 mL), to give 3-methyl-3-(2-thienyl)-2-oxobutanoic acid as a yellow oil. This material (4 g, 20.18 mmol) is heated at 50° C. with methylamine (22.2 mL, 44.4 mL, 2 M solution in tetrahydrofuran, Aldrich) in anhydrous tetrahydrofuran (50 mL), followed by treatment with borane-pyridine complex (3.1 mL, 30.27 mmol, Aldrich). The product is purified by triturating in THF, to give N-methyl-3-thien-2-ylvaline as a brownish yellow solid (2.303 g, 61.3% from 2-oxo-3-(2-thienyl)propanoic acid). MS (ES):m/z 214.1 (M+H) IR cm⁻¹: 3414, 2970, 1722, 1654, 1544.

### REFERENCE EXAMPLE 26

### (4Z)-2-Methyl-4-(3-thienylmethylene)-1,3-oxazol-5(4H)-one

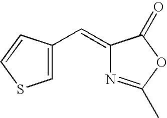

By using a procedure analogous to that described in General Procedure Ia, thiophene-3-aldehyde (10 g, 89.2 mmol), acetylglycine (10.5 g, 89.2 mmol) and sodium acetate (7.32 g, 89.2 mmol) in acetic anhydride (40 mL) are heated at reflux to give (4Z)-2-methyl-4-(3-thienylmethylene)-1,3-oxazol-5(4H)-one (8.78 g, 50.9%) as a brown solid. MS (ES):m/z 194.1 (M+H) IR cm⁻¹: 3099, 1770, 1656. Analysis for C₉H₇NO₂S Calcd: C, 55.94; H, 3.65; N, 7.25. Found: C, 55.46;H, 3.72; N, 6.85.

### REFERENCE EXAMPLE 27

### 2-Oxo-3-(3-thienyl)propanoic Acid

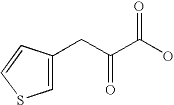

By using a procedure analogous to that described in General Procedure Ib, (4Z)-2-methyl-4-(3-thienylmethylene)-1,3-oxazol-5(4H)-one (7.9 g, 40.9 mmol, obtained from Reference Example 26) is converted to 2-oxo-3-(3-thienyl)propanoic acid (5.905 g, 84.8%, brown solid). MS (ES): m/z 169.1 (M−H) IR cm⁻¹: 3472, 3125, 1680.

### REFERENCE EXAMPLE 28

### N-Methyl-3-(3-thienyl)valine

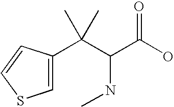

By using analogous procedures to those described in General Procedure II and III, 2-oxo-3-(3-thienyl)propanoic acid (3.0 g, 17.63 mmol, obtained from Reference Example 27) is treated with methyl iodide (3.3 mL, 52.88 mmol) and 5N sodium hydroxide aqueous solution (12.3 mL, 61.71 mmol) in tetrahydrofuran (30 mL), to provide 3-methyl-3-(3-thienyl)-2-oxobutanoic acid (2.069 g, 59.2%, a dark red oil). This material (2.069 g, 10.44 mmol) is treated with methylamine (11.5 mL, 22.96 mmol, 2 M solution in tetrahydrofuran, Aldrich) in anhydrous tetrahydrofuran (20 mL), followed by borane-pyridine complex (1.3 mL, 12.53 mmol). The product is precipitated from tetrahydrofuran, to provide N-methyl-3-(3-thienyl)valine as a pale brown solid (866 mg, 23.0% overall from 2-oxo-3-(3-thienyl)propanoic acid). MS (ES): m/z 212.1 (M−H) IR cm⁻¹: 3066, 1612, 1581. Analysis for C₁₀H₁₅NO₂S: Calcd: C, 56.31; H, 7.09; N, 6.57. Found: C, 55.60;H, 7.70; N, 6.39.

### REFERENCE EXAMPLE 29

### (4Z)-4-(1-Benzothien-3-ylmethylene)-2-methyl-1,3-oxazol-5(4H)-one

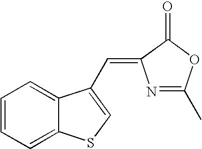

By using a procedure analogous to that described in General Procedure Ia, thianaphthene-3-carboxaldehyde (20 g, 123 mmol, Aldrich), acetylglycine (14.4 g, 123 mmol) and sodium acetate (10.1 g, 123 mmol) in acetic anhydride (80 mL) are heated at reflux for 4 hours, to provide 11.68 g (39%) of (4Z)-4-(1-benzothien-3-ylmethylene)-2-methyl-1,3-oxazol-5(4H)-one as a tan solid. MS (ES): m/z 244.2 (M+H). IR cm⁻¹: 3431.85, 1768.44, 1655.06.

### REFERENCE EXAMPLE 30

### 3-(1-Benzothien-3-yl)-2-oxopropanoic Acid

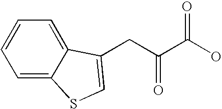

By using a procedure analogous to that described in General Procedure Ib, (4Z)-4-(1-benzothien-3-ylmethylene)-2-methyl-1,3-oxazol-5(4H)-one (16 g, 65.8 mmol, obtained from Reference Example 29) is converted to 3-(1-benzothien-3-yl)-2-oxopropanoic acid (13.54 g, 93%, reddish brown solid). MS (ES): m/z 219.0 (M−H). IR cm⁻¹: 3449.19, 3061.64, 1674.63, 1450.61, 1266.09.

### REFERENCE EXAMPLE 31

### 3-(1-Benzothien-3-yl)-3-methyl-2-oxobutanoic Acid

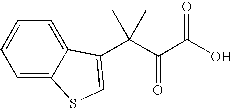

By using a procedure analogous to that described in General Procedure II, 3-(1-benzothien-3-yl)-2-oxopropanoic acid (13.54 g, 61.5 mmol, obtained from Reference Example 30) is treated with methyl iodide (9.96 mL, 160 mmol) and 5N aqueous sodium hydroxide solution (44 mL, 222 mmol) in tetrahydrofuran (61 mL), to give 3-(1-benzothien-3-yl)-3-methyl-2-oxobutanoic acid (5.77 g, 38%) as a light orange oil. MS (ES): m/z 247.2 (M−H).

### REFERENCE EXAMPLE 32

### 3-(1-Benzothien-3-yl)-3-methyl-2-oxobutanoic Acid

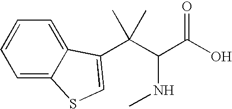

By using a procedure analogous to that described in General Procedure III, 3-(1-benzothien-3-yl)-3-methyl-2-oxobutanoic acid (0.67 g, 2.7 mmol, obtained from Reference Example 31) is heated at 50° C. with methylamine (3 mL, 5.9 mmol, 2 M solution in tetrahydrofuran, Aldrich) in anhydrous tetrahydrofuran (7 mL), followed by treatment with borane-pyridine complex (0.405 mL, 3.2 mmol, Aldrich). The product is purified by triturating in Et₂O, to provide 3-(1-benzothien-3-yl)-3-methyl-2-oxobutanoic acid as a pale yellow solid (0.628 g, 88%). MS (ES): m/z 264.2 (M+H).

### REFERENCE EXAMPLE 33

### (4Z)-4-(1-Benzothien-2-ylmethylene)-2-methyl-1,3-oxazol-5(4H)-one

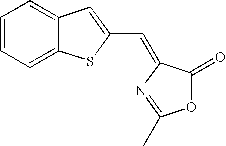

By using a procedure analogous to that described in General Procedure Ia, thianaphthene-2-carboxaldehyde (6.65 g, 41 mmol, prepared by using the literature procedure. Ref. Hsiao, C.; Bhagavatula, L.; Pariza, R. J. Syn Commun., 1990, 20, 1687-1695.), acetylglycine (4.8 g, 41 mmol) and sodium acetate (3.36 g, 41 mmol) in acetic anhydride (20 mL) are heated at reflux for 5 hours, to provide (4Z)-4-(1-benzothien-2-ylmethylene)-2-methyl-1,3-oxazol-5(4H)-one (8.14 g, 82%) as a reddish-orange solid. MS (ES): m/z 244.07 (M+H). IR cm⁻¹: 3433.67, 1762.48, 1655.86. Analysis for C₁₃H₉NO₂S: Calcd: C, 64.18; H, 3.73; N, 5.76. Found: C, 64.12; H, 3.73; N, 5.64.

### REFERENCE EXAMPLE 34

### (2Z)-3-(1-Benzothien-2-yl)-2-hydroxy-2-propenoic Acid

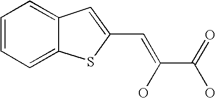

By using a procedure analogous to that described in General Procedure Ib, (4Z)4-(1-benzothien-2-ylmethylene)-2-methyl-1,3-oxazol-5(4H)-one (8 g, 32.9 mmol, obtained from Reference Example 33) is converted to 3-(1-benzothien-2-yl)-2-oxopropanoic acid (6.87 g, 95%, tan solid). MS (ES): m/z 218.93 (M−H). IR cm⁻¹: 3506.96, 3021.21, 1666.58, 1448.77, 1223.34. Analysis for C₁₁H₈O₃S: Calcd: C, 59.99; H, 3.66. Found: C, 60.22; H, 3.49.

### REFERENCE EXAMPLE 35

### 3-(1-Benzothien-2-yl)-3-methyl-2-oxobutanoic Acid

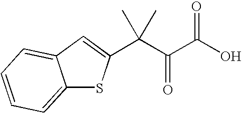

By using a procedure analogous to that described in General Procedure II, 3-(1-benzothien-2-yl)-2-oxopropanoic acid (6.77 g, 30.77 mmol, obtained from Reference Example 34) is treated with methyl iodide (5.23 mL, 84 mmol) and 5N aqueous sodium hydroxide solution (22 mL, 109 mmol) in tetrahydrofuran (30 mL), to give 3-(1-benzothien-2-yl)-3-methyl-2-oxobutanoic acid as a brown oil (4.54 g, 59%). MS (ES): m/z 247.2 (M−H).

### REFERENCE EXAMPLE 36

### 3-(1-Benzothien-2-yl)-3-methyl-2-oxobutanoic Acid

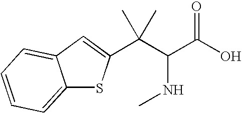

By using a procedure analogous to that described in General Procedure III, 3-(1-benzothien-2-yl)-3-methyl-2-oxobutanoic acid (1 g, 4.03 mmol, obtained from Reference Example 35) is heated at 50° C. with methylamine (4.43 mL, 8.86 mmol, 2 M solution in tetrahydrofuran, Aldrich) in anhydrous tetrahydrofuran (10 mL), followed by treatment with borane-pyridine complex (0.604 mL, 4.8 mmol, Aldrich). The product is purified by triturating in acetonitrile/diethyl ether, to give 3-(1-benzothien-2-yl)-3-methyl-2-oxobutanoic acid as a pale yellow solid (0.503 g, 47% ). MS (ES): m/z 264.4 (M+H).

### REFERENCE EXAMPLE 37

### (4Z) 4-(4-tert-Butylbenzylidene)-2-methyl-1,3-oxazol-5(4H)-one

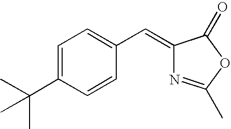

By using a procedure analogous to that described in General Procedure Ia, para-tert-butylbenzaldehyde (20 g, 123 mmol, Aldrich), acetylglycine (14.4 g, 123 mmol) and sodium acetate (10.11 g, 123 mmol) in acetic anhydride (20 mL) is heated at reflux for 5 hours, to provide 14.78 g (49%) of (4Z) 4-(4-tert-butylbenzylidene)-2-methyl-1,3-oxazol-5(4H)-one as a brown oil. ¹H NMR (δ, DMSO-d₆): 1.3394 (9H, s), 2.3578 (3H, s), 7.1468 (1H, s), 7.4747 (2H, d, J=6.75 Hz), 8.0116 (2H, d),

### REFERENCE EXAMPLE 38

### (2Z)-3-(4-tert-Butylphenyl)-2-hydroxy-2-propenoic Acid

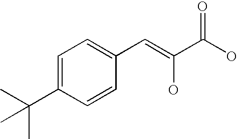

By using a procedure analogous to that described in General Procedure Ib, (4Z) 4-(4-tert-butylbenzylidene)-2-methyl-1,3-oxazol-5(4H)-one (14.78 g, 61 mmol, obtained from Reference Example 37) is converted to (2Z)-3-(4-tert-butylphenyl)-2-hydroxy-2-propenoic acid (13.47 g, 100%, brown solid). MS (ES): m/z 219.07 (M−H).

### REFERENCE EXAMPLE 39

### 3-(4-tert-Butylphenyl)-3-methyl-2-oxobutanoic Acid

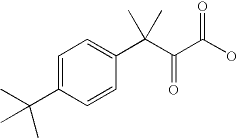

By using a procedure analogous to that described in General Procedure II, (2Z)-3-(4-tert-butylphenyl)-2-hydroxy-2-propenoic acid (3.8 g, 17.2 mmol, obtained from Reference Example 38) is treated with methyl iodide (2.8 mL, 44.8 mmol) and 5N aqueous sodium hydroxide solution (12.4 mL, 62 mmol) in tetrahydrofuran (17 mL), to give 3-(4-tert-butylphenyl)-3-methyl-2-oxobutanoic acid as a brown oil (794 mg, 19%). MS (ES): m/z 247.09 (M−H).

### REFERENCE EXAMPLE 40

### 4-tert-Butyl-N,β,β-trimethylphenylalanine

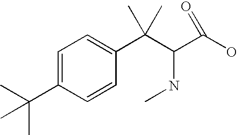

By using a procedure analogous to that described in General Procedure III, 3-(4-tert-butylphenyl)-3-methyl-2-oxobutanoic acid (1.4 g, 5.6 mmol, obtained from Reference Example 39) is heated at 50° C. with methylamine (6.2 mL, 12.4 mmol, 2 M solution in tetrahydrofuran, Aldrich) in anhydrous tetrahydrofuran (30 mL), followed by treatment with borane-pyridine complex (0.847 mL, 6.77 mmol, Aldrich). The product is purified by triturating in tetrahydrofuran, to give 4-tert-butyl-N,β,β-trimethylphenylalanine as a white solid (0.118 g, 8%). MS (ES): m/z 264.2 (M+H) IR cm⁻¹: 3433.84, 2964.07, 1630.21, 1388.22.

### REFERENCE EXAMPLE 41

### β,β-Dimethyl-N-ethyl-phenylalanine

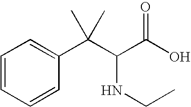

By using procedures analogous to those described in General Procedures Ia, Ib, II and III, treatment of 3-phenyl-2-oxopropionic acid (4.99 g, 30.4 mmol, Aldrich) with methyl iodide (4.92 mL, 79 mmol) and 5N aqueous sodium hydroxide (22 mL, 0.109 mol) in tetrahydrofuran (50 mL) provided , 3-methyl-3-phenyl-2-oxobutanoic acid (2.6 g, 45%) as a yellow oil.

By using a procedure analogous to that described in General Procedure III, 3-methyl-3-phenyl-2-oxobutanoic acid (2.0 g, 10.41 mmol) is heated at 50° C. with ethylamine (12 mL, 22.9 mmol, 2 M solution in tetrahydrofuran, Aldrich) in anhydrous tetrahydrofuran (50 mL), followed by treatment with borane-pyridine complex (1.6 mL, 15.6 mmol, Aldrich). The product is purified by triturating in tetrahydrofuran/ethyl ether at 0° C., to give β,β-dimethyl-N-ethyl-phenylalanine (1.48 g, 64.3%) as a white solid. MS (ES): m/z 222.2 (M+H).

### REFERENCE EXAMPLE 42

### 2,2-Dimethyl-3-o-tolyl-oxirane

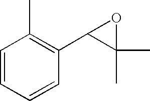

Isopropyldiphenylsulfonium tetrafluorobarate is prepared according to the literature procedure (Corey, E. J.; Jautelat, M.; Oppolzer, W., Tetrahedron Letts., 1967, 2325-2328; Matsuyama, H.; Nakamura, T.; Iyoda, M., J. Org. Chem, 2000, 65, 4796-4803.). A solution of isopropyldiphenylsulfonium tetrafluorobarate (8 g, 25.3 mmol) in anhydrous methylene chloride (1.78 mL) and ethylene glycol dimethyl ether (127 mL) is cooled to −78° C. under nitrogen atmosphere. To this solution is added a solution of lithium diisopropylamide (13.9 mL, 27.8 mmol, 2.0 M heptane/tetrahydrofuran/ethylbenzene, Aldrich) via syringe over a period of 10 minutes. The resulting cloudy yellow solution is stirred for 1 hour, and o-tolualdehyde (2.93 mL, 25.3 mmol) is introduced dropwise. The resulting solution is warmed to −40° C. and stirred for 15 hours, and then quenched with water (240 mL) at −40° C. The resulting mixture is extracted with hexanes (3×75 mL). The combined extracts are washed with saturated aqueous sodium bisulfite and water, dried over anhydrous sodium sulfate, filtered and concentrated in vacuo. The product is purified (flash column, silica gel, 93:7 hexanes:diethyl ether) to provide 2,2-dimethyl-3-o-tolyl-oxirane (2.76 g, 68%) as a pale yellow oil. MS (ES): m/z 162.1 (M+H). ¹H NMR (δ, CDCl₃): 1.007 (3H, s), 1.531 (3H, s), 2.308 (3H, s), 7.131-7.514 (4H, m).

### REFERENCE EXAMPLE 43

### 2-Methyl-2-o-tolyl-propionaldehyde

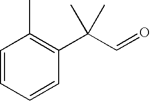

By using a procedure analogous to that described in the literature (Ishihara, K.; Hanaki, N.; Yamamoto, H., Synlett., 1995, 721-722.), to a solution of 2,2-dimethyl-3-o-tolyl-oxirane (2.66 g, 16.4 mmol, obtained from Reference Example 42) in anhydrous benzene (60 mL) at room temperature, under nitrogen atmosphere is added tris(pentafluorophenyl)borane (0.42 g, 0.82 mmol, Aldrich). The resulting light yellow solution is heated at 60° C. for 6 hours and then stirred at room temperature for an additional 15 hours. The mixture is diluted with saturated aqueous sodium bicarbonate (12 mL) and the organic layer is separated, dried over magnesium sulfate, filtered and concentrated in vacuo to provide 2-methyl-2-o-tolyl-propionaldehyde (2.37 g , 89%) as a yellow oil. MS (ES): m/z 162.1 (M+H). ¹H NMR (δ, CDCl₃): 1.450 (6H, s), 2.190 (3H, s), 7.21-7.37 (4H, m), 9.60 (1H, s).

### REFERENCE EXAMPLE 44

### 3-Methyl-2-methylamino-3-o-tolyl-butyronitrile

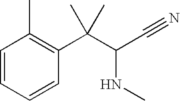

2-Methyl-2-o-tolyl-propionaldehyde (2.27 g, 14 mmol, obtained from Reference Example 43) in methanol (10 mL) is added dropwise to a solution of potassium cyanide (967 mg, 14.9 mmol) and methylamine hydrochloride (1.01 g, 15 mmol) in water (10 mL). The resulting solution is stirred at room temperature for 2 days, then diluted with water (50 mL) and extracted with methylene chloride (3×50 mL). The combined extracts are washed with saturated aqueous sodium chloride, dried over anhydrous sodium sulfate, filtered and concentrated in vacuo to provide 3-methyl-2-methylamino-3-o-tolyl-butyronitrile as a yellow oil (2.67 g, 94%). MS (ES): m/z 203.2 (M+H).

### REFERENCE EXAMPLE 45

### N,β,β,2-Tetramethylphenylalanine

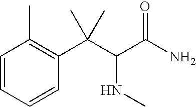

3-Methyl-2-methylamino-3-o-tolyl-butyronitrile (583 mg, 2.89 mmol, obtained from Reference Example 44) in a solution of aqueous lithium hydroxide (1 N, 11.5 mmol) is cooled to 0° C. A solution of aqueous hydrogen peroxide (30%, 2.1 mL, 20.2 mmol) is added dropwise. The resulting mixture is warmed to room temperature and stirred for 2 days. A saturated solution of sodium bisulfite is added and the mixture is extracted with ethyl acetate (3×50 mL). The combined extracts are washed with saturated aqueous sodium chloride, dried over anhydrous sodium sulfate, filtered and concentrated in vacuo. Purification (flash column, silica gel, ethyl acetate) provided N,β,β,2-tetramethylphenylalanine as a white solid (159 mg, 25%). MS (ES): m/z 221.19 (M−H). IR cm⁻¹: 3314.12, 3194.12, 1691.42, 1657.75. Analysis for C₁₃H₂₀N₂O: Calcd: C, 70.87; H, 9.15; N, 12.72. Found: C, 71.04; H, 9.22; N, 12.58.

### REFERENCE EXAMPLE 46

### 2-(N-tert-Butoxycarbonyl-N-methylamino)-3-methyl-3-o-tolyl-[N,N-di-(tert-butoxycarbonyl)]butyramide

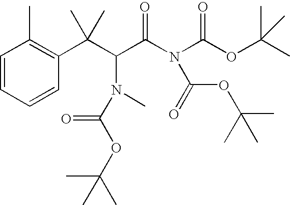

N,β,β,2-Tetramethylphenylalanine (263 mg, 1.2 mmol, obtained from Reference Example 45) in acetonitrile (3.5 mL) is treated with di-t-butyl dicarbonate (522 mg, 2.4 mmol) and stirred at room temperature for 15 hours. Additional di-t-butyl dicarbonate (522 mg, 2.4 mmol) is then added. After 15 hours, additional di-t-butyl dicarbonate (300 mg, 1.32 mmol) is added. After 3 days, additional di-t-butyl dicarbonate (522 mg, 2.4 mmol), followed by 4-(dimethylamino)pyridine (146 mg, 1.2 mmol) and N,N-diisopropylethylamine (0.229 mL, 1.3 mmol). The resulting red solution is stirred for 15 hours at room temperature, concentrated in vacuo, diluted with ethyl acetate, washed with water (2×40 mL) and saturated aqueous sodium chloride, dried over anhydrous sodium sulfate, filtered and concentrated in vacuo to provide 2-(N-tert-butoxycarbonyl-N-methylamino)-3-methyl-3-o-tolyl-[N,N-di-(tert-butoxycarbonyl)]butyramide as an orange oil (806 mg, >100%). MS (ES): m/z 521.7 (M+H).

### REFERENCE EXAMPLE 47

### 2-(N-tert-Butoxycarbonyl-N-methylamino)-3-methyl-3-o-tolyl-butyric Acid

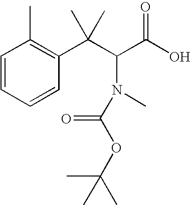

2-(N-tert-Butoxycarbonyl-N-methylamino)-3-methyl-3-o-tolyl-[N,N-di-(tert-butoxycarbonyl)]butyramide (806 mg, 1.5 mmol, obtained from Reference Example 46) in anhydrous tetrahydrofuran (2.5 mL) is treated with aqueous sodium hydroxide (5N, 1.4 mL, 7 mmol) and stirred at room temperature for 15 hours. The resulting solution is diluted with water, concentrated in vacuo, extracted with hexanes (3×50 mL). The aqueous layer is acidified to pH 5-6 with aqueous citric acid (1 M solution) and extracted with ethyl acetate (3×50 mL). The combined organic extracts are washed with aqueous saturated sodium chloride, dried over anhydrous sodium sulfate, filtered and concentrated in vacuo to provide 2-(N-tert-Butoxycarbonyl-N-methylamino)-3-methyl-3-o-tolyl-butyric acid as an orange oil (339 mg, 70%). MS (ES): m/z 322.5 (M+H).

### REFERENCE EXAMPLE 48

### (5E)-5-(3-Bromobenzylidene)-2,4-imidazolidinedione

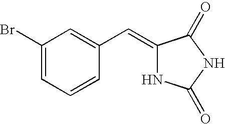

By using a procedure analogous to that described in the literature (Rapp, J.; Nieuwenhuis, S.; Creemers, A.; Hexspoor, S.; Kragl, U.; Lugtenburg, J. Eur J Org Chem, 1999, 10, 2609-2622), a mixture of hydantoin (8.1 g, 0.081 mol), m-bromobenzaldehyde (10 g, 0.054 mol), and sodium acetate (11 g, 0.130 mol) in glacial acetic acid (43 mL) is heated at reflux for 4-5 hours under a nitrogen atmosphere. During this process, all solid is dissolved and a clear solution is obtained. The hot reaction mixture is poured into 350 mL of ice-water. The solid precipitated and is collected by filtration. The solid is washed with cold water and dried under house vacuum to afford the title compound as a pale yellow powder (13 g, 93%). MS (ES+): m/z (M+H) =267.0, 269.0

### REFERENCE EXAMPLE 49

### 3-(3-Bromophenyl)-2-oxopropanoic Acid

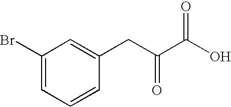

By using a procedure analogous to that described in the literature (Rapp, J.; Nieuwenhuis, S.; Creemers, A.; Hexspoor, S.; Kragl, U.; Lugtenburg, J. Eur J Org Chem, 1999, 10, 2609-2622), a solution of (5E)-5-(3-bromobenzylidene)-2,4-imidazolidinedione (13 g, 0.05 mol, from Reference Example 48) in aqueous 5N sodium hydroxide solution (250 mL) is heated at reflux for 3.5 hours under an nitrogen atmosphere. The mixture is cooled with an ice water bath, and acidified with concentrated aqueous hydrogen chloride to pH 1.0. Some of the product precipitated directly, and the solid is collected by filtration. The remainder is extracted from the aqueous filtrate with ethyl acetate, and the combined extracts are washed with saturated aqueous sodium chloride, dried over anhydrous sodium sulfate, and concentrated in vacuo, to give the product as a beige powder (1.5 g, 13%). MS (ES⁺): m/z (M+H) =243.9, 245.9

### REFERENCE EXAMPLE 50

### 3-Methyl-3-(3-bromophenyl)-2-oxobutanoic Acid

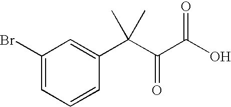

According to General Procedure II, to a solution of 3-(3-bromophenyl)-2-oxopropanoic acid (1.5 g, 6.2 mmol, from Reference Example 49) in tetrahydrofuran (5 mL) and water (2 mL) is added methyl iodide (2.4 g, 17 mmol) and 5N aqueous sodium hydroxide (4.3 mL) under nitrogen atmosphere with cooling with an ice-water bath. The cooling bath is removed, and the resulting mixture is heated at reflux overnight. The reaction mixture is allowed to cool to room temperature and the volatiles are removed under reduced pressure (bath temperature is kept below 35° C.). The residual aqueous solution is extracted with ethyl acetate to remove non-acidic components. The aqueous layer is cooled and acidifed with 10% aqueous hydrogen chloride solution to pH 1.0, and the resulting aqueous layer is extracted with ethyl acetate (3 times). The combined organic extracts are washed with saturated aqueous sodium chloride, dried over anhydrous sodium sulfate, filtered and concentrated in vacuo to afford a brown solid (0.6 g, 35%).

### REFERENCE EXAMPLE 51

### 3-Bromo-N,β,β-trimethylphenylalanine

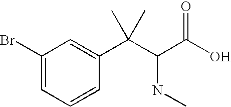

According to General Procedure III, to a solution of 3-methyl-3-(3-bromophenyl)-2-oxobutanoic acid (0.60 g, 2.2 mmol, from Reference Example 50) in anhydrous tetrahydrofuran (4.4 mL) is added methylamine (2.4 mL, 2 M solution in anhydrous tetrahydrofuran, Aldrich) at room temperature under a nitrogen atmosphere. During this procedure, the color of the pyruvate solution lightened from rust to a light brown and is accompanied by slight exothermicity. The resulting mixture is stirred for 2 hours, and then a solution of borane-pyridine complex (0.27 mL, 8M solution, Aldrich) is introduced. The mixture is then heated at 45-55° C. (bath temperature) for 2 hours, and cooled to room temperature. Methanol (2 mL) is added to the mixture with stirring, and the volatiles are removed in vacuo. The resulting syrupy residue is triturated in tetrahydrofuran. The product is precipitated upon cooling with an ice water bath. The solid is collected by filtration, and dried under high vacuum to afford a white powder (0.19 g, 30%). MS (ES⁺): (M+H) =286.3, 288.3

### REFERENCE EXAMPLE 52a AND 52b

### 3-Bromo-N,β,β-trimethyl-L-phenylalanyl-N¹-[(1S,2E)4-ethoxy-1-isopropyl-3-methyl-4-oxo-2-butenyl]-N¹,3-dimethyl-L-valinamide and (52b) 3-Bromo-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide

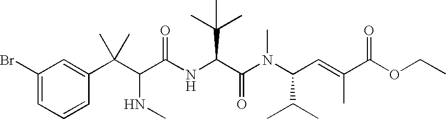

According to General Procedure IV (method IVa), to a suspension of 3-bromo-N,β,β-trimethylphenylalanine (0.29 g, 1.0 mmol, from Reference Procedure 51) and benzotriazole-1-yl-oxy-tris-pyrrolidinophosphonium hexafluorophosphate (0.57 g, 1.1 mmol) in dichloromethane (5 mL, Aldrich) is added diisopropylethylamine (0.52 mL, 3.0 mmol) under a nitrogen atmosphere. To this solution is added a solution of ethyl (2E,4S)-2,5-dimethyl-4-[methyl(3-methyl-L-valyl)amino]hex-2-enoate hydrochloride (0.35 g, 1.0 mmol) in anhydrous dichloromethane (2 mL). After stirring overnight, the clear, pale pink reaction mixture is concentrated under reduced pressure. The mixture is diluted with ethyl acetate/toluene (2:1) and washed with saturated aqueous sodium hydrogen carbonate, saturated aqueous sodium chloride, dried over anhydrous sodium sulfate, filtered and concentrated in vacuo. The crude product [(0.92 g, MS (ES⁺): (M+H)=580.3, 582.3] is purified by flash chromatography (hexanes and ethyl acetate) to give 3-bromo-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (0.12 g, 21%), 3-bromo-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (0.16 g, 28%), and a mixture of the two (0.13 g, 22%).

### REFERENCE EXAMPLE 53a AND 53b

### 3-Phenyl-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide and (53b) 3-Phenyl-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide

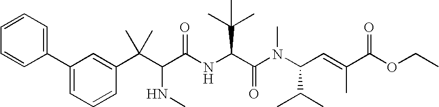

A mixture of 3-bromo-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide and 3-bromo-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (0.43 g, 0.74 mmol, from Reference Example 52) is taken up in ethylene glycol dimethyl ether (15 mL) and water (7.5 mL). Phenylboronic acid (0.18 g, 1.5 mmol), sodium carbonate (0.23 g, 2.2 mmol), and (tetrakis)triphenylphosphine palladium (0.086 g, 0.074 mmol) is added to the solution. The reaction mixture is heated at reflux overnight. The cooled reaction mixture is extracted thrice with ether. The combined organic extracts are washed with saturated aqueous sodium hydrogen carbonate and saturated aqueous sodium chloride, dried over anhydrous sodium sulfate, decanted, and concentrated under reduced pressure, to afford a white semisolid contaminated with black palladium. The crude product is purified by flash chromatography (silica gel, ethyl acetate/hexanes) to furnish 3-phenyl-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (0.18 g) and 3-phenyl-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (0.18 g). MS (ES⁺): m/z (M+H)=578.5

### REFERENCE EXAMPLE 54

### N,β,β-Trimethyl-3-vinyl-L-phenylalanyl-N¹-[(1S,2E)-4-ethoxy-1-isopropyl-3-methyl-4-oxo-2-butenyl]-N¹,3-dimethyl-L-valinamide

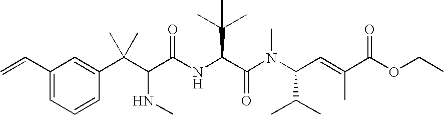

To a solution of 3-bromo-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (0.50, 0.86 mmol, from Reference Example 2) in anhydrous acetonitrile (2 mL) is added palladium (tetrakis)triphenylphosphine (20 mg, 0.02 mmol). The rust-colored suspension is stirred under a nitrogen atmosphere while tri-n-butyl(vinyl)tin (0.33 mL, 1.1 mmol) is added via syringe. The reaction mixture is heated at reflux for 20 hours and then allowed to cool to room temperature. Solvents are removed under reduced pressure. The black oily residue is purified by flash chromatography to afford the title compound as a hard white foam (0.29 g, 64%). MS (ES⁺): m/z (M+H)=528.3

### REFERENCE EXAMPLE 55

### 3-Methyl-3-phenyl-2-oxobutanoic Acid

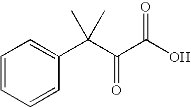

According to General Procedure II, to a solution of 3-phenylpyruvic acid (Aldrich, 25 g, 0.15 mol) in tetrahydrofuran (130 mL) and water (50 mL) is added methyl iodide (57 g, 0.40 mol) and 5N aqueous sodium hydroxide solution (80 mL) under nitrogen atmosphere with cooling with an ice-water bath. The cooling bath is removed, and the resulting mixture is heated at reflux for 5 hours. The reaction mixture is allowed to cool to room temperature and methyl iodide (23 g, 0.16 mol) is added, followed by 5N aqueous sodium hydroxide solution (50 mL). Stirring is continued overnight at room temperature. The volatiles are removed under reduced pressure (bath temperature is kept below 35° C.), and the residual aqueous solution is extracted with ethyl acetate to remove non-acidic components. The residue is cooled and acidifed with 10% hydrogen chloride to pH 1.0, and the resulting aqueous layer is extracted with ethyl acetate (3 times). The combined organic extracts are washed with saturated aqueous sodium chloride, dried over anhydrous sodium sulfate, filtered and concentrated in vacuo to afford a brown oil (31 g, >100%). This material could be triturated with hexanes to afford white needles. MS (ES⁻): m/z(M−H)=190.9

### REFERENCE EXAMPLE 56

### 3-Methyl-3-(4-bromophenyl)-2-oxobutanoic Acid

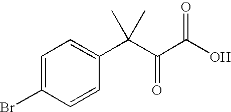

To a solution of 3-methyl-3-phenyl-2-oxobutanoic acid (1.0 g, 5.2 mmol, from Reference Example 55) in carbon tetrachloride (3.5 mL), is added bromine (0.86 g, 5.4 mmol) and iron powder (0.007 g). The resulting mixture is heated at reflux overnight. The reaction mixture is allowed to cool to room temperature. The deep red reaction mixture is washed with cold 10% hydrochloric acid and then water. The organic phase is then extracted with 2.5 M sodium hydroxide (40 mL). The basic extracts are then acidified to pH of 1.0 with concentrated aqueous hydrochloric acid. A dark red liquid oiled out, which is isolated by extraction with ethyl acetate (×3). The combined organic extracts are washed with brine, dried over anhydrous sodium sulfate, decanted, and concentrated under reduced pressure to afford a straw-colored semisolid (1.1 g, 79%). MS (ES⁻): m/z (M−H)=269.1, 271.1

### REFERENCE EXAMPLE 57

### 4-Bromo-N,β,β-trimethylphenylalanine

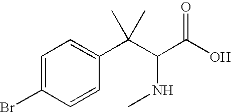

According to General Procedure III, to a solution of 3-methyl-3-(4-bromophenyl)-2-oxobutanoic acid (61% pure, 1.0 g, 3.7 mmol, from Reference Example 56) in anhydrous tetrahydrofuran (8 mL) is added methylamine (4.1 mL, 2 M solution in anhydrous tetrahydrofuran, Aldrich) at room temperature under a nitrogen atmosphere. During this procedure, the color of the pyruvate solution lightened to a straw color and is accompanied by slight exothermicity. After about 45 minutes, a thick white precipitate formed. The resulting mixture is stirred for 1 hour total, and then a solution of borane-pyridine complex (0.45 mL, 8 M solution, Aldrich) is introduced. The mixture is then heated at 45-55° C. (bath temperature) for 2.0-2.5 hours, and cooled to room temperature. Methanol (2 mL) is added to the mixture with stirring, and the volatiles are removed in vacuo. The resulting syrupy white precipitate is triturated in tetrahydrofuran. The product is precipitated upon cooling with an ice water bath. The solid is collected by filtration, and dried under high vacuum to afford a white powder (0.32 g, 49%). MS (ES⁻): m/z (M−H)=284.2, 286.2

### REFERENCE EXAMPLE 58

### 4-Bromo-N,β,β-trimethylphenylalanyl-N¹-[(1S,2E)-4-ethoxy-1-isopropyl-3-methyl-4-oxo-2-butenyl]-N¹,3-dimethyl-L-valinamide

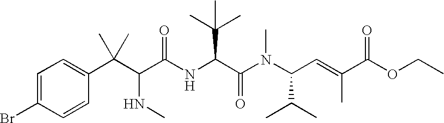

In accordance with General Procedure IVa, to a suspension of 4-bromo-N,β,β-trimethylphenylalanine (0.29 g, 1.5 mmol, from Reference Example 57) and benzotriazole-1-yl-oxy-tris-pyrrolidinophosphonium hexafluorophosphate (0.57 g, 1.1 mmol) in dichloromethane (7 mL, Aldrich) under a nitrogen atmosphere is added diisopropylethylamine (0.35 mL, 2.0 mmol). To this solution is added a solution of ethyl (2E,4S)-2,5-dimethyl-4-[methyl(3-methyl-L-valyl)amino]hex-2-enoate hydrochloride (0.38 g, 1.1 mmol) in anhydrous dichloromethane (3 mL). After stirring for 30 minutes, the suspension turned clear, and the resulting reaction mixture is stirred at room temperature for 21 hours. Volatiles are evaporated under reduced pressure. The residue is partitioned between ethyl acetate and water. The aqueous layer is extracted with ethyl acetate (3 times). The combined extracts are washed with saturated sodium hydrogen carbonate, saturated aqueous sodium chloride, dried over anhydrous sodium sulfate, filtered and concentrated in vacuo. The crude product (0.91 g) is purified by preparative reverse-phase HPLC, eluting from 5% acetonitrile/95% water/0.1% trifluoroacetic acid to 100% acetonitrile over one hour to yield the title compound as a trifluoroacetic acid salt. MS (ES⁺): (M+H)=580.6, 582.6

### REFERENCE EXAMPLE 59

### 4-Phenyl-N,β,β-trimethylphenylalanine

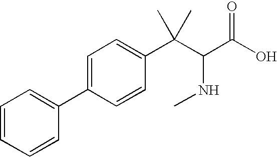

To a solution of 4-bromo-N,β,β-trimethylphenylalanine (0.21 g, 0.75 mmol, from Reference Example 57) in ethylene glycol dimethyl ether (15 mL) and water (7.5 mL), is added phenylboronic acid (0.18 g, 1.5 mmol), sodium carbonate (0.24 g, 2.3 mmol), and (tetrakis)triphenylphosphine palladium (0.087 mg, 0.08). The reaction mixture is shaken at 85° C. for 80 minutes and then at 80° C. for 2 hours. LC/MS analysis of the reaction mixture revealed the presence of starting material, so heating is continued overnight at 75° C. The reaction mixture is then allowed to cool to room temperature and then concentrated under reduced pressure. Dimethylformamide and water is added to the residue. The pH is adjusted to 6 by the addition of 1 M citric acid solution. The aqueous phase is extracted four times with ethyl acetate. The combined organic extracts are filtered through a Diatomaceous earth pad and then concentrated under reduced pressure to afford a dark brown liquid, which is purified by preparative reverse-phase HPLC, eluting from 5% acetonitrile/95% water/0.1% trifluoroacetic acid to 100% acetonitrile over one hour, to afford a white powder (30 mg, 14%) MS (ES⁺): m/z (M+H)=284.1

### REFERENCE EXAMPLE 60

### 4-Phenyl-N,β,β-trimethylphenylalanyl-N¹-[(1S,2E)-4-ethoxy-1-isopropyl-3-methyl-4-oxo-2-butenyl]-N¹,3-dimethyl-L-valinamide

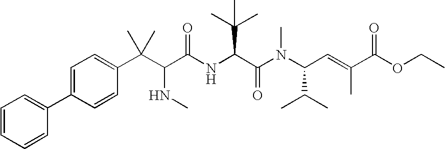

To a solution of a 1:1 mixture of 4-bromo-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide and 4-bromo-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (0.25 g, 0.43 mmol, from Reference Example 58) in ethylene glycol dimethyl ether (9 mL) and water (4 mL) is added phenylboronic acid (0.10 g, 0.86 mmol), sodium carbonate (0.14 g, 1.3 mmol), and (tetrakis)triphenylphosphine palladium (0.05 mg, 0.04). The reaction mixture is heated at reflux for 18 hours and then allowed to cool to room temperature. Volatiles are evaporated under reduced pressure and the residue is partitioned between ether and water. The aqueous phase is extracted thrice with ether. The combined organic extracts are washed with water and saturated sodium chloride solution, dried over anhydrous sodium sulfate, decanted, and concentrated under reduced pressure to afford a reddish-brown semisolid (0.20 g). MS (ES⁺): m/z (M+H)=578.5 According to General Procedure IV (method IVa), to a suspension of 4-phenyl-N,β,β-trimethylphenylalanine (0.05 g, 0.18 mmol, from Reference Example 59) and benzotriazole-1-yl-oxy-tris-pyrrolidinophosphonium hexafluorophosphate (0.99 g, 0.19 mmol) in dichloromethane (2 mL, Aldrich) is added diisopropylethylamine (0.06 mL, 0.35 mmol) under a nitrogen atmosphere. To this solution is added a solution of ethyl (2E,4S)-2,5-dimethyl-4-[methyl(3-methyl-L-valyl)amino]hex-2-enoate hydrochloride (0.06 g, 0.18 mmol) in anhydrous dichloromethane (2 mL). After stirring for 10 minutes, the suspension turned clear, and the resulting reaction mixture is stirred at room temperature for 48 hours. The volatiles are evaporated under reduced pressure. The crude product is combined with the above material (0.20 g) and purified by preparative reverse-phase HPLC, eluting from 5% acetonitrile/95% water/0.1% trifluoroacetic acid to 100% acetonitrile over one hour, to yield the two diastereomers as their trifluoroacetic acid salts. The first isomer to elute is 4-phenyl-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (40 mg). The second isomer to elute is 4-phenyl-N,β,β,-trimethyl-L-phenylalanyl-N¹-[(1S,2E)-4-ethoxy-1-isopropyl-3-methyl-4-oxo-2-butenyl]-N¹,3-dimethyl-L-valinamide (60 mg). MS (ES⁺): (M+H)=578.5

### REFERENCE EXAMPLE 61

### 4-Bromo-β,β-dimethylphenylalanine

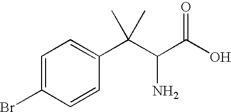

According to a modified version of General Procedure III, to a solution of 3-methyl-3-(4-bromophenyl)-2-oxobutanoic acid (16 g, 60 mmol, from Reference Example 56) in anhydrous tetrahydrofuran (120 mL) is added ammonia (270 mL, 0.5 M solution in anhydrous dioxane, Aldrich) at room temperature under a nitrogen atmosphere. During this procedure, a thick white slurry formed. The resulting mixture is stirred for 1.5 hours, and then a solution of borane-pyridine complex (7.3 mL, 8 M solution, Aldrich) is introduced. The mixture is then heated at 45-55° C. (bath temperature) for 2 hours, and cooled to room temperature. Methanol (10 mL) is added to the mixture with stirring, and the volatiles are removed in vacuo. The resulting syrupy white precipitate is triturated with tetrahydrofuran. The product precipitated upon cooling with an ice water bath. The solid is collected by filtration, and dried over high vacuum to afford a white powder (5.6 g, 35%). MS (ES⁺): (M+H)=272.0, 274.0

### REFERENCE EXAMPLE 62

### 4-Bromo-N-(tert-butoxycarbonyl) β,β-dimethylphenylalanine

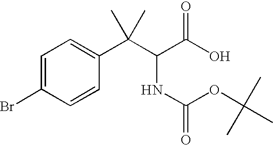

To a suspension of 4-bromo-β,β-dimethylphenylalanine (6.5 g, 24 mmol, from Reference Example 61) in 1:1 acetone/water (100 mL) is added potassium carbonate (9.9 g, 72 mmol) to dissolve the solid. Di-t-butyl dicarbonate (10 g, 48 mmol) is added and the reaction mixture is stirred under a nitrogen atmosphere overnight. The reaction mixture is cooled to 0° C. in an ice-water bath and acidified to pH=4 with 1 M aqueous citric acid. The acidified solution is extracted thrice with dichloromethane. The combined organic extracts are washed with saturated aqueous sodium chloride, dried over anhydrous sodium sulfate, decanted, and concentrated under reduced pressure to afford 7.8 g (88% crude) of a sticky white foam. MS (ES⁺): m/z (M+Na)=394.0, 396.0

### REFERENCE EXAMPLE 63

### Methyl 4-bromo-N-(tert-butoxycarbonyl)-N,β,β-trimethylphenylalanine

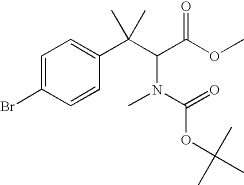

To an ice-cooled solution of 4-bromo-N-(tert-butoxycarbonyl) β,β-dimethylphenylalanine (7.8 g, 21 mmol, from Reference Example 62) in anhydrous dimethylformamide (250 mL), sodium hydride (60% dispersion in mineral oil, 4.2 g, 110 mmol) is added with stirring under a nitrogen atmosphere. After stirring for 1 hour at 0° C., to the clear, peach-colored solution is added methyl iodide (13 mL, 210 mmol). The reaction mixture is allowed to stir overnight under nitrogen while gradually warming to room temperature. The reaction mixture is cooled to 0° C. in an ice-water bath and quenched by the addition of glacial acetic acid to pH=5. The acidified solution is extracted thrice with diethyl ether. The combined organic extracts are washed with saturated aqueous sodium chloride, dried over anhydrous sodium sulfate, decanted, and concentrated under reduced pressure to afford approximately 10 g of a blond oil. Flash chromatography (ethyl acetate/hexanes) of the oil afforded 7.6 g (90%) of the title compound as a pale blond oil. MS (ES⁺): m/z (M+Na)=422.0, 424.0

### REFERENCE EXAMPLE 64

### Methyl 4-vinyl-N-(tert-butoxycarbonyl)-N,β,β-trimethylphenylalanine

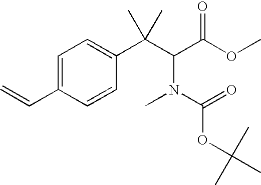

To a suspension of methyl 4-bromo-N-(tert-butoxycarbonyl)-N,β,β-trimethylphenylalanine (1.0 g, 2.5 mmol, from Reference Example 63) in anhydrous acetonitrile (5 mL) is added tetrakis(triphenylphosphine)palladium (0.058 mg, 0.05 mmol) under a nitrogen atmosphere. To the stirred suspension is added tri-n-butyl(vinyl)tin (1.0 g, 3.3 mmol). The reaction mixture is heated at reflux for 20 hours, over the course of which the bromide dissolved. The reaction mixture is cooled to room temperature and the volatile components are evaporated under reduced pressure. The residue is subjected to flash chromatography (ethyl acetate/hexanes) to afford the title compound (0.69 g, 79%) as a clear, colorless oil. MS (ES⁺): m/z (M+H)=348.5

### REFERENCE EXAMPLE 65

### Methyl 4-formyl-N-(tert-butoxycarbonyl)-N,β,β-trimethylphenylalanine

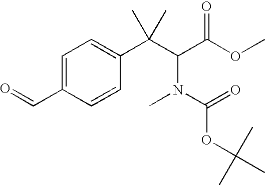

Ozone is bubbled into a −78° C. solution of methyl 4-vinyl-N-(tert-butoxycarbonyl)-N, β,β-trimethylphenylalanine (0.69 g, 2.0 mmol, from Reference Example 64) in dichloromethane (10 mL). After 5 minutes, the blue color of an ozone saturated solution is observed. Influx of ozone is continued for one minute and then replaced with a flow of nitrogen. After 5 minutes, the blue color had disappeared and the nitrogen line is removed. Dimethylsulfide (0.44 mL, 6 mmol) is added, the cooling bath is removed, and the reaction mixture is allowed to warm to room temperature. Volatiles are then evaporated under reduced pressure, leaving a clear, colorless oil (0.79 g). The material is used for the next step without further purification. MS (ES⁺): m/z (M+dimethylsulfide)=396.5

### REFERENCE EXAMPLE 66

### Methyl 4-carboxyl-N-(tert-butoxycarbonyl)-N,β,β-trimethylphenylalanine

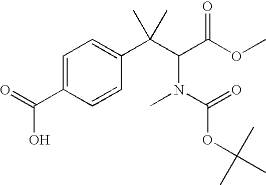

To a suspension of crude methyl 4-formyl-N-(tert-butoxycarbonyl)-N,β,β-trimethylphenylalanine (2.0 mmol max., from Reference Example 65) in dioxane (8 mL) and water (4 mL) is added sulfamic acid (1.1 g) in a single portion. Once it dissolved, a solution of sodium chlorite (80% tech, 0.32 g) in water (4 mL) is added dropwise over 2 minutes. During this process, color of the reaction mixture turned from a light rose to bright yellow. The mixture is stirred an additional 25 minutes at room temperature, and then poured into water (75 mL) and extracted thrice with ethyl acetate. The combined extracts are washed with brine, dried over anhydrous sodium sulfate, decanted, and concentrated under reduced pressure to a clear, colorless oil (0.78 g, >100%). The material is used for the next step without further purification. MS (ES⁺): m/z (M+H)=366.6

### REFERENCE EXAMPLE 67

### Methyl 4-(tert-butoxycarbonyl)-N-(tert-butoxycarbonyl)-N,β,β-trimethylphenylalanine

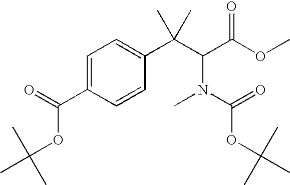

To a solution of the crude methyl 4-carboxyl-(N-butoxycarbonyl)-N,β,β-trimethylphenylalanine (2.0 mmol max, from Reference Example 66) in toluene (5 mL) is added dimethylformamide di-t-butyl acetal (1.9 mL, 8.0 mmol). The mixture is heated at reflux while stirring under a nitrogen atmosphere for 90 minutes. The reaction mixture is allowed to cool to room temperature and then poured into saturated aqueous sodium chloride. The aqueous phase is extracted thrice with diethyl ether. The combined organic extracts are washed with saturated aqueous sodium chloride, dried over anhydrous sodium sulfate, decanted, and concentrated under reduced pressure to a reddish-orange semi-solid (0.77 g). The crude material is purified by flash chromatography (0.29 g, 35% over 3 steps) to give a clear, colorless foaming oil.

### REFERENCE EXAMPLE 68

### 4-(tert-Butoxycarbonyl)-N-(tert-butoxycarbonyl)-N,β,β-trimethylphenylalanine

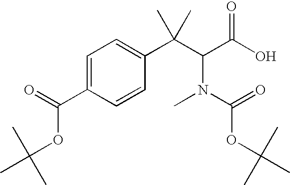

Methyl 4-(tert-butoxycarbonyl)-N-(tert-butoxycarbonyl)-N,β,β-trimethylphenylalanine (0.29 g, 0.69 mmol, from Reference Example 67) is dissolved in tetrahydrofuran (2 mL), methanol (2 mL), water (1 mL). To this solution is added lithium hydroxide monohydrate (0.071 g, 1.7 mmol). The resulting mixture is briefly stirred at 60° C. to dissolve all solids and then allowed to stir at 35-40° C. overnight. On the following day, the reaction mixture is concentrated to white foam under reduced pressure. Following trituration with acetonitrile and 5% aqueous citric acid solution, solids are filtered and dried on house vacuum. The filtrate is concentrated under reduced pressure and acidified with additional 5% aqueous citric acid solution. A white gum precipitated which is collected and dried under high vacuum. A total of 0.25 g (89%) is collected and used in the following step without further purification. MS (ES⁻): m/z (M−H)=406.5

### REFERENCE EXAMPLE 69

### (5E)-5-(3-Methoxybenzylidene)-2,4-imidazolidinedione

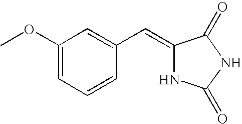

By using a procedure analogous to that described in the literature (Rapp, J.; Nieuwenhuis, S.; Creemers, A.; Hexspoor, S.; Kragl, U.; Lugtenburg, J. Eur J Org Chem, 1999, 10, 2609-2622), a mixture of hydantoin (4.4 g, 0.044 mol), m-anisaldehyde (4.0 g, 0.029 mol), and sodium acetate (5.8 g, 0.071 mol) in glacial acetic acid (23 mL) is is heated at reflux for 4-5 hours under a nitrogen atmosphere. During this process, all solid is dissolved and a clear solution is obtained. The hot reaction mixture is poured into 140 mL of ice-water. The solid precipitated and is collected by filtration. The solid is washed with cold water and dried under house vacuum. MS (ES⁻): m/z (M−H)=217.1

### REFERENCE EXAMPLE 70

### 3-(3-Methoxyphenyl)-2-oxopropanoic Acid

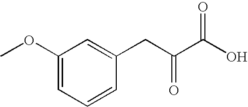

By using a procedure analogous to that described in the literature (Rapp, J.; Nieuwenhuis, S.; Creemers, A.; Hexspoor, S.; Kragl, U.; Lugtenburg, J. Eur J Org Chem, 1999, 10, 2609-2622), a solution of (5E)-5-(3-methoxybenzylidene)-2,4-imidazolidinedione (0.029 mol, from Reference Example 69) in aqueous 5N sodium hydroxide solution (140 mL) is heated at reflux for 1-2 hours under a nitrogen atmosphere. The mixture is cooled with an ice water bath, and acidified with concentrated aqueous hydrogen chloride to pH 1.0. Some of the product precipitated directly, and the solid is collected by filtration. The remainder is extracted from aqueous filtrate with ethyl acetate, and the combined extracts are washed with saturated aqueous sodium chloride, dried over magnesium sulfate, and concentrated in vacuo, to give the product as a yellow powder (2.0 g, 35% yield). MS (ES⁻):m/z (M−H)=193.1

### REFERENCE EXAMPLE 71

### 3-Methyl-3-(3-methoxyphenyl)-2-oxobutanoic Acid

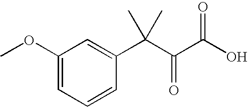

According to General Procedure II, to a solution of 3-(3-methoxyphenyl)-2-oxopropanoic acid (2.0 g, 0.010 mol, from Reference Example 70) in tetrahydrofuran (9 mL) and water (3.5 mL) is added methyl iodide (3.9 g, 0.027 mol) and 5N aqueous sodium hydroxide (5.2 mL) under a nitrogen atmosphere with cooling with an ice-water bath. The cooling bath is removed, and the resulting mixture is heated at reflux for 5 hours. At this time, the reaction mixture is allowed to cool to room temperature and methyl iodide (1.6 g, 0.011 mol) is added, followed by 5N aqueous sodium hydroxide (3.5 mL). Stirring is continued overnight at room temperature. The volatiles are removed under reduced pressure (bath temperature is kept below 35° C.), and the residual aqueous solution is extracted with ethyl acetate to remove non-acidic components. The residue is cooled and acidifed with 10% aqueous hydrogen chloride solution to pH 1.0, and the resulting aqueous layer is extracted with ethyl acetate (3 times). The combined extracts are washed with aqueous saturated sodium chloride, dried over anhydrous sodium sulfate, filtered and concentrated in vacuo to afford the title compound as a brown oil (1.7 g, 74%).

### REFERENCE EXAMPLE 72

### 3-Methoxy-N-β,β-trimethylphenylalanine

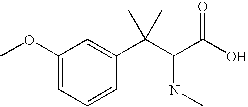

According to General Procedure III, to a solution of 3-methyl-3-(3-methoxyphenyl)-2-oxobutanoic acid (0.84 g, 0.004 mol, from Reference Example 71) in anhydrous tetrahydrofuran (8 mL) is added methylamine (4.2 mL, 2 M solution in anhydrous tetrahydrofuran, Aldrich) at room temperature under a nitrogen atmosphere. During this procedure, the color of the pyruvate solution lightened to a straw color and is accompanied by slight exothermicity. The resulting mixture is stirred for 1 hour, and then a solution of borane-pyridine complex (0.46 mL, 8 M solution, Aldrich) is introduced. The mixture is then heated at 45-55° C. (bath temperature) for 2.0-2.5 hours, and cooled to room temperature. Methanol (2 mL) is added to the mixture with stirring, and the volatiles are removed in vacuo. The resulting syrupy residue is triturated with tetrahydrofuran. The product precipitated upon cooling with an ice water bath. The solid is collected by filtration, and dried under high vacuum to afford the title compound as a white powder (0.39 g, 43%). MS (ES⁺): m/z (M+H)=238.3

### REFERENCE EXAMPLE 73

### 3-Methoxy-N,β,β-trimethylphenylalanyl-N¹-[(1S,2E)-4-ethoxy-1-isopropyl-3-methyl-4-oxo-2-butenyl]-N¹,3-dimethyl-L-valinamide

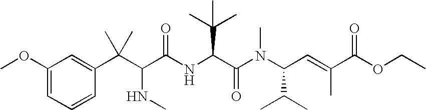

According to General Procedure IVa, to a cooled (0° C., ice bath) solution of 3-methoxy-N-β,β-trimethylphenylalanine (0.35 g, 1.5 mmol, from Reference Example 72) and benzotriazole-1-yl-oxy-tris-pyrrolidinophosphonium hexafluorophosphate (1.1 mmol) in dichloromethane (2 mL, Aldrich) is added diisopropylethylamine (0.52 mL, 3.0 mmol) under a nitrogen atmosphere. To this solution is added a solution of ethyl (2E,4S)-2,5-dimethyl-4-[methyl(3-methyl-L-valyl)amino]hex-2-enoate (0.53 g, 1.7 mmol) in anhydrous dichloromethane (5 mL). After stirring at 0° C. for 5-10 minutes, the cooling bath is removed, and the resulting reaction mixture is stirred at room temperature for 15-20 hours. The mixture is diluted with water, and the aqueous layer is extracted with ethyl acetate (3 times). The combined organic extracts are washed with saturated aqueous sodium chloride, dried over anhydrous sodium sulfate, filtered and concentrated in vacuo. The crude product (1.5 g) is used for the next step without further purification. MS (ES⁺): m/z (M+H)=532.4

### REFERENCE EXAMPLE 74

### 3-Methyl-3-(3-hydroxyphenyl)-2-oxobutanoic Acid

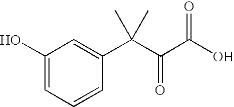

Crude 3-methyl-3-(3-methoxyphenyl)-2-oxobutanoic acid (0.90 g, 4.0 mmol, from Reference Example 71) is dissolved in anhydrous dichloromethane (12 mL) in a 50 mL round-bottom flask. Under nitrogen atmosphere, the solution is cooled to −78° C. in a dry ice/acetone bath. Boron tribromide (1.0 M solution in dichloromethane, 6.8 mL, 6.8 mmol) is added dropwise. An exothermic reaction ensued in which the color of the reaction mixture turned a deep red. After the addition, the reaction mixture is allowed to warm to room temperature while stirring overnight under nitrogen. On the following day, a deep green reaction mixture containing a white precipitate is observed. It is quenched by the careful dropwise addition of water (20 mL) and the layers are partitioned. The aqueous phase is extracted with diethyl ether (×3). The combined organic extracts are washed with brine (×2), dried over anhydrous sodium sulfate, decanted, and concentrated under reduced pressure to afford the title compound as a dark red liquid (0.89 g, >100%), which is used in the next step without further purification. MS (ES⁻): m/z (M−H)=207.1

### REFERENCE EXAMPLE 75

### 3-Hydroxy-N,β,β-trimethylphenylalanine

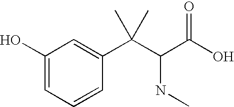

According to General Procedure III, to a solution of the crude 3-methyl-3-(3-hydroxyphenyl)-2-oxobutanoic acid (0.004 mol max., from Reference Example 74) in anhydrous tetrahydrofuran (8 mL) is added methylamine (4.4 mL, 2 M solution in anhydrous tetrahydrofuran, Aldrich) at room temperature under a nitrogen atmosphere. During this procedure, the color of the pyruvate solution lightened to a straw color and is accompanied by slight exothermicity. The resulting mixture is stirred for 1 hour, and then a solution of borane-pyridine complex (0.46 mL, 8 M solution, Aldrich) is introduced. The mixture is then heated at 45-55° C. (bath temperature) for 2.0-2.5 hours, and cooled to room temperature. Methanol (2 mL) is added to the mixture with stirring, and the volatiles are removed in vacuo. The resulting syrupy residue is triturated with tetrahydrofuran. The product precipitated upon cooling with an ice water bath. The solid is collected by filtration, and dried over high vacuum to afford the title compound as a white powder (0.28 g, 31% for two steps). MS (ES⁻): m/z (M−H)=222.2

### REFERENCE EXAMPLE 76

### 3-Hydroxy-N,β,β-trimethylphenylalanyl-N¹-[(1S,2E)-4-ethoxy-1-isopropyl-3-methyl-4-oxo-2-butenyl]-N¹,3-dimethyl-L-valinamide

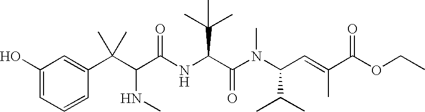

According to General Procedure IVa, to a cooled (0° C., ice bath) to a suspension of 3-hydroxy-N,β,β-trimethylphenylalanine (0.25 g, 1.1 mmol, from Reference Example 75) and benzotriazole-1-yl-oxy-tris-pyrrolidinophosphonium hexafluorophosphate (0.64 g, 1.2 mmol) in dichloromethane (5 mL, Aldrich) is added diisopropylethylamine (0.38 mL, 2.2 mmol) under a nitrogen atmosphere. To this solution is added a solution of ethyl (2E,4S)-2,5-dimethyl-4-[methyl(3-methyl-L-valyl)amino]hex-2-enoate (0.38 g, 1.2 mmol) in anhydrous dichloromethane (2 mL). After stirring at 0° C. for 5-10 minutes, the cooling bath is removed, and the resulting reaction mixture is stirred at room temperature for 3-4 hours, after which a suspension remained. Anhydrous dimethylformamide (2 mL) is added and the reaction mixture is allowed to stir overnight under nitrogen atmosphere. The mixture is diluted with water, and the aqueous layer is extracted with ethyl acetate (3 times). The combined organic extracts are washed with 10% hydrochloric acid and saturated aqueous sodium chloride, dried over anhydrous sodium sulfate, decanted, and concentrated in vacuo. The crude title compound (0.11 g) is used in the next step without further purification. MS (ES⁺): m/z (M+H)=518.5

### REFERENCE EXAMPLE 77

### 3,3-Dimethyl-2-oxo-4-phenyl-butyric Acid

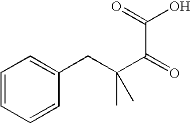

By the method described in General Procedure II, commercially available 2-oxo-4-phenyl-butyric acid (5.0 g, 0.024 mol,) is treated with iodomethane (4.05 mL, 0.065 mol), and 5N aqueous sodium hydroxide (12.2 mL, 0.061 mol) in tetrahydrofuran (20 mL)/water (8 mL) to give 3,3-dimethyl-2-oxo4-phenyl-butyric acid as a dark oil (4.0 g). MS (ES⁺): (M+H)=207.

### REFERENCE EXAMPLE 78

### N,3-Dimethyl-4-phenylvaline

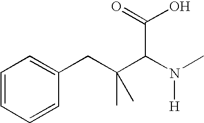

3,3-Dimethyl-2-oxo-4-phenyl-butyric acid (2.1 g, from Reference Example 77) is treated with methylamine (2M in tetrahydrofuran, 11.5 mL, 23 mmol) and borane-pyridine complex (1.53 mL, 12.2 mmol, 8 M solution) in tetrahydrofuran (20 mL) as described in General Procedure III, to give N, 3-dimethyl-4-phenylvaline as a white solid. MS (ES): m/z 222.14897 (M+H). (calc'd exact mass=221.14166)

### REFERENCE EXAMPLE 79

### 3,3-Dimethyl-2-oxo-octanoic Acid

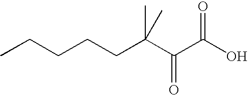

By the method described in General Procedure II, commercially available 2-oxo-octanoic acid (3.0 g, 17.4 mmol,) is treated with iodomethane (2.92 mL, 47 mmol), and 5N aqueous sodium hydroxide (8.7 mL, 43 mmol) in tetrahydrofuran (14.4 mL)/water (6 mL), to give 3,3-dimethyl-2-oxo-octanoic acid. MS (ES): m/z 185.1 (M−H).

### REFERENCE EXAMPLE 80

### 3,3-Dimethyl-2-(methylamino)-octanoic Acid

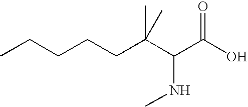

3,3-Dimethyl-2-oxo-octanoic acid (2.35 g) from Reference Example 79 is treated with methylamine (2M in tetrahydrofuran, 14 mL, 27.7 mmol) and borane-pyridine complex (1.9 mL, 15 mmol, 8 M solution) in tetrahydrofuran (20 mL) as described in General Procedure III to give 3,3-dimethyl-2-(methylamino)-octanoic acid as a white solid. MS (ES): m/z 202.18047 (M+H). (calc'd exact mass=201.17298)

### REFERENCE EXAMPLE 81a AND 81b

### N-(tert-Butoxycarbonyl)-2-methoxy-N,β,β-trimethyl-L-phenylalanine and (81 b)N-(tert-butoxycarbonyl)-N,O,β,β-tetramethyl-L-tyrosine

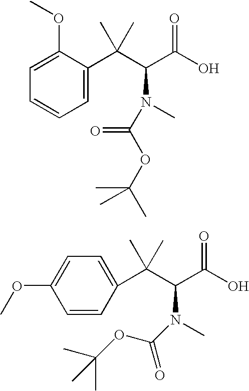

Starting from methoxybenzene, N-(tert-butoxycarbonyl)-2-methoxy-N,β,β-trimethyl-L-phenylalanine and N-(tert-butoxycarbonyl)-N,O,β,β-tetramethyl-L-tyrosine are synthesized by a method analogous to that described in Andersen, R. et. al. WO 99/32509. N-(tert-butoxycarbonyl)-2-methoxy-N,β,β-trimethyl-L-phenylaIanine: HRMS (ESI) calcd for C₁₈H₂₇NO₅ (M−H⁺) 336.1816, found 336.1817; N-(tert-butoxycarbonyl)-N,O,β,β-tetramethyl-L-tyrosine HRMS (ESI) calcd for C₁₈H₂₇NO₅ (M−H⁺) 336.1816, found 336.1817.

### REFERENCE EXAMPLE 82

### N-(tert-Butoxycarbonyl)-2-methoxy-N,β,β-trimethyl-L-phenylalanyl-N1-[(1S,2E)-4-ethoxy-1-isopropyl-3-methyl-4-oxo-2-butenyl]-N¹,3-dimethyl-L-valinamide

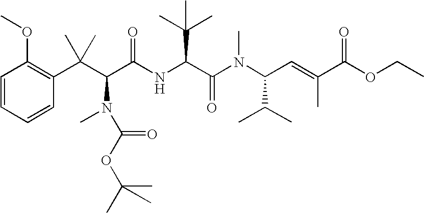

According to the General Procedure IV, to a solution of N-(tert-butoxycarbonyl)-2-methoxy-N,β,β-trimethyl-L-phenylalanine (202 mg, 0.6 mmol, from Reference Example 81) and benzotriazole-1-yl-oxy-tris-pyrrolidinophosphonium hexafluorophosphate (406 mg, 0.78 mmol) in dichloromethane (10 mL) is added diisopropylethylamine (0.31 mL, 1.8 mmol), followed by addition of ethyl (2E,4S)-2,5-dimethyl-4-[methyl(3-methyl-L-valyl)amino]hex-2-enoate hydrochloride (209 mg, 0.6 mmoL). Purification by chromatography (2: 1 hexanes: ethyl acetate) gave the title compound as a white solid (211 mg, 56% yield). HRMS (ESI) calcd for C₃₅H₅₇N₃O₇ (M+H⁺) 632.4269, found 632.4268.

### REFERENCE EXAMPLE 83

### N-(tert-Butoxycarbonyl)-2-methoxy-N,β,β-trimethyl-L-phenylalanyl-N¹-[(1S,2E)-3-carboxy-1-isopropyl-2-butenyl]-N¹,3-dimethyl-L-valinamide

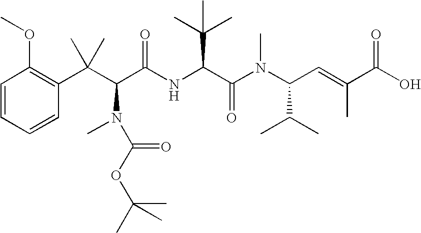

According to General Procedure V, N-(tert-butoxycarbonyl)-2-methoxy-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (101 mg, 0.16 mmol, from Reference Example 82) is dissolved in methanol (4 mL). To this solution is added water (1.3 mL) and 1.0 M aqueous lithium hydroxide (1.3 mL, 1.3 mmol). Purification by chromatography (1:1: 0.01 hexanes: ether: acetic acid) provided the title compound (92 mg, 95%) as a white solid. HRMS (ESI) calcd for C₃₃H₅₃N₃O₇ (M+H⁺) 604.3956, found 604.3949.

### REFERENCE EXAMPLE 84

### N-(tert-Butoxycarbonyl)-N,O,β,β-tetramethyl-L-tyrosyl-N¹-[(1S,2E)-4-ethoxy-1-isopropyl-3-methyl4-oxo-2-butenyl]-N¹,3-dimethyl-L-valinamide

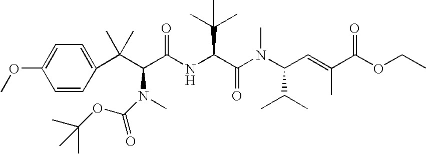

According to the General Procedure IV, to a solution of N-(tert-butoxycarbonyl)-N,O, β,β-tetramethyl-L-tyrosine (135 mg, 0.4 mmol, from Reference Example 81) and benzotriazole-1-yl-oxy-tris-pyrrolidinophosphonium hexafluorophosphate (271 mg, 0.52 mmol) in dichloromethane (5 mL) is added diisopropylethylamine (0.21 mL, 1.2 mmol) followed by the addition of ethyl (2E,4S)-2,5-dimethyl-4-[methyl(3-methyl-L-valyl)amino]hex-2-enoate hydrochloride (139 mg, 0.4 mmoL). Purification by chromatography (4:1 hexanes : ethyl acetate) provided the title compound (180 mg, 71%) as a white solid. HRMS (ESI) calcd for C₃₅H₅₇N₃O₇ (M+H⁺) 632.4269, found 632.4272.

### REFERENCE EXAMPLE 85

### N-(tert-Butoxycarbonyl)-N,O,β,β-tetramethyl-L-tyrosyl-N¹-[(1S,2E)-3-carboxy-1-isopropyl-2-butenyl]-N¹,3-dimethyl-L-valinamide

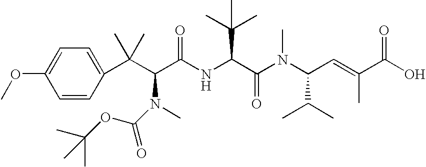

According to General Procedure V, N-(tert-butoxycarbonyl)-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide (100 mg, 0.16 mmol) from Reference Example 84 is dissolved in methanol (4 mL). To this solution is added water (1.3 mL) and 1.0 M aqueous lithium hydroxide (1.3 mL, 1.3 mmol). Chromatography (1:1: 0.01 hexanes: ether: acetic acid) provided 82 mg (86%) of the title compound as a white solid. HRMS (ESI) calcd for C₃₃H₅₃N₃O₇ (M+H⁺) 604.3956, found 604.3958.

### REFERENCE EXAMPLE 86

### N-(tert-Butoxycarbonyl)-2-methoxy-N,O,β,β-tetramethyl-L-tyrosine

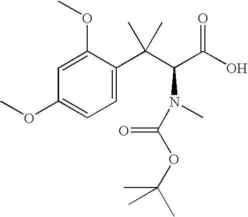

Starting from 1,3-bismethoxybenzene, N-(tert-butoxycarbonyl)-2-methoxy-N,O,β,β-tetramethyl-L-tyrosine is synthesized by a method analogous to that described in Andersen, R. et. al. WO 99/32509. HRMS (ESI) calcd for C₁₉H₂₉NO₆ (M−H⁺) 366.1922, found 366.1925.

### REFERENCE EXAMPLE 87

### N-(tert-Butoxycarbonyl)-2-methoxy-N,O,β,β-tetramethyl-L-tyrosyl-N¹-[(1S,2E)4-ethoxy-1-isopropyl-3-methyl4-oxo-2-butenyl]-N¹,3-dimethyl-L-valinamide

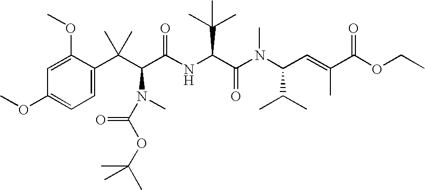

According to the General Procedure IV, to a solution of N-(tert-butoxycarbonyl)-2-methoxy-N,O,β,β-tetramethyl-L-tyrosine (304 mg, 0.83 mmol, from Reference Example 86) and benzotriazole-1-yl-oxy-tris-pyrrolidinophosphonium hexafluorophosphate (560 mg, 1.08 mmol) in dichloromethane (15 mL) is added diisopropylethylamine (0.43 mL, 1.2 mmol) followed by the addition of ethyl (2E,4S)-2,5-dimethyl-4-[methyl(3-methyl-L-valyl)amino]hex-2-enoate hydrochloride (288 mg, 0.83 mmoL). Chromatography (2: 1 hexanes: ethyl acetate) provided 350 mg (63%) of the title compound as a colorless sticky oil. HRMS (ESI) calcd for C₃₆H₅₉N₃O₈ (M+H⁺) 662.4375, found 662.4371.

### REFERENCE EXAMPLE 88

### N-(tert-Butoxycarbonyl)-2-methoxy-N,O,β,β-tetramethyl-L-tyrosyl-N¹-[(1S,2E)-3-carboxy-1-isopropyl-2-butenyl]-N¹,3-dimethyl-L-valinamide

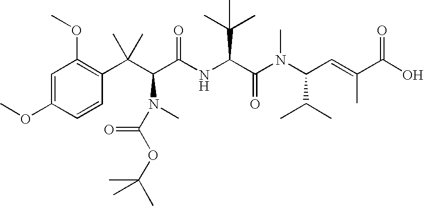

According to General Procedure V, N-(tert-butoxycarbonyl)-2-methoxy-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide (350 mg, 0.53 mmol, from Reference Example 87) is dissolved in methanol (12 mL). To this is added water (4 mL) and 1.0 M lithium hydroxide solution (4.24 mL, 4.24 mmol). Chromatography (1:1: 0.01 hexanes: ether: acetic acid) provided 290 mg (87%) of the title compound as a white solid. MS (ESI) calcd for C₃₄H₅₅N₃O₈ (M+H⁺) 634, found 634.

### REFERENCE EXAMPLE 89

### 3-fluoro-N,β,β-trimethylphenylalanine

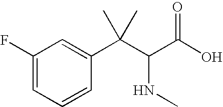

Starting from m-fluorobenzaldehyde, 3-fluoro-N,β,β-trimethylphenylalanine is synthesized by following General Procedures I, II and III. MS (ESI) calcd for C₁₂H₁₆FNO₂ (M+H) 226, found 226.

### REFERENCE EXAMPLE 90a AND 90b

### 3-Fluoro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide and (90b)3-fluoro-N,β,β-trimethyl-D-phenylalanyl-N¹-[(1S,2E)4-ethoxy-1-isopropyl-3-methyl4-oxo-2-butenyl]-N¹,3-dimethyl-L-valinamide

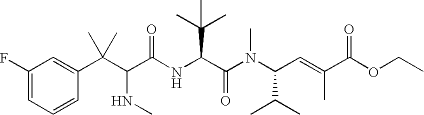

According to the General Procedure IV, to a solution of 3-fluoro-N,β,β-trimethylphenylalanine (235 mg, 1.0 mmol, from Reference Example 89) and benzotriazole-1-yl-oxy-tris-pyrrolidinophosphonium hexafluorophosphate (677 mg, 1.3 mmol) in dichloromethane (10 mL) is added diisopropylethylamine (0.53 mL, 3.0 mmol) followed by the addition of ethyl (2E,4S)-2,5-dimethyl-4-[methyl(3-methyl-L-valyl)amino]hex-2-enoate hydrochloride (348 mg, 1.0 mmoL). Chromatography (Dichloromethane, 9: 1 Dichloromethane: Ethyl acetate) provided 77 mg (15%) of 3-fluoro-N,β,β-trimethyl-L-phenylalanyl-N¹-[(1S,2E)4-ethoxy-1-isopropyl-3-methyl-4-oxo-2-butenyl]-N¹,3-dimethyl-L-valinamide as a white solid and 66 mg (13%) of 3-fluoro-N,β,β-trimethyl-D-phenylalanyl-N¹-[(1S,2E)4-ethoxy-1-isopropyl-3-methyl-4-oxo-2-butenyl]-N¹,3-dimethyl-L-valinamide as a white solid. 3-fluoro-N,β,β-trimethyl-L-phenylalanyl-N¹-[(1S,2E)4-ethoxy-1-isopropyl-3-methyl-4-oxo-2-butenyl]-N¹,3-dimethyl-L-valinamide: HRMS (ESI) calcd for C₂₉H₄₆FN₃O₄ (M+H⁺) 520.3545, found 520.3548; 3-fluoro-N,β,β-trimethyl-D-phenylalanyl-N¹-[(1S,2E)4-ethoxy-1-isopropyl-3-methyl-4-oxo-2-butenyl]-N¹,3-dimethyl-L-valinamide: HRMS (ESI) calcd for C₂₉H₄₆FN₃O₄ (M+H⁺) 520.3545, found 520.3549.

### REFERENCE EXAMPLE 91

### N,β,β-trimethyl-3-(trifluoromethyl)phenylalanine

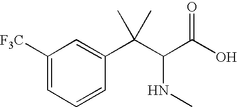

Starting from m-trifluoromethylbenzaldehyde, N,β,β-trimethyl-3-(trifluoromethyl)phenylalanine is synthesized by following General Procedures I, II and III. MS (ESI) calcd for C₁₃H₁₆F₃NO₂ (M+H⁺) 276, found 276.

### REFERENCE EXAMPLE 92a AND 92b

### N,β,β-trimethyl-3-(trifluoromethyl)-L-phenylalanyl-N¹-[(1S,2E)-4-ethoxy-1-isopropyl-3-methyl-4-oxo-2-bute-nyl]-N¹,3-dimethyl-L-valinamide and (92b)N,β,β-trimethyl-3-(trifluoromethyl)-D-phenylalanyl-N¹-[(1S,2E)-4-ethoxy-1-isopropyl-3-methyl-4-oxo-2-bute-nyl]-N¹,3-dimethyl-L-valinamide

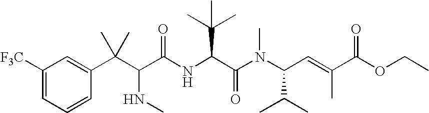

According to the General Procedure IV, to a solution of N,β,β-trimethyl-3-(trifluoromethyl)phenylalanine (295 mg, 1.0 mmol, from Reference Example 91) and benzotriazole-1-yl-oxy-tris-pyrrolidinophosphonium hexafluorophosphate (677 mg, 1.3 mmol) in dichloromethane (10 mL) is added diisopropylethylamine (0.53 mL, 3.0 mmol) followed by the addition of ethyl (2E,4S)-2,5-dimethyl4-[methyl(3-methyl-L-valyl)amino]hex-2-enoate hydrochloride (348 mg, 1.0 mmoL). Chromatography (Dichloromethane, 9: 1 Dichloromethane: Ethyl acetate) provided 127 mg (22%) of N,β,β-trimethyl-3-(trifluoromethyl)-L-phenylalanyl-N¹-[(1S,2E)4-ethoxy-1-isopropyl-3-methyl4-oxo-2-butenyl]-N¹,3-dimethyl-L-valinamide as a white solid and 78 mg (14%) of N,β,β-trimethyl-3-(trifluoromethyl)-D-phenylalanyl-N¹-[(1S,2E)4-ethoxy-1-isopropyl-3-methyl4-oxo-2-butenyl]-N¹,3-dimethyl-L-valinamide as a white solid. N, β,β-trimethyl-3-(trifluoromethyl)-L-phenylalanyl-N¹-[(1S,2E)-4-ethoxy-1-isopropyl-3-methyl-4-oxo-2-butenyl]-N¹,3-dimethyl-L-valinamide: HRMS (ESI) calcd for C₃₀H₄₆F₃N₃O₄ (M+H⁺) 570.3513, found 570.3523; N,β,β-trimethyl-3-(trifluoromethyl)-D-phenylalanyl-N¹-[(1S,2E)4-ethoxy-1-isopropyl-3-methyl-4-oxo-2-butenyl]-N¹,3-dimethyl-L-valinamide: HRMS (ESI) calcd for C₃₀H46F₃N₃O₄ (M+H⁺) 570.3513, found 570.3514.

### REFERENCE EXAMPLE 93

### 3,5-difluoro-N,β,β,-trimethylphenylalanine

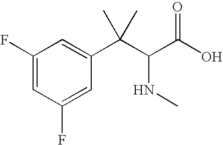

Starting from 3,5-bis-fluorobenzaldehyde, 3,5-difluoro-N,β,β-trimethylphenylalanine is synthesized by following General Procedures I, II and III. HRMS (ESI) calcd for C₁₂H₁₅F₂NO₂ (M+H⁺) 244.1144, found 244.1144.

### REFERENCE EXAMPLE 94a AND 94b

### 3,5-difluoro-N,β,β-trimethyl-L-phenylalanyl-N¹-[(1S,2E)-4-ethoxy-1-isopropyl-3-methyl-4-oxobut-2-enyl]-N,3-dimethyl-L-valinamide and (94b)3,5-difluoro-N,β,β-trimethyl-D-phenylalanyl-N¹-[(1S,2E)-4-ethoxy-1-isopropyl-3-methyl-4-oxobut-2-enyl]-N¹,3-dimethyl-L-valinamide

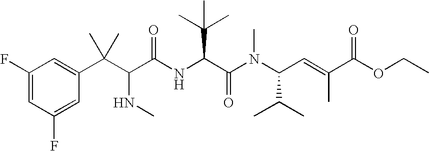

According to the General Procedure IV, to a solution of 3,5-difluoro-N,β,β-trimethylphenylalanine (243 mg, 1.0 mmol, from Reference Example 93) and benzotriazole-1-yl-oxy-tris-pyrrolidinophosphonium hexafluorophosphate (677 mg, 1.3 mmol) in dichloromethane (10 mL) is added diisopropylethylamine (0.53 mL, 3.0 mmol) followed by the addition of ethyl (2E,4S)-2,5-dimethyl-4-[methyl(3-methyl-L-valyl)amino]hex-2-enoate hydrochloride (348 mg, 1.0 mmoL). Chromatography (17: 3 Dichloromethane: Ethyl acetate) provided 112 mg (21%) of 3,5-difluoro-N,β,β-trimethyl-L-phenylalanyl-N¹-[(1S,2E)-4-ethoxy-1-isopropyl-3-methyl4-oxobut-2-enyl]-N¹,3-dimethyl-L-valinamide as a white solid and 90 mg (17%) of 3,5-difluoro-N,β,β-trimethyl-D-phenylalanyl-N¹-[(1S,2E)-4-ethoxy-1-isopropyl-3-methyl4-oxobut-2-enyl]-N¹,3-dimethyl-L-valinamide as a white solid as well as 114 mg (21%) of a mixture of the two. 3,5-difluoro-N,β,β-trimethyl-L-phenylalanyl-N¹-[(1S,2E)-4-ethoxy-1-isopropyl-3-methyl4-oxobut-2-enyl]-N¹,3-dimethyl-L-valinamide: HRMS (ESI) calcd for C₂₉H₄₅F₂N₃O₄ (M+H⁺) 538.3451, found 538.3452; 3,5-difluoro-N,β,β-trimethyl-D-phenylalanyl-N¹-[(1S,2E)-4-ethoxy-1-isopropyl-3-methyl-4-oxobut-2-enyl]-N¹,3-dimethyl-L-valinamide: HRMS (ESI) calcd for C₂₉H₄₅F₂N₃O₄ (M+H⁺) 538.3451, found 538.3446.

### REFERENCE EXAMPLE 95

### N,β,β-trimethyl-3,5-bis(trifluoromethyl)phenylalanine

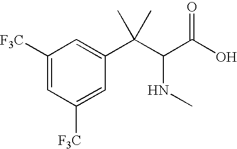

Starting from 3,5-bis-trifluoromethylbenzaldehyde, N,β,β-trimethyl-3,5-bis(trifluoromethyl)phenylalanine is synthesized by following General Procedures I, II and III. HRMS (ESI) calcd for C₁₄H₁₅F₆NO₂ (M+H⁺) 344.1080, found 344.1077.

### REFERENCE EXAMPLE 96a AND 96b

### N,β,β-trimethyl-3,5-bis(trifluoromethyl)-L-phenylalanyl-N¹-[(1S,2E)4-ethoxy-1-isopropyl-3-methyl-4-oxobut-2-enyl]-N¹,3-dimethyl-L-valinamide and (96b) N,β,β-trimethyl-3,5-bis(trifluoromethyl)-D-phenylalanyl-N¹-[(1S,2E)4-ethoxy-1-isopropyl-3-methyl4-oxobut-2-enyl]-N¹,3-dimethyl-L-valinamide

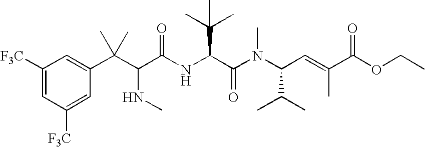

According to the General Procedure IV, to a solution of N,β,β-trimethyl-3,5-bis(trifluoromethyl)phenylalanine (343 mg, 1.0 mmol, from Reference Example 95) and benzotriazole-1-yl-oxy-tris-pyrrolidinophosphonium hexafluorophosphate (677 mg, 1.3 mmol) in dichloromethane (10 mL) is added diisopropylethylamine (0.52 mL, 3.0 mmol) followed by the addition of ethyl (2E,4S)-2,5-dimethyl-4-[methyl(3-methyl-L-valyl)amino]hex-2-enoate hydrochloride (348 mg, 1.0 mmoL). Chromatography (9: 1 Dichloromethane: Ethyl acetate) provided 79 mg (12%) of N,β,β-trimethyl-3,5-bis(trifluoromethyl)-L-phenylalanyl-N¹-[(1S,2E)-4-ethoxy-1-isopropyl-3-methyl-4-oxobut-2-enyl]-N¹,3-dimethyl-L-valinamide as a white solid and 72 mg (11%) of N, β,β-trimethyl-3,5-bis(trifluoromethyl)-D-phenylalanyl-N¹-[(1S,2E)-4-ethoxy-1-isopropyl-3-methyl-4-oxobut-2-enyl]-N¹,3-dimethyl-L-valinamide as a white solid. N,β,β-trimethyl-3,5-bis(trifluoromethyl)-L-phenylalanyl-N¹-[(1S,2E)-4-ethoxy-1-isopropyl-3-methyl-4-oxobut-2-enyl]-N¹,3-dimethyl-L-valinamide: HRMS (ESI) calcd for C₃₁H₄₅F₆N₃O₄ (M+H⁺) 638.3387, found 638.3390; N,β,β-trimethyl-3,5-bis(trifluoromethyl)-D-phenylalanyl-N¹-[(1S,2E)4-ethoxy-1-isopropyl-3-methyl-4-oxobut-2-enyl]-N¹,3-dimethyl-L-valinamide: HRMS (ESI) calcd for C₃₁H45F₆N₃O₄ (M+H⁺) 638.3387, found 638.3379.

### REFERENCE EXAMPLE 97

### O-isopropyl-N,β,β-trimethyltyrosine

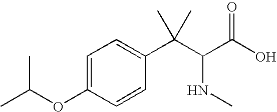

Starting from 4-isopropoxybenzaldehyde, O-isopropyl-N,β,βtrimethyltyrosine is synthesized by following General Procedures I, II and III. MS (ESI) calcd for C₁₅H₂₃NO₃ (M+H⁺) 266, found 266.

### REFERENCE EXAMPLE 98a AND 98b

### O-isopropyl-N,β,β-trimethyl-L-tyrosyl-N¹-[(1S,2E)4-ethoxy-1-isopropyl-3-methyl-4-oxobut-2-enyl]-N¹,3-dimethyl-L-valinamide and (98b)O-isopropyl-N,β,β-trimethyl-D-tyrosyl-N¹-[(1S,2E)-4-ethoxy-1-isopropyl-3-methyl-4-oxobut-2-enyl]-N¹,3-dimethyl-L-valinamide

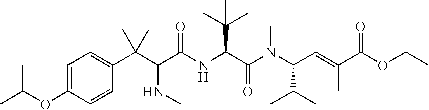

According to the General Procedure IV, to a solution of crude O-isopropyl-N,β,β-trimethyltyrosine (2.0 mmol, from Reference Example 97) and benzotriazole-1-yl-oxy-tris-pyrrolidinophosphonium hexafluorophosphate (1.04 g, 2.0 mmol) in dichloromethane (16 mL) is added diisopropylethylamine (0.78 mL, 4.5 mmol) followed by the addition of ethyl (2E,4S)-2,5-dimethyl-4-[methyl(3-methyl-L-valyl)amino]hex-2-enoate hydrochloride (522 mg, 1.5 mmoL). Chromatography (3:1 Dichloromethane: Ethyl acetate) provided 3 mg of O-isopropyl-N,β,β-trimethyl-L-tyrosyl-N¹-[(1S,2E)-4-ethoxy-1-isopropyl-3-methyl-4-oxobut-2-enyl]-N¹,3-dimethyl-L-valinamide and 5 mg of O-isopropyl-N,β,β-trimethyl-D-tyrosyl-N¹-[(1S,2E)-4-ethoxy-1-isopropyl-3-methyl-4-oxobut-2-enyl]-N¹,3-dimethyl-L-valinamide as well as 80 mg of a mixture of the two. O-isopropyl-N,β,β-trimethyl-L-tyrosyl-N¹-[(1S,2E)-4-ethoxy-1-isopropyl-3-methyl-4-oxobut-2-enyl]-N¹,3-dimethyl-L-valinamide: HRMS (ESI) calcd for C₃₂H₅₃N₃O₅ (M+H⁺) 560.4058, found 560.4059; O-isopropyl-N,β,β-trimethyl-D-tyrosyl-N¹-[(1S,2E)-4-ethoxy-1-isopropyl-3-methyl-4-oxobut-2-enyl]-N¹,3-dimethyl-L-valinamide: HRMS (ESI) calcd for C₃₂H53N₃O₅ (M+H⁺) 560.4058, found 560.4053.

### REFERENCE EXAMPLE 99

### (2S)-2-(tert-Butoxycarbonyl-methyl-amino)-3-cyclohexyl-3-methyl-butyric Acid

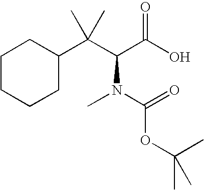

To a solution of (2S)-2-[(tert-butoxycarbonyl)aminol-3-methyl-3-phenylbutanoic acid (3.0 g, 9.76 mmol) in 75 mL glacial acetic acid is added platinum oxide (0.75 g). This mixture is hydrogenated in a Parr apparatus at 50 psi until hydrogen uptake ceased. The mixture is filtered and the used catalyst rinsed with fresh acetic acid. The combined filtrate and wash is evaporated in vacuo and the resulting clear oil is placed under high vacuum overnight. The clear oil slowly crystallized and the product is isolated by trituration with hexanes to give a white solid, 2.13 g (70%) ¹H NMR (CDCl₃, d): 5.00 and 4.86 (combined 1H, vbr singlets), 4.95-3.00 (3H, br, NMe), 1.70-1.83 (4H, br m), 1.61-1.70 (2H, br m), 1.47 (9H, s, t-Bu) over lap with 1.36-1.5 (1H, br m), 1.06 (3H, br s, Me), 0.95 (3H, s, Me) overlap with 0.94-1.32 (4H, m). MS: m/z 312.2 (M+H).

### REFERENCE EXAMPLE 100

### 3-Cyclohexyl-N-methyl-L-valyl-N¹-[(1S,2E)-4-ethoxy-1-isopropyl-3-methyl-4-oxo-2-butenyl]-N¹,3-dimethyl-L-valinamide

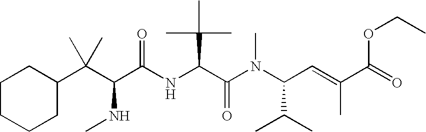

Following General Procedure IVa (2S)-2-(tert-Butoxycarbonyl-methyl-amino)-3-cyclohexyl-3-methyl-butyric acid (0.806 g, 2.62 mmol, from Reference Example 99) is coupled using dichloromethane (5.5 mL), dimethylformamide (5.5 mL), benzotriazole-1-yl-oxy-tris-pyrrolidinophosphonium hexafluorophosphate (1.38 g, 2.62 mmol), ethyl (2E,4S)-2,5-dimethyl4-[methyl(3-methyl-L-valyl)amino]hex-2-enoate (0.875 g, 2.62 mmol) and diisopropylethylamine (1.1 mL, 6.31 mmole). The reaction mixture is evaporated in vacuo and the residue dissolved in 10 mL dichloromethane, treated with 4M hydrogen chloride in dioxane (6 mL) and stirred for 4 hours. The solvents are removed in vacuo and the residue is chromatographed by reverse phase HPLC (0.01% trifluoroacetic acid in water/acetonitrile) to give the title compound as a white solid, 0.845 g. ¹H NMR (DMSOd₆, d): 8.64 (1H, br), 8.53 (1H, d, J=8.1 Hz), 8.38 (1H, br), 6.67 (1H, d, J=9.4 Hz), 4.89 (1H, t, J=10.1 Hz), 4.74 (1H, d, J=8.2 Hz), 4.14 (2H, q, J=6.9 Hz), 4.00 (1H, d, J=9.9 Hz), 2.99 (3H, s), 2.44 (3H, br), 1.95-2.08 (1H, m), 1.81 (3H, s), 1.58-1.79 (5H, m), 1.22 (3H, t, J=7.0 Hz) and 0.95 (9H, s, t-Bu) and 0.93 (3H, s, Me) overlap with 0.85-1.43 (5H, m), 0.79 (3H, d, J=6.3 Hz), 0.74 (3H, d, J=6.4 Hz), 0.70 (3H, s). MS: m/z 508.36 (M+H).

### REFERENCE EXAMPLE 101

### Oxo-(1-phenyl-cyclopentyl)-acetic Acid

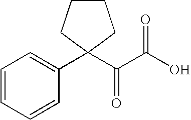

Following General Procedure II phenylpyruvic acid sodium salt (19.4 g, 0.095 mole) is alkylated using 1,4-diiodobutane (32.39 g, 0.105 mole), tetrahydrofuran (80 mL), 5N aqueous sodium hydroxide solution (46 mL, 1.48 mol eq.) and water (31 mL). The title compound is isolated as a yellow gum (9.74 g). MS: m/z 217.2 (M -1)

### REFERENCE EXAMPLE 102

### Methylamino-(1-phenyl-cyclopentyl)-acetic Acid

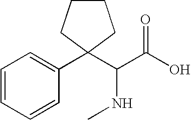

Following General Procedure III, oxo-(1-phenyl-cyclopentyl)-acetic acid (9.74 g, 44.6 mmol, from Reference Example 101) is converted to the title compound (white solid, 5.5 g). MS: m/z 234.3 (M+H).

### REFERENCE EXAMPLE 103

### Ethyl (2E,4S)-2,5-dimethyl-4-(methyl{3-methyl-N-[2-(methylamino)-2-(1-phenylcyclopentyl)ethanoyl]-L-valyl}amino)-2-hexenoate

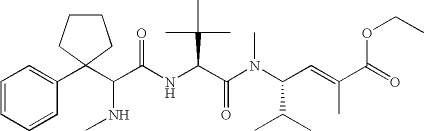

Following General Procedure IV, methylamino-(1-phenyl-cyclopentyl)-acetic acid (0.756 g, 3.24 mmol, from Reference Example 102) is coupled using dichloromethane (5.5 mL), dimethylformamide (5.5 mL), benzotriazole-1-yl-oxy-tris-pyrrolidinophosphonium hexafluorophosphate (1.38 g, 2.62 mmol), ethyl (2E,4S)-2,5-dimethyl-4-[methyl(3-methyl-L-valyl)amino]hex-2-enoate (0.875 g, 2.62 mmol) and diisopropylethylamine (1.1 mL, 6.31 mmole), giving a white solid (85 mg). MS: m/z 528.4 (M+H).

### REFERENCE EXAMPLE 104

### Oxo-(1-phenyl-cyclohexyl)-acetic Acid

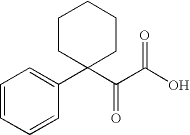

Following General Procedure II, phenylpyruvic acid sodium salt (25.0 g, 0.122 mole) is alkylated using 1,5-diiodopentane (41.38 g, 0.128 mole), tetrahydrofuran (100 mL), 5N aqueous sodium hydroxide solution (60 mL, 1.48 mol eq.) and water (40 mL). The title compound is isolated as a brown oil (9.4 g). MS: m/z 231.1 (M−1)

### REFERENCE EXAMPLE 105

### Methylamino-(1-phenyl-cyclohexyl)-acetic Acid

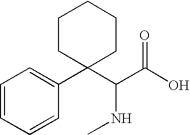

Following General Procedure III oxo-(1-phenyl-cyclohexyl)-acetic (9.4 g, 40.5 mmol, from Reference Example 104) is converted to methylamino-(1-phenyl-cyclohexyl)-acetic acid (white solid, 6.6 g). MS: m/z 248.2 (M+H).

### REFERENCE EXAMPLE 106

### Ethyl (2E,4R)-2,5-dimethyl-4-(methyl{3-methyl-N-[(methylamino)(1-phenylcyclohexyl)acetyl]-L-valyl}amino)-2-hexenoate

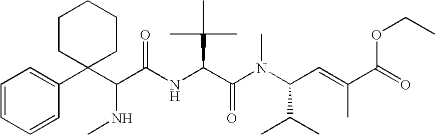

Following General Procedure IV, methylamino-(1-phenyl-cyclohexyl)-acetic acid (0.30 g, 1.21 mmol, from Reference Example 105) is converted to the title compound, giving after chromatography a white solid (50 mg). MS: m/z 542.44 (M+H).

### REFERENCE EXAMPLE 107

### Ethyl (E,4S)-4-[{(2S)-2-[(tert-butoxycarbonyl)amino]-3-phenylpropanoyl}(methyl)amino]-2,5-dimethyl-2-hexenoate

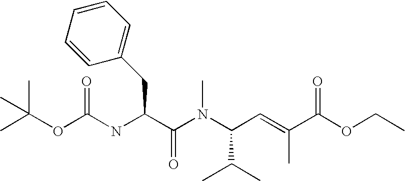

By using a procedure analogous to that described in General Procedure IVa, ethyl (2E,4S)-4-methylamino-2-methyl-5-methyl-2-hexenoate trifluoroacetic acid salt (0.826 mmol) is treated with N-tert-butoxycarbonyl-L-phenylalanine (219 mg, 0.825 mmol, Aldrich) in the presence of benzotriazole-1-yl-oxy-tris-(dimethylamino)-phosphonium hexafluorophosphate (402 mg, 0.91 mmol) and triethylamine (0.345 mL, 2.48 mmol) in methylene chloride (3.5 mL) at room temperature for 24 hours under argon atmosphere. The product is purified by chromatography (silica gel, flash column, 2.5% methanol in methylene chloride), to provide ethyl (E,4S)-4-[{(2S)-2-[(tert-butoxycarbonyl)amino]-3-phenylpropanoyl}(methyl)amino]-2,5-dimethyl-2-hexenoate (284 mg, 77%) as a colorless oil. MS (ES): m/z 447.2 (M+H). IR cm⁻¹: 3301.35, 2975.92, 1711.43, 1633.87.

### REFERENCE EXAMPLE 108

### Ethyl (6S,9S,12S,13E)-9-benzyl-12-isopropyl-2,2,5,11,14-pentamethyl-6-(1-methyl-1-phenylethyl)4,7,10-trioxo-3-oxa-5,8,11-triazapentadec-13-en-15-oate

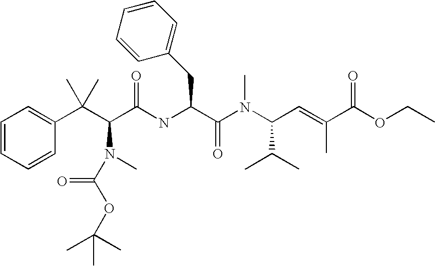

Ethyl (E,4S)4-[{(2S)-2-[(tert-butoxycarbonyl)amino]-3-phenylpropanoyl}(methyl)amino]-2,5-dimethyl-2-hexenoate (175 mg, 0.392 mmol, obtained from Reference Example 107) is treated with a solution of hydrogen chloride in para-dioxane (2 mL, 4 M solution, Aldrich), to give ethyl (E,4S)-4-[{(2S)-2-amino-3-phenylpropanoyl}(methyl)amino]-2,5-dimethyl-2-hexenoate hydrogen chloride. In a manner analogous to that described in General Procedure IVb this material is treated with (2S)-2-[(tert-butoxycarbonyl)(methyl)amino]-3-methyl-3-phenylbutanoic acid (120 mg, 0.392 mmol) in the presence of hydroxybenzotriazole (64 mg, 0.47 mmol), 1-(3-dimethylaminopropyl)-3-ethylcarbodiimide hydrochloride (105 mg, 0.55 mmol), N-methylmorpholine (0.065 mL, 0.59 mmol) in anhydrous N,N-dimethylformamide (5 mL) at room temperature for 15 hours under argon atmosphere. The product is purified by chromatography (silica gel, flash column, 50% ethyl acetate in hexanes), to provide ethyl (6S,9S,12S,13E)-9-benzyl-12-isopropyl-2,2,5,11,14-pentamethyl-6-(1-methyl-1-phenylethyl)-4,7,10-trioxo-3-oxa-5,8,11-triazapentadec-13-en-15-oate (92 mg, 37%) as a pale yellow oil. MS (ES): m/z 635.9 (M+H). IR cm⁻¹: 3326.59, 2973.15, 1692.40, 1649.44. Analytical HPLC: (4.6×150 mm Prodigy ODS3 column eluted with 30 to 70% acetonitrile in water containing 0.02% TFA over 50 minutes-isocratic method): 83.61% of ethyl (6S,9S,12S,13E)-9-benzyl-l 2-isopropyl-2,2,5,11,14-pentamethyl-6-(1-methyl-1-phenylethyl)-4,7,10-trioxo-3-oxa-5,8,11-triazapentadec-13-en-15-oate at 26.1 minutes, and two other diastereomers, 0.87% (at 27.7 minutes) and 15.52% (at 29.0 minutes).

### REFERENCE EXAMPLE 109

### Ethyl (E,4S)-4-[(tert-butoxycarbonyl)(methyl)amino]-2-methyl-2-octenoate

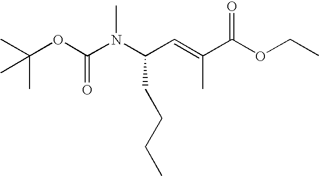

(2S)-2-(N-tert-Butoxycarbonyl-N-methyl)hexanal is prepared in 2 steps via tert-butyl (1S)-1-{[methoxy(methyl)amino]carbonyl}pentyl(methyl)carbamate by using the literature procedure (Andersen, R., WO 96/33211). Reaction between (2S)-2-(N-tert-Butoxycarbonyl-N-methyl)hexanal (1.0 g, 4.359 mmol) and (carboethoxyetylidene)triphenyl phosphorane (2.1 g, 5.667 mmol, Aldrich) in anhydrous methylene chloride (10 mL) is carried out at room temperature for 24 hours under argon atmosphere. The product is purified by chromatography (silica gel, flash column, methylene chloride : ethyl ether: methanol=95:3:2) to provide ethyl (E,4S)-4-[(tert-butoxycarbonyl)(methyl)amino]-2-methyl-2-octenoate (1.1357 g, 79.6%) as a clear oil. MS (ES): m/z 314.3 (M+H). IR cm⁻¹: 2960, 2931, 2872, 1714, 1693. Analysis for C₁₇H₃₁N₁O₄: Calcd: C, 65.14;H, 9.97; N, 4.47. Found: C, 64.82;H, 9.98; N, 4.30. Optical Rotation (MeOH): [α]_{D}=[−34]+4

### REFERENCE EXAMPLE 110

### Ethyl (E,4S)-4-[{(2S)-2-[(tert-butoxycarbonyl)amino]-3,3-dimethylbutanoyl}(methyl)amino]-2-methyl-2-octenoate

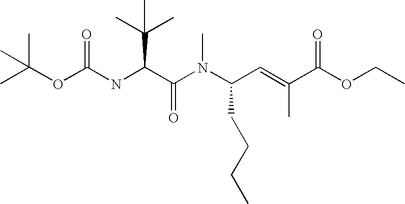

Ethyl (E,4S)-4-[(tert-butoxycarbonyl)(methyl)amino]-2-methyl-2-octenoate (557 mg, 1.70 mmol, obtained from Reference Example 109) is treated with a solution of hydrogen chloride in para-dioxane (26.4 mL, 5.5 mmol; 4 M solution, Aldrich), to give ethyl (E,4S)-4-(N-methylamino)-2-methyl-2-octenoate hydrogen chloride. This material is treated with N-tert-butoxycarbonyl-tert-butylglycine (222 mg, 0.96 mmol, Aldrich) in the presence of hydroxybenzothriazole (129.7 mg, 0.96 mmol), 1-(3-dimethylaminopropyl)-3-ethylcarbodiimide hydrochloride (215 mg, 1.12 mmol), N-methylmorpholine (0.123 mL, 1.2 mmol) in anhydrous N,N-dimethylforrnamide (17 mL) at room temperature for 15 hours under argon atmosphere. The product is purified by chromatography (silica gel, flash column, methylene chloride: ethyl ether: methanol=92:5:3), to provide ethyl (E,4S)-4-[{(2S)-2-[(tert-butoxycarbonyl)amino]-3,3-dimethylbutanoyl}(methyl)amino]-2-methyl-2-octenoate (251.3 mg, 73.6%) as a colorless oil. MS (ES): m/z 853.3 (2M+H), 427.0 (M+H). IR cm⁻¹: 3351, 2961, 2933, 2873, 1715, 1639.

### REFERENCE EXAMPLE 111

### N-(tert-Butoxycarbonyl)-N,β,β-trimethyl-L-phenylalanyl-N¹-[(1S,2E)-1-butyl-4-ethoxy-3-methyl4-oxobut-2-enyl]-N¹,3-dimethyl-L-valinamide

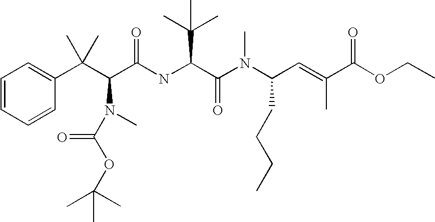

Ethyl (E,4S)-4-[{(2S)-2-[(tert-butoxycarbonyl)amino]-3,3-dimethylbutanoyl}(methyl)amino]-2-methyl-2-octenoate (202.8 mg, 0.48 mmol, obtained from Reference Example 110) is treated with a solution of hydrogen chloride in para-dioxane (1.8 mL, 7.2 mmol, 4 M solution, Aldrich), to give ethyl (E,4S)-4-[{(2S)-2-amino-3,3-dimethylbutanoyl}(methyl)amino]-2-methyl-2-octenoate hydrogen chloride. By using a procedure analogous described in General Procedure lVb, this material is treated with (2S)-2-[(tert-butoxycarbonyl)(methyl)amino]-3-methyl-3-phenylbutanoic acid (177 mg, 0.576 mmol) in the presence of hydroxybenzothriazole (78 mg, 0.576 mmol), 1-(3-dimethylaminopropyl)-3-ethylcarbodiimide hydrochloride (129 mg, 0.672 mmol), N-methylmorpholine (0.08 mL, 0.72 mmol) in anhydrous N,N-dimethylformamide (10 mL) at room temperature for 24 hours under argon atmosphere. The product is purified by chromatography (silica gel, flash column, methylene chloride : ethyl ether: methanol=93:5:2), to provide N-(tert-butoxycarbonyl)-N,β,β-trimethyl-L-phenylalanyl-N¹′-[(1S,2E)-1-butyl-4-ethoxy-3-methyl-4-oxobut-2-enyl]-N¹,3-dimethyl-L-valinamide (233.2 mg, 78.9%) as a clear oily material. MS (ES): m/z 616.5 (M+H). IR cm⁻¹: 3412, 2963, 2932, 2872, 1714, 1684, 1641. Analytical HPLC: (4.6×150 mm Luna C18 column eluted with a liner gradient of 15-100% actonitrile in water containing 0.02% trofluoroactic acid over 35 min): 76.94% (at 23.6 minutes of N-(tert-butoxycarbonyl)-N,β,β-trimethyl-L-phenylalanyl-N¹-[(1S,2E)-1-butyl-4-ethoxy-3-methyl4-oxobut-2-enyl]-N¹,3-dimethyl-L-valinamide, and 9.4% (at 23.9 minutes) of other diastereomer.

### REFERENCE EXAMPLE 112

### N-(tert-Butoxycarbonyl)-N,β,β-trimethyl-L-phenylalanyl-N¹-[(1S,2E)-1-butyl-3-carboxybut-2-enyl]-N¹,3-dimethyl-L-valinamide

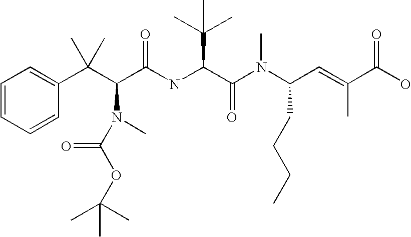

By using a procedure analogous to that described in General Procedure V, N-(tert-butoxycarbonyl)-N,β,β-trimethyl-L-phenylalanyl-N¹-[(1S,2E)-3-(ethoxycarbonyl)-1-n-butyl-2-butenyl]-N¹,3-dimethyl-L-valinamide (151 mg, 0.245 mmol, obtained from Reference Example 111) is treated with lithium hydroxide (1.96 mmol) in water (1.96 mL) and methanol (6 mL) at room temperature for 15 hours, to provide N-(tert-butoxycarbonyl)-N,β,β-trimethyl-L-phenylalanyl-N¹-[(1S,2E)-3-carboxy-1-n-butyl-2-butenyl]-N¹,3-dimethyl-L-valinamide (134.0 mg, 93.1%). MS (ES): m/z 1173.1 (2M−H). IR cm⁻¹: 2964, 2933, 2873, 1693, 1649. Analytical HPLC: (4.6×150 mm Prodigy ODS3 C18 column eluted with a liner gradient of 10-90% actonitrile in water containing 0.02% trifluoroacetic acid over 35 min): 90.26% (at 23.79 minutes) of N-(tert-butoxycarbonyl)-N,β,β-trimethyl-L-phenylalanyl-N¹-[(1S,2E)-3-carboxy-1-n-butyl-2-butenyl]-N¹,3-dimethyl-L-valinamide, and 1.16% (at 24.27 minutes) and 8.55% (at 24.46 minutes) of two other diastereomers.

### REFERENCE EXAMPLE 113

### N²-(tert-Butoxycarbonyl)-N¹-methoxy-N¹,N²-dimethyl-L-leucinamide

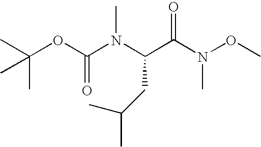

N-(tert-butoxycarbonyl)-L-leucine N,O-dimethylhydroxamide (4 g, 14.6 mmol, Advanced ChemTech) in anhydrous tetrahydrofuran (90 mL) is added to sodium hydride (577 mg, 24 mmol) in tetrahydrofuran (3 mL) and stirred at 0° C. for 20 minutes. The resulting mixture is treated with methyl iodide (1.81 mL, 29 mmol) and stirred at room temperature 16 hours. The product is chromatographed (silica gel, flash column, 20% ethyl acetate in hexanes), to provide N²-(tert-butoxycarbonyl)-N¹-methoxy-N¹,N²-dimethyl-L-leucinamide (2.91 g, 69%) as a yellow oil. MS (ES): m/z 289.2 (M+H). IR cm⁻¹: 2958.97, 1695.11, 1674.43, 1455.77.

### REFERENCE EXAMPLE 114

### Ethyl (2E,4S)-4-[(tert-butoxycarbonyl)(methyl)amino]-2-ethyl-6-methyl-2-heptenoate

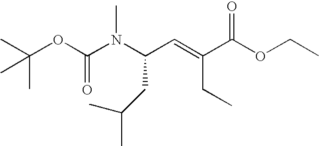

(Carboethoxyethyl)triphenylphosphonium bromide is prepared by using the literature procedure (Ref. Portulas, J.; Sanchez-Ferrando, F.; Sanchez-Pardo, J., Tetrahedron Letts., 1976, 3617-3618), and converted to (carboethoxyetylidene)triphenyl phosphorane (Bestmann, von H. J.; Schulz, H., J. Liebigs Ann. Chem., 1964, 11-17). N-tert-Butoxycarbonyl-N-methyl-L-leucinal is prepared from N²-(tert-butoxycarbonyl)-N¹-methoxy-N¹, N²-dimethyl-L-leucinamide (from Reference Example 113) by treatment with lithium aluminum tetrahydride in anhydrous tetrahydrofuran according to the literature procedure (Andersen, R., WO 96/33211).

Reaction between N-tert-butoxycarbonyl-N-methyl-L-leucinal (500 mg, 2.18 mmol) and (carboethoxyetylidene)triphenyl phosphorane (1.23 g, 3.27 mmol) in anhydrous methylene chloride (5 mL) is carried out at room temperature for 15 hours under argon atmosphere. The product is purified by chromatography (silica gel, flash column, methylene chloride: ethyl ether: methanol=95:3:2), to provide ethyl (2E,4S)-4-[(tert-butoxycarbonyl)(methyl)amino]-2-ethyl-6-methyl-2-heptenoate (270 mg, 38%) as a yellow oil. MS (ES): m/z 481.9 (M+H).

### REFERENCE EXAMPLE 115

### Ethyl (2E,4S)4-[[N-(tert-butoxycarbonyl)-3-methyl-L-valyl](methyl)amino]-2-ethyl-6-methyl-2-heptenoate

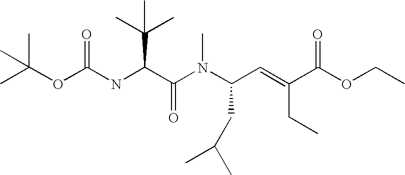

By using a procedure analogous to that described in General Procedure IVb, ethyl (2E,4S)-4-[(tert-butoxycarbonyl)(methyl)amino]-2-ethyl-6-methyl-2-heptenoate (243 mg, 0.742 mmol, obtained from Reference Example 114) is treated with a solution of hydrogen chloride in para-dioxane (2.2 mL, 8.9 mmol, 4 M solution, Aldrich), to give ethyl (2E,4S)-4-methylamino-2-ethyl-6-methyl-2-heptenoate hydrogen chloride. This material is treated with N-tert-butoxycarbonyl-tert-butylglycine (194 mg, 0.84 mmol, Aldrich) in the presence of hydroxybenzotriazole (113 mg, 0.84 mmol), 1-(3-dimethylaminopropyl)-3-ethylcarbodiimide hydrochloride (188 mg, 0.98 mmol), N-methylmorpholine (0.115 mL, 1.05 mmol) in anhydrous N,N-dimethylformamide (3 mL) at room temperature for 15 hours under argon atmosphere. The product is purified by chromatography (silica gel, flash column, 20% ethyl acetate in hexanes), to provide ethyl (2E,4S)-4-[[N-(tert-butoxycarbonyl)-3-methyl-L-valyl](methyl)amino]-2-ethyl-6-methyl-2-heptenoate (128 mg, 39%) as a colorless oil. MS (ES): m/z441.4 (M+H). IR cm⁻: 3358.22, 2961.44, 1715.27, 1639.69, 1493.02. Analytical HPLC: (4.6×150 mm YMC Pack Pro C 18 column eluted with 15 to 100% acetonitrile in water containing 0.02% formic acid over 35 minutes): 88.26% (at 24.6 minutes) of ethyl (2E,4S)4-[[N-(tert-butoxycarbonyl)-3-methyl-L-valyl](methyl)amino]-2-ethyl-6-methyl-2-heptenoate, and 9.23% (at 24.9 minutes) of other diastereomer.

### REFERENCE EXAMPLE 116

### N-(tert-Butoxycarbonyl)-N,β,β-trimethyl-L-phenylalanyl-N¹-[(1S,2E)-3-(ethoxycarbonyl)-1-isobutyl-2-pentenyl]-N¹,3-dimethyl-L-valinamide

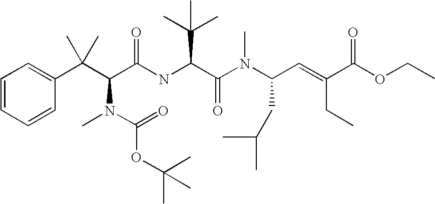

Ethyl (2E,4S)-4-[[N-(tert-butoxycarbonyl)-3-methyl-L-valyl](methyl)amino]-2-ethyl-6-methyl-2-heptenoate (106 mg, 0.24 mmol, obtained from Reference Example 115) is treated with a solution of hydrogen chloride in para-dioxane (1 mL, 4 M solution, Aldrich), to give ethyl (2E,4S)4-[[N-3-methyl-L-valyl](methyl)amino]-2-ethyl-6-methyl-2-heptenoate hydrogen chloride. By using a procedure analogous to that described in General Procedure IVb, this material is treated with (2S)-2-[(tert-butoxycarbonyl)(methyl)amino]-3-methyl-3-phenylbutanoic acid (89 mg, 0.29 mmol, Andersen, R. WO 99/32509) in the presence of hydroxybenzotriazole (39 mg, 0.29 mmol), 1-(3-dimethylaminopropyl)-3-ethylcarbodiimide hydrochloride (65 mg, 0.34 mmol), N-methylmorpholine (0.04 mL, 0.36 mmol) in anhydrous N,N-dimethylformamide (2 mL) at room temperature for 15 hours under argon atmosphere. The product is purified by chromatography (silica gel, flash column), to provide N-(tert-butoxycarbonyl)-N,β,β-trimethyl-L-phenylalanyl-N¹-[(1S,2E)-3-(ethoxycarbonyl)-1-isobutyl-2-pentenyl]-N¹,3-dimethyl-L-valinamide (75 mg, 50%) as a colorless oil. MS (ES): m/z 630.5 (M+H). IR cm⁻¹: 3414.98, 2965.37, 1684.03, 1641.78, 1480.48.

Analytical HPLC: (4.6×150 mm YMC Pack Pro C18 column eluted with 15 to 100% acetonitrile in water containing 0.02% formic acid over 35 minutes): 87.69% (at 29.1 minutes) of N-(tert-butoxycarbonyl)-N,β,β-trimethyl-L-phenylalanyl-N¹-[(1S,2E)-3-(ethoxycarbonyl)-1-isobutyl-2-pentenyl]-N¹,3-dimethyl-L-valinamide, and 11.02% (at 29.6 minutes) of other diastereomer.

### REFERENCE EXAMPLE 117

### Ethyl (E,4S)4-[(tert-butoxycarbonyl)amino]-2-butyl-5-methyl-2-hexenoate

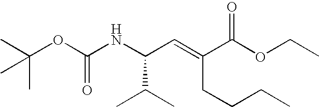

(Carboethoxy-n-butyl)triphenylphosphonium bromide is prepared by using the literature procedure (Ref. Portulas, J.; Sanchez-Ferrando, F.; Sanchez-Pardo, J., Tetrahedron Letts., 1976, 3617-3618), and converted to (carboethoxy-n-butylidene)triphenyl phosphorane (Bestmann, von H. J.; Schulz, H., J. Liebigs Ann. Chem., 1964, 11-17). N-tert-Butoxycarbonyl-L-valinal is prepared in 2 steps from commercially available N-tert-butoxycarbonyl-L-valine via N-α,α-tert-butoxycarbonyl-L-valine-N-methoxy-N-methylamide by using the literature procedure (Andersen, R., WO 96/33211).

N-tert-Butoxycarbonyl-L-valinal (330 mg, 1.6 mmol) and (carboethoxy-n-butylidene)triphenyl phosphorane (1.0 g, 2.47 mmol) in anhydrous methylene chloride (20 mL) is carried out at room temperature for 15 hours under argon atmosphere. The product is purified by chromatography (silica gel, flash column, methylene chloride: ethyl ether: methanol=95: 4: 1) to provide ethyl (E,4S)-4-[(tert-butoxycarbonyl)amino]-2-butyl-5-methyl-2-hexenoate (288.4 mg, 53.7%) as a clear oil. MS (ES): m/z 655.0 (2M+H), 327.9 (M+H). IR cm⁻¹: 3365, 2961, 2932, 2873, 1714.

1H-NMR: (d, CDCl3) 0.884-0.960 (6H, m), 1.298 (3H, t, J=7.11 Hz), 1.423 (9H, s), 1.344-1.454 (4H, m), 1.732-1.788 (1H, m), 2.351-2.448 (2H, m), 4.175 (2H, q), 4.429-4.527 (1H, m), 6.465 (1H, d, J=9.9 Hz).

### REFERENCE EXAMPLE 118

### Ethyl (E,4S)4-({(2S)-2-[(tert-butoxycarbonyl)amino]-3,3-dimethylbutanoyl}amino)-2-butyl-5-methyl-2-hexenoate

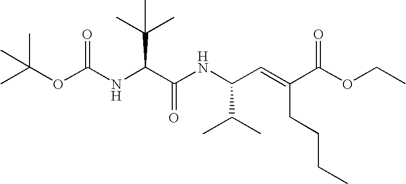

Ethyl (E,4S)4-[(tert-butoxycarbonyl)amino]-2-butyl-5-methyl-2-hexenoate (190 mg, 0.580 mmol, obtained from Reference Example 117) is treated with a solution of hydrogen chloride in para-dioxane (4 M solution, Aldrich), to give ethyl (E,4S)4-amino-2-butyl-5-methyl-2-hexenoate hydrogen chloride. This material is treated with N-tert-butoxycarbonyl-tert-butylglycine (161 mg, 0.696 mmol, Aldrich) in the presence of hydroxybenzothriazole (94.1 mg, 0.696 mmol), 1-(3-dimethylaminopropyl)-3-ethylcarbodiimide hydrochloride (156 mg, 0.812 mmol), N-methylmorpholine (0.096 mL, 0.870 mmol) in anhydrous N,N-dimethylformamide (14 mL) at room temperature for 15 hours under argon atmosphere. The product is purified by chromatography (silica gel, flash column, methylene chloride: ethyl ether: methanol=96:3:1), to provide ethyl (E,4S)-4-({(2S)-2-[(tert-butoxycarbonyl)amino]-3,3-dimethylbutanoyl}amino)-2-butyl-5-methyl-2-hexenoate (250 mg, 100%) as a pale yellow oil. MS (ES): m/z 881.2 (2M+H), 441.0 (M+H). IR cm⁻¹: 3325, 2962, 2934, 2874, 1781, 1714, 1671 Optical Rotation (MeOH): [α]_{D}=[+29±16

### REFERENCE EXAMPLE 119

### Ethyl (6S,9S,12S,13E)-14-butyl-9-(tert-butyl)-12-isopropyl-2,2,5-trimethyl-6-(1-methyl-1-phenylethyl)4,7,10-trioxo-3-oxa-5,8,11-triazapentadec-13-en-15-oate

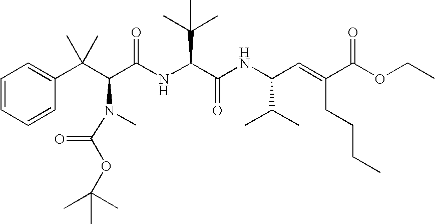

Ethyl (E,4S)4-({(2S)-2-[(tert-butoxycarbonyl)amino]-3,3-dimethylbutanoyl}amino)-2-butyl-5-methyl-2-hexenoate (141.0 mg, 0.320 mmol, obtained from Reference Example 118) is treated with a solution of hydrogen chloride in para-dioxane (0.8 mL, 4 M solution, Aldrich), to give ethyl (E,4S)-4-[(2S)-2-amino-3,3-(dimethylbutanoyl)amino]-2-butyl-5-methyl-2-hexenoate hydrogen chloride. By using a procedure analogous described in General Procedure IVb, this material is treated with (2S)-2-[(tert-butoxycarbonyl)(methyl)amino]-3-methyl-3-phenylbutanoic acid (118 mg, 0.384 mmol, Andersen, R. 99/32509) in the presence of hydroxybenzothriazole (52 mg, 0.384 mmol), 1-(3-dimethylaminopropyl)-3-ethylcarbodiimide hydrochloride (86 mg, 0.448 mmol), N-methylmorpholine (0.053 mL, 0.480 mmol) in anhydrous N,N-dimethylformamide (10 mL) at room temperature for 15 hours under argon atmosphere. The product is purified by chromatography (silica gel, flash column, methylene chloride: ethyl ether: methanol=94:5:1), to provide ethyl (6S,9S,12S,13E)-14-butyl-9-(tert-butyl)-12-isopropyl-2,2,5-trimethyl-6-(1-methyl-1-phenylethyl)-4,7,10-trioxo-3-oxa-5,8,11-triazapentadec-13-en-15-oate (200 mg, 100 %) as a pale yellow oil. MS (ES): m/z 1259.6 (2M+H), 630.3 (M+H). IR cm⁻¹: 3347, 2963, 2934, 2873, 1666.

Optical Rotation (MeOH): [α]_{D}=[−42]16

### REFERENCE EXAMPLE 120

### (6S,9S,12S,13E)-14-Butyl-9-(tert-butyl)-12-isopropyl-2,2,5-trimethyl-6-(I -methyl-1-phenylethyl)4,7,10-trioxo-3-oxa-5,8,11-triazapentadec-13-en-15-oic Acid

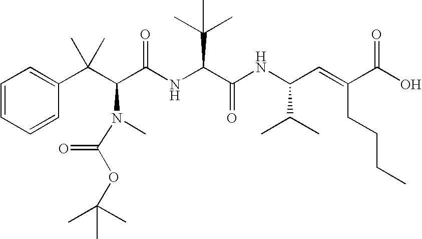

By using a procedure analogous to that described in General Procedure V, ethyl (6S,9S,12S,13E)-14-butyl-9-(tert-butyl)-12-isopropyl-2,2,5-trimethyl-6-(1-methyl-1-phenylethyl)-4,7,10-trioxo-3-oxa-5,8,11-triazapentadec-13-en-15-oate (152 mg, 0.2412 mmol, obtained from Reference Example 119) is treated with lithium hydroxide (1.93 mmol) in water (1.93 mL) and methanol (5.8 mL) at room temperature for 15 hours, to provide (6S,9S,12S,13E)-14-butyl-9-(tert-butyl)-12-isopropyl-2,2,5-trimethyl-6-(1-methyl-1-phenylethyl)-4,7,10-trioxo-3-oxa-5,8,11-triazapentadec-13-en-15-oic acid (104 mg, 74%) as an clear oil. MS (ES): m/z 335.2 (M+H).

### REFERENCE EXAMPLE 121

### Ethyl (2E,4S)4-[(tert-butoxycarbonyl)(methyl)amino]-2-ethyl-5-methyl-2-hexenoate

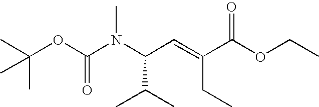

(Carboethoxyethyl)triphenylphosphonium bromide is prepared by using the literature procedure (Ref. Portulas, J.; Sanchez-Ferrando, F.; Sanchez-Pardo, J., Tetrahedron Letts., 1976, 3617-3618), and converted to (carboethoxyetylidene)triphenyl phosphorane (Bestmann, von H. J.; Schulz, H., J. Liebigs Ann. Chem., 1964, 11-17). N-tert-Butoxycarbonyl-N-methyl-L-valinal is prepared in 2 steps from commercially available N-tert-butoxycarbonyl-N-methyl-L-valine via N-α,α-tert-butoxycarbonyl-N-methyl-L-valine-N-methoxy-N-methylamide by using the literature procedures (Andersen, R., WO 96/33211). Reaction between N-tert-butoxycarbonyl-N-methyl-L-valinal (488.7 mg, 2.27 mmol) and (carboethoxyetylidene)triphenyl phosphorane (1.1 g, 2.95 mmol) in anhydrous methylene chloride (8 mL) is carried out at room temperature for 15 hours under argon atmosphere. The product is purified by chromatography (silica gel, flash column, methylene chloride: ethyl ether: methanol=95:3:2), to provide ethyl (2E,4S)-4-[(tert-butoxycarbonyl)(methyl)amino]-2-ethyl-5-methyl-2-hexenoate (523.0 mg, 67.8%) as a colorless oil. MS (ES): m/z 314.0 (M+H). IR cm⁻¹: 2970, 2936, 2875, 1713, 1693. Optical Rotation (MeOH): [α]_{D}=[−72±]2

### REFERENCE EXAMPLE 122

### Ethyl (2E,4S)-4-[[N-(tert-butoxycarbonyl)-3-methyl-L-valyl](methyl)amino]-2-ethyl-5-methyl-2-hexenoate

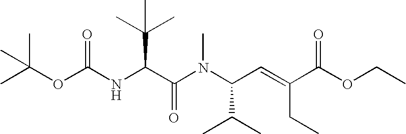

By using a procedure analogous to that described in General Procedure IVb, ethyl (2E,4S)-4-[(tert-butoxycarbonyl)(methyl)amino]-2-ethyl-5-methyl-2-hexenoate (384.3 mg, 1.226 mmol, obtained from Reference Example 121) is treated with a solution of hydrogen chloride in para-dioxane (4 M solution, Aldrich), to give ethyl (2E,4S)-4-N-methylamino-2-ethyl-5-methyl-2-hexenoate hydrogen chloride. This material is treated with N-tert-butoxycarbonyl-tert-butylglycine (340.3 mg, 0.1.47 mmol, Aldrich) in the presence of hydroxybenzothriazole (199 mg, 1.47 mmol), 1-(3-dimethylaminopropyl)-3-ethylcarbodiimide hydrochloride (329 mg, 1.72 mmol), N-methylmorpholine (0.2 mL, 1.839 mmol) in anhydrous N,N-dimethylformamide (10 mL) at room temperature for 15 hours under argon atmosphere. The product is purified by chromatography (silica gel, flash column, methylene chloride: ethyl ether: methanol=95:4:1), to provide ethyl (2E,4S)-4-[[N-(tert-butoxycarbonyl)-3-methyl-L-valyl](methyl)amino]-2-ethyl-5-methyl-2-hexenoate (330 mg, 65.2%) as a colorless oil. MS (ES): m/z 427.0 (M+H). IR cm⁻¹: 2967, 2936, 2875, 1716, 1686.

### REFERENCE EXAMPLE 123

### N-(tert-Butoxycarbonyl)-N,β,β-trimethyl-L-phenylalanyl-N¹-[(1S,2E)-3-(ethoxycarbonyl)-1-isopropyl-2-pentenyl]-N¹,3-dimethyl-L-valinamide

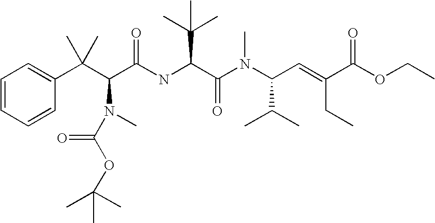

Ethyl (2E,4S)-4-[[N-(tert-butoxycarbonyl)-3-methyl-L-valyl](methyl)amino]-2-ethyl-5-methyl-2-hexenoate (250 mg, 0.606 mmol, obtained from Reference Example 122) is treated with a solution of hydrogen chloride in para-dioxane (2.3 mL, 4 M solution, Aldrich), to give ethyl (2E,4S)-4-[[N-methyl-L-valyl](methyl)amino]-2-ethyl-5-methyl-2-hexenoate hydrogen chloride. By using a procedure analogous described in General Procedure IVb, this material is treated with (2S)-2-[(tert-butoxycarbonyl)(methyl)amino]-3-methyl-3-phenylbutanoic acid (223.4 mg, 0.727 mmol) in the presence of hydroxybenzothiazole (98.3 mg, 0.727 mmol), 1-(3-dimethylaminopropyl)-3-ethylcarbodiimide hydrochloride (162.6 mg, 0.848 mmol), N-methylmorpholine (0.1 mL, 0.909 mmol) in anhydrous N,N-dimethylformamide (10 mL) at room temperature for 15 hours under argon atmosphere. The product is purified by chromatography (silica gel, flash column, methylene chloride: ethyl ether: methanol=95:4:1), to provide N-(tert-butoxycarbonyl)-N,β,β-trimethyl-L-phenylalanyl-N¹-[(1S,2E)-3-(ethoxycarbonyl)-1-isopropyl-2-pentenyl]-N¹,3-dimethyl-L-valinamide (295.6 mg, 79.2%) as a colorless amorphous solid. MS (ES): m/z 616.4 (M+H). IR cm⁻: 2963, 2934, 2873, 1714, 1666. Analytical HPLC (4.6×150 mm Prodigy ODS3 column eluted with a liner gradient of 20-100% actonitrile in water containing 0.02% trofluoroactic acid over 35 min): 69.4% (at 20.69 minutes) of N-(tert-butoxycarbonyl)-N,β,β-trimethyl-L-phenylalanyl-N¹-[(1S,2E)-3-(ethoxycarbonyl)-1-isopropyl-2-pentenyl]-N¹,3-dimethyl-L-valinamide, and 7.8% of the isomer (at 21.389 minutes).

### REFERENCE EXAMPLE 124

### N-(tert-Butoxycarbonyl)-N,β,β-trimethyl-L-phenylalanyl-N¹-[(1S,2E)-3-carboxy-1-isopropyl-2-pentenyl]-N¹,3-dimethyl-L-valinamide

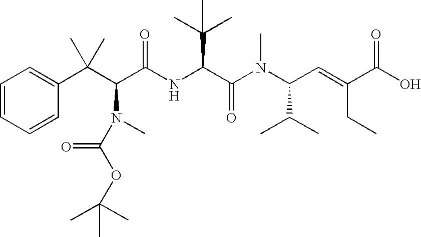

By using a procedure analogous to that described in General Procedure V, N-(tert-butoxycarbonyl)-N,β,β-trimethyl-L-phenylalanyl-N¹-[(1S,2E)-3-(ethoxycarbonyl)-1-isopropyl-2-pentenyl]-N¹,3-dimethyl-L-valinamide (250.9 mg, 0.4074 mmol, obtained from Reference Example 123) is treated with lithium hydroxide (3.3 mmol) in water (3.3 mL) and methanol (9.8mL) at room temperature for 15 hours, to provide N-(tert-butoxycarbonyl)-N,β,β-trimethyl-L-phenylalanyl-N¹-[(IS,2E)-3-carboxy-1-isopropyl-2-pentenyl]-N¹,3-dimethyl-L-valinamide (239 mg, 100%) as a white amorphous solid. MS (ES): m/z 588.4 (M+H). IR cm¹: 2968, 2936, 2876, 1714, 1691.

Analytical HPLC (4.6×150 mm Prodigy ODS3 column eluted with a liner gradient of 15-100% actonitrile in water containing 0.02% formic acid over 35 min) 53.5% (at 23.4 minutes) of N-(tert-butoxycarbonyl)-N,β,β-trimethyl-L-phenylalanyl-N¹-[(1S,2E)-3-carboxy-1-isopropyl-2-pentenyl]-N¹,3-dimethyl-L-valina, and 1.24% (23.7 minutes) and 4.32% (24.0 minutes) of two other diastereomers.

### REFERENCE EXAMPLE 125

### Ethyl (2E,4S)-4-[[N-(tert-butoxycarbonyl)-3-(methylsulfanyl)-L-valyl](methyl)amino]-2,5-dimethyl-2-hexenoate

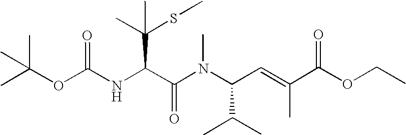

By a method analogous to General Procedure IVa, to a solution of N-t-Boc-S-methyl-L-penicillamine dicyclohexylammonium salt (780 mg, 1.75 mmol) and benzotriazole-1-yl-oxy-tris-pyrrolidinophosphonium hexafluorophosphate (1.187 g, 2.28 mmol) in dichloromethane (18 mL) is added diisopropylethylamine (0.92 mL, 5.26 mmol) followed by the addition of ethyl (2E,4S)-2,5-dimethyl-4-methylamino-hex-2-enoate hydrochloride (412 mg, 1.75 mmoL). The reaction mixture is stirred at room temperature for 36 hours, quenched with saturated aqueous sodium bicarbonate and extracted with dichloromethane. The combined organic layers are washed with water, brine and dried (sodium sulfate). After filtration and concentration, the residue is purified by silica gel column (4: 1 hexanes : ethyl acetate) to give 713 mg (91%) of the title compound as a pale-yellow oil. MS (ESI) calcd for C₂₂H₄₀N₂O₅S (M+H⁺) 445, found 445.

### REFERENCE EXAMPLE 126

### Ethyl (E,4S)-4-[[(2R)-2-amino-3-methyl-3-(methylsulfanyl)butanoyl](methyl)amino]-2,5-dimethyl-2-hexenoate

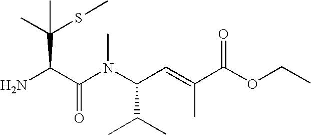

Ethyl (2E,4S)-4-[[N-(tert-butoxycarbonyl)-3-(methylsulfanyl)-L-valyl](methyl)amino]-2,5-dimethyl-2-hexenoate (680 mg, 1.53 mmol, from Reference Example 125) is treated with 4N hydrochloric acid/dioxane (5 mL) at room temperature for 4 hours, then concentrated to dryness. The residual solid is washed with ether and dried in vacuo to give 576 mg (99%) of title compound as a white solid. HRMS (ESI) calcd for C₁₇H₃₂N₂O₃S (M+H⁺) 345.2206, found 345.2201.

### REFERENCE EXAMPLE 127

### Ethyl (6S,9R,12S,13E)-12-isopropyl-2,2,5,11,14-pentamethyl-9-[l -methyl-1-(methylsulfanyl)ethyl]-6-(1-methyl-1-phenylethyl)-4,7,10-trioxo-3-oxa-5,8,11-triazapentadec-13-en-15-oate

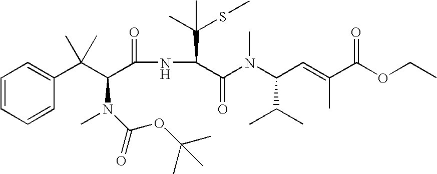

According to the General Procedure IVa, to a solution of (2S)-2-[(tert-butoxycarbonyl)(methyl)amino]-3-methyl-3-phenylbutanoic acid (466 mg, 1.52 mmol) and benzotriazole-1-yl-oxy-tris-pyrrolidinophosphonium hexafluorophosphate (1.025 g, 1.97 mmol) in dichloromethane (15 mL) is added diisopropylethylamine (0.8 mL, 4.55 mmol) followed by ethyl (E,4S)-4-[[(2R)-2-amino-3-methyl-3-(methylsulfanyl)butanoyl](methyl)amino]-2,5-dimethyl-2-hexenoate hydrochloride (576 mg, 1.52 mmoL, from Reference Example 126). Chromatography on silica gel (4:1 hexanes: ethyl acetate provided 650 mg (68%) of the title compound as a pale-yellow oil. HRMS (ESI) calcd for C₃₄H₅₅N₃O₆S (M+H⁺) 634.3884, found 634.3882.

### REFERENCE EXAMPLE 128

### (6S,9R,12S,13E)-12-isopropyl-2,2,5,11,14-pentamethyl-9-[1-methyl-1-(methylsulfanyl)ethyl]-6-(1-methyl-1-phenylethyl)-4,7,10-trioxo-3-oxa-5,8,11-triazapentadec-13-en-15-oic Acid

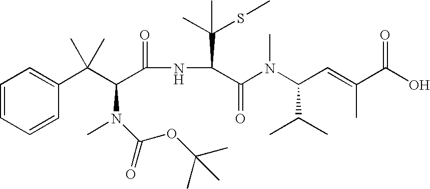

According to General Procedure V, ethyl (6S,9R,12S,13E)-12-isopropyl-2,2,5,11,14-pentamethyl-9-[1-methyl-1-(methylsulfanyl)ethyl]-6-(1-methyl-1-phenylethyl)-4,7,10-trioxo-3-oxa-5,8,11-triazapentadec-13-en-15-oate (275 mg, 0.43 mmol, from Reference Example 127) in methanol (10 mL) and water (3.3 mL) is treated with 1.0 M aqueous lithium hydroxide (3.5 mL, 3.5 mmol) to give 250 mg (95%) of the title compound as a white solid. HRMS (ESI) calcd for C₃₂H₅₁N₃O₆S (M+H⁺) 606.3571, found 606.3560.

### REFERENCE EXAMPLE 129a AND 129b

### N-(tert-butoxycarbonyl)-N,β,β-trimethyl-L-phenylalanyl-N¹-[(1S,2E)-3-carboxy-1-isopropyl-2-butenyl]-N¹-methyl-3-(methylsulfonyl)-L-valinamide and (129b) N-(tert-butoxycarbonyl)-N,β,β-trimethyl-L-phenylalanyl-N¹-[(IS,2E)-3-carboxy-1-isopropyl-2-butenyl]-N¹-methyl-3-(methylsulfinyl)-L-valinamide

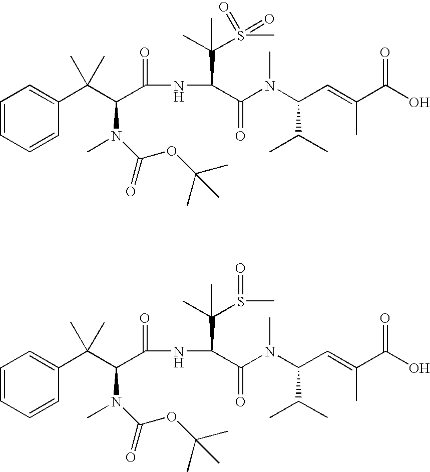

(6S,9R,12S,13E)-12-Isopropyl-2,2,5,11,14-pentamethyl-9-[1-methyl-1-(methylsulfanyl)ethyl]-6-(1-methyl-1-phenylethyl)-4,7,10-trioxo-3-oxa-5,8,11-triazapentadec-13-en-15-oic acid (61 mg, 0.1 mmol, from Reference Example 128) in dichloromethane (2 mL) is treated with 3-chloroperbenzoic acid (34 mg, 0.15 mmol) at room temperature for 3 hours. Chromatography (silica gel, 9:1 ether:HOAc) gave 40 mg (63%) of N-(tert-butoxycarbonyl)-N,β,β-trimethyl-L-phenylalanyl-N¹-[(1S,2E)-3-carboxy-1-isopropyl-2-butenyl]-N¹-methyl-3-(methylsulfonyl)-L-valinamide as a white solid (MS (ESI) calcd for C₃₂H₅₁N₃O₈S (M−H⁺) 636, found 636) and 18 mg (29%) of N-(tert-butoxycarbonyl)-N,β,β-trimethyl-L-phenylalanyl-N¹-[(1S,2E)-3-carboxy-1-isopropyl-2-butenyl]-N¹-methyl-3-(methylsulfinyl)-L-valinamide as a white solid (MS (ESI) calcd for C₃₂H₅₁N₃O₇S (M+H⁺) 622, found 622).

### REFERENCE EXAMPLE 130

### Ethyl (2E,4R)4-[{N-(tert-butoxycarbonyl)-3-[(4-methoxybenzyl)sulfanyl]-L-valyl}(methyl)amino]-2,5-dimethyl-2-hexenoate

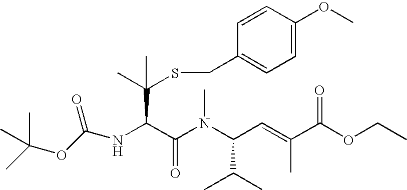

By a method analogous to General Procedure IVa, to a solution of N-t-Boc-S-(p-methoxybenzyl)-L-penicillamine (185 mg, 0.5 mmol) and benzotriazole-1-yl-oxy-tris-pyrrolidinophosphonium hexafluorophosphate (338 mg, 0.65 mmol) in dichloromethane (5 mL) is added diisopropylethylamine (0.26 mL, 1.5 mmol) and ethyl (2E,4S)-2,5-dimethyl4-methylamino-hex-2-enoate hydrochloride (118 mg, 0.5 mmoL). Chromatography gave 235 mg (85%) of the title compound as a colorless oil. MS (ESI) calcd for C₂₉H₄₆N₂O₆S (M+H⁺) 551, found 551.

### REFERENCE EXAMPLE 131

### Ethyl (2E,4R)-4-[{3-[(4-methoxybenzyl)sulfanyl]-L-valyl}(methyl)amino]-2,5-dimethyl-2-hexenoate

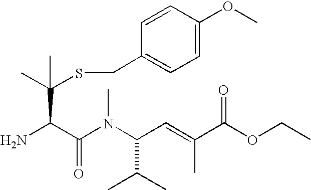

Ethyl(2E,4R)-4-[{N-(tert-butoxycarbonyl)-3-[(4-methoxybenzyl)sulfanyl]-L-valyl}(methyl)amino]-2,5-dimethyl-2-hexenoate (210 mg, 0.38 mmol, from Reference Example 130) is treated with 4N hydrochloric acid/dioxane (2.5 mL) at room temperature for 4 hours, producing 190 mg (100%) of the hydrochloric acid salt of the title compound as a white solid. HRMS (ESI) calcd for C₂₄H₃₈N₂O₄S (M+H⁺) 451.2625, found 451.2635.

### REFERENCE EXAMPLE 132

### N-(tert-butoxycarbonyl)-N,β,β-trimethyl-L-phenylalanyl-N¹-[(1R,2E)-4-ethoxy-1-isopropyl-3-methyl-4-oxo-2-butenyl]-3-[(4-methoxybenzyl)-sulfanyl]-N¹-methyl-L-valinamide

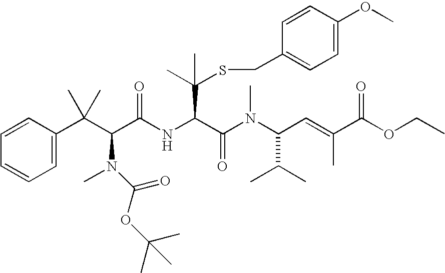

According to the General Procedure IV, to a solution of (2S)-2-[(tert-butoxycarbonyl)(methyl)amino]-3-methyl-3-phenylbutanoic acid (117 mg, 0.38 mmol) and benzotriazole-1-yl-oxy-tris-pyrrolidinophosphonium hexafluorophosphate (258 mg, 0.5 mmol) in dichloromethane (5 mL) is added diisopropylethylamine (0.2 mL, 1.15 mmol) and ethyl (2E,4R)-4-[{3-[(4-methoxybenzyl)sulfanyl]-L-valyl}(methyl)amino]-2,5-dimethyl-2-hexenoate (172 mg, 0.35 mmol, from Reference Example 131). Chromatography gave 216 mg (77%) of the title compound as a white solid. MS (ESI) calcd for C₄₁H₆₁N₃O₇S (M+H⁺) 740, found 740.

### REFERENCE EXAMPLE 133

### N-(tert-butoxycarbonyl)-N,β,β-trimethyl-L-phenylalanyl-N¹-[(1R,2E)-3-carboxy-1-isopropyl-2-butenyl]-3-[(4-methoxybenzyl)sulfanyl]-N¹-methyl-L-valinamide

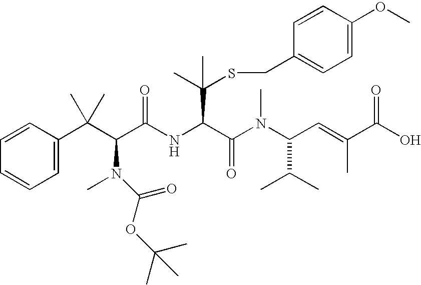

According to General Procedure V, N-(tert-butoxycarbonyl)-N,β,β-trimethyl-L-phenylalanyl-N¹-[(1R,2E)-4-ethoxy-1-isopropyl-3-methyl-4-oxo-2-butenyl]-3-[(4-methoxybenzyl)-sulfanyl]-N¹-methyl-L-valinamide (74 mg, 0.1 mmol, from Reference Example 132) in methanol (3 mL) and water (1 mL) is treated with 1.0 M aqueous lithium hydroxide (1 mL, 1 mmol) to give 60 mg (85%) of the title compound as a white solid. MS (ESI) calcd for C₃₉H₅₇N₃O₇S (M+H⁺) 712, found 712.

### REFERENCE EXAMPLE 134

### N,β,β-trimethyl-L-phenylalanyl-N¹-[(1R,2E)4-ethoxy-1-isopropyl-3-methyl-4-oxo-2-butenyl]-3-[(4-methoxybenzyl)sulfanyl]-N¹-methyl-L-valinamide

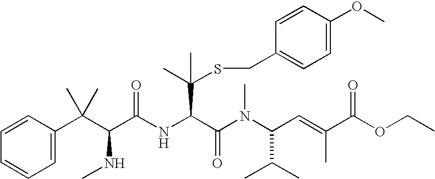

N-(tert-butoxycarbonyl)-N,β,β-trimethyl-L-phenylalanyl-N¹-[(1R,2E)-3-carboxy-1-isopropyl-2-butenyl]-3-[(4-methoxybenzyl)sulfanyl]-N¹-methyl-L-valinamide (30 mg, 0.04 mmol, from Reference Example 133) is treated with 4N hydrochloric acid/dioxane (1 mL), to give 21 mg (77%) of the title compound as a white solid. HRMS (ESI) calcd for C₃₆H₅₃N₃O₅S (M+H⁺) 640.3779, found 640.3758.

### REFERENCE EXAMPLE 135

### N-(tert-butoxycarbonyl)-N,O,β,β-tetramethyl-L-tyrosyl-N¹-[(1S,2E)-4-ethoxy-1-isopropyl-3-methyl-4-oxo-2-butenyl]-3-[(4-methoxybenzyl)sulfanyl]-N¹-methyl-L-valinamide

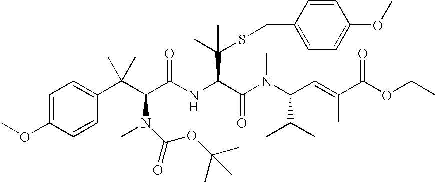

According to the General Procedure IV, to a solution of N-(tert-butoxycarbonyl)-N,O, β,β,-tetramethyl-L-tyrosine (152 mg, 0.4 mmol, from Reference Example 81) and benzotriazole-1-yl-oxy-tris-pyrrolidinophosphonium hexafluorophosphate (271 mg, 0.52 mmol) in dichloromethane (5 mL) is added diisopropylethylamine (0.21 mL, 1.2 mmol) and ethyl (2E,4R)-4-[{3-[(4-methoxybenzyl)sulfanyl]-L-valyl}(methyl)amino]-2,5-dimethyl-2-hexenoate hydrochloride (135 mg, 0.4 mmoL, from Reference Example 131). Chromatography (2:1 hexanes: ethyl acetate) provided 225 mg (85%) of the title compound as a white solid. HRMS (ESI) calcd for C₄₂H₆₃N₃O₈S (M+H⁺) 770.4409, found 770.4395.

### REFERENCE EXAMPLE 136

### N-(tert-butoxycarbonyl)-N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide

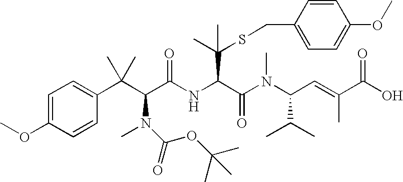

According to General Procedure V, N-(tert-butoxycarbonyl)-N,O,β,β-tetramethyl-L-tyrosyl-N¹-[(1S,2E)-4-ethoxy-1-isopropyl-3-methyl-4-oxo-2-butenyl]-3-[(4-methoxybenzyl)sulfanyl]-N¹-methyl-L-valinamide (177 mg, 0.27 mmol, from Reference Example 135) is dissolved in methanol (7.0 mL) and water (2.2 mL) and treated with 1.0 M aqueous lithium hydroxide (2.3 mL, 2.3 mmol) to give 156 mg (92%) of the title compound as a white solid. MS (ESI) calcd for C₄₀H₅₉N₃O₈S (M+H⁺) 743, found 743.

### REFERENCE EXAMPLE 137

### N,β,β-trimethyl-L-phenylalanyl-N¹-[(1S,2E)-4-ethoxy-1-isopropyl-3-methyl-4-oxo-2-butenyl]-N¹,O-dimethyl-L-allothreoninamide

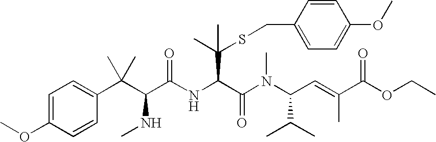

N-(tert-butoxycarbonyl)-N,O,β,β-tetramethyl-L-tyrosyl-N¹-[(1S,2E)-4-ethoxy-1-isopropyl-3-methyl-4-oxo-2-butenyl]-3-[(4-methoxybenzyl)sulfanyl]-N¹-methyl-L-valinamide (34 mg, 0.051 mmol, from Reference Example 135) is treated with 4N hydrochloric acid/dioxane (1.5 mL) at room temperature for 2 hours, then concentrated in vacuo. The residue is washed with ether (3×1 mL) and dried in vacuo to give 21 mg (68%) of the title compound as a white solid. MS (ESI) calcd for C₃₇H₅₅N₃O₆S (M+H⁺) 671, found 671.

### REFERENCE EXAMPLE 138

### N-(tert-butoxycarbonyl)-N,O,β,β-tetramethyl-L-tyrosyl-N¹-[(1S,2E)-4-ethoxy-1-isopropyl-3-methyl-4-oxo-2-butenyl]-N¹-methyl-3-(methylsulfanyl)-L-valinamide

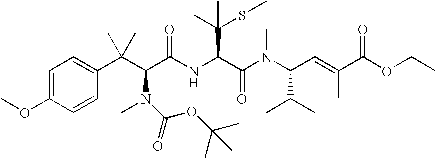

According to the General Procedure IV, to a solution of N-(tert-butoxycarbonyl)-N,O, β,β-tetramethyl-L-tyrosine (121 mg, 0.36 mmol, from Reference Example 81) and benzotriazole-1-yl-oxy-tris-pyrrolidinophosphonium hexafluorophosphate (244 mg, 0.47 mmol) in dichloromethane (6 mL) is added diisopropylethylamine (0.19 mL, 1.08 mmol) and ethyl (2E,4S)-4-[[(2R)-2-amino-3-methyl-3-(methylsulfanyl)butanoyl](methyl)amino]-2,5-dimethyl-2-hexenoate (137 mg, 0.36 mmol, from Reference Example 126). Chromatography (4:1 hexanes: ethyl acetate) provided 154 mg (64%) of the title compound as a white solid. HRMS (ESI) calcd for C₃₅H₅₇N₃O₇S (M+H⁺) 664.3990, found 664.3981.

### REFERENCE EXAMPLE 139

### N-(tert-butoxycarbonyl)-N,O,β,β-tetramethyl-L-tyrosyl-N¹-[(1S,2E)-3-carboxy-1-isopropyl-2-butenyl]-N¹-methyl-3-(methylsulfanyl)-L-valinamide

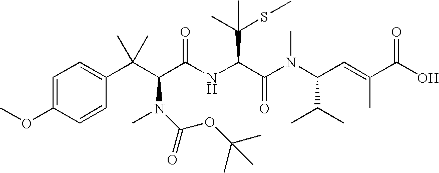

According to General Procedure V, N-(tert-butoxycarbonyl)-N,O,β,β-tetramethyl-L-tyrosyl-N¹-[(1S,2E)-4-ethoxy-1-isopropyl-3-methyl-4-oxo-2-butenyl]-N¹-methyl-3-(methylsulfanyl)-L-valinamide (135 mg, 0.204 mmol, from General Procedure 138) is dissolved in methanol (5.0 mL). and water (1.6 mL) and treated with 1.0 M aqueous lithium hydroxide (1.6 mL, 1.6 mmol) to give 126 mg (97%) of the title compound as a white solid. MS (ESI) calcd for C₄₀H₅₉N₃O₈S (M+H⁺) 743, found 743.

### REFERENCE EXAMPLE 140

### Ethyl (2E,4S)-4-[[N-(tert-butoxycarbonyl)-L-allothreonyl](methyl)amino]-2,5-dimethyl-2-hexenoate

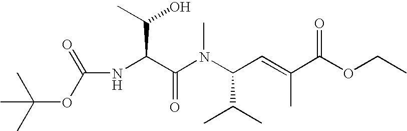

By a method analogous to General Procedure IVa, to a solution of N-t-Boc-L-allo-threonine (329 mg, 1.5 mmol) and benzotriazole-1-yl-oxy-tris-pyrrolidinophosphonium hexafluorophosphate (781 mg, 1.5 mmol) in dichloromethane (10 mL) is added diisopropylethylamine (0.52 mL, 3 mmol) and ethyl (2E,4S)-2,5-dimethyl-4-methylamino-hex-2-enoate hydrochloride (235 mg, 1 mmoL). The reaction mixture is stirred at room temperature over the weekend. Chromatography (silica gel, 7:3 hexanes: ethyl acetate) provided 175 mg (44%) of the title compound as a white solid. HRMS (ESI) calcd for C₂OH₃6N₂O₆ (M+H⁺) 401.2646, found 401.2649.

### REFERENCE EXAMPLE 141

### Ethyl (2E,4S)-4-[L-allothreonyl(methyl)amino]-2,5-dimethyl-2-hexenoate

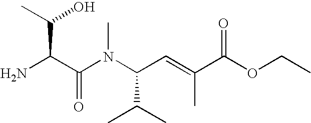

### Ethyl (2E,4S)4-[[N-(tert-butoxycarbonyl)-L-allothreonyl](methyl)amino]-2,5-dimethyl-2-hexenoate (260 mg, 0.65 mmol, from Reference Example 140) is treated with 4N hydrochloric acid/dioxane (2.7 mL) for 3 hours, to give the title compound as a pale-yellow solid in quantitative yield. MS (ESI) calcd for C₁₅H₂₈N₂O₄ (M+H⁺) 301, found 301.

### REFERENCE EXAMPLE 142

### N-(tert-butoxycarbonyl)-N,β,β-trimethyl-L-phenylalanyl-N¹-[(1R,2E)-4-ethoxy-1-isopropyl-3-methyl-4-oxo-2-butenyl]-N¹-methyl-L-allothreoninamide

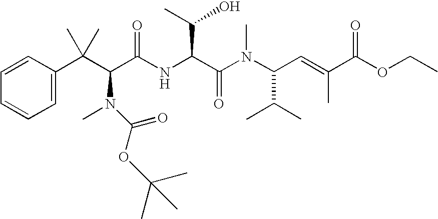

According to the General Procedure IV, to a solution of (2S)-2-[(tert-butoxycarbonyl)(methyl)amino]-3-methyl-3-phenylbutanoic acid (200 mg, 0.65 mmol) and benzotriazole-1-yl-oxy-tris-pyrrolidinophosphonium hexafluorophosphate (440 mg, 0.85 mmol) in dichloromethane (10 mL) is added diisopropylethylamine (0.34 mL, 1.95 mmol) and ethyl (2E,4S)-4-[L-allothreonyl(methyl)amino]-2,5-dimethyl-2-hexenoate hydrochloride (from Reference Example 141). Chromatography (silica gel, 2:1 hexanes: ethyl acetate) gave 287 mg (75%) of the title compound as a colorless sticky oil. HRMS (ESI) calcd for C₃₂H₅₁N₃O₇ (M+H⁺) 590.3799, found 590.3787.

### REFERENCE EXAMPLE 143

### N-(tert-butoxycarbonyl)-N,β,β-trimethyl-L-phenylalanyl-N¹-[(1R,2E)-3-carboxy-1-isopropyl-2-butenyl]-N¹-methyl-L-allothreoninamide

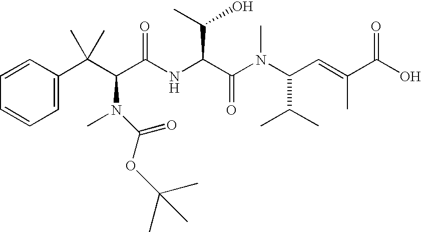

According to General Procedure V, N-(tert-butoxycarbonyl)-N,β,β-trimethyl-L-phenylalanyl-N¹-[(1R,2E)-4-ethoxy-1-isopropyl-3-methyl-4-oxo-2-butenyl]-N¹-methyl-L-allothreoninamide (118 mg, 0.2 mmol, from Reference Example 142) is dissolved in methanol (5 mL) and water (1.6 mL) and treated with 1.0 M aqueous lithium hydroxide (1.6 mL, 1.6 mmol) to give 101 mg (90%) of the title compound as a white solid. HRMS (ESI) calcd for C₃₀H₄₇N₃O₇ (M+H⁺) 562.3487, found 562.3478.

### REFERENCE EXAMPLE 144

### Ethyl (2E,4R)-4-[[N-(tert-butoxycarbonyl)-O,β,β-trimethyl-L-tyrosyl](methyl)amino]-2,5-dimethyl-2-hexenoate

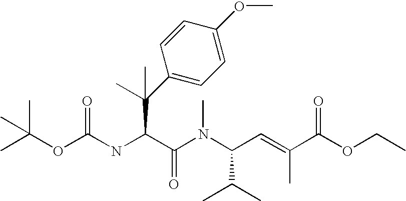

By a method analogous to General Procedure IVa, to a solution of N-(tert-butoxycarbonyl)-O,β,β-trimethyl-L-tyrosine (323 mg, 1.0 mmol) and benzotriazole-1-yl-oxy-tris-pyrrolidinophosphonium hexafluorophosphate (676 mg, 1.3 mmol) in dichloromethane (10 mL) is added diisopropylethylamine (0.52 mL, 3.0 mmol) and ethyl (2E,4S)-2,5-dimethyl-4-methylamino-hex-2-enoate hydrochloride (235 mg, 1.0 mmoL). The reaction mixture is stirred at room temperature for 24 hours. Chromatography (silica gel, 4: 1 hexanes : ethyl acetate) provided 455 mg (90%) of the title compound as a white solid. HRMS (ESI) calcd for C₂₈H44N₂O₆ (M+H⁺) 505.3272, found 505.3254.

### REFERENCE EXAMPLE 145

### Ethyl (2E,4R)-4-[(N—O,β,β-trimethyl-L-tyrosyl)methylamino]-2,5-dimethyl-2-hexenoate

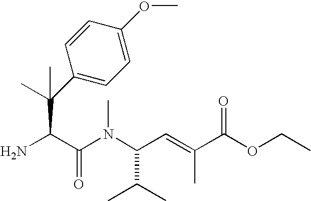

Ethyl(2E,4R)-4-[[N-(tert-butoxycarbonyl)-O,N,β,β-trimethyl-L-tyrosyl](methyl)amino]-2,5-dimethyl-2-hexenoate (400 mg, 0.79 mmol, from Reference Example 144) is treated with 4N hydrochloric acid/dioxane (2 mL) at room temperature for 4 hours, to give 332 mg (95%) of the title compound as a white solid. MS (ESI) calcd for C₂₃H₃₆N₂O₄ (M+H⁺) 405, found 405.

### REFERENCE EXAMPLE 146

### N-(tert-butoxycarbonyl)-N,β,β-trimethyl-L-phenylalanyl-N-[(1R,2E)-4-ethoxy-1-isopropyl-3-methyl-4-oxo-2-butenyl]-N,O,β,β-tetramethyl-L-tyrosinamide

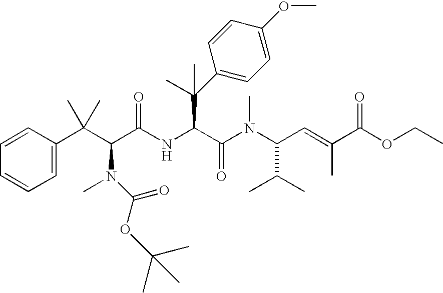

According to the General Procedure IV, to a solution of (2S)-2-[(tert-butoxycarbonyl)(methyl)amino]-3-methyl-3-phenylbutanoic acid (244 mg, 0.79 mmol) and benzotriazole-1-yl-oxy-tris-pyrrolidinophosphonium hexafluorophosphate (537 mg, 1.03 mmol) in dichloromethane (10 mL) is added diisopropylethylamine (0.42 mL, 2.38 mmol) and ethyl (2E,4R)-4-[(N-O,β,3-trimethyl-L-tyrosyl)methylamino]-2,5-dimethyl-2-hexenoate (316 mg, 0.72 mmol, from Reference Example 145). Chromatography on silica gel (4:1 hexanes: ethyl acetate) gave 370 mg (74%) of the title compound as a white solid. HRMS (ESI) calcd for C₄₀H₅₉N₃O₇ (M+H⁺) 694.4426, found 694.4413.

### REFERENCE EXAMPLE 147

### N-(tert-butoxycarbonyl)-N,β,β-trimethyl-L-phenylalanyl-N-[(1S,2E)-3-carboxy-1-isopropyl-2-butenyl]-N,O,β,β-tetramethyl-L-tyrosinamide

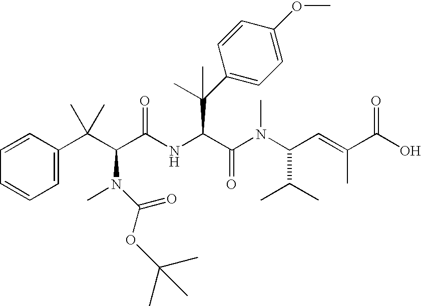

According to General Procedure V, N-(tert-butoxycarbonyl)-N,β,β-trimethyl-L-phenylalanyl-N-[(1R,2E)-4-ethoxy-1-isopropyl-3-methyl-4-oxo-2-butenyl]-N,O,β,β-tetramethyl-L-tyrosinamide (90 mg, 0.13 mmol, from Reference Example 146) is dissolved in methanol (3 mL) and water (1 mL) and treated with 1.0 M lithium hydroxide solution (1 mL, 1 mmol) to give 75 mg (87%) of the title compound as a white solid. HRMS (ESI) calcd for C₃₈H₅₅N₃O₇ (M+H⁺) 666.4113, found 666.4112.

### REFERENCE EXAMPLE 148

### N,β,β-trimethyl-L-phenylalanyl-N-[(1S,2E)4-ethoxy-1-isopropyl-3-methyl4-oxo-2-butenyl]-N,O,β,β-tetramethyl-L-tyrosinamide

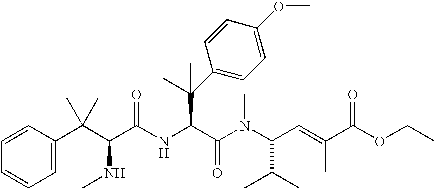

N-(tert-Butoxycarbonyl)-N,β,β-trimethyl-L-phenylalanyl-N-[(1R,2E)4-ethoxy-1-isopropyl-3-methyl4-oxo-2-butenyl]-N,O,β,β-tetramethyl-L-tyrosinamide (35 mg, 0.05 mmol, from Reference Example 146) is treated with 4N hydrochloric acid/dioxane (1 mL) at room temperature for 2 hours, to give 22 mg (69%) of the title compound as a white solid. HRMS (ESI) calcd for C₃₅H₅₁N₃O₅ (M+H⁺) 594.3902, found 594.3900.

### REFERENCE EXAMPLE 149

### Ethyl (2E,4S)-4-[[N-(tert-butoxycarbonyl)-O-methyl-L-allothreonyl](methyl)amino]-2,5-dimethyl-2-hexenoate

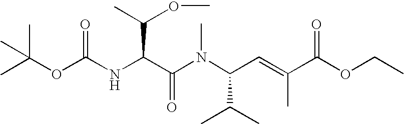

By a method analogous to General Procedure IVa, to a solution of N-t-Boc—O-methyl-L-threonine (233 mg, 1.0 mmol) and benzotriazole-1-yl-oxy-tris-pyrrolidinophosphonium hexafluorophosphate (677 mg, 1.3 mmol) in dichloromethane (10 mL) is added diisopropylethylamine (0.52 mL, 3.0 mmol) and ethyl (2E,4S)-2,5-dimethyl-4-methylamino-hex-2-enoate hydrochloride (235 mg, 1.0 mmol). The reaction mixture is stirred at room temperature for 24 hours. Chromatogrbaphy on silica gel (2:1 hexanes: ethyl acetate) provided 401 mg (97%) of the title compound as a yellow oil. MS (ESI) calcd for C₂₁H₃₈N₂O₆ (M+H⁺) 415, found 415.

### REFERENCE EXAMPLE 150

### Ethyl (2E,4S)-2,5-dimethyl-4-[methyl(O-methyl-L-allothreonyl)amino]-2-hexenoate

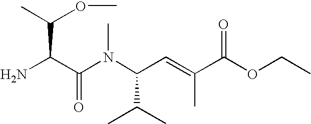

Ethyl (2E,4S)-4-[[N-(tert-butoxycarbonyl)-O-methyl-L-allothreonyl](methyl)amino]-2,5-dimethyl-2-hexenoate (160 mg, 0.39 mmol, from Reference Example 149) is treated with 4N hydrochloric acid/dioxane (2 mL) for 1 hour, to give the title compound as a white solid in quantitative yield. MS (ESI) calcd for C₁₆H₃₀N₂O₄ (M+H⁺) 315, found 315.

### REFERENCE EXAMPLE 151

### N-(tert-butoxycarbonyl)-N,β,β-trimethyl-L-phenylalanyl-N¹-[(1S,2E)-4-ethoxy-1-isopropyl-3-methyl-4-oxo-2-butenyl]-N¹,O-dimethyl-L-allothreoninamide

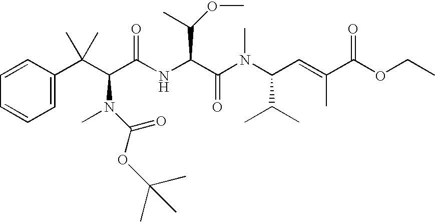

According to the General Procedure IV, to a solution of (2S)-2-[(tert-butoxycarbonyl)(methyl)amino]-3-methyl-3-phenylbutanoic acid (105 mg, 0.34 mmol) and benzotriazole-1-yl-oxy-tris-pyrrolidinophosphonium hexafluorophosphate (232 mg, 0.45 mmol) in dichloromethane (5 mL) is added diisopropylethylamine (0.18 mL, 1.03 mmol) and ethyl (2E,4S)-2,5-dimethyl-4-[methyl(O-methyl-L-allothreonyl)amino]-2-hexenoate (120 mg, 0.34 mmol, from Reference Example 150). Chromatography on silica gel (2:1 hexanes: ethyl acetate) gave 159 mg (79%) of the title compound as a colorless sticky oil. HRMS (ESI) calcd for C₃₃H₅₃N₃O₇ (M+H⁺) 604.3956, found 604.3952.

### REFERENCE EXAMPLE 152

### N-(tert-butoxycarbonyl)-N,β,β-trimethyl-L-phenylalanyl-N¹-[(1S,2E)-3-carboxy-1-isopropyl-2-butenyl]-N¹,O-dimethyl-L-allothreoninamide

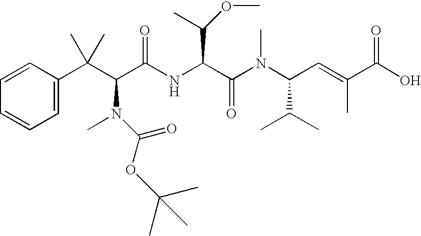

According to General Procedure V, N-(tert-butoxycarbonyl)-N,β,β-trimethyl-L-phenylalanyl-N¹-[(1S,2E)-4-ethoxy-1-isopropyl-3-methyl-4-oxo-2-butenyl]-N¹,O-dimethyl-L-allothreoninamide (144 mg, 0.24 mmol, from Reference Example 151) is dissolved in methanol (6 mL) and water (2 mL) and treated with 1.0 M lithium hydroxide solution (2 mL, 2 mmol). Chromatography on silica gel (1:1:0.01 hexanes: ether: HOAc) provided 118 mg (86%) of the title compound as a white solid. MS (ESI) calcd for C₃₁H₄₉N₃O₇ (M+H⁺) 576, found 576.

### REFERENCE EXAMPLE 153

### N,β,β-trimethyl-L-phenylalanyl-N¹-[(1S,2E)-4-ethoxy-1-isopropyl-3-methyl4-oxo-2-butenyl]-N¹,O-dimethyl-L-allothreoninamide

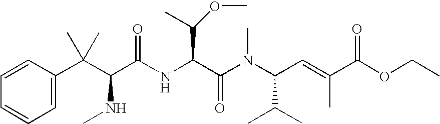

N-(tert-butoxycarbonyl)-N,β,β-trimethyl-L-phenylalanyl-N¹-[(1S,2E)-4-ethoxy-1-isopropyl-3-methyl4-oxo-2-butenyl]-N¹,O-dimethyl-L-allothreoninamide (31 mg, 0.05 mmol, from Reference Example 151) is treated with 4N hydrochloric acid/dioxane (1.5 mL) at room temperature for 1.5 hours to give 20 mg (72%) of title compound as a white solid. MS (ESI) calcd for C₂₈H45N₃O₅ (M+H⁺) 504, found 504.

### REFERENCE EXAMPLE 154

### Ethyl (E,4S)-4-[{(2S)-2-[(tert-butoxycarbonyl)aminol-4-phenylbutanoyl}(methyl)amino]-2,5-dimethyl-2-hexenoate

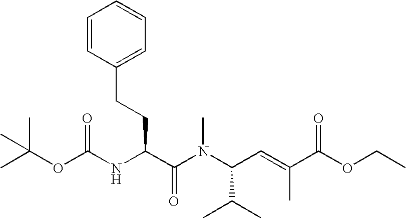

Ethyl (2E,4S)-4-[(N-tert-butoxycarbonyl)(methyl)amino]-2-methyl-5-methyl-2-hexenoate (500 mg, 1.67 mmol) is treated with a solution of hydrogen chloride in para-dioxane (5 mL, 4M solution, Aldrich), to give ethyl (2E,4S)-4-methylamino-2-methyl-5-methyl-2-hexenoate hydrochloride. By a procedure analogous to that described in General Procedure IVb, this material is treated with Boc-homo-L-phenylalanine (467 mg, 1.67 mmol) in the presence of hydroxybenzotriazole (271 mg, 2.0 mmol), 1-(3-dimethylaminopropyl)-3-ethyl-carbodiimide hydrochloride (449 mg, 2.3 mmol and N-methylmorpholine (0.276 mL, 2.5 mmol) in anhydrous N,N-dimethylformamide (13 mL) at room temperature for 15 hours under nitrogen atmosphere. The product is purified by chromatography (silica gel, flash column, 30% ethyl acetate in hexanes), to provide ethyl (E,4S)4-[{(2S)-2-[(tert-butoxycarbonyl)amino]-4-phenylbutanoyl})(methyl)amino]-2,5-dimethyl-2-hexenoate (350 mg, 46%) as a yellow oil. MS (ES): m/z 461.0 (M+H).

### REFERENCE EXAMPLE 155

### Ethyl (6S,9S,12S,13E)-12-isopropyl-2,2,5,11,14-pentamethyl-6-(1-methyl-1-phenylethyl)-4,7,10-trioxo-9-(2-phenylethyl)-3-oxa-5,8,11-triazapentadec-13-en-15-oate

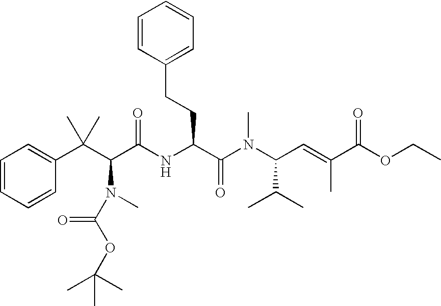

Ethyl (E,4S)-4-[{(2S)-2-[(tert-butoxycarbonyl)amino]-4-phenylbutanoyl}(methyl)amino]-2,5-dimethyl-2-hexenoate (311 mg, 0.68 mmol, obtained from Reference Example 154) is treated with a solution of hydrogen chloride in para-dioxane (2 mL, 4 M solution, Aldrich), to give ethyl (E,4S)-4-[{(2S)-2-amino4-phenylbutanoyl}(methyl)amino]-2,5-dimethyl-2-hexenoate hydrochloride. By using a procedure analogous to that described in General Procedure IVb, this material is treated with (2S)-2-[(tert-butoxycarbonyl)(methyl)amino]-3-methyl-3-phenylbutanoic acid (208 mg, 0.68 mmol, Andersen, R. WO 99/32509) in the presence of hydroxybenzotriazole (110 mg, 0.81 mmol), 1-(3-dimethylaminopropyl)-3-ethylcarbodiimide hydrochloride (181 mg, 0..95 mmol), N-methylmorpholine (0.111 mL, 1.01 mmol) in anhydrous N,N-dimethylformamide (7 mL) at room temperature for 15 hours under nitrogen atmosphere. The product is purified by chromatography (silica gel, flash column, 30% ethyl acetate in hexanes), to provide ethyl (6S,9S,12S, 13E)-1 2-isopropyl-2,2,5,11,14-pentamethyl-6-(1-methyl-1-phenylethyl)-4,7,10-trioxo-9-(2-phenylethyl)-3-oxa-5,8,11-triazapentadec-13-en-15-oate (244 mg, 55%) as a colorless oil. MS (ES): m/z 650.6 (M+H).

Analytical HPLC: (4.6×150 mm Luna C18 column eluted with 15 to 95% acetonitrile in water containing 0.02% TFA over 28 minutes): 63.27% (at 22.6 minutes) of ethyl (6S,9S,12S,13E)-12-isopropyl-2,2,5,11,14-pentamethyl-6-(1-methyl-1-phenylethyl)-4,7,10-trioxo-9-(2-phenylethyl)-3-oxa-5,8,11-triazapentadec-13-en-15-oate, and 10.12% (at 22.9 minutes) of the other diastereomer.

### REFERENCE EXAMPLE 156

### methyl 3-methyl-3-[(4-benzoyl)phenyl]butanoate

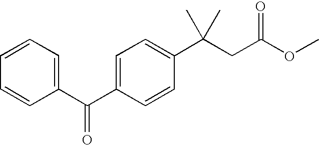

A round-bottomed flask is charged with methyl 3-methyl 3-phenyl butanoate (0.25 g, 1.3 mmol), benzoyl chloride (0.15 mL, 1.3 mmol), and carbon disulfide (1.6 mL). While stirring under nitrogen atmosphere, the reaction mixture is cooled to 0° C. in an ice-water bath. Aluminum chloride (0.35 g, 2.6 mmol) is added in a single portion and the cooling bath is removed. The reaction mixture is heated at reflux 4 hours and then allowed to cool to room temperature. The reaction mixture transferred dropwise into ice-water. The aqueous phase is extracted thrice with dichloromethane. The combined organic extracts are washed with water and 5% aqueous potassium carbonate, dried over sodium sulfate, decanted, and concentrated under reduced pressure to give a brown liquid. MS (ES⁺): m/z (M+H)=297.5

### REFERENCE EXAMPLE 157

### methyl 3-methyl-3-[4-(2-phenyl-1,3-dioxolan-2-yl)phenyl]butanoate

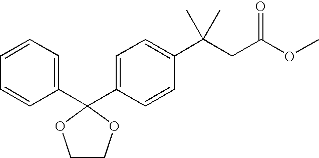

To a solution of methyl 3-methyl-3-[(4-benzoyl)phenyl]butanoate (26 mmol max, from Reference Example 156) in toluene (100 mL), is added ethylene glycol (3.2 g, 52 mmol) and p-toluenesulfonic acid monohydrate (˜10 mg). After heating at reflux for 2 hours, an additional 5 mL ethylene glycol is added, a Dean-Stark trap is added and reflux is re-started. Azeotropic distillation of water is allowed to proceed overnight. A mixture of ethylene glycol and water (8 mL) is observed in the Dean-Stark trap. The LC/MS of an aliquot of reaction mixture revealed the presence of both starting material and desired product. The reaction mixture is concentrated under reduced pressure and the residue is partitioned between diethyl ether and saturated aqueous sodium hydrogen carbonate. The aqueous phase is extracted thrice with diethyl ether. The combined extracts are washed with saturated aqueous sodium hydrogen carbonate and saturated aqueous sodium chloride, dried over sodium sulfate, decanted, and concentrated under reduced pressure. The crude product is taken up in toluene (100 mL) and ethylene glycol (3.2 g, 52 mmol) and p-toluenesulfonic acid monohydrate (˜10 mg) is added. The reaction mixture is heated at reflux for 4 hours without Dean-Stark apparatus and then overnight with the trap. After cooling the reaction mixture to room temperature, the aqueous work-up above is performed. The crude residue is purified by flash chromatography (ethyl acetate/hexanes) to furnish (2.6 g, 29% for 2 steps) of an amorphous white solid. TOF MS (ES⁺): (M+H)=341.3

### REFERENCE EXAMPLE 158

### 3-methyl-3-[4-(2-phenyl-1,3-dioxolan-2-yl)phenyl]butanoic Acid

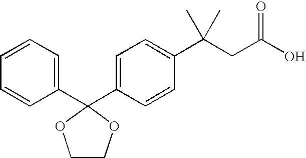

To a suspension of methyl 3-methyl-3-[4-(2-phenyl-1,3-dioxolan-2-yl)phenyl]butanoate (2.6 g, 7.6 mmol, from Reference Example 157) in tetrahydrofuran (20 mL), methanol (20 mL), and water (10 mL) is added lithium hydroxide monohydrate (0.48 g, 11 mmol). The mixture is heated at 55° C. for 5 hours, during which all solids dissolved. The reaction mixture is then allowed to cool to room temperature and solvents are evaporated under reduced pressure. The white solid is partitioned between ethyl acetate and water. Most of the material remained undissolved. The biphasic mixture is cooled to 0° C. in an ice-water bath. Glacial acetic acid is added in portions until pH=5. At this point, white solid precipitated, leaving a clear, colorless supernatant. Ethyl acetate is then removed under reduced pressure, and the solids are isolated by filtration of the aqueous phase and washed with cold water. After drying a white solid is obtained (2.3 g, 92%). TOF MS (ES⁺): (M+H)=327.2

### REFERENCE EXAMPLE 159

### (4S)-3-{(2S)-2-azido-3-methyl-3-[4-(2-phenyl-1,3-dioxolan-2-yl)phenyl]butanoyl}4-benzyl-1,3-oxazolidin-2-one and (4S)-3-{(2S)-2-azido-3-(4-benzoylphenyl)-3-methylbutanoyl]-4-benzyl-1,3-oxazolidin-2-one

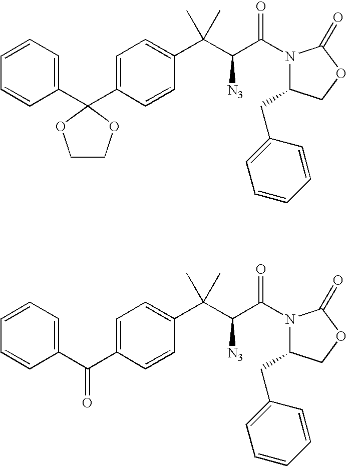

To a solution of 3-methyl-3-[4-(2-phenyl-1,3-dioxolan-2-yl)phenyl]butanoic acid (2.3 g, 7.0 mmol, from Reference Example 158) in anhydrous tetrahydrofuran (14 mL) under a nitrogen atmosphere is added triethylamine (1.2 mL) and the mixture is cooled to −78° C. in a dry-ice acetone bath. Pivaloyl chloride (0.91 mL, 7.4 mmol) is added dropwise, causing the immediate formation of a white precipitate. The reaction mixture is allowed to sit for 20 minutes at −78° C. and is then stirred at 0° C. in an ice-water bath. In a separate flask, a solution of S-benzyloxazolidinone (1.2 g, 6.9 mmol) in anhydrous tetrahydrofuran is prepared under a nitrogen atmosphere and cooled to −35° C. in a dry-ice/acetone bath. A small amount of triphenylmethane (<5 mg) is added as an indicator of deprotonation. n-Butyllithium (1.6 M solution in hexanes, 4.5 mL, 7.2 mmol) is added dropwise via syringe. At the end of this addition, the characteristic pinkish orange color of the anion of triphenylmethane is not yet observed. However, after the introduction of an additional 0.2 mL of n-butyllithium, this color is achieved. After 30 minutes of stirring at 0° C., the flask containing the mixed anhydride is re-cooled to −78° C. in a dry-ice/acetone bath. The solution of the lithium anion of the oxazolidinone is added to the mixed anhydride solution via cannula. The source flask is washed twice with tetrahydrofuran (4 mL×2) and these washings are also transferred via cannula to the mixed anhydride solution. The reaction mixture is stirred at −78° C. for 1 hour, 0° C. for 1 hour, and allowed to warm to room temperature overnight. Water (˜15 mL) is added and stirring is continued for 10 minutes. The aqueous phase is extracted thrice with diethyl ether. The combined extracts are washed with saturated aqueous sodium hydrogen carbonate and saturated aqueous sodium chloride, dried over sodium sulfate, decanted, and concentrated under reduced pressure to afford (3.6 g, >100 % crude) of a white foam. MS (ES⁺): m/z (M+H)=486.2 A solution of this crude benzophenone ketal oxazolidinone (7.0 mmol maximum) in anhydrous tetrahydrofuran (40 mL) is cooled to −78° C. in a dry-ice/acetone bath while stirring under a nitrogen atmosphere. Potassium hexamethylsilazide (0.5 M solution in toluene, 18 mL, 9.0 mmol) is added dropwise to the solution via syringe. A deep orange-red color resulted from this addition. After stirring for 1 hour at −78° C., a pre-cooled solution of triisopropylsulfonyl azide (3.0 g, 9.8 mmol) in tetrahydrofuran (20 mL) of the same temperature is added rapidly via cannula. After stirring for 3 minutes at −78° C., the reaction mixture is quenched by the addition of glacial acetic acid (1.8 mL), which caused a color change from deep red to pale yellow. The cooling bath is removed and the reaction is stirred at room temperature for 20 minutes, followed by 1 hour at 40° C. After cooling to room temperature, the reaction mixture is diluted with water and extracted thrice with diethyl ether. The combined extracts are washed with saturated aqueous sodium hydrogen carbonate and saturated aqueous sodium chloride, dried over sodium sulfate, decanted, and concentrated under reduced pressure to afford a pale yellow oil, which is inert to hydrolysis of the ketal by two methods: treatment with p-toluenesulfonic acid in aqueous acetone and with aqueous hydrochloric acid in tetrahydrofuran. The unaffected crude material is purified by flash chromatography (hexanes/ethyl acetate) to afford a clean separation of (4S)-3-{(2S)-2-azido-3-methyl-3-[4-(2-phenyl-1,3-dioxolan-2-yl)phenyl]butanoyl}-4-benzyl-1,3-oxazolidin-2-one (0.75 g, 1.4 mmol) and (4S)-3-{(2S)-2-azido-3-(4-benzoylphenyl)-3-methylbutanoyl]-4-benzyl-1,3-oxazolidin-2-one (0.82 g, 1.7 mmol), giving a total yield (3.1 mmol/7.0 mmol) of 44% for three steps -- the formation of the mixed anhydride, displacement with the lithium oxazolidinone, and preparation of the azide. (4S)-3-{(2S)-2-azido-3-methyl-3-[4-(2-phenyl-1,3-dioxolan-2-yl)phenyl]butanoyl}4-benzyl-1,3-oxazolidin-2-one: TOF MS (ES⁺)=527.4 (4S)-3-{(2S)-2-azido-3-(4-benzoylphenyl)-3-methylbutanoyl]-4-benzyl-1,3-oxazolidin-2-one: TOF MS m/z (ES⁺)=483.4

### REFERENCE EXAMPLE 160

### (alphaS)4-Benzoyl-N-{(4S)-3-[4-benzoyl-N-(tert-butoxycarbonyl)-β,β-dimethyl-L-phenylalanyl]-4-benzyl-2-oxo-1,3-oxazolidin-2-yl}-N-{(4S)-3-[4-benzoyl-N-(tert-butoxycarbonyl)-β,β-dimethyl-L-phenylalanyl]-4-benzyl-2-oxo-1,3-oxazolidin-4-yl}-N-(tert-butoxycarbonyl)-β,β-dimethyl-L-phenylaninamide

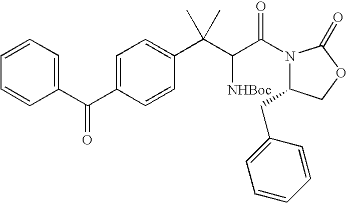

A solution of (4S)-3-{(2S)-2-azido-3-(4-benzoylphenyl)-3-methylbutanoyl]-4-benzyl-1,3-oxazolidin-2-one (0.80 g, 1.7 mmol, from Reference Example 159) in ethyl acetate (8 mL) is degassed with a small piece of dry ice. When all effervescence had subsided, palladium on carbon (Pd/C, 10%, 10 mg) is added in a single portion, followed by di-t-butyl dicarbonate (0.74 g, 3.4 mmol). The reaction flask is evacuated under weak house vacuum and then flushed with hydrogen (balloon pressure). This process is repeated thrice. Finally, the reaction mixture is allowed to stir under hydrogen atmosphere. After 30 minutes, the reaction is incomplete according to thin-layer chromatography (TLC, 20% ethyl acetate/hexanes); hence, stirring under hydrogen is continued over the weekend (˜64 hours). Following this interval, TLC showed a complete disappearance of starting material and the emergence of two new spots. LC/MS analysis revealed these products to be both the desired material and the benzyl phenyl alcohol, the by-product of ketone reduction. The reaction mixture is filtered through a Diatomaceous earth pad to remove Pd/C. The filtrate is concentrated under reduced pressure to afford a clear, colorless oil. This material is subjected to manganese (IV) oxide in dichloromethane in order to oxidize the alcohol by-product back to the benzophenone. This method, however, proved to be very sluggish is aborted shortly. The crude mixture (1.7 mmol max. of alcohol) is then taken up in dichloromethane (10 mL). To the solution is added pyridinium dichromate (0.96 g, 2.6 mmol) and the rust-colored mixture is stirred overnight at room temperature. TLC showed a complete conversion of by-product to desired product. The reaction mixture is filtered through a Diatomaceous earth pad to remove most of the chromium salts. The filtrate is concentrated under reduced pressure to a dark brown oil, and this crude material is purified by flash chromatography (ethyl acetate/hexanes) to afford (0.52 g, 55%) of a hard, white foam. An additional 0.24 g of slightly impure material is also recovered and set aside.

TOF MS m/z (ES⁺)=557.5

### REFERENCE EXAMPLE 161

### 4-benzoyl-N-(tert-butoxycarbonyl)-β,β-dimethyl-L-phenylalanine

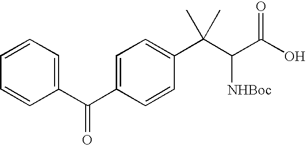

To a 0° C. solution of (alphaS)-4-benzoyl-N-{(4S)-3-[4-benzoyl-N-(tert-butoxycarbonyl)-β,β-dimethyl-L-phenylalanyl]4-benzyl-2-oxo-1,3-oxazolidin-2-yl}-N-{(4S)-3-[4-benzoyl-N-(tert-butoxycarbonyl)-β,β-dimethyl-L-phenylalanyl]-4-benzyl-2-oxo-1,3-oxazolidin-4-yl}-N-(tert-butoxycarbonyl)-β,β-dimethyl-L-phenylalaninamide (0.49 g, 0.88 mmol, from Reference Example 160) in tetrahydrofuran (11 mL) and water (3 mL) is added hydrogen peroxide (30% aqueous solution, 0.76 mL, 7.9 mmol), followed by lithium hydroxide monohydrate (0.11 g, 2.6 mmol). The reaction mixture is allowed to continue stirring for 23 hours while gradually warming to room temperature. The reaction is quenched by the addition of sodium sulfite (1.5 M aqueous solution, 10 mL, 15 mmol), which is accompanied by slight exothermicity. The quenched mixture is stirred for 1 hour at room temperature and then cooled to 0° C. in an ice-water bath. The pH of the mixture is adjusted to 4 by the addition of citric acid (1 M aqueous solution). The acidified mixture is then extracted thrice with ethyl acetate. The combined extracts are washed with saturated aqueous sodium chloride solution, dried over anhydrous sodium sulfate, decanted, and concentrated under reduced pressure to afford a white foam (0.47 g). This crude product is dissolved in acetonitrile/water (1:1) and purified by semi-preparative reverse-phase HPLC, employing a gradient elution of 5% acetonitrile/95% water to 100% acetonitrile over 1 hour. A hard, white foam (0.23 g, 66%) is obtained after collection and concentration. TOF MS (ES⁻): m/z (M−H)=396.2

### REFERENCE EXAMPLE 162

### N-(tert-butoxycarbonyl)-β,β-dimethyl-4-(2-phenyl-1,3-dioxolan-2-yl)-L-phenylalanine

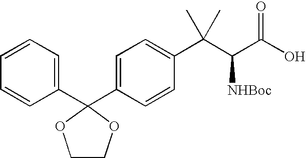

A solution of (4S)-3-{(2S)-2-azido-3-methyl-3-[4-(2-phenyl-1,3-dioxolan-2-yl)phenyl]butanoyl}-4-benzyl-1,3-oxazolidin-2-one (0.74 g, 1.4 mmol) from Reference Example 159 in ethyl acetate (25 mL) is degassed with a small piece of dry ice. When all effervescence had subsided, palladium on carbon (Pd/C, 10%, 20 mg) is added in a single portion, followed by di-t-butyl dicarbonate (0.61 g, 2.8 mmol). The reaction flask is evacuated under weak house vacuum and then flushed with hydrogen (balloon pressure). This process is repeated thrice. Finally, the reaction mixture is allowed to stir under hydrogen atmosphere. After 4 hours, TLC showed a complete disappearance of starting material and the emergence of a single new spot of lower retention factor. The reaction mixture is filtered through a Diatomaceous earth pad to remove Pd/C. The filtrate is concentrated under reduced pressure to afford (1.3 g, >100%) of a clear, light blond oil. To a 0° C. solution of this crude material (1.3 g, 1.4 mmol maximum) in tetrahydrofuran (17 mL) and water (4 mL) is added hydrogen peroxide (30% aqueous solution, 1.4 mL, 13 mmol), followed by lithium hydroxide monohydrate (0.18 g, 4.2 mmol). The reaction mixture is stirred for 23 hours at room temperature. LC/MS analysis revealed the reaction to be incomplete. The mixture is cooled to 0° C. and an additional 4 mL of hydrogen peroxide solution and 0.18 g of lithium hydroxide monohydrate is added. Stirring is continued for 60 hours while the mixture gradually warmed to room temperature. The reaction is quenched by the addition of sodium sulfite (1.5 M aqueous solution, 25 mL, 38 mmol), which is accompanied by slight exothermicity. The quenched mixture is stirred for 1 hour at room temperature and then cooled to 0° C. in an ice-water bath. The pH of the mixture is adjusted to 4 by the addition of citric acid (1 M aqueous solution). The acidified mixture is then extracted thrice with ethyl acetate. The combined extracts are washed with saturated aqueous sodium chloride solution, dried over anhydrous sodium sulfate, decanted, and concentrated under reduced pressure to afford a white foam (0.90 g). This crude product is dissolved in dimethylsulfoxide and purified by semi-preparative reverse-phase HPLC, employing a gradient elution of 5% acetonitrile/95% water to 100% acetonitrile over 1 hour. N-(tert-butoxycarbonyl)-β,β-dimethyl-4-(2-phenyl-1,3-dioxolan-2-yl)-L-phenylalanine is obtained as a white powder (0.37 g, 60%) after collection and concentration. TOF MS (ES⁻): (M−H)=440.1

### REFERENCE EXAMPLE 163

### N-(tert-butoxycarbonyl)-N,β,β-trimethyl-4-(2-phenyl-1,3-dioxolan-2-yl)-L-phenylalanine

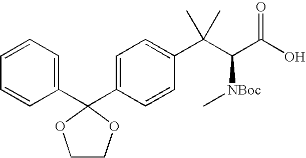

A solution of N-(tert-butoxycarbonyl)-β,β-dimethyl-4-(2-phenyl-1,3-dioxolan-2-yl)-L-phenylalanine (0.34 g, 0.77 mmol, from Reference Example 162) in anhydrous dimethylformamide (11 mL) is cooled to 0 IC in an ice-water bath under a nitrogen atmosphere. Sodium hydride (60% dispersion in mineral oil, 0.15 g, 3.9 mmol) is added slowly. After effervescence had ceased, methyl iodide (0.49 g, 7.8 mmol) is added via syringe. The reaction mixture is then allowed to warm to room temperature gradually while stirring overnight. The next morning, the mixture is cooled to 0° C. in an ice-water bath. Glacial acetic acid (1 mL) is added to adjust the pH to 4. The reaction mixture is partitioned between ethyl acetate and water. The aqueous phase is extracted thrice with diethyl ether. The combined extracts are washed with saturated aqueous sodium hydrogen carbonate and saturated aqueous sodium chloride, dried over sodium sulfate, decanted, and concentrated under reduced pressure to a blond oil (0.49 g, >100%).

MS (ES⁺): m/z (M+Na)=492.3 The crude blond oil (0.49 g, 0.77 mmol maximum) is taken up in tetrahydrofuran (2 mL), methanol (2 mL) and water (1 mL). To this solution, lithium hydroxide monohydrate (81 mg, 1.9 mmol) is added. The reaction mixture is stirred for 24 hours at room temperature and an additional quantity of lithium hydroxide monohydrate (20 mg, 0.48 mmol) is added. Stirring is resumed for 60 hours, following which the solvent is evaporated under reduced pressure to give a white solid (0.62 g), which is purified by semi-preparative reverse-phase HPLC, employing a gradient elution of 5% acetonitrile/95% water to 100% acetonitrile over 1 hour. N-(tert-butoxycarbonyl)-N,β,β-trimethyl-4-(2-phenyl-1,3-dioxolan-2-yl)-L-phenylalanine is obtained as a hard white foam (0.26 g, 74% over 2 steps) after collection and concentration. TOF MS (ES⁻): m/z (M−H)=454.1

### REFERENCE EXAMPLE 164

### N-{1-[(1 —Formyl-2-methyl-propyl)-methyl-carbamoyl]-2,2-dimethyl-propyl}-3-methyl-2-methylamino-3-phenyl-butyramide

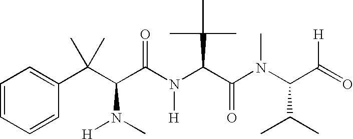

A solution of N,β,β-trimethyl-L-phenylalanyl-N1-[(1S,2E)-3-carboxy-1-isopropylbut-2-enyl]-N¹,3-dimethyl-L-valinamide (1.0 g) in methanol (20 mL) is added to 100 mL of methanol at −78° C. which had been treated with ozone until blue. The resulting colorless reaction mixture is treated with ozone at −78° C. until the blue color remained. After 5 min dimethylsulfide (2 mL) is added. The reaction mixture is allowed to warm to room temperature and then concentrated in vacuo to give N-{1-[(1 —Formyl-2-methyl-propyl)-methyl-carbamoyl]-2,2-dimethyl-propyl}-3-methyl-2-methylamino-3-phenyl-butyramide as an oil. MS (ES): m/z 418.4 (M+H).

### REFERENCE EXAMPLE 165

### tert-Butyl 1-{[methoxy(methyl)amino]carbonyl}-3-butenylcarbamate

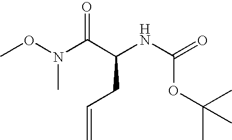

In a manner analogous to that described in the literature (Andersen, R. et. al. WO 96/33211), to a cold solution (0° C., ice bath) of commercially available (S)-2-tert-butoxycarbonylamino-pent-4-enoic acid (2.0g, 9.29 mmol), N,O-dimethyl hydroxyl amine (0.90g, 9.29 mmol) and benzotriazole-1-yl-oxy-tris-pyrrolidinophosphonium hexafluorophosphate (5.07g, 9.75) in dichloromethane (9.29 mL) is added diisopropylethylamine (4.84 mL, 27.8 mmol). After 1 minute, the reaction mixture is warmed to room temperature and stirring is continued for 1 hour. If the pH of the mixture is less than 7, the mixture is treated with a few drops of diisopropylethylamine. To the organic layer is added ether and it is washed 3× with 3N aqueous hydrogen chloride, aqueous sodium bicarbonate, and brine. The organic layer is dried with anhydrous sodium sulfate and concentrated in vacuo. The crude material is chromatographed on a silica gel column eluting with ethyl acetate/hexane to give the title compound as a yellow tinted semisolid (1.87 g, 78%) MS (ES): m/z 259.4 [M+H]

### REFERENCE EXAMPLE 166

### tert-Butyl 1-{[methoxy(methyl)amino]carbonyl}-3-butenyl(methyl)carbamate

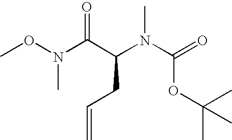

In a manner analogous to that described in the literature (Andersen, R. et. al. WO 96/33211), to a vigorously stirred solution of tert-butyl 1-{[methoxy(methyl)amino]carbonyl}-3-butenylcarbamate (2.0g, 7.75 mmol, from Reference Example 165) in dry dimethylformamide (108.5 mL, 14mUmmol) at 0° C. (ice bath) is added 60% sodium hydride (0.560g, 23.3 mmol, 3eq) and iodomethane (4.82 mL, 77.5 mmol). The resulting gray suspension is allowed to warm to room temperature. After 12 hours, the excess sodium hydride is quenched by cautious addition of water and the mixture is acidified by dropwise addition of 1.0M aqueous citric acid. The acidic mixture is extracted 3× with ethyl acetate. The combined organic layers are dried over sodium sulfate and concentrated in vacuo to give a yellow oil (1.51 g, 72%). MS (ES): m/z [M+H] 273.14

### REFERENCE EXAMPLE 167

### tert-Butyl 1-formyl-3-butenyl(methyl)carbamate

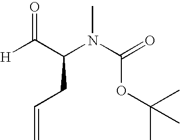

In a manner analogous to that described in the literature (Andersen, R. et. al. WO 96/33211), to a cooled solution (0° C., ice bath) of tert-butyl 1-{[methoxy(methyl)amino]carbonyl}-3-butenyl(methyl)carbamate (1.5 g, 5.51mmol, from Reference Example 166) in anhydrous tetrahydrofuran (5.51 mL), 1.0M lithium aluminum hydride (16.6 mL, 16.5mmol) in tetrahydrofuran is added dropwise over 30 minutes. The reaction mixture stirred at room temperature for 30 minutes and is treated with 5% potassium hydrogen sulfate and stirred at room temperature for 15 minutes. The organic layer is then washed with 3N aqueous hydrogen chloride, saturated aqueous sodium bicarbonate, and brine. The organic layer is then collected and dried with sodium sulfate and concentrated in vacuo to give colorless oil (0.940 g, 80%). The crude product is used in the next step without further purification. MS (ES): m/z [M+H] 214.3

### REFERENCE EXAMPLE 168

### Ethyl (2E)4-[(tert-butoxycarbonyl)(methyl)amino]-2-methyl-2,6-heptadienoate

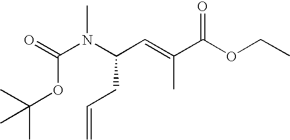

In a manner analogous to that described in the literature (Andersen, R. et. al. WO 96/33211), to a solution of tert-butyl 1-formyl-3-butenyl(methyl)carbamate (1.0 g, 4.7 mmol, from Reference Example 167) in dry dichloromethane (4.7 mL, 1.0 mL/mmol of N-boc-amino acid) under nitrogen atmosphere at room temperature is added (carbethoxyethylidene)triphenylphospharane (2.04 g, 5.64 mmol). After 12 hours the reaction mixture is diluted with water and extracted with ether. The organic layers are then collected and dried with sodium sulfate and concentrated in vacuo. The crude oil is then purified by silica gel chromatography eluting with ethyl acetate/hexane to give exclusively the E-isomer as a colorless oil (1.0 g, 72%). MS (ES): m/z [M+H] 298.10

### REFERENCE EXAMPLE 169

### (2E, 4S)-2-Methyl-4-methylamino-hepta-2,6-dienoic Acid Ethyl Ester

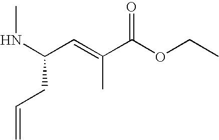

Ethyl (2E)-4-[(tert-butoxycarbonyl)(methyl)amino]-2-methyl-2,6-heptadienoate (1.0 g, 2.40 mmol, from Reference Example 168) is dissolved in dichloromethane (8.4 mL, 3.5 mL/mmol) and treated with 4.0N hydrogen chloride/dioxane (2.3 mL, 9.3 mmol) in dichloromethane (8.4 mL, 3.5 mL/mmol) at room temperature for 30 minutes. The reaction mixture is then concentrated and triturated with ether to afford a white solid (0.44 g, 94%). The crude material is used for further coupling reactions. MS (ES): m/z [M+H] 198.15 [M+ACN+H] 239.04

### REFERENCE EXAMPLE 170

### Ethyl(2E)4-[[N-(tert-butoxycarbonyl)-3-methyl-L-valyl](methyl)amino]-2-methyl-2,6-heptadienoate

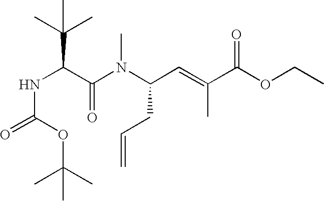

In a manner analogous to that described in General Method IVa, to a cooled (0° C., ice bath) solution of L-tert-Boc Leucine (840 mg, 4.3 mmol, Aldrich) and benzotriazole-1-yl-oxy-tris-pyrrolidinophosphonium hexafluorophosphate (850 mg, 4.3 mmol) in anhydrous dimethylformamide (3-5 mL, Aldrich) is added diisopropylethylamine (2.2 mL, 12.9 mmol) under a nitrogen atmosphere. To this solution is added a solution of the (2E, 4S)-2-methyl4-methylamino-hepta-2,6-dienoic acid ethyl ester (840 mg, 4.3 mmol, from Reference Example 169) in anhydrous dimethylformamide (10 mL). After stirring at 0° C. for 5-10 minutes, the cooling bath is removed, and the resulting reaction mixture is stirred at room temperature for 15 hours. The mixture is diluted with water, and the aqueous layer is extracted with ethyl acetate (3 times). The combined extracts are washed with saturated aqueous sodium chloride, dried over sodium sulfate, filtered and concentrated in vacuo. The residue is chromatographed (silica gel, flash column) to give the product as an oil (1.25 g, 71%). MS (ES): m/z [M+H] 411.28

### REFERENCE EXAMPLE 171

### Ethyl (2E)-2-methyl-4-[methyl(3-methyl-L-valyl)amino]-2,6-heptadienoate

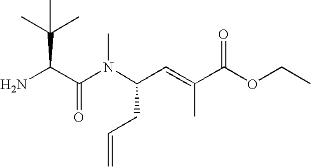

Ethyl (2E)-4-[[N-(tert-butoxycarbonyl)-3-methyl-L-valyl](methyl)amino]-2-methyl-2,6-heptadienoate (500 mg, 1.21 mmol, from Reference Example 170) is dissolved in dichloromethane (4.23 mL, 3.5mUmmol) and treated with 4.0N hydrogen chloride/dioxane (0.9 mL, 3.6 mmol) at room temperature for 30 minutes. The reaction mixture is then concentrated and triturated with ether to afford a white solid (0.322 g, 86%). The crude material is used for further coupling reactions. MS (ES): m/z [M+H] 311.28

### REFERENCE EXAMPLE 172

### (2E ,4S)-4-({2-[2-(tert-Butoxycarbonyl-methyl-amino)-3-methyl-3-phenyl-butyrylamino]-3,3-dimethyl-butyryl}-methyl-amino)-2-methyl-hepta-2,6-dienoic Acid

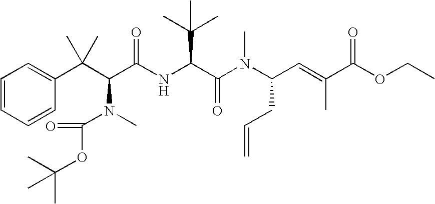

In a manner analogous to that described in General Method lVb, to a cooled (0° C., ice bath) solution of (2S)-2-[(tert-butoxycarbonyl)(methyl)amino)]-3-methyl-3-phenylbutanoic acid (147.4 mg, 0.48 mmol), hydroxybenzotriazole (71.3 mg, 0.53 mmol) and 1-(3-dimethylaminopropyl)-3-ethylcarbodiimine hydrochloride (110.4 mg, 0.57 mmol) in anhydrous dimethylformamide (˜3 mL) is added N-methylmorpholine (0.074 mL, 0.67 mmol) via syringe under a nitrogen atmosphere. After stirring for 15 minutes at 0° C., the cooling bath is removed, and the resulting mixture is stirred for ˜2 hours. The solution is cooled at 0° C. (ice water bath), and to this mixture is added a solution ethyl (2E)-2-methyl-4-[methyl(3-methyl-L-valyl)amino]-2,6-heptadienoate (150 mg, 0.48 mmol) from Reference Example 171 in anhydrous dimethylformamide (3 mL). The cooling bath is removed, and the resulting mixture is stirred for ˜15 hours at room temperature under a nitrogen atmosphere. The mixture is diluted with water, and the aqueous layer is extracted with ethyl acetate (3 times). The combined extracts are washed with saturated aqueoussodium chloride, dried over sodium sulfate, filtered and concentrated in vacuo. The residue is chromatographed (silica gel, flash column) to give a white foam (0.249 g, 87%). MS (ES): m/z [M+H] 598.41

### REFERENCE EXAMPLE 173

### N-(tert-butoxycarbonyl)-N,β,β-trimethyl-L-phenylalanyl-N¹-[(2E)-1-allyl-3-carboxy-2-butenyl]-N¹,3-dimethyl-L-valinamide

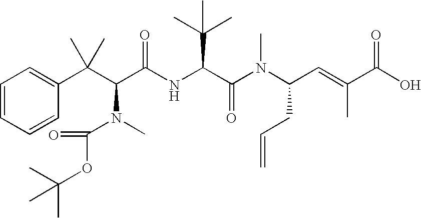

In a manner analogous to that described in General Method V (2E, 4S)--4-({2-[2-(tert-butoxycarbonyl-methyl-amino)-3-methyl-3-phenyl-butyrylamino]-3,3-dimethyl-butyryl}-methyl-amino)-2-methyl-hepta-2,6-dienoic acid ethyl ester (112 mg, 0.19 mmol, from Reference Example 172) is dissolved in methanol (4.56 mL, 24 mL/mmol), and cooled to 0° C. (ice water bath). To this solution is added water (1.5 mL) and 1.0M aqueous lithium hydroxide (1.52 mL). The cooling bath is removed, and the resulting mixture is stirred at room temperature for 15 hours. Methanol is removed in vacuo, and the residual aqueous mixture is cooled with an ice water bath, and acidified with aqueous 1 M hydrogen chloride solution and washed 3× with ethyl acetate and water. The organic layers are then collected and concentrated in vacuo to give the crude product (82 mg, 75%). MS (ES): m/z [M+H] 570.38

### REFERENCE EXAMPLE 173

### 3-Methyl-2-butenyl [(tert-butoxycarbonyl)amino]acetate

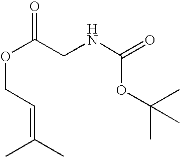

By using a procedure analogous to that described in the literature, (Angew. Chem. Int. Ed. Engl. 1978, 522-524), to a solution of N-boc glycine (5.0 g, 28.5 mmole) in dichloromethane (10.0 mL/5.7 mmol) is added 4-(dimethylamino)pyridine (400 mg) and 3-methyl-2-buten-1-ol (3.3 g, 38.0 mmol) under a nitrogen atmosphere. The resulting mixture is cooled (0° C., ice bath) and treated with 1,3-dicyclohexylcarbodiimide (8.2 g, 1.4 eq), then stirred for 3 hours at room temperature. The reaction mixture is concentrated and taken up in dichloromethane. The resulting precipitate is filtered off and the organic layer is washed with 1 N aqueous hydrogen chloride, saturated sodium bicarbonate, brine, and then dried over anhydrous sodium sulfate. The crude product is chromatographed (silica gel) to give a colorless oil (5 g, 81%). MS (ES): m/z [M+H] 244.15

### REFERENCE EXAMPLE 174

### (2S)-2-[(tert-butoxycarbonyl)amino]-3,3-dimethyl-4-pentenoic acid

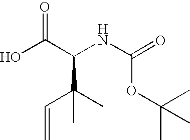

By using a procedure analogous to that described in the literature (Kazmaier, U. J. Org. Chem. 1996, 61, 3694 and A. and Kazmaier, U. Tetrahedron Letters 1996, 37, 7945), to lithium diisopropylamide (92.5 mmol) in hexane/heptane/tetrahydrofuran at −78° C., is added 3-methyl-2-butenyl [(tert-butoxycarbonyl)amino]acetate (5 g, 20.6 mmol, from Reference Example 173), followed by zinc chloride (0.5 M tetrahydrofuran, 41 mL, 20.6 mmol) and quinidine (13.4 g, 41.2 mmol). The resulting orange mixture is allowed to warm to room temperature and stirred overnight. The mixture is poured into a cooled (0° C., ice bath) solution of 1M aqueous potassium hydrogen sulfate and the pH is adjusted to 3. This mixture is extracted with ether and the organic layer is dried over anhydrous sodium sulfate. The crude residue obtained after solvent removal in vacuo, is washed with hexanes and ether leaving insoluble quinidine as a solid. The combined organic washings are concentrated to give the crude product as a colorless oil (4.0 g, 80%). The reaction produced a 35% enantiomeric excess of the (S) isomer. MS (ES): m/z [M+H] 244.15

### REFERENCE EXAMPLE 175

### Ethyl (2E,4S)4-[{2-[(tert-butoxycarbonyl)amino]-3,3-dimethyl4-pentenoyl}(methyl)amino]-2,5-dimethyl-2-hexenoate

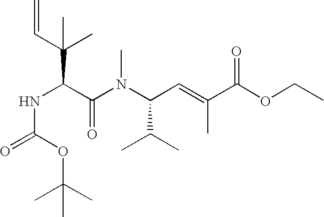

By a method analogous to that described in General Method IVa, to a cooled solution (0° C., ice bath) of (2S)-2-[(tert-butoxycarbonyl)amino]-3,3-dimethyl-4-pentenoic acid (98 mg, 0.40 mmol, 35% enantiomeric excess, from Reference Example 174) and benzotriazole-1-yl-oxy-tris-pyrrolidinophosphonium hexafluorophosphate (208 mg, 0.40 mmol) in anhydrous dimethylformamide (3-5 mL, Aldrich) is added diisopropylethylamine (0.77 mL, 0.44 mmol). To this solution is added a solution of ethyl (2E, 4S)-2,5-dimethyl-4-(methylamino)-2-hexenoate (3 mL). After stirring at 0° C. for 5-10 minutes, the cooling bath is removed, and the resulting reaction mixture is stirred at room temperature for 15-20 hours. The mixture is diluted with water, and the aqueous layer is extracted with ethyl acetate (3 times). The combined extracts are washed with saturated aqueous sodium chloride, dried over sodium sulfate, filtered and concentrated in vacuo. The residue is chromatographed (silica gel, flash column), to give the title compound as the major isomer (130 mg, 76%). MS (ES): m/z [M+H] 425.29

### REFERENCE EXAMPLE 176

### Ethyl (2E,4S)-4-[(2-amino-3,3-dimethyl-4-pentenoyl)(methyl)amino]-2,5-dimethyl-2-hexenoate

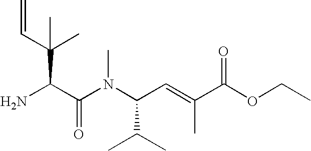

Ethyl (2E,4S)-4-[{2-[(tert-butoxycarbonyl)amino]-3,3-dimethyl-4-pentenoyl}(methyl)amino]-2,5-dimethyl-2-hexenoate (175 mg, 2.59 mmol, from Reference Example 175) is dissolved in dichloromethane at room temperature and treated with 4.ON hydrogen chloride in dioxane (0.206 mL, 5.18 mmol) for 30 minutes. The reaction mixture is then concentrated and triturated with ether to give a white solid (0.115 g, 78%, mixture of isomers, title compound predominates). MS (ES): m/z [M+H] 325.24

### REFERENCE EXAMPLE 177

### Ethyl (2E,4S)-4-[(2-{[N-(tert-butoxycarbonyl)-N,β,β-trimethylphenylalanyl]amino}-3,3-dimethyl-4-pentenoyl)(methyl)amino]-2,5-dimethyl-2-hexenoate

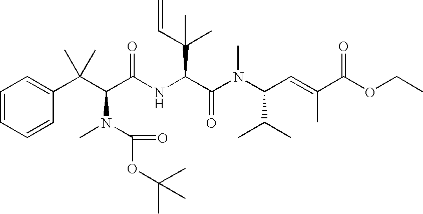

By a method analogous to that described in General Method IVa, to (2S)-2-[(tert-butoxycarbonyl)(methyl)amino)]-3-methyl-3-phenylbutanoic acid (77 mg, 0.25 mmol), hydroxybenzotriazole (41 mg, 0.30 mmol), 1-(3-dimethylaminopropyl)-3-ethylcarbodiimine hydrochloride (67 mg, 0.35 mmol) and N-methylmorpholine (0.028 mL, 0.25 mmol) in anhydrous dimethylformamide (1 mL) is added a solution of ethyl (2E,4S)-4-[(2-amino-3,3-dimethyl-4-pentenoyl)(methyl)amino]-2,5-dimethyl-2-hexenoate (90 mg, 0.25 mmol, from Reference Example 176) in anhydrous dimethylformamide (1 mL). The title compound is obtained after workup (100 mg, 65%). MS (ES): m/z [M+H] 612.43

### REFERENCE EXAMPLE 178

### (2E,4S)4-({2-[2-(tert-butoxycarbonyl-methyl-amino)-3-methyl-3-phenyl-butyrylamino]-(2S)-3,3-dimethyl-butyryl}-methyl-amino)-2-methyl-hepta-2,6-dienoic Acid

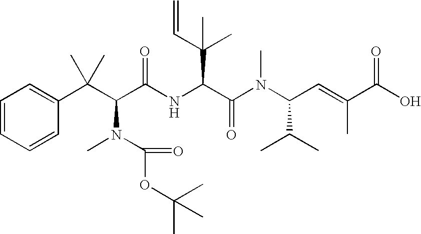

Ethyl (2E,4S)-4-[(2-{[N-(tert-butoxycarbonyl)-N,β,β-trimethylphenylalanyl]amino}-3,3-dimethyl-4-pentenoyl)(methyl)amino]-2,5-dimethyl-2-hexenoate (100 mg, 0.17 mmol, from Reference Example 177) is dissolved in methanol (1 mL), tetrahydrofuran (1 mL) and cooled to 0° C. (ice water bath). To this solution is added water (0.5 mL) and solid lithium hydroxide solution (8 mg, 0.34 mmol). The cooling bath is removed, and the resulting mixture is stirred at room temperature for 15 hours. Methanol is removed in vacuo, and the residual aqueous mixture is cooled with an ice water bath, and acidified with aqueous 1 M aqueous hydrogen chloride to pH 2-3 and washed 3× with ethyl acetate. The combined organic layers are dried and concentrated in vacuo to give the title compound (60 mg, 60%). MS (ES): m/z [M+H] 586.38

### REFERENCE EXAMPLE 179

### Methyl-2-[(tert-butoxycarbonyl)(methyl)amino]-3,3-dimethyl-4-entenoate

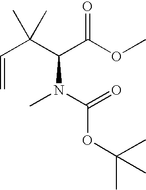

To a cooled (0° C.) mixture of sodium hydride (200 mg, 0.82 mmol) in dimethylformamide (10 mL) is added (2S)-2-[(tert-butoxycarbonyl)amino]-3,3-dimethyl-4-pentenoic acid (from Reference Example 174) and iodomethane (1.16 g, 8.20 mmol). The resulting mixture is stirred for 10 hours at room temperature. Water is added to the mixture and it is acidifed with 5% aqueous citric acid to pH 3. The aqueous layer is extracted with ethyl acetate and the organic layer washed with saturated aqueous sodium thiosulfate and brine and dried over anhydrous sodium sulfate. The solvent is removed in vacuo to give the title compound (115.0 mg, 52 %). MS (ES): m/z [M+H] 272.1

### REFERENCE EXAMPLE 180

### 2-(tert-Butoxycarbonyl-methyl-amino)-3,3-dimethyl-pent-4-enoic Acid

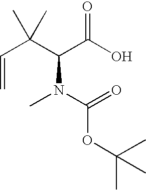

To a solution of methyl-2-[(tert-butoxycarbonyl)(methyl)amino]-3,3-dimethyl-4-entenoate (85 mg, 0.31 mmol, from Reference Example 179) in tetrahydrofuran (2 mL), methanol (2 mL), and water (1 mL) is added lithium hydroxide hydrate (15 mg, 0.63 mmol). After 15 hours the solvents are removed in vacuo and resulting film dissolved in water. The mixture is then acidified to pH 2 with 1N hydrogen chloride. The aqueous layer is extracted with ethyl acetate and the organic layers are dried with sodium sulfate and concentrated to give the title compound (60 mg, 75%). MS (ES): m/z [M+H] 258.3

### REFERENCE EXAMPLE 181

### Ethyl (9S,12S,13E)-9-tert-butyl-6-(1,1-dimethyl-2-propenyl)-12-isopropyl-2,2,11,14-tetramethyl-4,7,10-trioxo-3-oxa-5,8,11-triazapentadec-13-en-15-oate

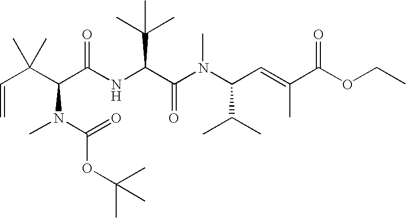

By a method analogous to that described in General Method IVb, to a cooled (0° C., ice bath) solution of 2-(tert-butoxycarbonyl-methyl-amino)-3,3-dimethyl-pent-4-enoic acid (61 mg, 0.18 mmol), hydroxybenzotriazole (29 mg, 0.22 mmol) and 1-(3-dimethylaminopropyl)-3-ethylcarbodiimine hydrochloride (48 mg, 0.25 mmol) in anhydrous dimethylformamide (0.5 mL) is added N-methylmorpholine (0.020 mL, 0.18 mmol) via syringe under a nitrogen atmosphere. After stirring for 15 minutes at 0° C., the cooling bath is removed, and the resulting mixture is stirred for 15 hours. The solution is cooled to 0° C. (ice water bath), and to this mixture is added a solution of ethyl (E, 4S) -4-[[(2S)-2-amino-3,3-dimethylbutanoyl](methyl)amino]-2,5-dimethyl-2-hexenoate (45 mg, 0.18 mmol) in anhydrous dimethylformamide (0.5 mL). The title compound is obtained after workup (70 mg, 71%). MS (ES): m/z [M+H] 552.3

### REFERENCE EXAMPLE 182

### (9S,12S,13E)-9-tert-butyl-6-(1,1-dimethyl-2-propenyl)-12-isopropyl-2,2,5,11,14-pentamethyl-4,7,10-trioxo-3-oxa-5,8,11-triazapentadec-13-en-15-oic Acid

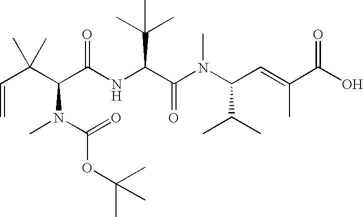

By a method analogous to that described in General Procedure V, a solution of ethyl (9S,12S,13E)-9-tert-butyl-6-(1,1-dimethyl-2-propenyl)-1 2-isopropyl-2,2,11,14-tetramethyl-4,7,10-trioxo-3-oxa-5,8,11-triazapentadec-13-en-15-oate (65 mg, 0.12 mmol, from Reference Example 181) in methanol (1 mL), tetrahydrofuran (1 mL) and water (0.5 mL) is treated with lithium hydroxide hydrate (6 mg, 0.24 mmol) to give the title compound (40 mg, 64%). MS (ES): m/z [M−H] 524.34

### REFERENCE EXAMPLE 183

### N-(tert-butoxycarbonyl)-3-methyl-L-isoleucine

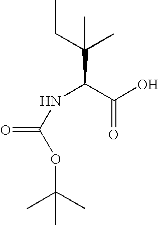

A solution of (2S)-2-[(tert-butoxycarbonyl)amino]-3,3-dimethyl-4-pentenoic acid (400 mg, 1.6 mmol, from Reference Example 174) in ethyl acetate (10 mL) is treated with 10% Pd-C (175 mg, 0.16 mmol) and 1 atmosphere of hydrogen for 8 hours. The catalyst is removed by filtration. The organic solvent is removed under reduced pressure to give the title compound (325 mg, 83%, 35% ee). MS (ES): m/z [M+H] 246.7

### REFERENCE EXAMPLE 184

### Ethyl (2E,4S)4-[[N-(tert-butoxycarbonyl)-3-methylisoleucyl](methyl)amino]-2,5-dimethyl-2-hexenoate

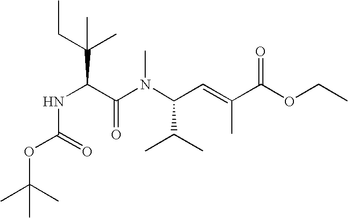

By a method analogous to general method IV, to a cooled (0° C., ice bath) solution of N-(tert-butoxycarbonyl)-3-methyl-L-isoleucine (325 mg, 1.33 mmol, from Reference Example 183) and benzotriazole-1-yl-oxy-tris-pyrrolidinophosphonium hexafluorophosphate (692 mg, 1.33 mmol) in anhydrous dimethylformamide (3-5 mL) is added diisopropylethylamine (0.254 mL, 1.46 mmol) under a nitrogen atmosphere. To this solution is added a solution of ethyl ethyl (E, 4S) 4-[[(2S)-2-amino-3,3-dimethylbutanoyl](methyl)amino]-2,5-dimethyl-2-hexenoate (264 mg, 1.33 mmol) in anhydrous dichloromethane (15 mL). Flash chromatography gave the desired isomer (200 mg). MS (ES): m/z [M+H] 427.29

### REFERENCE EXAMPLE 185

### Ethyl (2E ,4S)-2,5-dimethyl-4-[methyl(3-methylisoleucyl)amino]hex-2-enoate

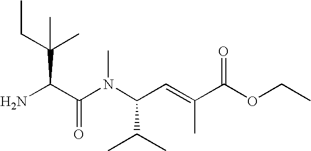

Ethyl (2E,4S)-4-[[N-(tert-butoxycarbonyl)-3-methylisoleucyl](methyl)amino]-2,5-dimethyl-2-hexenoate (0.170 g, 0.4 mmol, from Reference Example 184) is dissolved in dichloromethane (4 mL) and treated with 4.ON hydrogen chloride in dioxane (0.20 mL, 0.80 mmol) at room temperature for 30 minutes. The reaction mixture is then concentrated in vacuo and washed with ether to give the title compound as a white solid (125 mg, 95%). MS (ES): m/z [M+H] 327.24

### REFERENCE EXAMPLE 186

### N-(tert-butoxycarbonyl)-N,β,β-trimethyl-L-phenylalanyl-N¹-[(1S,2E)-4-ethoxy-1-isopropyl-3-methyl-4-oxobut-2-enyl]-N¹,3-dimethylisoleucinamide

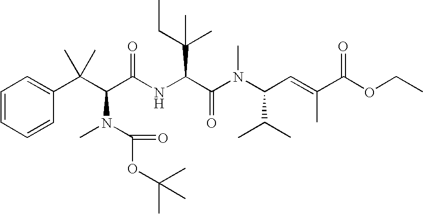

By a method analogous to that described in General Procedure Ivb, to a cooled (0° C., ice bath) solution of (2S)-2-[(tert-butoxycarbonyl)(methyl)amino)]-3-methyl-3-phenylbutanoic acid (115 mg, 0.32 mmol), hydroxybenzotriazole (52 mg, 0.38 mmol) and 1-(3-dimethylaminopropyl)-3-ethylcarbodiimine hydrochloride (86 mg, 0.45 mmol) in anhydrous dimethylformamide (3-5 mL) under a nitrogen atmosphere is added N-methylmorpholine (0.070 mL, 0.64 mmol) via syringe. After stirring for 1 hour at 0° C., to this mixture is added a solution of ethyl ethyl (2E,4S)-2,5-dimethyl-4-[methyl(3-methylisoleucyl)amino]hex-2-enoate (115 mg, 0.32 mmol, from Reference Example 185) in anhydrous dimethylformamide (1.5 mL). The cooling bath is removed, and the resulting mixture is stirred for 15 hours at room temperature. Chromatography (silica gel, flash column) gave the title compound (120 mg, 51%). MS (ES): m/z [M+H] 616.84

### REFERENCE EXAMPLE 187

### N-(tert-butoxycarbonyl)-N,β,β-trimethyl-L-phenylalanyl-N¹-[(1S,2E)-3-carboxy-1-isopropylbut-2-enyl]-N¹,3-dimethylisoleucinamide

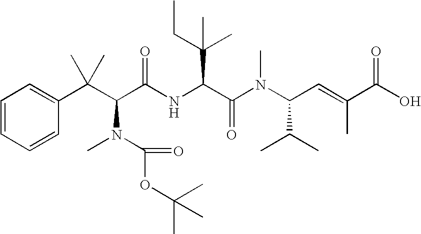

By a method analogous to that described in Reference Procedure V, N-(tert-butoxycarbonyl)-N,β,β-trimethyl-L-phenylalanyl-N¹-[(1S,2E)-4-ethoxy-1-isopropyl-3-methyl-4-oxobut-2-enyl]-N¹,3-dimethylisoleucinamide (150 mg, 0.17 mmol, from Reference Example 186) is dissolved in methanol (0.8 mL), tetrahydrofuran (0.8 mL) and water (0.4 mL) and treated with lithium hydroxide (8 mg, 0.34 mmol) to give the title compound (80 mg, 90%). MS (ES): m/z [M−H] 528.34

### REFERENCE EXAMPLE 188

### Methyl (betaS)-N-(tert-butoxycarbonyl)-N,beta-dimethyl-L-phenylalaninate

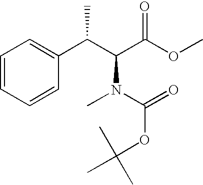

Commercially available (BetaS)-N-(tert-butoxycarbonyl)-beta-methyl-L-phenylalanine is converted to methyl (BetaS)-N-(tert-butoxycarbonyl)-N,beta-dimethyl-L-phenylalaninate by a procedure analogous to that described in Reference Example 63. MS (ES): m/z 308.18577(M+H) (Calc'd MW=307.17836)

### REFERENCE EXAMPLE 189

### O-benzyl-N-(tert-butoxycarbonyl)-N-methyl-L-threonyl-N¹-[(1S,2E)4-ethoxy-1-isopropyl-3-methyl-4-oxobut-2-enyl]-N¹,3-dimethyl-L-valinamide

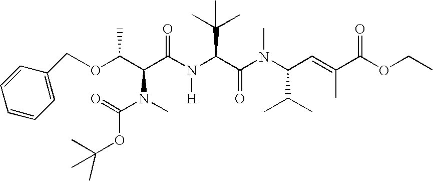

By a method analogous to that described in General Procedures Ivb, commercially available 3-benzyloxy-2-methylamino-butyric acid (500 mg, 1.55 mmol) is converted to the title compound (1.0 g, colorless oil). MS (ES): m/z 618.4 (M+H)

### REFERENCE EXAMPLE 190

### O-benzyl-N-methyl-L-threonyl-N¹-[(1S,2E)-4-ethoxy-1-isopropyl-3-methyl-4-oxobut-2-enyl]-N ,3-dimethyl-L-valinamide

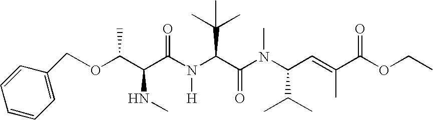

A solution of O-benzyl-N-(tert-butoxycarbonyl)-N-methyl-L-threonyl-N¹-[(1S,2E)4-ethoxy-1-isopropyl-3-methyl-4-oxobut-2-enyl]-N¹,3-dimethyl-L-valinamide (˜0.6 g, from Reference Example 189) in dichloromethane (6 mL) is treated with trifluoroacetic acid (1 mL). After 18 hours concentration in vacuo gave the title compound as a pale yellow oil. MS (ES): m/z 518.7 (M+H)

### REFERENCE EXAMPLE 191

### Ethyl (6S,9S,12S,13E)-9-tert-butyl-12-isopropyl-2,2,11,14-tetramethyl-6-(1-naphthylmethyl)-4,7,10-trioxo-3-oxa-5,8,11-triazapentadec-13-en-15-oate

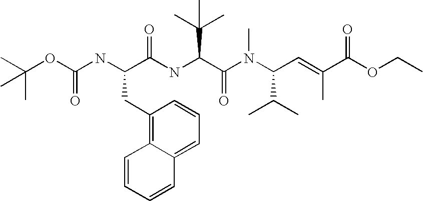

By using an analogous procedure to that described in General Procedure IVb, N-(tert-butoxycarbonyl)-3-(1-naththyl)-L-alanine (165 mg, 0.52 mmol, Advanced ChemTech Co.) is treated with ethyl (2E,4S)-2,5-dimethyl4-[methyl(3-methyl-L-valyl)amino]hex-2-enoate hydrochloride (178 mg, 0.52 mmol), in the presence of hydroxybenzotriazole (85 mg, 0.63 mmol), 1-(3-dimethylaminopropyl)-3-ethylcarbodiimide hydrochloride (140 mg, 0.73 mmol), N-methylmorpholine (0.086 ml, 0.78 mmol) in anhydrous N,N-dimethylformamide (1.5 ml). The products are purified by chromatography (silica gel, flash column, 2.5% methanol in methylene chloride), to provide ethyl (6S,9S,12S,13E)-9-tert-butyl-12-isopropyl-2,2,11,14-tetramethyl-6-(1-naphthylmethyl)-4,7,10-trioxo-3-oxa-5,8,11-triazapentadec-13-en-15-oate (135 mg, 43%) as a white colorless glass.

### REFERENCE EXAMPLE 192

### (6S,9S,12S,13E)-9-tert-Butyl-1 2-isopropyl-2,2,11,14-tetramethyl-6-(1-naphthylmethyl)-4,7,10-trioxo-3-oxa-5,8,11-triazapentadec-13-en-15-oic Acid

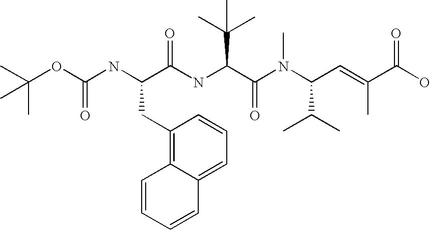

By using an analogous procedure to that described in General Procedure V, ethyl (6S,9S,12S,13E)-9-tert-butyl-12-isopropyl-2,2,11,14-tetramethyl-6-(1-naphthylmethyl)-4,7,10-trioxo-3-oxa-5,8,11-triazapentadec-13-en-15-oate (100 mg, 0.164 mmol, obtained from Reference Example 191) is treated with aqueous lithium hydroxide (1.28 mmol) in water (1.3 ml) and methanol (5 ml) at room temperature for 15 hours, to provide (6S,9S,12S,13E)-9-tert-butyl-12-isopropyl-2,2,11,14-tetramethyl-6-(1-naphthylmethyl)4,7,10-trioxo-3-oxa-5,8,11-triazapentadec-13-en-15-oic acid (80 mg, 84%) as a white powder. MS (ES): m/z 582.0 (M+H). Analytical HPLC: (4.6×150 mm Prodigy ODS3 column eluted with 45 to 55% acetonitrile in water containing 0.02% TFA over 24 minutes-isocratic method): 88.7% (at 18.0 minutes ) of (6S,9S,12S,13E)-9-tert-butyl-12-isopropyl-2,2,11,14-tetramethyl-6-(1-naphthylmethyl)-4,7,10-trioxo-3-oxa-5,8,11-triazapentadec-13-en-15-oic acid, and 8.2% (at 18.5minutes) of the other diastereomer.

### REFERENCE EXAMPLE 193

### N-cyclohex-1-en-1-yl-2-[formyl(methyl)amino]-2-(1-phenylcyclopropyl)acetamide

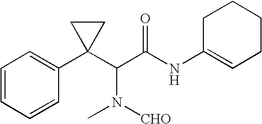

A mixture of 1-phenyl-cyclopropanecarbaldehyde (H. Keuntzel, et.al.; Helv. Chim. Acta.; 54, 868 (1971)) (1.46 g, 10.0 mmol), methylamine (6.25 mL of 2M solution in methanol, 12.5 mmole) and methanol (7 mL) was stirred for 15 minutes. Then 1-Isocyano-cyclohexene (1.07 g, 10.0 mmole) and formic acid (600 mg of 96%, 12.5 mmole) were added. After 18 h the reaction mixture was concentrated in vacuo and triturated with 10:1 hexane/ethyl acetate, filtered and dried to give a white solid, 1.23 g mg, MS (ES): m/z 313.2 (M+H).

### REFERENCE EXAMPLE 194

### (Methylamino)(1-phenylcyclopropyl)acetic Acid

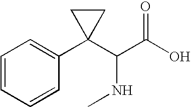

N-cyclohex-1-en-1-yl-2-[formyl(methyl)amino]-2-(1-phenylcyclopropyl)acetamide (400 mg, 1.23 mmole) was refluxed with aqueous 6N HCL (3 mL) for 1 hour. The mixture was concentrated in vacuo and the residue purified by HPLC to give a white solid, 224 mg, MS (ES): m/z 220.1 (M−H).

### EXAMPLE 1a AND 1b

### 3-Chloro-N,β,β-trimethyl-L-phenylalanyl-N¹-[(1S,2E)4-ethoxy-1-isopropyl-3-methyl-4-oxo-2-butenyl]-N¹,3-dimethyl-L-valinamide and Example 1b, 3-chloro-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide

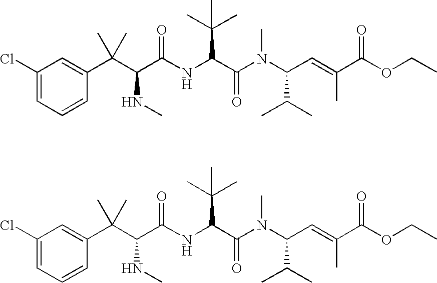

By a procedure analogous to that described in General Procedure IVa, 3-chloro-N,-trimethylphenylalanine (330 mg, 1.37 mmol, obtained from Reference Example 4) is treated with ethyl (2E,4S)-2,5-dimethyl-4-[methyl(3-methyl-L-valyl)amino]hex-2-enoate hydrochloride (426 mg, 1.37 mmol) in the presence of benzotriazole-1-yl-oxy-tris-pyrrolidinophosphonium hexafluorophosphate (746 mg, 1.43 mmol) and diisopropylethylamine (0.713 mL, 4.1 mmol) in anhydrous methylene chloride (10 mL). The products are purified by chromatography (silica gel, flash column, 40% ethyl acetate in hexanes), to provide 3-chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (263 mg, 36%) as a white amorphous solid, 3-chloro- N,β,β-trimethyl-D-phenylalanyl-N¹-[(1S,2E)4-ethoxy-1-isopropyl-3-methyl-4-oxo-2-butenyl]-N¹,3-dimethyl-L-valinamide (232 mg, 32%) as a white amorphous solid, and a mixture of non-separated two isomers mentioned above(157 mg, 21%). 3-Chloro- N,β,β-trimethyl-L-phenylalanyl-N¹-[(1S,2E)-4-ethoxy-1-isopropyl-3-methyl-4-oxo-2-butenyl]-N¹,3-dimethyl-L-valinamide MS (ES): m/z 536.39 (M+H). IR cm⁻¹: 3372.07, 2966.12, 1714.64, 1635.74. Analytical HPLC (4.6×150 mm YMC Pack Pro C18 column eluted with 10-100% acetonitrile in water containing 0.02% formic acid over 35 minutes): 98.86% at 25.0 minutes.

3-Chloro- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide MS (ES): m/z 536.35 (M+H). IR cm⁻¹: 3379.96, 2965.68, 1714.87, 1639.15. Analytical HPLC (4.6×150 mm YMC Pack Pro C18 column eluted with 10-100% acetonitrile in water containing 0.02% formic acid over 35 minutes): 94.85% (at 24.7 minutes) of 3-chloro- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide, and 0.41% (at 25.5 minutes) of 3-chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide.

### EXAMPLE 2

### 3-Chloro- N,β,β-trimethyl-L-phenylalanyl-N¹-[(1S,2E)-3-carboxy-1-isopropyl-2-butenyl]-N¹,3-dimethyl-L-valinamide

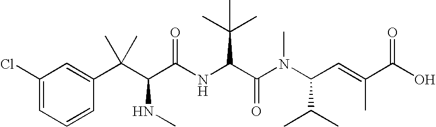

By using a procedure analogous to that described in General Procedure V, 3-chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (233 mg, 0.43 mmol, obtained from Example 1) is treated with aqueous lithium hydroxide solution (1.3 mmol), in water (0.66 mL) and methanol (3 mL), to provide 3-chloro-_N,β,β-trimethyl-L-phenylalanyl-N¹-[(1S,2E)-3-carboxy-1-isopropyl-2-butenyl]-N¹,3-dimethyl-L-valinamide (140 mg, 64%) as a white solid. MS (ES): m/z 508.3 (M+H). IR cm⁻¹: 3348.63, 2965.72, 1635.61. Analytical HPLC (4.6×150 mm YMC Pack Pro C18 column eluted with 10-100% acetonitrile in water containing 0.02% formic acid over 30 minutes): 91.91% (at 14.5 minutes) of 3-chloro-N,β,β-trimethyl-L-phenylalanyl-N¹-[(1S,2E)-3-carboxy-1-isopropyl-2-butenyl]-N¹,3-dimethyl-L-valinamide, and 7.30% (at 14.9 minutes)of 3-chloro- N,β,β-trimethyl-D-phenylalanyl-N¹-[(1S,2E)-3-carboxy-1-isopropyl-2-butenyl]-N¹,3-dimethyl-L-valinamide.

### EXAMPLE 3

### 3-Chloro- N,β,β-trimethyl-D-phenylalanyl-N¹-[(1S,2E)-3-carboxy-1-isopropyl-2-butenyl]-N¹,3-dimethyl-L-valinamide

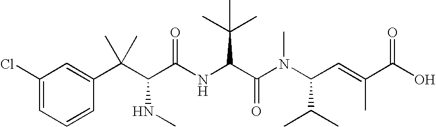

By using a procedure analogous to that described in Example V, 3-chloro- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (200 mg, 0.37 mmol, obtained from Example 1) is treated with aqueous lithium hydroxide solution (1.2 mmol), in water (0.58 mL) and methanol (2.8 mL), to provide 3-chloro- N,β,β-trimethyl-D-phenylalanyl-N¹-[(1S,2E)-3-carboxy-1-isopropyl-2-butenyl]-N¹,3-dimethyl-L-valinamide (167 mg, 89%) as a white solid. MS (ES): m/z 508.29 (M+H). IR cm⁻¹: 3424.60, 2966.31, 1682.00, 1630.01. Analytical HPLC (4.6×150 mm YMC Pack Pro C18 column eluted with 10-100% acetonitrile in water containing 0.02% formic acid over 35 minutes): 92.60% (at 19.1 minutes) of 3-chloro- N,β,β-trimethyl-D-phenylalanyl-N¹-[(1S,2E)-3-carboxy-1-isopropyl-2-butenyl]-N¹,3-dimethyl-L-valinamide, and 2.33% (at 18.8 minutes) of3-chloro-_N,β,β-trimethyl-L-phenylalanyl-N¹-[(1S,2E)-3-carboxy-1-isopropyl-2-butenyl]-N¹,3-dimethyl-L-valinamide.

### EXAMPLE 4a AND 4b

### 4-Chloro- N,β,β-trimethyl-L-phenylalanyl-N¹-[(1S,2E)-4-ethoxy-1-isopropyl-3-methyl-4-oxo-2-butenyl]-N¹,3-dimethyl-L-valinamide and Example 4b, 4-chloro- N,β,β-trimethyl-D-phenylalanyl-N¹-[(1S,2E)4-ethoxy-1-isopropyl-3-methyl-4-oxo-2-butenyl]-N¹,3-dimethyl-L-valinamide

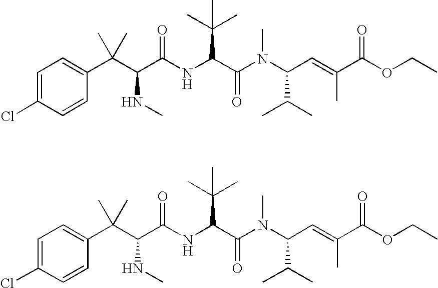

By a procedure analogous to that described in General Procedure IVa, 4-chloro-N,β,β-trimethylphenylalanine (347 mg, 1.4 mmol, obtained from Reference Example 8) is treated with ethyl (2E,4S)-2,5-dimethyl-4-[methyl(3-methyl-L-valyl)amino]hex-2-enoate hydrochloride (500 mg, 1.4 mmol) in the presence of benzotriazole-1-yl-oxy-tris-pyrrolidinophosphonium hexafluorophosphate (785 mg, 1.5 mmol) and diisopropylethylamine (0.750 mL, 4.3 mmol) in methylene chloride (10 mL). The products are purified by chromatography (silica gel, flash column, 40% ethyl acetate in hexanes), to provide 4-chloro- N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (295 mg, 39%) as a colorless glass, 4-chloro- N,β,β-methyl-D-phenylalanyl-N¹-[(1S,2E)-4-ethoxy-1-isopropyl-3-methyl-4-oxo-2-butenyl]-N¹,3-dimethyl-L-valinamide (276 mg, 37%) as a colorless glass.

4-Chloro N,β,β-trimethyl-L-phenylalanyl-N¹-[(1S,2E)-4-ethoxy-1-isopropyl-3-methyl4-oxo-2-butenyl]-N¹,3-dimethyl-L-valinamide MS (ES): m/z 536.31 (M+H). IR cm⁻¹: 3346.36, 2964.87, 1713.60, 1671.37, 1634.45. Analytical HPLC (4.6×150 mm Prodigy ODS3 column eluted with 10-95% acetonitrile in water containing 0.02% trifluoroacetic acid over 22 minutes): 93.20% at 15.3 minutes. 4-Chloro-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide MS (ES): m/z 536.37 (M+H). IR cm⁻¹: 3350.40, 2964.60, 1713.80, 1670.77, 1638.39. Analytical HPLC (4.6×150 mm Prodigy ODS3 column eluted with 10-95% acetonitrile in water containing 0.02% trifluoroacetic acid over 22 minutes): 99.07% at 15.1 minutes.

### EXAMPLE 5

### 4-Chloro- N,β,β-triethyl-L-phenylalanyl-N¹-[(1S,2E)-3-carboxy-1-isopropyl-2-butenyl]-N¹,3-dimethyl-L-valinamide

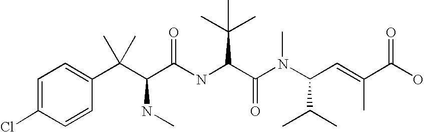

By using a procedure analogous to that described in General Procedure V, 4-chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (247 mg, 0.46 mmol, obtained from Example 4) is treated with aqueous lithium hydroxide solution (2 mmol), in water (1 mL) and tetrahydrofuran (4 mL), to provide 4-chloro- N,β,β-trimethyl-L-phenylalanyl-N¹-[(1S,2E)-3-carboxy-1-isopropyl-2-butenyl]-N¹,3-dimethyl-L-valinamide (95 mg, 41%) as a white glassy solid. MS (ES): m/z 506.4 (M−H), 1013.6 (2M−H). IR cm⁻¹: 3340.30, 2965.48, 1633.17. Analytical HPLC (4.6×150 mm YMC Pack Pro C18 column eluted with 10-100% acetonitrile in water containing 0.02% formic acid over 35 minutes): 99.4% at 15.5 minutes.

### EXAMPLE 6

### 4-Chloro-N,β,β-trimethyl-D-phenylalanyl-N¹-[(1S,2E)-3-carboxy-1-isopropyl-2-butenyl]-N¹,3-dimethyl-L-valinamide

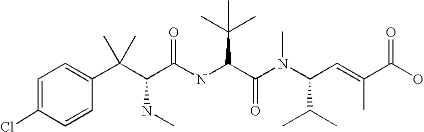

By using a procedure analogous to that described in General Procedure V, 4-chloro-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (232 mg, 0.433 mmol, obtained from Example 4) is treated with aqueous lithium hydroxide solution (1.3 mmol), in water (0.652 mL) and tetrahydrofuran (3 mL), to provide 4-chloro-_N,β,β-trimethyl-D-phenylalanyl-N¹-[(1S,2E)-3-carboxy-1-isopropyl-2-butenyl]-N¹,3-dimethyl-L-valinamide (139 mg, 63%) as a white solid. MS (ES): m/z 508.28 (M+H). IR cm⁻¹: 3375.86, 2964.03, 1633.46, 1397.85. Analytical HPLC (4.6×150 mm YMC Pack Pro C18 column eluted with 12-100% acetonitrile in water containing 0.02% formic acid over 35 minutes): 3.01% at 23.9 minutes, 93.92% at 24.4 minutes.

### EXAMPLE 7a AND 7b

### N,β,β,3-Tetramethyl-L-phenylalanyl-N¹-[(1S,2E)4-ethoxy-1-isopropyl-3-methyl-4-oxo-2-butenyl]-N¹,3-dimethyl-L-valinamide and Example 7b, N,β,β,3-tetramethyl-D-phenylalanyl-N¹-[(1S,2E)-4-ethoxy-1-isopropyl-3-methyl4-oxo-2-butenyl]-N¹,3-dimethyl-L-valinamide

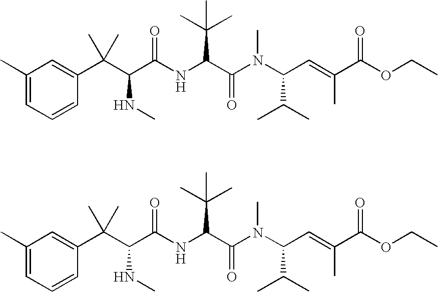

By a procedure analogous to that described in General Procedure IVb, N,β,β,3-tetramethylphenylalanine (317 mg, 1.43 mmol, obtained from Reference Example 12) is treated with ethyl (2E,4S)-2,5-dimethyl4-[methyl(3-methyl-L-valyl)amino]hex-2-enoate hydrochloride (500 mg, 1.43 mmol) in the presence of benzotriazole-1-yl-oxy-tris-pyrrolidinophosphonium hexafluorophosphate (783 mg, 1.5 mmol) and diisopropylethylamine (0.75 mL, 4.3 mmol) in anhydrous dimethylformamide (3 mL). The products are purified by chromatography (silica gel, flash column, 40% ethyl acetate in hexanes), to provide N,β,β,3-tetramethyl-L-phenylalanyl-N¹-[(1S,2E)-4-ethoxy-1-isopropyl-3-methyl-4-oxo-2-butenyl]-N¹,3-dimethyl-L-valinamide (141 mg, 19%) as a colorless glass, N,β,β,3-tetramethyl-D-phenylalanyl-N¹-[(1S,2E)4-ethoxy-1-isopropyl-3-methyl4-oxo-2-butenyl]-N¹,3-dimethyl-L-valinamide (125 mg, 17%) as a colorless glass, and a mixture of non-separated two isomers mentioned above (36 mg, 5%). N,β,β,3-Tetramethyl-L-phenylalanyl-N¹-[(1S,2E)4-ethoxy-1-isopropyl-3-methyl4-oxo-2-butenyl]-N¹,3-dimethyl-L-valinamide MS (ES): m/z 516.68 (M+H). Analytical HPLC: (4.6×150 mm YMC Pro Pack C18 column eluted with 30 to 100% acetonitrile in water containing 0.02% formic acid over 30 minutes): 94.19% (at 13.8 minutes) of N,β,β,3-tetramethyl-L-phenylalanyl-N¹-[(1S,2E)4-ethoxy-1-isopropyl-3-methyl-4-oxo-2-butenyl]-N¹,3-dimethyl-L-valinamide, and 5.81% (at 16.1 minutes)of N,β,β,3-tetramethyl-D-phenylalanyl-N¹-[(1S,2E)-4-ethoxy-1-isopropyl-3-methyl-4-oxo-2-butenyl]-N¹,3-dimethyl-L-valinamide. N,β,β,3-Tetramethyl-D-phenylalanyl-N¹-[(1S,2E)-4-ethoxy-1-isopropyl-3-methyl-4-oxo-2-butenyl]-N¹,3-dimethyl-L-valinamide MS (ES): m/z 516.69 (M+H). Analytical HPLC: (4.6×150 mm YMC Pro Pack C18 column eluted with 30 to 100% acetonitrile in water containing 0.02% formic acid over 30 minutes): 6.12% (at 14.9 minutes) of N,β,β,3-tetramethyl-L-phenylalanyl-N¹-[(1S,2E)-4-ethoxy-1-isopropyl-3-methyl-4-oxo-2-butenyl]-N¹,3-dimethyl-L-valinamide, and 93.88% (at 15.4 minutes) of N,β,β,3-tetramethyl-D-phenylalanyl-N¹-[(1S,2E)-4-ethoxy-1-isopropyl-3-methyl-4-oxo-2-butenyl]-N¹,3-dimethyl-L-valinamide.

### EXAMPLE 8

### N,β,β,3-Tetramethyl-L-phenylalanyl-N¹-[(1S,2E)-3-carboxy-1-isopropyl-2-butenyl]-N¹,3-dimethyl-L-valinamide

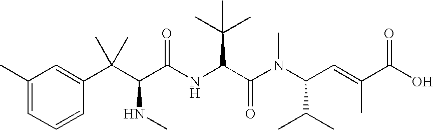

By using a procedure analogous to that described in General Procedure V, N,β,β,3-tetramethyl-L-phenylalanyl-N¹-[(1S,2E)-4-ethoxy-1-isopropyl-3-methyl-4-oxo-2-butenyl]-N¹,3-dimethyl-L-valinamide (95 mg, 0.184 mmol, obtained from Example 7) is treated with aqueous lithium hydroxide solution (0.976 mmol), in water (0.486 mL) and methanol (2 mL), to provide N,β,β,3-tetramethyl-L-phenylalanyl-N¹-[(1S,2E)-3-carboxy-1-isopropyl-2-butenyl]-N¹,3-dimethyl-L-valinamide as a pale yellow solid (350 mg). Analytical sample is obtained from purification of 250 mg of the above sample by using preparative HPLC, to give the corresponding trifluoroacetic acid salt (40 mg). MS (ES): m/z 488.36 (M+H). IR cm⁻¹: 2969.69, 1679.56, 1203.71. Analytical HPLC: (4.6×150 mm YMC Pro Pack C18 column eluted with 10 to 100% acetonitrile in water containing 0.02% formic acid over 35 minutes): 98.93% at 14.2 minutes.

### EXAMPLE 9

### N,β,β,3-Tetramethyl-D-phenylalanyl-N¹-[(1S,2E)-3-carboxy-1-isopropyl-2-butenyl]-N¹,3-dimethyl-L-valinamide

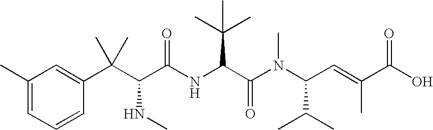

By using a procedure analogous to that described in General Procedure V, N,β,β,3-tetramethyl-D-phenylalanyl-N¹-[(1S,2E)-4-ethoxy-1-isopropyl-3-methyl-4-oxo-2-butenyl]-N¹,3-dimethyl-L-valinamide (111 mg, 0.216 mmol, obtained from Example 7) is treated with aqueous lithium hydroxide solution (0.976 mmol), in water (0.486 mL) and methanol (2 mL), to provide N,β,β,3-tetramethyl-D-phenylalanyl-N¹-[(1S,2E)-3-carboxy-1-isopropyl-2-butenyl]-N¹,3-dimethyl-L-valinamide (400 mg) as a pale yellow solid. Analytical sample is obtained by purification of the above sample (250 mg) with preparative HPLC, to give the corresponding trifluoroacetic acid salt (61 mg). MS (ES): m/z 488.39 (M+H) IR cm⁻¹: 3407.88, 2969.67, 1679.82. Analytical HPLC: (4.6×150 mm YMC Pro Pack C18 column eluted with 10 to 100% acetonitrile in water containing 0.02% formic acid over 35 minutes): 6.97% (at 14.3 minutes) of N,β,β,3-tetramethyl-L-phenylalanyl-N¹-[(1S,2E)-3-carboxy-1-isopropyl-2-butenyl]-N¹,3-dimethyl-L-valinamide (other isomer), 90.69% (at 14.8 minutes) of N,β,β,3-tetramethyl-D-phenylalanyl-N¹-[(1S,2E)-3-carboxy-1-isopropyl-2-butenyl]-N¹,3-dimethyl-L-valinamide.

### EXAMPLE 10a AND 10b

### N,β,β,4-Tetramethyl-L-phenylalanyl-N¹-[(1S,2E)-4-ethoxy-1-isopropyl-3-methyl4-oxo-2-butenyl]-N¹,3-dimethyl-L-valinamide and (10b) N,β,β,4-tetramethyl-D-phenylalanyl-N¹-[(1S,2E)-4-ethoxy-1-isopropyl-3-methyl-4-oxo-2-butenyl]-N¹,3-dimethyl-L-valinamide

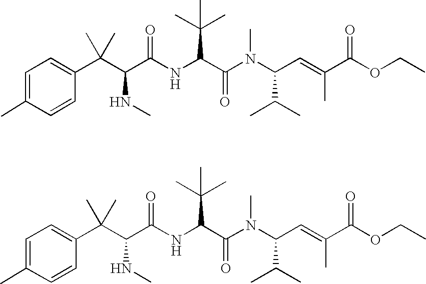

By a procedure analogous to that described in General Procedure IVa, N,β,β,4-tetramethylphenylalanine (252 mg, 1.14 mmol, obtained from Reference Example 16) is treated with ethyl (2E,4S)-2,5-dimethyl-4-[methyl(3-methyl-L-valyl)amino]hex-2-enoate hydrochloride (398 mg, 1.14 mmol, in the presence of benzotriazole-1-yl-oxy-tris-pyrrolidinophosphonium hexafluorophosphate (623 mg, 1.2 mmol) and diisopropylethylamine (0.60 mL, 3.4 mmol) in anhydrous dimethylformamide (5 mL). The products are purified by chromatography (silica gel, flash column, 40% ethyl acetate in hexanes), to provide N,β,P,4-tetramethyl-L-phenylalanyl-N¹-[(1S,2E)-4-ethoxy-1-isopropyl-3-methyl-4-oxo-2-butenyl]-N¹,3-dimethyl-L-valinamide (105 mg, 18%) as a pale yellow glass, N,β,β,4-tetramethyl-D-phenylalanyl-N¹-[(1S,2E)-4-ethoxy-1-isopropyl-3-methyl-4-oxo-2-butenyl]-N¹,3-dimethyl-L-valinamide (94 mg, 16%) as a colorless glass, and a mixture of non-separated two isomers mentioned above (94 mg, 16%).

N,β,β,4-Tetramethyl-L-phenylalanyl-N¹-[(1S,2E)-4-ethoxy-1-isopropyl-3-methyl-4-oxo-2-butenyl]-N¹,3-dimethyl-L-valinamide MS (ES): m/z 516.58 (M+H).

IR cm⁻¹: 3344.24, 2965.00, 1637.00.

Analytical HPLC (4.6×150 mm YMC Pack Pro C18 column eluted with 17-100% acetonitrile in water containing 0.02% formic acid over 35 minutes): 87.41% (at 16.9 minutes) of N,β,β,4-Tetramethyl-L-phenylalanyl-N¹-[(1S,2E)-4-ethoxy-1-isopropyl-3-methyl-4-oxo-2-butenyl]-N¹,3-dimethyl-L-valinamide, and 3.24% (at 17.2 minutes) of N,β,β,4-Tetramethyl-D-phenylalanyl-N¹-[(1S,2E)4-ethoxy-1-isopropyl-3-methyl-4-oxo-2-butenyl]-N¹,3-dimethyl-L-valinamide. N,β,β,4-Tetramethyl-D-phenylalanyl-N¹-[(1S,2E)-4-ethoxy-1-isopropyl-3-methyl-4-oxo-2-butenyl]-N¹,3-dimethyl-L-valinamide MS (ES): m/z 516.60 (M+H). Analytical HPLC (4.6×150 mm YMC Pack Pro C18 column eluted with 17-100% acetonitrile in water containing 0.02% formic acid over 35 minutes): 91.26% at 16.9 minutes.

### EXAMPLE 11

### N,β,β,4-Tetramethyl-L-phenylalanyl-N¹-[(1S,2E)-3-carboxy-1-isopropyl-2-butenyl]-N¹,3-dimethyl-L-valinamide

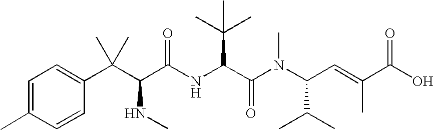

By using a procedure analogous to that described in General Procedure V, N,β,β,4-tetramethyl-L-phenylalanyl-N¹-[(1S,2E)-4-ethoxy-1-isopropyl-3-methyl-4-oxo-2-butenyl]-N¹,3-dimethyl-L-valinamide (171 mg, 0.332 mmol, obtained from Example 10) is treated with aqueous lithium hydroxide solution (1 mmol), in water (0.5 mL) and methanol (2.5 mL). The product is purified by preparative HPLC to provide N,β,β,4-tetramethyl-L-phenylalanyl-N¹-[(1S,2E)-3-carboxy-1-isopropyl-2-butenyl]-N¹,3-dimethyl-L-valinamide trifluoroacetic acid salt (80 mg, 40%) as a white solid. MS (ES): m/z 488.40 (M+H). IR cm⁻¹: 2969.06, 1679.95, 1203.34. Analytical HPLC (4.6×150 mm YMC Pack Pro C18 column eluted with 17-100% acetonitrile in water containing 0.02% formic acid over 30 minutes): 95.50% (at 13.0 minutes) of N,β,β,4-tetramethyl-L-phenylalanyl-N¹-[(1S,2E)-3-carboxy-1-isopropyl-2-butenyl]-N¹,3-dimethyl-L-valinamide, and 3.24% (at 14.3 minutes) of N,β,β,4-tetramethyl-D-phenylalanyl-N¹-[(1S,2E)-3-carboxy-1-isopropyl-2-butenyl]-N¹,3-dimethyl-L-valinamide.

### EXAMPLE 12

### N,β,β,4-Tetramethyl-D-phenylalanyl-N¹-[(1S,2E)-3-carboxy-1-isopropyl-2-butenyl]-N¹,3-dimethyl-L-valinamide

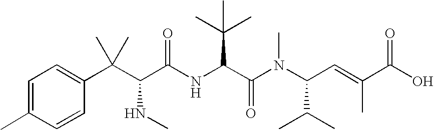

By using a procedure analogous to that described in General Procedure V, N,β,β,4-tetramethyl-D-phenylalanyl-N¹-[(1S,2E)-4-ethoxy-1-isopropyl-3-methyl4-oxo-2-butenyl]-N¹,3-dimethyl-L-valinamide (155 mg, 0.3 mmol, obtained from Example 10) is treated with aqueous lithium hydroxide solution (1 mmol), in water (0.5 mL) and methanol (2.5 mL). The product is purified by preparative HPLC to provide N,β,β,4-tetramethyl-D-phenylalanyl-N¹-[(1S,2E)-3-carboxy-1-isopropyl-2-butenyl]-N¹,3-dimethyl-L-valinamide trifluoroacetic acid salt (106 mg, 59%) as a white solid. MS (ES): m/z 488.35 (M+H). IR cm⁻¹: 3413.35, 2969.12, 1680.82. Analytical HPLC (4.6×150 mm YMC Pack Pro C18 column eluted with 15-100% acetonitrile in water containing 0.02% formic acid over 35 minutes): 4.36% (at 12.1 minutes) of N,β,β,4-tetramethyl-L-phenylalanyl-N¹-[(1S,2E)-3-carboxy-1-isopropyl-2-butenyl]-N¹,3-dimethyl-L-valinamide, and 92.75% (at 12.6 minutes) of N,β,β,4-tetramethyl-D-phenylalanyl-N¹-[(1S,2E)-3-carboxy-1-isopropyl-2-butenyl]-N¹,3-dimethyl-L-valinamide.

### EXAMPLE 13a AND 13b

### N,β, β,3,4-Pentamethyl-L-phenylalanyl-N¹-[(1S,2E)4-ethoxy-1-isopropyl-3-methyl-4-oxo-2-butenyl]-N¹,3-dimethyl-L-valinamide and Example 13b, N,β,β-3,4-pentamethyl-D-phenylalanyl-N¹-[(1S,2E)4-ethoxy-1-isopropyl-3-methyl4-oxo-2-butenyl]-N¹,3-dimethyl-L-valinamide

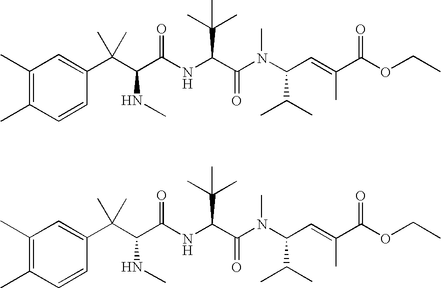

By a procedure analogous to that described in General Procedure IVb, N,β,β-3,4-pentamethylphenylalanine (940 mg, 4.01 mmol, obtained from Reference Example 19) is treated with ethyl (2E,4S)-2,5-dimethyl-4-[methyl(3-methyl-L-valyl)amino]hex-2-enoate hydrochloride (830 mg, 2.67 mmol, in the presence of hydroxybenzothriazole (540 mg, 4.01 mmol), 1-(3-dimethylaminopropyl)-3-ethylcarbodiimide hydrochloride (870 mg, 4.54 mmol), N-methylmorpholine (0.53 mL, 4.80 mmol) in anhydrous N,N-dimethylformamide (20 mL). The products are purified by chromatography (silica gel, flash column, 0.5% methanol in methylene chloride), to provide N,β,β-3,4-pentamethyl-L-phenylalanyl-N¹-[(1S,2E)4-ethoxy-1-isopropyl-3-methyl4-oxo-2-butenyl]-N¹,3-dimethyl-L-valinamide (404.4 mg, 28.7%) as a white amorphous solid. Further elution by 1-5% methanol in methylene chloride provided N,β,β,3,4-pentamethyl-D-phenylalanyl-N¹-[(1S,2E)4-ethoxy-1-isopropyl-3-methyl4-oxo-2-butenyl]-N¹,3-dimethyl-L-valinamide (480 mg, 34.0%) as a white amorphous solid. A mixture of non-separated two diastereomers mentioned above is also obtained (124.7 mg, 8.8%). N,β,β-3,4-Pentamethyl-L-phenylalanyl-N¹-[(1S,2E)4-ethoxy-1-isopropyl-3-methyl-4-oxo-2-butenyl]-N¹,3-dimethyl-L-valinamide MS (ES):m/z 530.4 (M+H) IR cm⁻¹: 2967, 1715, 1673, 1638. Analysis for C₃,H₅,N₃O₄: Calcd: C, 70.28; H, 9.70; N, 7.93. Found: C, 69.78;H, 9.51; N, 7.75.

Analytical HPLC (4.6×150 mm YMC pack Pro C18 column eluted with a liner gradient of 10-100% actonitrile in water containing 0.02% formic acid over 35 min): 96.9% at 20.414 minutes.

N,β,β-3,4-Pentamethyl-D-phenylalanyl-N¹-[(1S,2E)4-ethoxy-1-isopropyl-3-methyl-4-oxo-2-butenyl]-N¹,3-dimethyl-L-valinamide MS (ES): m/z 530.4 (M+H) IR cm⁻¹: 2966, 1715, 1672, 1641 Analysis for C₃₁H₅₁N₃O₄: Calcd: C, 70.28;H, 9.70; N, 7.93.

Found: C, 69.88;H, 9.51; N, 7.82.

Analytical HPLC: (4.6×150 mm YMC pack Pro C18 column eluted with a liner gradient of 10-100% actonitrile in water containing 0.02% formic acid over 35 min): 97.23% at 20.292 minutes.

### EXAMPLE 14

### N,β,β,3,4-Pentamethyl-L-phenylalanyl-N¹-[(1S,2E)-3-carboxy-1-isopropyl-2-butenyl]-N¹,3-dimethyl-L-valinamide

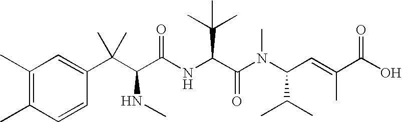

By using a procedure analogous to that described in General Procedure V, N,β,β,3,4-pentamethyl-L-phenylalanyl-N¹-[(1S,2E)-4-ethoxy-1-isopropyl-3-methyl-4-oxo-2-butenyl]-N¹,3-dimethyl-L-valinamide (350 mg, 0.661 mmol, obtained from Example 13) is treated with aqueous lithium hydroxide solution (5.29 mmol) in water (5 mL) and methanol (15 mL), to provide N,β,β,3,4-pentamethyl-L-phenylalanyl-N¹-[(1S,2E)-3-carboxy-1-isopropyl-2-butenyl]-N¹,3-dimethyl-L-valinamide (272 mg, 82.1%) as a white amorphous solid. MS (ES): m/z 500.3 (M−H), 1001.7 (2M−H), 502.3 (M+H). IR cm⁻¹: 2966, 1637. Analytical HPLC (4.6×150 mm YMC pack Pro C18 column eluted with a liner gradient of 10-100% actonitrile in water containing 0.02% formic acid over 35 min): 97.34% at 15.287 minutes.

### EXAMPLE 15

### N,β,β,3,4-Pentamethyl-D-phenylalanyl-N¹-[(1S,2E)-3-carboxy-1-isopropyl-2-butenyl]-N¹,3-dimethyl-L-valinamide

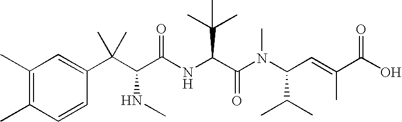

By using a procedure analogous to that described in General Procedure V, N,β,β,3,4-pentamethyl-D-phenylalanyl-N¹-[(1S,2E)-4-ethoxy-1-isopropyl-3-methyl-4-oxo-2-butenyl]-N¹,3-dimethyl-L-valinamide (422 mg, 0.766 mmol, obtained from Example 13) is treated with aqueous lithium hydroxide solution (6.13 mmol) in water (6 mL) and methanol (19 mL), to provide N,β,β,3,4-pentamethyl-D-phenylalanyl-N¹-[(1S,2E)-3-carboxy-1-isopropyl-2-butenyl]-N¹,3-dimethyl-L-valinamide (406.3 mg, 100%) as a white amorphous solid. MS (ES): m/z 502.36 (M+H). IR cm⁻¹: 2965, 1635. Analytical HPLC: (4.6×150 mm YMC pack Pro C18 column eluted with a liner gradient of 10-100% actonitrile in water containing 0.02% formic acid over 35 min): 96.20% (at 15.609 minutes) of N,β,β,3,4-pentamethyl-D-phenylalanyl-N¹-[(1S,2E)-3-carboxy-1-isopropyl-2-butenyl]-N¹,3-dimethyl-L-valinamide, and 0.54% (at 15.372 minutes) of N ,β,β,3,4-pentamethyl-L-phenylalanyl-N¹-[(1S,2E)-3-carboxy-1-isopropyl-2-butenyl]-N¹,3-dimethyl-L-valinamide.

### EXAMPLE 16a AND 16b

### N,β,β,3,5-Pentamethyl-L-phenylalanyl-N¹-[(1S,2E)-4-ethoxy-1-isopropyl-3-methyl-4-oxo-2-butenyl]-N¹,3-dimethyl-L-valinamide and Example 16b, N,β,β,3,5-pentamethyl-D-phenylalanyl-N¹-[(1S,2E)-4-ethoxy-1-isopropyl-3-methyl-4-oxo-2-butenyl]-N¹,3-dimethyl-L-valinamide

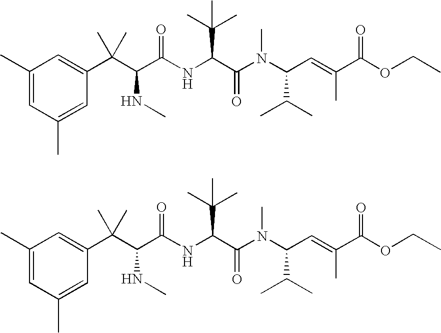

By a procedure analogous to that described in General Procedure IVb, N,β,β,3,5-pentamethylphenylalanine (940 mg, 4.01 mmol, obtained from Reference Example 22) is treated with ethyl (2E,4S)-2,5-dimethyl4-[methyl(3-methyl-L-valyl)amino]hex-2-enoate hydrochloride (830 mg, 2.67 mmol), in the presence of hydroxybenzothriazole (540 mg, 4.01 mmol), 1-(3-dimethylaminopropyl)-3-ethylcarbodiimide hydrochloride (870 mg, 4.54 mmol), N-methylmorpholine (0.53 mL, 4.80 mmol) in anhydrous N,N-dimethylformamide (20 mL). The products are purified by chromatography (silica gel, flash column, 0.5-0.6% methanol in methylene chloride), to provide N,β,β,3,5-pentamethyl-L-phenylalanyl-N¹-[(1S,2E)-4-ethoxy-1-isopropyl-3-methyl-4-oxo-2-butenyl]-N¹,3-dimethyl-L-valinamide (338.7 mg, 24.0%) as a white amorphous solid, N,β,β,3,5-pentamethyl-D-phenylalanyl-N¹-[(1S,2E)4-ethoxy-1-isopropyl-3-methyl-4-oxo-2-butenyl]-N¹,3-dimethyl-L-valinamide (330.8 mg, 23.5%) as a white amorphous solid, and a mixture of non-separated two diastereomers mentioned above (391.7 mg, 27.8%). N,β,β,3,5-Pentamethyl-L-phenylalanyl-N¹-[(1S,2E)-4-ethoxy-1-isopropyl-3-methyl-4-oxo-2-butenyl]-N¹,3-dimethyl-L-valinamide MS (ES): m/z 530.4 (M+H) IR cm⁻¹: 2966, 1715, 1673, 1638. Analysis for C₃₁H₅₁N₃O₄: Calcd: C, 70.28; H, 9.70; N, 7.93. Found: C, 70.00;H, 9.55; N, 8.06.

Analytical HPLC (4.6×150 mm YMC pack Pro C18 column eluted with a liner gradient of 10-100% actonitrile in water containing 0.02% formic acid over 35 min): 95.89% at 20.680 minutes.

N,β,β,3,5-Pentamethyl-D-phenylalanyl-N¹-[(1S,2E)-4-ethoxy-1-isopropyl-3-methyl-4-oxo-2-butenyl]-N¹,3-dimethyl-L-valinamide MS (ES): m/z 530.4 (M+H) IR cm⁻¹: 2966, 1715, 1672, 1641. Analysis for C₃₁H₅₁N₃O₄: Calcd: C, 70.28;H, 9.70; N, 7.93.

Found: C, 69.58;H, 9.36; N, 7.53.

Analytical HPLC: (4.6×150 mm YMC pack Pro C18 column eluted with a liner gradient of 10-100% actonitrile in water containing 0.02% formic acid over 35 min): 95.43% at 20.639 minutes.

### EXAMPLE 17

### N,β,β,3,5-Pentamethyl-L-phenylalanyl-N¹-[(1S,2E)-3-carboxy-1-isopropyl-2-butenyl]-N¹,3-dimethyl-L-valinamide

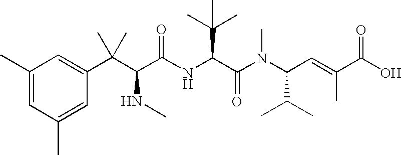

By using a procedure analogous to that described in General Procedure V, N,β,β,3,5-pentamethyl-L-phenylalanyl-N¹-[(1S,2E)-4-ethoxy-1-isopropyl-3-methyl-4-oxo-2-butenyl]-N¹,3-dimethyl-L-valinamide (285 mg, 0.538 mmol, obtained from Example 16) is treated with aqueous lithium hydroxide solution (4.3 mmol) in water (4.3 mL) and methanol (12 mL), to provide N,β,β,3,5-pentamethyl-L-phenylalanyl-N¹-[(1S,2E)-3-carboxy-1-isopropyl-2-butenyl]-N¹,3-dimethyl-L-valinamide (160.7 mg, 59.5%) as a white amorphous solid. MS (ES): m/z 502.3 (M+H). IR cm⁻¹: 2965, 1637.

Analytical HPLC (4.6×150 mm YMC pack Pro C18 column eluted with a liner gradient of 10-100% actonitrile in water containing 0.002% formic acid over 35 min): 96.59% at 16.269 minutes.

### EXAMPLE 18

### N,β,β,3,5-Pentamethyl-D-phenylalanyl-N¹-[(1S,2E)-3-carboxy-1-isopropyl-2-butenyl]-N¹,3-dimethyl-L-valinamide

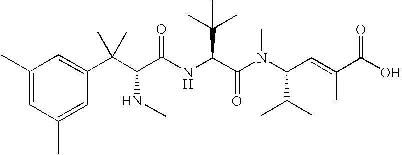

By using a procedure analogous to that described in General Procedure V, N,β,β,3,5-pentamethyl-D-phenylalanyl-N¹-[(1S,2E)-4-ethoxy-1-isopropyl-3-methyl-4-oxo-2-butenyl]-N¹,3-dimethyl-L-valinamide (341 mg, 0.644 mmol, obtained from Example 16) is treated with aqueous lithium hydroxide solution (5.2 mmol) in water (5 mL) and methanol (15 mL), to provide N,β,β,3,5-pentamethyl-D-phenylalanyl-N¹-[(1S,2E)-3-carboxy-1-isopropyl-2-butenyl]-N¹,3-dimethyl-L-valinamide (215.4 mg, 66.7%) as a white amorphous solid. MS (ES): m/z 502.3 (M+H). IR cm⁻¹: 2965, 1640. Analytical HPLC (4.6×150 mm YMC pack Pro C18 column eluted with a liner gradient of 10-100% actonitrile in water containing 0.02% formic acid over 35 min): 93.47% (at 15.947 minutes) of N,β,β,3,5-pentamethyl-D-phenylalanyl-N¹-[(1S,2E)-3-carboxy-1-isopropyl-2-butenyl]-N¹,3-dimethyl-L-valinamide, and 4.11% (at 15.602 minutes) of N,β,β,3,5-pentamethyl-D-phenylalanyl-N¹-[(1S,2E)-3-carboxy-1-isopropyl-2-butenyl]-N¹,3-dimethyl-L-valinamide.

### EXAMPLE 19a AND 19b

### N-Methyl-3-(2-thienyl)-L-valyl-N1-[(1S,2E)4-ethoxy-1-isopropyl-3-methyl4-oxo-2-butenyl]-N¹,3-dimethyl-L-valinamide and (19b) N-methyl-3-(2-thienyl)-D-valyl-N¹-[(1S,2E)4-ethoxy-1-isopropyl-3-methyl4-oxo-2-butenyl]-N¹,3-dimethyl-L-valinamide

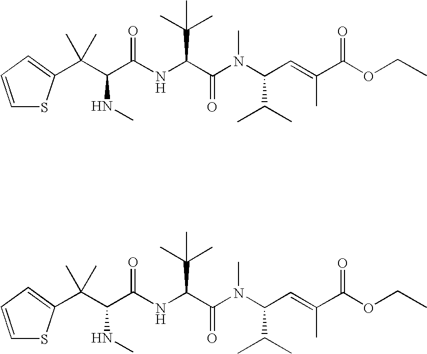

By a procedure analogous to that described in General Procedure IVb, N-methyl-3-thien-2-ylvaline (667 mg, 3.14 mmol, obtained from Reference Example 25) is treated with ethyl (2E,4S)-2,5-dimethyl4-[methyl(3-methyl-L-valyl)amino]hex-2-enoate hydrochloride (1.1 g, 3.15 mmol) in the presence of hydroxybenzothriazole (425 mg, 3.14 mmol), 1-(3-dimethylaminopropyl)-3-ethylcarbodiimide hydrochloride (843 mg, 4.4 mmol), N-methylmorpholine (0.52 mL, 4.71 mmol) in anhydrous dimethylformamide (20 mL). The products are purified by chromatography (silica gel, flash column, 0.5% methanol in methylene chloride), to provide N-methyl-3-(2-thienyl)-L-valyl-N¹-[(1S,2E)4-ethoxy-1-isopropyl-3-methyl-4-oxo-2-butenyl]-N¹,3-dimethyl-L-valinamide (318.3 mg, 20%) as a yellow oil, N-methyl-3-(2-thienyl)-D-valyl-N¹-[(1S,2E)4-ethoxy-1-isopropyl-3-methyl-4-oxo-2-butenyl]-N¹,3-dimethyl-L-valinamide (587.4 mg, 37.0%) as an oil, and a mixture of non-separated two isomers mentioned above (295.8 mg, 18.6%). N-Methyl-3-(2-thienyl)-L-valyl-N¹-[(1S,2E)4-ethoxy-1-isopropyl-3-methyl-4-oxo-2-butenyl]-N¹,3-dimethyl-L-valinamide MS (ES): m/z 508.28 (M+H) IR cm⁻¹: 3340, 2964, 2873, 1714, 1638. Analytical HPLC (4.6×150 mm YMC pack Pro C18 column eluted with a liner gradient of 10-100% actonitrile in water containing 0.02% formic acid over 35 min): 77% (at 18.143 minutes) of N-methyl-3-(2-thienyl)-L-valyl-N¹-[(1S,2E)4-ethoxy-1-isopropyl-3-methyl-4-oxo-2-butenyl]-N¹,3-dimethyl-L-valinamide, and 20% (at 17.904 minutes) of N-methyl-3-(2-thienyl)-D-valyl-N¹-[(1S,2E)4-ethoxy-1-isopropyl-3-methyl4-oxo-2-butenyl]-N¹,3-dimethyl-L-valinamide. N-Methyl-3-(2-thienyl)-D-valyl-N¹-[(1S,2E)4-ethoxy-1-isopropyl-3-methyl-4-oxo-2-butenyl]-N¹,3-dimethyl-L-valinamide MS (ES):m/z 508.28 (M+H) IR cm⁻¹: 3339, 2958, 2931, 1706, 1674, 1628. Analysis for C₂₇H₄₅N₃O₄S Calcd: C, 63.87;H, 8.93; N, 8.28. Found: C, 64.19;H, 8.87; N, 8.33. Analytical HPLC (4.6×150 mm YMC pack Pro C18 column eluted with a liner gradient of 10-100% actonitrile in water containing 0.02% formic acid over 35 min): 95% (at 17.7 minutes) of N-methyl-3-(2-thienyl)-D-valyl-N¹-[(1S,2E)-4-ethoxy-1-isopropyl-3-methyl-4-oxo-2-butenyl]-N¹,3-dimethyl-L-valinamide, and 1.8% (at 18.1 minutes) of N-methyl-3-(2-thienyl)-L-valyl-N¹-[(1S,2E)4-ethoxy-1-isopropyl-3-methyl-4-oxo-2-butenyl]-N¹,3-dimethyl-L-valinamide.

### EXAMPLE 20

### N-Methyl-3-(2-thienyl)-L-valyl-N¹-[(1S,2E)-3-carboxy-1-isopropyl-2-butenyl]-N¹,3-dimethyl-L-valinamide

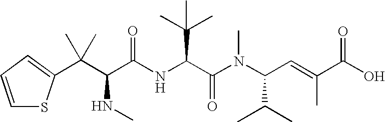

By using a procedure analogous to that described in General Procedure V, N-methyl-3-(2-thienyl)-L-valyl-N¹-[(1S,2E)-4-ethoxy-1-isopropyl-3-methyl-4-oxo-2-butenyl]-N¹,3-dimethyl-L-valinamide (263 mg, 0.518 mmol, obtained from Example 19) is treated with aqueous lithium hydroxide solution (4.14 mmol), in water (4 mL) and methanol (12 mL), to provide N-methyl-3-(2-thienyl)-L-valyl-N-[(1S,2E)-3-carboxy-1-isopropyl-2-butenyl]-N,3-dimethyl-L-valinamide (142 mg, 57.5%) as a white solid. MS (ES): m/z 480.3 (M+H) IR cm⁻¹: 3418, 2966, 1635. Analytical HPLC (4.6×150 mm YMC pack Pro C18 column eluted with a liner gradient of 10-100% actonitrile in water containing 0.02% formic acid over 35 min): 98.3% at 12.0 minutes.

### EXAMPLE 21

### N-Methyl-3-(2-thienyl)-D-valyl-N¹-[(1S,2E)-3-carboxy-1-isopropyl-2-butenyl]-N¹,3-dimethyl-L-valinamide

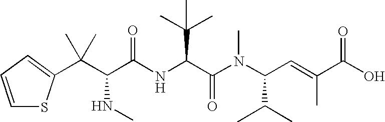

By using a procedure analogous to that described in General Procedure V, N-methyl-3-(2-thienyl)-D-valyl-N¹-[(1S,2E)4-ethoxy-1-isopropyl-3-methyl-4-oxo-2-butenyl]-N¹,3-dimethyl-L-valinamide (295 mg, 0.581 mmol, obtained from Example 19) is treated with aqueous lithium hydroxide solution (4.6 mmol), in water (4.6 mL) and methanol (15 mL), to provide N-methyl-3-(2-thienyl)-D-valyl-N¹-[(1S,2E)-3-carboxy-1-isopropyl-2-butenyl]-N¹,3-dimethyl-L-valinamide (135.1 mg, 48.5%) as a white solid. MS (ES): m/z 480.2 (M+H) IR cm⁻¹: 3409, 2966, 1674, 1639. Analytical HPLC (4.6×150 mm YMC pack Pro C18 column eluted with a liner gradient of 10-100% actonitrile in water containing 0.02% formic acid over 35 min): 78% (at 12.755 minutes) N-methyl-3-(2-thienyl)-D-valyl-N¹-[(1S,2E)-3-carboxy-1-isopropyl-2-butenyl]-N¹,3-dimethyl-L-valinamide, and 21.1% (at 12.0 minutes) of N-methyl-3-(2-thienyl)-L-valyl-N¹-[(1S,2E)-3-carboxy-1-isopropyl-2-butenyl]-N¹,3-dimethyl-L-valinamide.

### EXAMPLE 22a AND 22b

### N-Methyl-3-thien-3-yl-L-valyl-N¹-[(1S,2E)-4-ethoxy-1-isopropyl-3-methyl-4-oxobut-2-enyl]-N¹,3-dimethyl-L-valinamide and Example 22b, N-methyl-3-thien-3-yl-D-valyl-N¹-[(1S,2E)-4-ethoxy-1-isopropyl-3-methyl-4-oxobut-2-enyl]-N¹,3-dimethyl-L-valinamide

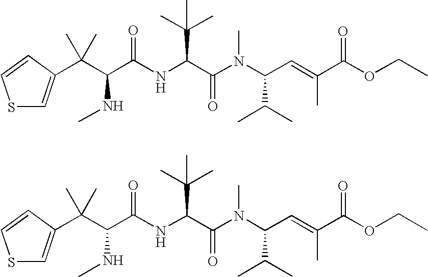

By a procedure analogous to that described in General Procedure IVb, N-methyl-3-thien-3-ylvaline (800 mg, 3.751 mmol, obtained from Reference Example 28) is treated with ethyl (2E,4S)-2,5-dimethyl-4-[methyl(3-methyl-L-valyl)amino]hex-2-enoate hydrochloride (1.31 g, 3.751 mmol), in the presence of hydroxybenzothriazole (532.14 mg, 3.94 mmol), 1-(3-dimethylaminopropyl)-3-ethylcarbodiimide hydrochloride (863 mg, 4.5 mmol), N-methylmorpholine (0.62 mL, 5.64 mmol) in anhydrous dimethylformaldehyde (20 mL). The products are purified by chromatography (silica gel, flash column, 0.5% methanol in methylene chloride), to provide N-methyl-3-thien-3-yl-L-valyl-N¹-[(1S,2E)-4-ethoxy-1-isopropyl-3-methyl-4-oxobut-2-enyl]-N¹,3-dimethyl-L-valinamide (338.0 mg, 17.7%) as a pale yellow oil, N-methyl-3-thien-3-yl-D-valyl-N¹-[(1S,2E)-4-ethoxy-1-isopropyl-3-methyl-4-oxobut-2-enyl]-N¹,3-dimethyl-L-valinamide (430.4 mg, 22.6%) as an oil, and a mixture of non-separated two isomers mentioned above (154.0 mg, 8.1%). N-Methyl-3-thien-3-yl-L-valyl-N¹-[(1S,2E)-4-ethoxy-1-isopropyl-3-methyl-4-oxobut-2-enyl]-N¹,3-dimethyl-L-valinamide MS (ES): m/z 508.28 (M+H) IR cm⁻¹: 2966, 1714, 1638, 1502. Analysis for C₂₇H₄₅N₃O₄S Calcd: C, 63.87;H, 8.93; N, 8.28. Found: C, 63.30;H, 8.55; N, 8.16. Analytical HPLC: (4.6×150 mm YMC pack Pro C18 column eluted with a liner gradient of 10-100% actonitrile in water containing 0.02% formic acid over 35 min): 87.3% (at 16.3 minutes) of N-methyl-3-thien-3-yl-L-valyl-N¹-[(IS,2E)4-ethoxy-1-isopropyl-3-methyl-4-oxobut-2-enyl-N¹,3-dimethyl-L-valinamide, and 3.9% (at 17.7 minutes) of N-methyl-3-thien-3-yl-D-valyl-N¹-[(1S,2E)-4-ethoxy-1-isopropyl-3-methyl-4-oxobut-2-enyl]-N¹,3-dimethyl-L-valinamide. N-Methyl-3-thien-3-yl-D-valyl-N¹-[(1S,2E)4-ethoxy-1-isopropyl-3-methyl-4-oxobut-2-enyl]-N¹,3-dimethyl-L-valinamide MS (ES): m/z 508.3 (M+H) IR cm⁻¹: 2966, 1715, 1639, 1502. Analysis for C₂₇H₄₅N₃O₄S Calcd: C, 63.87;H, 8.93; N, 8.28. Found: C, 63.59;H, 8.79; N, 8.15. Analytical HPLC: (4.6×150 mm YMC pack Pro C18 column eluted with a liner gradient of 15-100% actonitrile in water containing 0.02% formic acid over 35 min): 80.91% (at 16.972 minutes) of N-methyl-3-thien-3-yl-D-valyl-N¹-[(1S,2E)4-ethoxy-1-isopropyl-3-methyl4-oxobut-2-enyl]-N¹,3-dimethyl-L-valinamide, and 13.6% (at 16.66 minutes) of N-methyl-3-thien-3-yl-L-valyl-N¹-[(1S,2E)-4-ethoxy-1-isopropyl-3-methyl-4-oxobut-2-enyl]-N¹,3-dimethyl-L-valinamide.

### EXAMPLE 23

### N-Methyl-3-thien-3-yl-L-valyl-N¹-[(1S,2E)-3-carboxy-1-isopropylbut-2-enyl]-N¹,3-dimethyl-L-valinamide

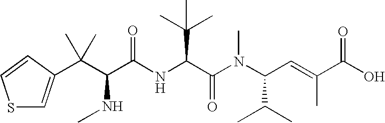

By using a procedure analogous to that described in General Procedure V, N-methyl-3-thien-3-yl-L-valyl-N¹-[(1S,2E)-4-ethoxy-1-isopropyl-3-methyl4-oxobut-2-enyl]-N¹,3-dimethyl-L-valinamide (276.4 mg, 0.544 mmole, obtained from Example 22) is treated with lithium hydroxide aqueous solution (4.3 mmol) in water (4.4 mL) and methanol (13 mL), to provide N-methyl-3-thien-3-yl-L-valyl-N¹-[(1S,2E)-3-carboxy-1-isopropylbut-2-enyl]-N¹,3-dimethyl-L-valinamide (261 mg, 100%) as a white amorphous solid. MS (ES): m/z 480.4 (M+H) IR cm⁻¹: 2966, 1679, 1633. Analytical HPLC (4.6×150 mm YMC pack Pro C18 column eluted with a liner gradient of 15-100% actonitrile in water containing 0.02% formic acid over 35 min): 85.43% (at 12.183 minutes) of N-methyl-3-thien-3-yl-L-valyl-N¹-[(1S,2E)-3-carboxy-1-isopropylbut-2-enyl]-N¹,3-dimethyl-L-valinamide, and 2.39% (at 13.146 minutes) of N-methyl-3-thien-3-yl-D-valyl-N¹-[(1S,2E)-3-carboxy-1-isopropylbut-2-enyl]-N¹,3-dimethyl-L-valinamide.

### EXAMPLE 24

### N-Methyl-3-thien-3-yl-D-valyl-N¹-[(1S,2E)-3-carboxy-1-isopropylbut-2-enyl]-N¹,3-dimethyl-L-valinamide

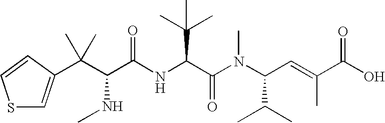

By using a procedure analogous to that described in General Procedure V, N-methyl-3-thien-3-yl-D-valyl-N¹-[(1S,2E)-4-ethoxy-1-isopropyl-3-methyl-4-oxobut-2-enyl]-N¹,3-dimethyl-L-valinamide (368.2 mg, 0.725 mmole, obtained from Example 22) is treated with lithium hydroxide aqueous solution (5.8 mmol) in water (5.8 mL) and methanol (17 mL), to provide N-methyl-3-thien-3-yl-D-valyl-N¹-[(1S,2E)-3-carboxy-1-isopropylbut-2-enyl]-N¹,3-dimethyl-L-valinamide (329 mg, 94.5%) as a white amorphous solid. MS (ES): m/z 480.3 (M+H) IR cm⁻¹: 2966, 1675, 1636, 1410. Analytical HPLC (4.6×150 mm YMC pack Pro C18 column eluted with a liner gradient of 15-100% actonitrile in water containing 0.02% formic acid over 35 min): 83.9% (at 12.974 minutes) of N-methyl-3-thien-3-yl-D-valyl-N¹-[(1S,2E)-3-carboxy-1-isopropylbut-2-enyl]-N¹,3-dimethyl-L-valinamide, and 11.8% (at 12.361 minutes) of N-methyl-3-thien-3-yl-L-valyl-N¹-[(1S,2E)-3-carboxy-1-isopropylbut-2-enyl]-N¹,3-dimethyl-L-valinamide.

### EXAMPLE 25

### 3-(1-Benzothien-3-yl)-N-methylvalyl-N¹-[(l S,2E)-4-ethoxy-1-isopropyl-3-methyl4-oxo-2-butenyl]-N¹,3-dimethyl-L-valinamide

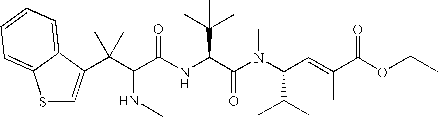

By using a procedure analogous to that described in General Procedure IVa, 3-(1-benzothien-3-yl)-3-methyl-2-oxobutanoic acid (400 mg, 1.52 mmol) obtained from Reference Example 32 is treated with ethyl (2E,4S)-2,5-dimethyl-4-[methyl(3-methyl-L-valyl)amino]hex-2-enoate hydrochloride (637 mg, 1.83 mmol) in the presence of benzotriazole-1-yl-oxy-tris-pyrrolidinophosphonium hexafluorophosphate (950 mg, 1.83 mmol) and diisopropylethylamine (0.8 mL, 4.6 mmol) in anhydrous dimethylformamide (6 mL). The products are purified by chromatography (silica gel, flash column, 40% ethyl acetate in hexanes), to provide a mixture of three diastereomers of 3-(1-benzothien-3-yl)-N-methylvalyl-N¹-[(1S,2E)-4-ethoxy-1-isopropyl-3-methyl-4-oxo-2-butenyl]-N¹,3-dimethyl-L-valinamide (213 mg, 25%) as a white amorphous solid. MS (ES): m/z 558.49 (M+H). IR cm⁻¹: 3382.48, 2964.62, 1638.60. Analytical HPLC (4.6×150 mm YMC Pack Pro C18 column eluted with 15 to 100% acetonitrile in water containing 0.02% formic acid over 35 minutes): 61.39% (at 19.3 minutes) of two diastereomers, 3-(1-benzothien-3-yl)-N-methylvalyl-N¹-[(1S,2E)-4-ethoxy-1-isopropyl-3-methyl-4-oxo-2-butenyl]-N¹,3-dimethyl-L-valinamide, and 0.90% (at 19.6 minutes) of the other isomer.

### EXAMPLE 26

### 3-(1-Benzothien-3-yl)-N-methylvalyl-N¹-[(1S,2E)-3-carboxy-1-isopropyl-2-butenyl]-N¹,3-dimethyl-L-valinamide

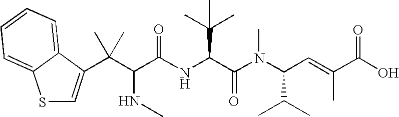

By using a procedure analogous to that described in General Procedure V, 3-(1-benzothien-3-yl)-N-methylvalyl-N¹-[(1S,2E)4-ethoxy-1-isopropyl-3-methyl4-oxo-2-butenyl]-N¹,3-dimethyl-L-valinamide (175 mg, 0.314 mmol, obtained from Example 25) is treated with aqueous lithium hydroxide solution (1.4 mmol) in water (0.706 mL) and methanol (3 mL), to provide a mixture of 3-(1-benzothien-3-yl)-N-methylvalyl-N¹-[(1S,2E)-3-carboxy-1-isopropyl-2-butenyl]-N¹,3-dimethyl-L-valinamide as a white solid (307 mg, >100%, approximate ratio of 5:3 by ¹H NMR). Analytical sample is obtained by purification of 250 mg of this material by preparative HPLC, to give 90 mg of the trifluoroacetic acid salt as white solid. MS (ES): m/z 530.46 (M+H) IR cm⁻¹: 3403.82, 2967.76, 1681.74. Analytical HPLC (4.6×150 mm YMC Pack Pro C18 column eluted with 15-100% acetonitrile in water containing 0.02% formic acid over 35 minutes): 98.52% (at 13.9 minutes) of two overlapping diastereomers.

### EXAMPLE 27

### 3-(1-Benzothien-2-yl)-N-methylvalyl-N¹-[(1S,2E)4-ethoxy-1-isopropyl-3-methyl4-oxo-2-butenyl]-N¹,3-dimethyl-L-valinamide

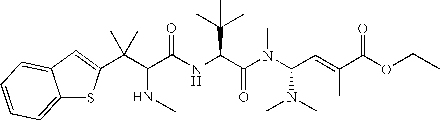

By a procedure analogous to that described in General Procedure IVa, 3-(1-benzothien-2-yl)-3-methyl-2-oxobutanoic acid (300 mg, 1.14 mmol, obtained from Reference Example 36) is treated with ethyl (2E,4S)-2,5-dimethyl4-[methyl(3-methyl-L-valyl)amino]hex-2-enoate hydrochloride (437 mg, 1.25 mmol) in the presence of benzotriazole-1-yl-oxy-tris-pyrrolidinophosphonium hexafluorophosphate (652 mg, 1.25 mmol) and diisopropylethylamine (0.6 mL, 3.4 mmol) in anhydrous dimethylformamide (5 mL). The products are purified by chromatography (silica gel, flash column, 40% ethyl acetate in hexanes), to provide a mixture of two diastereomers of 3-(1-benzothien-2-yl)-N-methylvalyl-N¹-[(1S,2E)4-ethoxy-1-isopropyl-3-methyl4-oxo-2-butenyl]-N¹,3-dimethyl-L-valinamide (379 mg, 59%) as a pale yellow amorphous solid. MS (ES): m/z 558.49 (M+H). IR cm⁻¹: 3380.46, 2965.39, 1713.78, 1638.02. Analytical HPLC (4.6×150 mm YMC Pack Pro C18 column eluted with 15-100% acetonitrile in water containing 0.02% formic acid over 35 minutes): 49.11% (at 20.2 minutes) of one diastereomer, 3-(1-benzothien-2-yl)-N-methylvalyl-N¹-[(1S,2E)-4-ethoxy-1-isopropyl-3-methyl-4-oxo-2-butenyl]-N¹,3-dimethyl-L-valinamide, and 33.84% (at 20.7 minutes) of the second diastereomer.

### EXAMPLE 28

### 3-(1-Benzothien-2-yl)-N-methylvalyl-N¹-[(1S,2E)-3-carboxy-1-isopropyl-2-butenyl]-N¹,3-dimethyl-L-valinamide

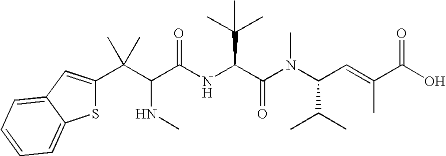

By using a procedure analogous to that described in General Procedure V, 3-(1-benzothien-2-yl)-N-methylvalyl-N¹-[(1S,2E)-4-ethoxy-1-isopropyl-3-methyl-4-oxo-2-butenyl]-N¹,3-dimethyl-L-valinamide (327 mg, 0.586 mmol, obtained from Example 27) is treated with aqueous lithium hydroxide solution (2.64 mmol), in water (1.32 mL) and methanol (6 mL), to provide a mixture of 3-(1-benzothien-2-yl)-N-methylvalyl-N¹-[(1S,2E)-3-carboxy-1-isopropyl-2-butenyl]-N¹,3-dimethyl-L-valinamide as a white solid (279 mg, 90%, approximate ratio of 6:4 by ¹H NMR). MS (ES): m/z 530.44 (M+H). IR cm: 3409.44, 2965.42, 1638.06. Analytical HPLC (4.6×150 mm YMC Pack Pro C18 column eluted with 15-100% acetonitrile in water containing 0.02% formic acid over 35 minutes): 81.19% (at 18.4 minutes) of two overlapping diastereomers.

### EXAMPLE 29

### 4-tert-Butyl-N,β,β-trimethylphenylalanyl-N¹-[(1S,2E)-4-ethoxy-1-isopropyl-3-methyl-4-oxo-2-butenyl]-N¹,3-dimethyl-L-valinamide

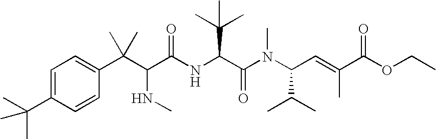

By using a procedure analogous to that described in General Procedure IVa, 4-tert-butyl-N,β,β-trimethylphenylalanine (234 mg, 0.89 mmol, obtained from Reference Example 40) is treated with ethyl (2E,4S)-2,5-dimethyl-4-[methyl(3-methyl-L-valyl)amino]hex-2-enoate hydrochloride (278 mg, 0.89 mmol) in the presence of benzotriazole-1-yl-oxy-tris-pyrrolidinophosphonium hexafluorophosphate (486 mg, 0.93 mmol), diisopropylethylamine (0.465 mL, 2.7 mmol) in methylene chloride (5 mL). The products are purified by chromatography (silica gel, flash column, 40% ethyl acetate in hexanes), to provide a mixture of two diastereomers of 4-tert-butyl-N,β,β-trimethylphenylalanyl-N¹-[(1S,2E)4-ethoxy-1-isopropyl-3-methyl-4-oxo-2-butenyl]-N¹,3-dimethyl-L-valinamide (252 mg, 51%) as a white amorphous solid. MS (ES): m/z 558.50 (M+H). IR cm⁻¹: 2964.60, 1640.20. Analytical HPLC (4.6×150 mm YMC Pack Pro C18 column eluted with 10-100% acetonitrile in water containing 0.02% formic acid over 30 minutes): 56.29% (at 18.8 minutes) of one diastereomer, and 39.44% (at 19.1 minutes) of the other diastereomer.

### EXAMPLE 30

### 4-tert-Butyl-N,β,β-trimethylphenylalanyl-N¹-[(1S,2E)-3-carboxy-1-isopropyl-2-butenyl]-N¹,3-dimethyl-L-valinamide

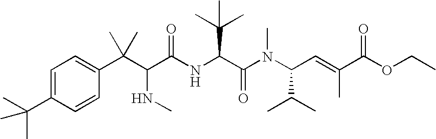

By using a procedure analogous to that described in General Procedure V, 4-tert-butyl-N,β,β-trimethylphenylalanyl-N¹-[(1S,2E)-4-ethoxy-1-isopropyl-3-methyl-4-oxo-2-butenyl]-N¹,3-dimethyl-L-valinamide (215 mg, 0.385 mmol, obtained from Example 29) is treated with aqueous lithium hydroxide solution (1.2 mmol), in water (0.580 mL) and methanol (2.8 mL), to provide a mixture of 4-tert-butyl- N,β,β-trimethylphenylalanyl-N¹-[(1S,2E)-3-carboxy-1-isopropyl-2-butenyl]-N¹,3-dimethyl-L-valinamide as a white solid (183 mg, 90%). MS (ES): m/z 530.37 (M+H). IR cm⁻¹: 3422.14, 2964.13, 1680.95, 1639.62. Analytical HPLC (4.6×150 mm YMC Pack Pro C18 column eluted with 15-100% acetonitrile in water containing 0.02% formic acid over 30 minutes): 39.20% (at 18.6 minutes) of the one diastereomer, and 57.44% (at 19.0 minutes) of the second diastereomer.

### EXAMPLE 31

### N-Ethyl-β,β-dimethylphenylalanyl-N¹-[(1S,2E)-4-ethoxy-1-isopropyl-3-methyl4-oxo-2-butenyl]-N¹,3-dimethyl-L-valinamide

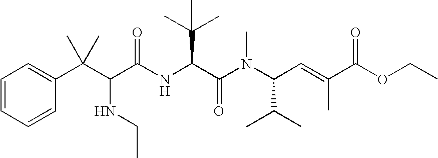

By a procedure analogous to that described in General Procedure IVb, β,β-dimethyl-N-ethyl-phenylalanine (553 mg, 2.5 mmol, obtained from Reference Example 41) is treated with ethyl (2E,4S)-2,5-dimethyl-4-[methyl(3-methyl-L-valyl)amino]hex-2-enoate hydrochloride (857 mg, 2.0 mmol) in the presence of hydroxybenzotriazole (338 mg, 2.5 mmol), 1-(3-dimethylaminopropyl)-3-ethylcarbodiimidazole hydrochloride (575 mg, 3.0 mmol) and N-methylmorpholine (0.385 mL, 3.5 mmol) in anhydrous dimethylformamide (5 mL). The products are purified by chromatography (silica gel, flash column, methylene chloride: ethyl ether: methanol=95:3.5:1.5), to provide N-ethyl-β,β-dimethylphenylalanyl-N¹-[(1S,2E)-4-ethoxy-1-isopropyl-3-methyl-4-oxo-2-butenyl]-N¹,3-dimethyl-L-valinamide (313.7 mg, 30.5%, an approximately 1:1 ratio of two diastereomers by ¹H NMR) as a colorless glass MS (ES): m/z516.5 (M+H). IR cm¹: 3334, 2966, 2935, 1714, 1671, 1640. Analysis for C₃₀H₄₉N₃O₄: Calcd: C, 69.87;H, 9.58; N, 8.15. Found: C, 69.10;H, 9.57; N, 8.73. Analytical HPLC: (4.6×150 mm YMC Pro Pack C18 column eluted with 15 to 100% acetonitrile in water containing 0.02% formic acid over 30 minutes): 76.4% (at 17.3 minutes) of two overlapping diastereomers.

### EXAMPLE 32

### N-Ethyl-β,β-dimethylphenylalanyl-N¹-[(1S,2E)-3-carboxy-1-isopropyl-2-butenyl]-N¹,3-dimethyl-L-valinamide

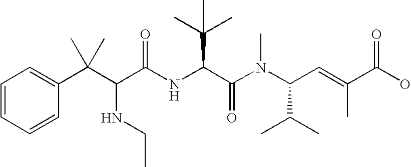

By using a procedure analogous to that described in General Procedure V, N-ethyl-β,β-dimethylphenylalanyl-N¹-[(1S,2E)4-ethoxy-1-isopropyl-3-methyl-4-oxo-2-butenyl]-N¹,3-dimethyl-L-valinamide (222 mg, 0.43 mmol, obtained from Example 31) is treated with aqueous lithium hydroxide solution (3.4 mmol), in water (3.4 mL) and methanol (10.3 mL), to provide N-ethyl-β,β-dimethylphenylalanyl-N¹-[(1S,2E)-3-carboxy-1-isopropyl-2-butenyl]-N¹,3-dimethyl-L-valinamide (100 mg, 47.7%, a mixture of two isomers) as a white solid. MS (ES): m/z 488.3 (M+H). IR cm⁻¹: 2966, 1672, 1639. Analytical HPLC: (4.6×150 mm YMC Pro Pack C18 column eluted with 15 to 100% acetonitrile in water containing 0.02% formic acid over 35 minutes): 41.39% (at 11.2 minutes) of one diasteromer, and 46.33% (at 11.6 minutes) of the second diasteromer.

### EXAMPLE 33

### N-(tert-Butoxycarbonyl)-N-β,β,2-tetramethylphenylalanyl-N¹-[(1S,2E)-4-ethoxy-1-isopropyl-3-methyl-4-oxo-2-butenyl]-N¹,3-dimethyl-L-valinamide

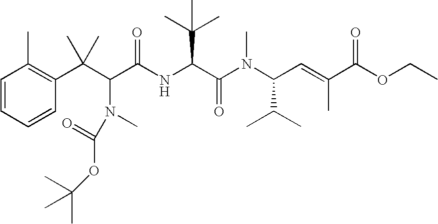

By a procedure analogous to that described in General Procedure IVa, 2-(N-tert-butoxycarbonyl-N-methylamino)-3-methyl-3-o-tolyl-butyric acid (97 mg, 0.3 mmol, obtained from Reference Example 47) is treated with ethyl (2E,4S)-2,5-dimethyl-4-[methyl(3-methyl-L-valyl)amino]hex-2-enoate hydrochloride (0.116 g, 0.33 mmol) in the presence of benzotriazole-1-yl-oxy-tris-pyrrolidinophosphonium hexafluorophosphate (173 mg, 0.33 mmol) and N,N-diisopropylethylamine (0.158 mL, 0.91 mmol) in anhydrous methylene chloride (1.5 mL). The products are purified by chromatography (silica gel, flash column, 20% ethyl acetate in hexanes), to provide a mixture of two diastereomers of N-(tert-butoxycarbonyl)-N,β,β2-tetramethylphenylalanyl-N¹-[(1S,2E)-4-ethoxy-1-isopropyl-3-methyl-4-oxo-2-butenyl]-N¹,3-dimethyl-L-valinamide (130 mg, 70%), as a colorless glass. MS (ES): m/z 616.4 (M+H). IR cm⁻¹: 2965.22, 1688.27. Analytical HPLC (4.6×150 mm ODS3 Prodigy column eluted with 10-95% acetonitrile in water containing 0.02% trifluoroacetic acid over 25 minutes): 52.3% (at 22.8 minutes) of the first diastereomer, and 36.2% (at 23.2 minutes) of the second diastereomer.

### EXAMPLE 34a AND 34b

### N,β,β,2-tetramethyl-L-phenylalanyl-N¹-[(1S,2E)4-ethoxy-1-isopropyl-3-methyl-4-oxo-2-butenyl]-N¹,3-dimethyl-L-valinamide and Example 34b, N,β,β,2-Tetramethyl-D-phenylalanyl-N¹-[(1S,2E)4-ethoxy-1-isopropyl-3-methyl-4-oxo-2-butenyl]-N¹,3-dimethyl-L-valinamide

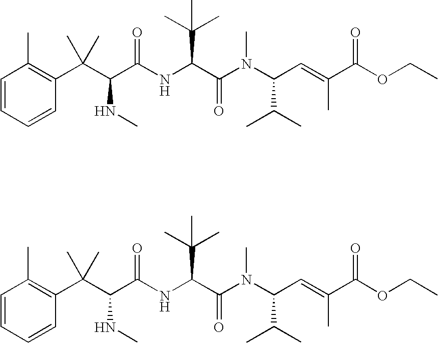

By a procedure analogous to that described in General Procedure VI, N-(tert-butoxycarbonyl)-N,β,β,2-tetramethylphenylalanyl-N¹-[(1S,2E)4-ethoxy-1-isopropyl-3-methyl-4-oxo-2-butenyl]-N¹,3-dimethyl-L-valinamide (382 mg, 0.62 mmol, obtained from Example 33) in anhydrous methylene chloride (1.5 mL) is cooled to 0° C. then treated with trifluoroacetic acid (1 mL, 13 mmol, Aldrich) at 0° C. for 30 minutes. The mixture is concentrated, diluted with water and aqueous saturated sodium bicarbonate, extracted with ethyl acetate (3×50 mL). The extracts are washed with aqueous saturated sodium chloride, dried over anhydrous sodium sulfate, filtered and concentrated in vacuo. The resulting product contained starting material and is resubjected to the above conditions for 15 minutes at 0° C. and 15 minutes at room temperature before working up in the same manner. The products are purified by chromatography (silica gel, flash column, 40% ethyl acetate in hexanes), to provide N,β,β,2-tetramethyl-L-phenylalanyl-N¹-[(1S,2E)-4-ethoxy-1-isopropyl-3-methyl-4-oxo-2-butenyl]-N¹,3-dimethyl-L-valinamide (129 mg, 40%) as a white solid, N,β,β,2-tetramethyl-D-phenylalanyl-N¹-[(1S,2E)4-ethoxy-1-isopropyl-3-methyl-4-oxo-2-butenyl]-N¹,3-dimethyl-L-valinamide (116 mg, 36%) a colorless glass, and a mixture of non-separated two isomers mentioned above(75 mg, 23%). N,β,β,2-Tetramethyl-L-phenylalanyl-N¹-[(1S,2E)-4-ethoxy-1-isopropyl-3-methyl-4-oxo-2-butenyl]-N¹,3-dimethyl-L-valinamide. MS (ES): m/z 516.37 (M+H). IR cm⁻¹: 2965.44, 1706.46, 1669.37. Analysis for C₃₀H₄₉N₃O₄: Calcd: C, 69.87;H, 9.58; N, 8.15. Found: C, 69.74;H, 9.43; N, 8.11. Analytical HPLC (4.6×150 mm YMC Pack Pro C18 column eluted with 10-100% acetonitrile in water containing 0.02% formic acid over 35 minutes): 99.50% (at 19.9 minutes). N,β,β,2-Tetramethyl-D-phenylalanyl-N¹-[(1S,2E)-4-ethoxy-1-isopropyl-3-methyl-4-oxo-2-butenyl]-N¹,3-dimethyl-L-valinamide MS (ES): m/z 516.38 (M+H). IR cm⁻¹: 2962.66, 1640.61. Analysis for C₃₀H₄₉N₃O₄: Calcd: C, 69.87;H, 9.58; N, 8.15. Found: C, 69.67;H, 9.59; N, 8.00. Analytical HPLC (4.6×150 mm YMC Pack Pro C18 column eluted with 10-100% acetonitrile in water containing 0.02% formic acid over 35 minutes): 99.07% (at 19.8 minutes).

### EXAMPLE 35

### N,β,β,2-Tetramethyl-L-phenylalanyl-N¹-[(1S,2E)-3-carboxy-1-isopropyl-2-butenyl]-N¹,3-dimethyl-L-valinamide

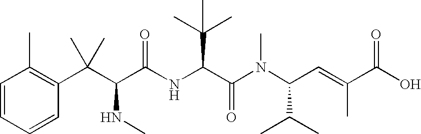

By using a procedure analogous to that described in General Procedure V, N,β,β,2-tetramethyl-L-phenylalanyl-N¹-[(1S,2E)4-ethoxy-1-isopropyl-3-methyl4-oxo-2-butenyl]-N¹,3-dimethyl-L-valinamide (96 mg, 0.186 mmol, obtained from Example 34) is treated with aqueous lithium hydroxide solution (0.529 mmol), in water (0.265 mL) and methanol (2 mL). Workup provided the methyl ester of the desired product. This material is dissolved in anhydrous tetrahydrofuran (1 mL), and water (0.162 mL) and treated with aqueous lithium hydroxide solution (0.323 mmol) for 15 hours, to provide N,β,β,2--tetramethyl-L-phenylalanyl-N¹-[(1S,2E)-3-carboxy-1-isopropyl-2-butenyl]-N¹,3-dimethyl-L-valinamide (28 mg, 31%) as a white solid. MS (ES): m/z 486.30 (M−H), 973.48 (2M−H). Analytical HPLC (4.6×150 mm YMC Pack Pro C18 column eluted with 10-100% acetonitrile in water containing 0.02% formic acid over 35 minutes): 92.33% (at 14.3 minutes) of N,β,β,2-tetramethyl-L-phenylalanyl-N¹-[(1S,2E)-3-carboxy-1-isopropyl-2-butenyl]-N¹,3-dimethyl-L-valinamide, and two other diastereomers of 2.85% (at 15.0 minutes) and 0.50% (at 15.5 minutes).

### EXAMPLE 36

### N,β,β,2-Tetramethyl-D-phenylalanyl-N¹-[(1S,2E)-3-carboxy-1-isopropyl-2-butenyl]-N¹,3-dimethyl-L-valinamide

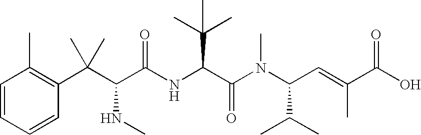

By using a procedure analogous to that described in General Procedure V, N,β,β,2-tetramethyl-D-phenylalanyl-N¹-[(1S,2E) 4ethoxy-1-isopropyl-3-methyl-4-oxo-2-butenyl]-N¹,3-dimethyl-L-valinamide (90 mg, 0.175 mmol, obtained from Example 34) is treated with aqueous lithium hydroxide solution (0.529 mmol), in water (0.265 mL) and methanol (2 mL), to provide N,β,β,2-tetamethyl-D-phenylalanyl-N¹-[(1S,2E)-3-carboxy-1-isopropyl-2-butenyl]-N¹3-dimethyl-L-valinamide (95 mg, >100%) as a white solid. MS (ES): m/z 488.43 (M+H). IR cm⁻¹: 3381.61, 2964.6, 1641.03. Analytical HPLC (4.6×150 mm YMC Pack Pro C18 column eluted with 10-100% acetonitrile in water containing 0.02% formic acid over 35 minutes): 94.70% (at 14.8 minutes) of provide N,β,β,2-tetramethyl-D-phenylalanyl-N¹[(1S,2E)-3-carboxy-1-isopropyl-2-butenyl]-N¹,3-dimethyl-L-valinamide, and two other diastereomers of 4.37% (at 14.4 minutes) and of 0.07% (at 16.2 minutes).

### EXAMPLE 37

### 3-bromo-N,β,β-trimethyl-L-phenylalanyl-N¹-[(1S,2E)-3-carboxy-1-isopropylbut-2-enyl]-N¹,3-dimethyl-L-valinamide

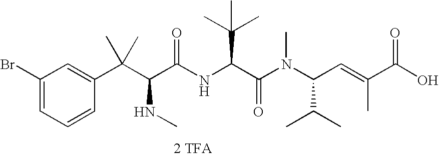

According to General Procedure V, 3-bromo- N,β,β-trimethyl-L-phenylalanyl-N¹-[(1S,2E)4ethoxy-1-isopropyl-3-methyl-4-oxo2-butenyl]-N¹,3-dimethyl-L-valinamide (0.16, 0.28 mmol, from Reference Example 52) is dissolved in tetrahydrofuran (0.75 mL), methanol (0.75 mL) and water (0.38 mL). To this solution is added lithium hydroxide monohydrate (0.025 g, 0.61 mmol). The resulting mixture is heated at 45-50° C. (bath temperature) for 18 hours, and then allowed to cool to room temperature. Solvent is evaporated in vacuo and the residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.097 g, 44%). TOF MS (ES⁻): m/z (M−H)=550.3, 552.3

### EXAMPLE 38

### 3-bromo-N,β,β-trimethyl-D-phenylalanyl-N¹-[(1S,2E)-3-carboxy-1-isopropylbut-2-enyl]-N¹,3-dimethyl-L-valinamide

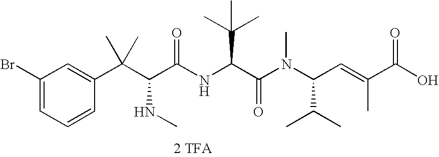

According to General Procedure V, 3-bromo- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (0.12, 0.21 mmol, from Reference Example 52) is dissolved in tetrahydrofuran (0.75 mL), methanol (0.75 mL) and water (0.38 mL). To this solution is added lithium hydroxide monohydrate (0.019 g, 0.45 mmol). The resulting mixture is heated at 45 -50° C. (bath temperature) for 18 hours and then allowed to cool to room temperature. Solvent is evaporated in vacuo and residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.13 g, 81%). MS (ES⁻): m/z (M−H)=550.6, 552.4

### EXAMPLE 39

### 3-phenyl-N,β,β-trimethyl-L-phenylalanyl-N¹-[(1S,2E)-3-carboxy-1-isopropylbut-2-enyl]-N¹,3-dimethyl-L-valinamide

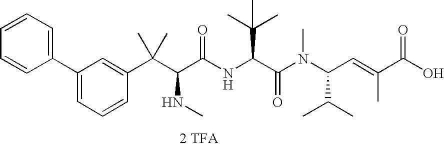

According to General Procedure V, 3-phenyl-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (0.18, 0.31 mmol, from Reference Example 53) is dissolved in tetrahydrofuran (1 mL), methanol (1 mL) and water (0.5 mL). To this solution is added lithium hydroxide monohydrate (0.029 g, 0.69 mmol). The resulting mixture is heated at 45-50° C. (bath temperature) for 18 hours and then allowed to cool to room temperature. Solvent is evaporated in vacuo and residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.13 g, 54%). MS (ES⁻): m/z (M−H)=548.1

### EXAMPLE 40

### 3-phenyl-N,β,β-trimethyl-D-phenylalanyl-N¹-[(1S,2E)-3-carboxy-1-isopropylbut-2-enyl]-N¹,3-dimethyl-L-valinamide

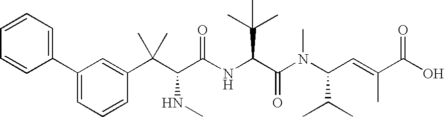

According to General Procedure V, 3-phenyl-N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (0.18, 0.31 mmol, from Reference Example 53) is dissolved in tetrahydrofuran (1 mL), methanol (1 mL) and water (0.5 mL). To this solution is added lithium hydroxide monohydrate (0.029 g, 0.69 mmol). The resulting mixture is heated at 45-50° C. (bath temperature) for 18 hours and then allowed to cool to room temperature. Solvent is evaporated in vacuo and residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.19 g, 79%). MS (ES⁻): m/z (M−H)=548.2

### EXAMPLE 41

### N,β,β-trimethyl-3-vinyl-L-phenylalanyl-N¹-[(1S,2E)-3-carboxy-1-isopropylbut-2-enyl]-N¹,3-dimethyl-L-valinamide

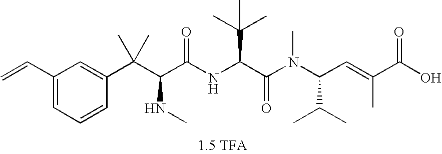

According to General Procedure V, 3-vinyl-N,β,β-trimethyl-L-phenylalanyl-N¹-[(1S,2E)4-ethoxy-1-isopropyl-3-methyl-4-oxo-2-butenyl]-N¹,3-dimethyl-L-valinamide (0.29, 0.55 mmol, from Reference Example 54) is dissolved in tetrahydrofuran (2 mL) methanol (2 mL), water (1 mL). To this solution is added lithium hydroxide monohydrate (0.051 g, 1.2 mmol). The resulting mixture is stirred in a 45-55° C. oil bath for 4 hours and then allowed to cool to room temperature. Solvent is evaporated in vacuo and residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.35 g, 95%). MS (ES⁻): m/z (M−H)=498.3

### EXAMPLE 42

### 3-ethyl- N,β,β-trimethyl-L-phenylalanyl-N¹-[(1S,2E)-3-carboxy-1-isopropylbut-2-enyl]-N¹,3-dimethyl-L-valinamide

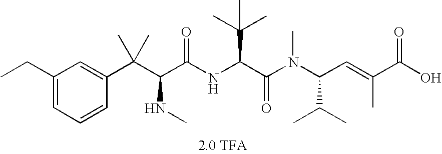

N,β,β-trimethyl-3-vinyl-L-phenylalanyl-N¹-[(1S,2E)-3-carboxy-1-isopropylbut-2-enyl]-N¹,3-dimethyl-L-valinamide (0.25 g, 0.37 mmol, from Example 41) is dissolved in absolute ethanol (12 mL). The solution is degassed by the addition of a small piece of dry ice. Palladium on carbon (10%, 50 mg) is added. The stirred solution is successively evacuated under weak vacuum and filled with hydrogen gas (balloon pressure). This procedure is repeated thrice before reaction mixture allowed to stir continuously under hydrogen atmosphere. After 30 minutes of stirring, reaction mixture is filtered through a Diatomaceous earth pad to remove palladium on carbon. Filtrate is concentrated under reduced pressure and purified by preparative reverse-phase HPLC (eluting from 5% acetonitrile/95% water/O. 1% trifluoroacetic acid to 100% acetonitrile over one hour). The title compound is obtained as its trifluoroacetic acid salt (0.15 g, 56%). MS (ES⁻): m/z (M−H)=500.2

### EXAMPLE 43

### 4-bromo-N,β,β-trimethylphenylalanyl-N¹-[(1S,2E)-3-carboxy-1-isopropyl-2-butenyl]-N¹,3-dimethyl-L-valinamide

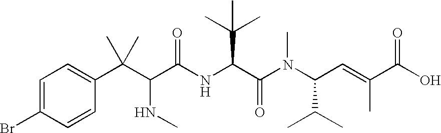

According to General Procedure V, 4-bromo-N,β,β-trimethylphenylalanyl-N¹-[(1S,2E)-4-ethoxy-1-isopropyl-3-methyl-4-oxo-2-butenyl]-N¹,3-dimethyl-L-valinamide (0.13 g, 0.15 mmol max., from Reference Example 58) is dissolved in tetrahydrofuran (2 mL), methanol (2 mL), water (1 mL). To this solution is added lithium hydroxide monohydrate (0.019 g, 0.45 mmol). The resulting mixture is stirred in a 60° C. oil bath for 18 hours and then allowed to cool to room temperature. Solvent is evaporated and residue is purified by preparative reverse-phase HPLC (eluting from 5 % acetonitrile/95% water/0.1% trifluoroacetic acid to 100% acetonitrile over one hour). The title compound is obtained as its trifluoroacetic acid salt (0.090 g, 69%). MS (ES⁺): m/z (M+H)=552.4, 554.4

### EXAMPLE 44

### 4-phenyl-N,β,β-trimethyl-L-phenylalanyl-N¹-[(1S,2E)-3-carboxy-1-isopropyl-2-butenyl]-N¹,3-dimethyl-L-valinamide

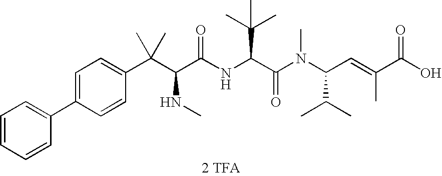

According to General Procedure V, 4-phenyl-N,β,β--trimethyl-L-phenylalanyl-N¹-[(1S,2E)-4-ethoxy-1-isopropyl-3-methyl-4-oxo-2-butenyl]-N¹,3-dimethyl-L-valinamide (0.06 g, 0.10 mmol, from Reference Example 60) is dissolved in tetrahydrofuran (1 mL), methanol (1 mL), water (0.5 mL). To this solution is added lithium hydroxide monohydrate (0.013 g, 0.30 mmol). The resulting suspension is heated briefly with a heat gun to dissolve all solids and is then allowed to sit at room temperature overnight. The solvent is evaporated and the residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.020 g, 26%). TOF MS (ES⁺): m/z (M+H)=550.4

### EXAMPLE 45

### 4-carboxy- N,β,β-trimethylphenylalanyl-N¹-[(1S,2E)-3-carboxy-1-isopropylbut-2-enyl]-N¹,3-dimethyl-L-valinamide

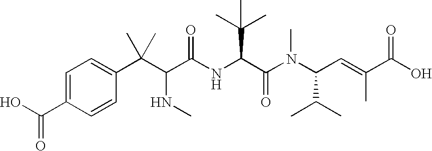

According to General Procedure IVb, to a solution of 4-(tert-butoxycarbonyl)-N-(tert-butoxycarbonyl)-N,β,β-trimethylphenylalanine (0.25 g, 0.61 mmol,) from Reference Example 68 and ethyl (2E,4S)-2,5-dimethyl-4-[methyl(3-methyl-L-valyl)amino]hex-2-enoate hydrochloride (0.32 g, 0.92 mmol) in anhydrous dimethylformamide (4 mL) is added hydroxybenzotriazole hydrate (0.17 g, 1.2 mmol), 1-(3-dimethylaminopropyl)-3-ethylcarbodiimine hydrochloride (0.24 g, 1.2 mmol), and N-methylmorpholine (0.27 mL, 2.4 mmol) under a nitrogen atmosphere. The resulting mixture is stirred for 24 hours at room temperature under a nitrogen atmosphere. The mixture is diluted with water, and the aqueous layer is extracted with diethyl ether (3 times). The combined extracts are washed with 2% aqueous hydrochloric acid and saturated sodium chloride solution, dried over anhydrous sodium sulfate, filtered and concentrated in vacuo. The residue is isolated as a hard white foam (0.40 g, 95%). MS (ES⁺): (M+H)=702.8

According to a modified version of General Procedure V, to a solution of the crude tripeptide (0.10 g, 0.14 mmol) from above in dichloromethane (3 mL) is added trifluoroacetic acid (1 mL). The resulting mixture is stirred overnight at room temperature. On the following day, solvent and excess acid are evaporated and residue is taken up in tetrahydrofuran (1 mL), methanol (1 mL), and water (0.5 mL). To this solution, lithium hydroxide monohydrate (0.053 g, 1.3 mmol) is added and the reaction mixture is stirred overnight at room temperature. The following day, solvent is evaporated under reduced pressure and the residue is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.090 g, 87%). MS (ES⁺): m/z (M+H)=518.3

### EXAMPLE 46

### 3-Methoxy- N,β,β-trimethylphenylalanyl-N¹-[(1S,2E)-3-carboxy-1-isopropyl-2-butenyl]-N¹,3-dimethyl-L-valinamide

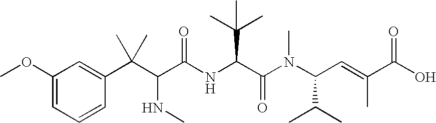

According to General Procedure V, a 1:1 mixture of 3-methoxy- N,β,β-trimethyl-L-phenylalanyl-N¹-[(1S,2E)4-ethoxy-1-isopropyl-3-methyl-4-oxo-2-butenyl]-N¹,3-dimethyl-L-valinamide and 3-methoxy- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (1.5 g crude, 1.5 mmol max., from Reference Example 73) is dissolved in tetrahydrofuran (5 mL) methanol (5 mL), water (2.5 mL). To this solution is added lithium hydroxide monohydrate (0.17 g, 4.0 mmol). The resulting mixture is stirred in a 60° C. oil bath for 18 hours and then allowed to cool to room temperature. Solvent is evaporated in vacuo to afford 1.5 g of a hard white foam, of which one-third is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.23 g, 43% for two steps). MS (ES⁺): m/z (M+H)=504.5

### EXAMPLE 47

### 3-Hydroxy- N,β,β-trimethylphenylalanyl-N¹-[(1S,2E)-3-carboxy-1-isopropyl-2-butenyl]-N¹,3-dimethyl-L-valinamide

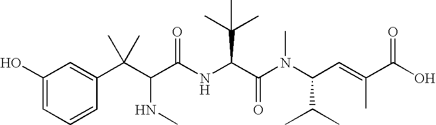

According to General Procedure V, the crude tripeptide ester (0.11 g crude, 0.21 mmol max., from Reference Example 76) is dissolved in tetrahydrofuran (1 mL), methanol (1 mL), water (0.5 mL). To this solution is added lithium hydroxide monohydrate (0.018 g, 0.42 mmol). The resulting mixture is stirred in a 55° C. oil bath for 19 hours and then allowed to cool to room temperature. Solvent is evaporated in vacuo to afford 0.12 g of a light yellow oil, which is purified by preparative reverse-phase HPLC (eluting from 5% acetonitrile/95% water/0.1% trifluoroacetic acid to 100% acetonitrile over one hour). The title compound is obtained as its trifluoroacetic acid salt (0.090, 56% g). MS (ES⁺): m/z (M+H)=490.4

### EXAMPLES 48 AND 49

### N,3-Dimethyl-4-phenyl-L-valyl-N¹-[(1S,2E)-3-carboxy-1-isopropyl-2-butenyl]-N¹,3-dimethyl-L-valinamide and Example 49 N,3-dimethyl-4-phenyl-D-valyl-N¹-[(1S,2E)-3-carboxy-1-isopropyl-2-butenyl]-N¹,3-dimethyl-L-valinamide

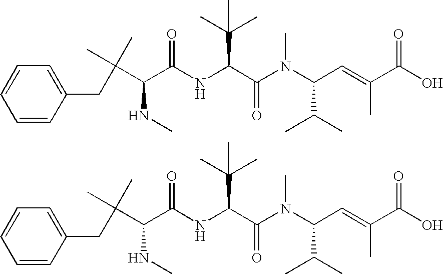

In a manner analogous to that described in General Procedure IVa, to N, 3-dimethyl-4-phenylvaline (300 mg, 1.36 mmol, from Reference Example 78) and ethyl (2E,4S)-2,5-dimethyl4-[methyl(3-methyl-L-valyl)amino]hex-2-enoate hydrochloride (523 mg, 1.5 mmol) in dichloromethane (10 mL) at 25° C. is added Hunig's base (0.71 mL, 4.1 mmol) followed by benzotriazole-1-yl-oxy-tris-pyrrolidinophosphonium hexafluorophosphate (781 mg, 1.5 mmol). After 5 hours the reaction mixture is concentrated in vacuo, dissolved in dimethyformamide, and purified by reverse phase HPLC (0.01% aqueous trifluoroacetic acid/acetonitrile gradient system) to give N,3-dimethyl-4-phenylvaline-N¹-[(1S,2E)-4-ethoxy-1-isopropyl-3-methyl-4-oxobut-2-enyl]-N¹,3-dimethyl-L-valinamide as an oil (76 mg). M+H =516.6. In a manner analogous to that described in General Procedure V, this material is treated with lithium hydroxide (0.266 mmol) in methanol (1 mL), tetrahydrofuran (1 mL) and water (0.5 mL) at 25° C. After 4 days the reaction mixture is concentrated in vacuo, taken up in dimethylformamide/water and purified by reverse phase HPLC (0.01% aqueous trifluoroacetic acid/acetonitrile gradient system) to give N,3-dimethyl-4-phenyl-L-valyl-N¹-[(1S,2E)-3-carboxy-1-isopropyl-2-butenyl]-N¹,3-dimethyl-L-valinamide (first isomer off column, 21 mg, white powder). MS (ES): m/z 488.6 (M+H). The epimer N,3-dimethyl4-phenyl-D-valyl-N¹-[(1S,2E)-3-carboxy-1-isopropyl-2-butenyl]-N¹,3-dimethyl-L-valinamide, is also obtained (second off column, 35 mg, white powder). MS (ES): m/z 488.11 (M+H).

### EXAMPLES 50 AND 51

### (2E,4S)-4-[((2S)-2-{[(2S)-3,3-dimethyl-2-(methylamino)octanoyl]amino}-3,3-dimethylbutanoyl)(methyl)amino]-2,5-dimethyl-2-hexenoic acid and Example 51 (2E,4S)-4-[((2S)-2-{[(2R)-3,3-dimethyl-2-(methylamino)octanoyl]amino}-3,3-dimethylbutanoyl)(methyl)amino]-2,5-dimethyl-2-hexenoic Acid

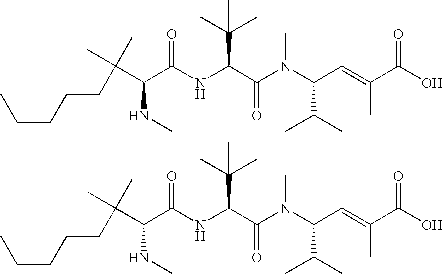

As described in General Procedure IVa, to 3,3-dimethyl-2-(methylamino)-octanoic acid (273 mg, 1.36 mmol, from Reference Example 80) and ethyl (2E,4S)-2,5-dimethyl-4-[methyl(3-methyl-L-valyl)amino]hex-2-enoate hydrochloride (523 mg, 1.5 mmol) in dichloromethane (10 mL) at 25° C. is added Hunig's base (0.71 mL, 4.1 mmol) followed by benzotriazole-1-yl-oxy-tris-pyrrolidinophosphonium hexafluorophosphate (781 mg, 1.5 mmol). After aqueous workup (2E,4S)-4-[((2S)-2-{[(2R1S)-3,3-dimethyl-2-(methylamino)octanoyl]amino}-3,3-dimethylbutanoyl)(methyl)amino]-2,5-dimethyl-2-hexenoic acid ethyl ester is obtained as a glass (109 mg). As described in General Procedure V, this material is treated with lithium hydroxide (0.39 mmol) in methanol (1 mL), tetrahydrofuran (1 mL) and water (0.5 mL) at 25° C. After 2 days the reaction mixture is concentrated in vacuo, taken up in dimethylformamide/water and purified by reverse phase HPLC (0.01% aqueous trifluoroacetic acid/acetonitrile gradient system) to give (2E,4S)4-[((2S)-2-{[(2S)-3,3-dimethyl-2-(methylamino)octanoyl]amino}-3,3-dimethylbutanoyl)(methyl)amino]-2,5-dimethyl-2-hexenoic acid, (first isomer off column, 3 mg, white powder). MS (ES): m/z 468.2 (M+H).

The epimer (2E,4S)-4-[((2S)-2-{[(2R)-3,3-dimethyl-2-(methylamino)octanoyl]amino}-3,3-dimethylbutanoyl)(methyl)amino]-2,5-dimethyl-2-hexenoic acid, is also obtained (second off column, 12.5 mg, white powder). MS (ES): m/z 468.2 (M+H).

### EXAMPLE 52

### N,N,β, β-Tetramethyl-L-phenylalanyl-N¹-[(1S,2E)-3-carboxy-1-isopropyl-2-butenyl]-N¹,3-dimethyl-L-valinamide

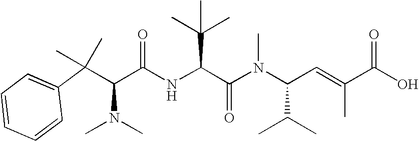

To ethyl (E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenoate hydrochloride (200 mg, 0.372 mmol, Andersen, R. WO 99/32509) in dimethylformamide (3 mL) is added iodomethane (29 uL, 0.44 mmol) and Hunig's base (200 uL, 1.12 mmol). After 30 min the reaction mixture is purified by reverse phase HPLC (0.01% aqueous trifluoroacetic acid/acetonitrile gradient system) to give the desired dimethylamine (99 mg). As described in General Procedure V, this material is treated with lithium hydroxide (0.47 mmol) in methanol (1 mL), tetrahydrofuran (1 mL) and water (0.5 mL) at 25° C. for 18 hours. Purification (2×) by reverse phase HPLC (0.01% aqueous trifluoroacetic acid/acetonitrile gradient system) gave the title compound as a white powder (20 mg). MS (ES): m/z 488.3412 (M+H). (calc'd exact mass=487.3412)

### EXAMPLE 53

### N-(2-hydroxyethyl)-N,β,β-trimethyl-L-phenylalanyl-N¹-[(1S,2E)-3-carboxy-1-isopropylbut-2-enyl]-N¹,3-dimethyl-L-valinamide

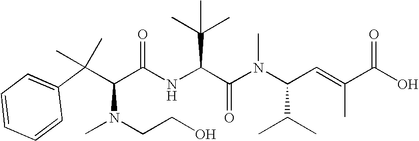

To ethyl (E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenoate hydrochloride (300 mg, 0.56 mmol, Andersen, R. WO 99/32509) in dimethylformamide (2 mL) is added 2-bromoethanol (0.04 mL, 0.56 mmol) and Hunig's base (0.195 mL, 1.12 mmol). After 18 hours tetrabutyl-ammonium iodide (10 mg) is added. After 4 hours 0.1 mL of 2-bromoethanol is added. After 18 hours the reaction mixture is heated at 70° C. and additional 2-bromoethanol and Hunig's base are added. The reaction mixture is purified by reverse phase HPLC (0.02% aqueous trifluoroacetic acid/methanol gradient system) to give the desired N-alkylated product. As described in General Procedure V, this material (50 mg, 0.0758 mmol) is treated with lithium hydroxide (0.23 mmol) in methanol (0.5 mL), tetrahydrofuran (0.5 mL) and water (0.25 mL) at 25° C. for 18 hours. Purification by reverse phase HPLC (0.01 % aqueous trifluoroacetic acid/acetonitrile gradient system) gave the title compound as a white powder (33 mg). MS (ES): m/z 518.35 (M+H).

### EXAMPLE 54

### 2-Methoxy-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide

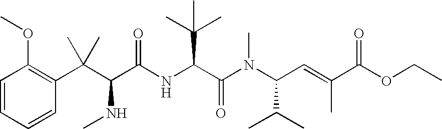

N-(tert-Butoxycarbonyl)-2-methoxy-N,β,β-trimethyl-L-phenylalanyl-N¹-[(1S,2E)-3-carboxy-1-isopropyl-2-butenyl]-N¹,3-dimethyl-L-valinamide (54 mg, 0.086 mmol, from Reference Example 82) is treated with 4N hydrochloric acid/dioxane (2 mL) at room temperature for 1.5 hours, then concentrated in vacuo. The residue is washed with ether (3×1 mL) and dried in vacuo to give 40 mg (88%) of the hydrochloric acid salt of the title compound as a white solid. MS (ESI) calcd for C₃₀H₄₉N₃O₅ (M+H⁺) 532, found 532.

### EXAMPLE 55

### 2-Methoxy-N,β,β-trimethyl-L-phenylalanyl-N¹-[(1S,2E)-3-carboxy-1-isopropyl-2-butenyl]-N¹,3-dimethyl-L-valinamide

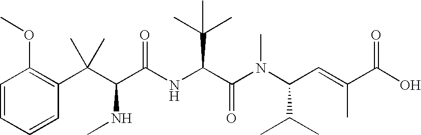

2-Methoxy-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (64 mg, 0.106 mmol, from Reference Example 83) is treated with 4N hydrochloric acid/dioxane (2 mL) at room temperature for 1.5 hours, then concentrated in vacuo. The residue is washed with ether (3×1 mL), then purified by preparative reverse phase HPLC to give 36 mg (67%) of the trifluoroacetic acid salt of the title compound as a white solid. HRMS (ESI) calcd for C₂₈H₄₅N₃O₅ (M+H⁺) 504.3432, found 504.3429.

### EXAMPLE 56

### N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide

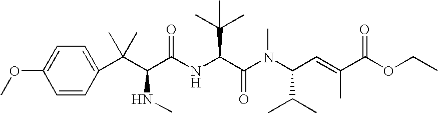

N-(tert-Butoxycarbonyl)-N,O,β,β-tetramethyl-L-tyrosyl-N¹-[(1S,2E)-3-carboxy-1-isopropyl-2-butenyl]-N¹,3-dimethyl-L-valinamide (40 mg, 0.063 mmol, from Reference Example 84) is treated with 4N hydrochloric acid/dioxane (2 mL) at room temperature for 2 hours, then concentrated in vacuo. The residue is washed with ether (3×1 mL) and dried in vacuo to give 21 mg (62%) of the hydrochloric acid salt of the title compound as a white solid. HRMS (ESI) calcd for C₃₀H₄₉N₃O₅ (M+H⁺) 532.3745, found 532.3746.

### EXAMPLE 57

### N,O,β,β-tetramethyl-L-tyrosyl-N¹-[(1S,2E)-3-carboxy-1-isopropyl-2-butenyl]-N¹,3-dimethyl-L-valinamide

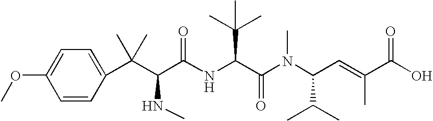

N-(tert-Butoxycarbonyl)-N,O,β,β-tetramethyl-L-tyrosyl-N¹-[(1S,2E)-3-carboxy-1-isopropyl-2-butenyl]-N¹,3-dimethyl-L-valinamide (217 mg, 0.36 mmol, from Reference Example 85) is treated with 4N hydrochloric acid/dioxane (10 mL) at room temperature for 2 hours, then concentrated in vacuo. The residue is washed with ether (3×5 mL), then purified by preparative reverse phase HPLC to give 117 mg (65%) of the trifluoroacetic acid salt of the title compound as a white solid. HRMS (ESI) calcd for C₂₈H₄₅N₃O₅ (M+H⁺) 504.3432, found 504.3427.

### EXAMPLE 58

### 2-Methoxy-N,O,β,β,tetramethyl-L-tyrosyl-N¹-[(1S,2E)-4-ethoxy-1-isopropyl-3-methyl-4-oxo-2-butenyl]-N¹,3-dimethyl-L-valinamide

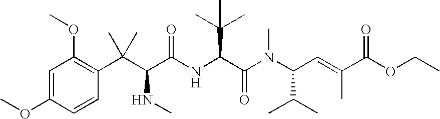

N-(tert-Butoxycarbonyl)-2-methoxy-N,O,β,β-tetramethyl-L-tyrosyl-N¹-[(1S,2E)-3-carboxy-1-isopropyl-2-butenyl]-N¹,3-dimethyl-L-valinamide (32 mg, 0.048 mmol, from Reference Example 87) is treated with 4N hydrochloric acid/dioxane (0.8 mL) at room temperature for 1 h, then concentrated in vacuo. The residue is washed with ether (3×1 mL) and dried in vacuo to give 21 mg (73%) of the hydrochloric acid salt of the title compound as a white solid. HRMS (ESI) calcd for C₃₁H₅₁N₃O₆ (M+H⁺) 562.3851, found 562.3845.

### EXAMPLE 59

### 2-Methoxy-N,O,β,β-tetramethyl-L-tyrosyl-N¹-[(1S,2E)-3-carboxy-1-isopropyl-2-butenyl]-N¹,3-dimethyl-L-valinamide

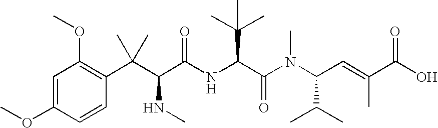

2-Methoxy-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide (90 mg, 0.142 mmol, from Example 88) is treated with 4N hydrochloric acid/dioxane (1.7 mL) at room temperature for 1.5 hours, then concentrated in vacuo. The residue is washed with ether (3×1 mL), then purified by preparative reverse phase HPLC to give 43 mg (47%) of the trifluoroacetic acid salt of the title compound as a white solid. HRMS (ESI) calcd for C₂₉H₄₇N₃O₆ (M+H⁺) 534.3538, found 534.3535.

### EXAMPLE 60

### 3-Fluoro-N,β,β-trimethyl-L-phenylalanyl-N¹-[(1S,2E)-3-carboxy-1-isopropyl-2-butenyl]-N¹,3-dimethyl-L-valinamide

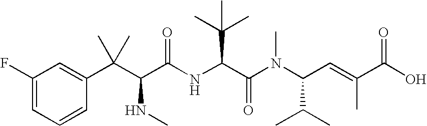

According to General Procedure V, 3-fluoro N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (40 mg, 0.08 mmol) from Reference Example 90 is dissolved in methanol (1.8 mL) and water (0.6 mL) and treated with 1.0 M aqueous lithium hydroxide (0.62 mL, 0.62 mmol) to give 39 mg (84%) of the trifluoroacetic acid salt of the title compound as a white solid after preparative reverse phase HPLC separation. HRMS (ESI) calcd for C₂₇H₄₂FN₃O₄ (M+H⁺) 492.3232, found 492.3223.

### EXAMPLE 61

### 3-Fluoro- N,β,β-trimethyl-D-phenylalanyl-N¹-[(1S,2E)-3-carboxy-1-isopropyl-2-butenyl]-N¹,3-dimethyl-L-valinamide

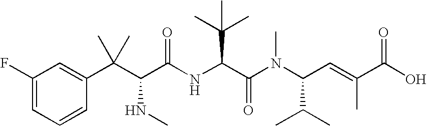

According to General Procedure V, 3-fluoro N,β,β-trimethyl-D-phenylalanyl-N¹-[(1S,2E)-4-ethoxy-1-isopropyl-3-methyl-4-oxo-2-butenyl]-N¹,3-dimethyl-L-valinamide (40 mg, 0.08 mmol, from Reference Example 90) is dissolved in methanol (1.8 mL) and water (0.6 mL) and treated with 1.0 M aqueous lithium hydroxide (0.62 mL, 0.62 mmol) to give 42 mg (90%) of the trifluoroacetic acid salt of the title compound as a white solid after preparative reverse phase HPLC separation. HRMS (ESI) calcd for C₂₇H₄₂FN₃O₄ (M+H⁺) 492.3232, found 492.3223.

### EXAMPLE 62

### N,β,β-Trimethyl-3-(trifluoromethyl)-L-phenylalanyl-N¹-[(1S,2E)-3-carboxy-1-isopropyl-2-butenyl]-N¹,3-dimethyl-L-valinamide

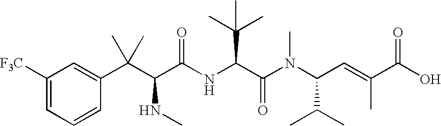

According to General Procedure V, N,β,β-trimethyl-3-(trifluoromethyl)-L-phenylalanyl-N¹-[(1S,2E)-4-ethoxy-1-isopropyl-3-methyl4-oxo-2-butenyl]-N¹,3-dimethyl-L-valinamide (57 mg, 0.1 mmol) from Reference Example 92 is dissolved in methanol (2.4 mL) and water (0.8 mL) and treated with 1.0 M aqueous lithium hydroxide (0.8 mL, 0.8 mmol). Preparative reverse phase HPLC purification provided 31 mg (47%) of the trifluoroacetic acid salt of the title compound as a white solid. HRMS (ESI) calcd for C₂₈H₄₂F₃N₃O₄ (M+H⁺) 542.3200, found 542.3189.

### EXAMPLE 63

### N,β,β-Trimethyl-3-(trifluoromethyl)-D-phenylalanyl-N¹-[(1S,2E)-3-carboxy-1-isopropyl-2-butenyl]-N¹,3-dimethyl-L-valinamide

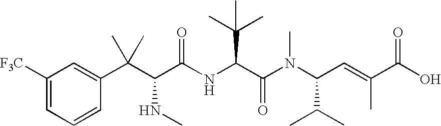

According to General Procedure V, N,β,β-trimethyl-3-(trifluoromethyl)-D-phenylalanyl-N¹-[(1S,2E)4-ethoxy-1-isopropyl-3-methyl-4-oxo-2-butenyl]-N¹,3-dimethyl-L-valinamide (57 mg, 0.1 mmol, from Reference Example 92) is dissolved in methanol (2.4 mL) and water (0.8 mL) and treated with 1.0 M aqueous lithium hydroxide (0.8 mL, 0.8 mmol). Preparative reverse phase HPLC purification provided 45 mg (69%) of the trifluoroacetic acid salt of the title compound as a white solid. HRMS (ESI) calcd for C₂₈H₄₂F₃N₃O₄ (M+H⁺) 542.3200, found 542.3194.

### EXAMPLE 64

### 3,5-Difluoro- N,β,β-trimethyl-L-phenylalanyl-N¹-[(1S,2E)-3-carboxy-1-isopropylbut-2-enyl]-N¹,3-dimethyl-L-valinamide

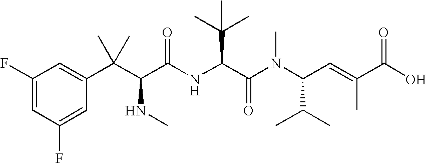

According to General Procedure V, 3,5-difluoro- N,β,β-trimethyl-L-phenylalanyl-N¹-[(1S,2E)-4-ethoxy-1-isopropyl-3-methyl-4-oxobut-2-enyl]-N¹,3-dimethyl-L-valinamide (54 mg, 0.1 mmol, from Reference Example 94) is dissolved in methanol (2.4 mL) and water (0.8 mL) and treated with 1.0 M aqueous lithium hydroxide (0.8 mL, 0.8 mmol) to give 55 mg (88%) of the trifluoroacetic acid salt of the title compound as a white solid after preparative reverse phase HPLC. HRMS (ESI) calcd for C₂₇H₄₁F₂N₃O₄ (M+H⁺) 510.3138, found 510.3138.

### EXAMPLE 65

### 3,5-Difluoro- N,β,β-trimethyl-D-phenylalanyl-N¹-[(1S,2E)-3-carboxy-1-isopropylbut-2-enyl]-N¹,3-dimethyl-L-valinamide

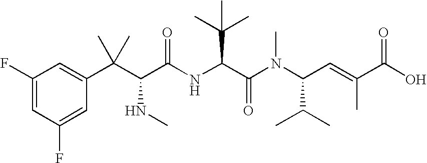

According to General Procedure V, 3,5-difluoro- N,β,β-trimethyl-D-phenylalanyl-N¹-[(1S,2E)-4-ethoxy-1-isopropyl-3-methyl-4-oxobut-2-enyl]-N¹,3-dimethyl-L-valinamide (54 mg, 0.1 mmol, from Reference Example 94) is dissolved in methanol (2.4 mL). To this is added water (0.8 mL) and 1.0 M aqueous lithium hydroxide (0.8 mL, 0.8 mmol). This provided 58 mg (93%) of the trifluoroacetic acid salt of the title compound as a white solid after preparative HPLC separation. HRMS (ESI) calcd for C₂₇H₄₁F₂N₃O₄ (M+H⁺) 510.3138, found 510.3130.

### EXAMPLE 66

### N,β,β-trimethyl-3,5-bis(trifluoromethyl)-L-phenylalanyl-N¹-[(1S,2E)-3-carboxy-1-isopropylbut-2-enyl]-N¹,3-dimethyl-L-valinamide

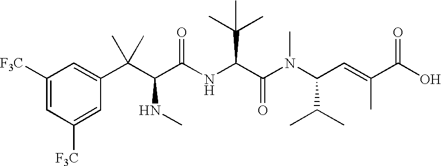

According to General Procedure V, N,β,β-trimethyl-3,5-bis(trifluoromethyl)-L-phenylalanyl-N¹-[(1S,2E)-4-ethoxy-1-isopropyl-3-methyl-4-oxobut-2-enyl]-N¹,3-dimethyl-L-valinamide (40 mg, 0.06 mmol, from Reference Example 96) is dissolved in methanol (1.8 mL). To this is added water (0.6 mL) and 1.0 M aqueous lithium hydroxide (0.5 mL, 0.5 mmol). Preparative reverse phase HPLC purification provided 25 mg (56%) of the trifluoroacetic acid salt of the title compound as a white solid. HRMS (ESI) calcd for C₂₉H₄₁F₆N₃O₄ (M+H⁺) 610.3074, found 610.3069.

### EXAMPLE 67

### N,β,β-trimethyl-3,5-bis(trifluoromethyl)-D-phenylalanyl-N¹-[(1S,2E)-3-carboxy-1-isopropylbut-2-enyl]-N¹,3-dimethyl-L-valinamide

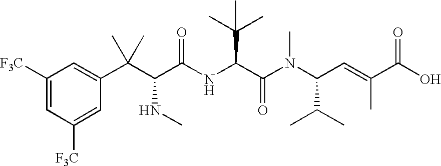

According to General Procedure V, N,β,β-trimethyl-3,5-bis(trifluoromethyl)-D-phenylalanyl-N¹-[(1S,2E)4-ethoxy-1-isopropyl-3-methyl-4-oxobut-2-enyl]-N¹,3-dimethyl-L-valinamide (50 mg, 0.08 mmol, from Reference Example 96) is dissolved in methanol (2.0 mL). To this is added water (0.64 mL) and 1.0 M aqueous lithium hydroxide (0.64 mL, 0.64 mmol). Preparative reverse phase HPLC purification provided 45 mg (79%) of the trifluoroacetic acid salt of the title compound as a white solid. HRMS (ESI) calcd for C₂₉H₄₁F₆N₃O₄ (M+H⁺) 610.3074, found 610.3072.

### EXAMPLES 68 AND 69

### O-isopropyl- N,β,β-trimethyl-L-tyrosyl-N¹-[(1S,2E)-3-carboxy-1-isopropylbut-2-enyl]-N¹,3-dimethyl-L-valinamide and Example 69 0-isopropyl- N,β,β-trimethyl-D-tyrosyl-N¹-[(1S,2E)-3-carboxy-1-isopropylbut-2-enyl]-N¹,3-dimethyl-L-valinamide

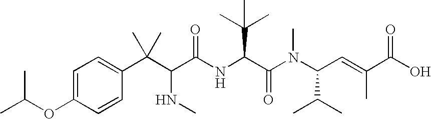

According to General Procedure V, O-isopropyl- N,β,β-trimethyltyrosyl-N¹-[(1S,2E)4-ethoxy-1-isopropyl-3-methyl-4-oxobut-2-enyl]-N¹,3-dimethyl-L-valinamide (80 mg, 0.14 mmol, from Reference Example 98) is dissolved in methanol (3.3 mL). To this is added water (1.1 mL) and 1.0 M aqueous lithium hydroxide (1.14 mL, 1.14 mmol). Preparative reverse phase HPLC purification provided 22 mg of the trifluoroacetic acid salt of O-isopropyl- N,β,β-trimethyl-L-tyrosyl-N¹-[(1S,2E)-3-carboxy-1-isopropylbut-2-enyl]-N¹,3-dimethyl-L-valinamide as a white solid and 5 mg of the trifluoroacetic acid salt of the 0-isopropyl- N,β,β-trimethyl-D-tyrosyl-N¹-[(1S,2E)-3-carboxy-1-isopropylbut-2-enyl]-N¹,3-dimethyl-L-valinamide as a white solid. O-isopropyl-N,β,β-trimethyl-L-tyrosyl-N¹-[(1S,2E)-3-carboxy-1-isopropylbut-2-enyl]-N¹,3-dimethyl-L-valinamide: HRMS (ESI) calcd for C₃₀H₄₉N₃O₅ (M+H⁺) 532.3745, found 532.3741; O-isopropyl- N,β,β-trimethyl-D-tyrosyl-N¹-[(1S,2E)-3-carboxy-1-isopropylbut-2-enyl]-N¹,3-dimethyl-L-valinamide: HRMS (ESI) calcd for C₃₀H₄₉N₃O₅ (M+H⁺) 532.3745, found 532.3741.

### EXAMPLE 70

### 3-Cyclohexyl-N-methyl-L-valyl-N-1-[(1S,2E)-3-carboxy-1-isopropyl-2-butenyl]-N-1,3-dimethyl-L-valinamide

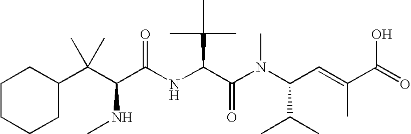

Following General Procedure V, 3-cyclohexyl-N-methyl-L-valyl-N-1-[(1S,2E)-4-ethoxy-1-isopropyl-3-methyl-4-oxo-2-butenyl]-N-1,3-dimethyl-L-valinamide (0.685 g, 1.35 mmol, from Reference Example 100) is hydrolyzed and the product isolated by reverse phase HPLC (0.01% trifluoroacetic acid in water/acetonitrile) to give the trifluoroacetic acid salt of the title compound as a white solid (0.408 g). ¹H NMR (DMSOd₆, d): 12.43 (1H, vbr), 8.64 (1H, br), 8.53 (1H, d, J=8.3 Hz), 8.37 (1H, br), 6.64 (1H, dd, J=9.6, 1.4 Hz), 4.91 (1H, t, J=10.1 Hz), 4.74 (1H, d, J=8.3 Hz), 4.01 (1H, d, J=9.7 Hz), 2.98 (3H, s), 2.44 (3H, brt, J=4.7 Hz) 1.92-2.05 (1H, m), 1.78 (3H, d, J=1.1 Hz) over lap with 1.59-1.8 (5H, m), 0.95 (9H, s, t-Bu) and 0.93 (3H, s, Me) overlap with 0.89-1.43 (5H, m), 0.79 (3H, d, J=6.6 Hz), 0.73 (3H, d, J=6.6 Hz), 0.71 (3H, s). MS: m/z 480.35 (M+H).

### EXAMPLES 71 AND 72

### (2E,4S)-2,5-dimethyl-4-(methyl{3-methyl-N-[(2S)-2-(methylamino)-2-(1-phenylcyclopentyl)ethanoyl]-L-valyl}amino)-2-hexenoic acid and Example 72 (2E,4S)-2,5-dimethyl-4-(methyl{3-methyl-N-[(2R)-2-(methylamino)-2-(1-phenylcyclopentyl)ethanoyl]-L-valyl}amino)-2-hexenoic Acid

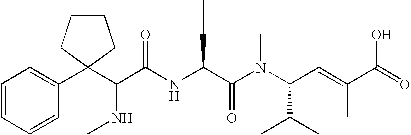

Following General Procedure V, ethyl (2E,4S)-2,5-dimethyl-4-(methyl{3-methyl-N-[2-(methylamino)-2-(1-phenylcyclopentyl)ethanoyl]-L-valyl}amino)-2-hexenoate (78 mg, 0.15 mmol, from Reference Example 103) is hydroyzed to give two components as their trifluoroacetic acid salts after reverse phase HPLC (0.01% trifluoroacetic acid in water/acetonitrile). (2E,4S)-2,5-Dimethyl4-(methyl{3-methyl-N-[(2S)-2-(methylamino)-2-(1-phenylcyclopentyl)ethanoyl]-L-valyl}amino)-2-hexenoic acid is isolated as a white glass (22 mg). MS: m/z 500.62 (M+H) (2E,4S)-2,5-Dimethyl4-(methyl{3-methyl-N-[(2R)-2-(methylamino)-2-(1-phenylcyclopentyl)ethanoyl]-L-valyl}amino)-2-hexenoic acid is isolated as a white glass (25 mg, contains 8% (2E,4S)-2,5-dimethyl4-(methyl{3-methyl-N-[(2S)-2-(methylamino)-2-(1-phenylcyclopentyl)ethanoyl]-L-valyl}amino)-2-hexenoic acid). MS: m/z 500.63 (M+H),

### EXAMPLE 73

### (2E,4R)-2,5-dimethyl4-(methyl{3-methyl-N-[(methylamino)(1-phenylcyclohexyl)acetyl]-L-valyl}amino)-2-hexenoic Acid

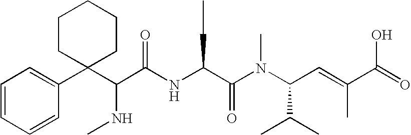

Following General Procedure V, ethyl (2E,4R)-2,5-dimethyl4-(methyl{3-methyl-N-[(methylamino)(1-phenylcyclohexyl)acetyl]-L-valyl}amino)-2-hexenoate (273 mg, 0.50 mmol, from Reference Example 106) is hydrolyzed. Reverse phase HPLC (0.01% trifluoroacetic acid in water/acetonitrile) gave the trifluoroacetic acid salt of the title compound as a white glass (10 mg). MS: m/z 514.15 (M+H)

### EXAMPLE 74

### (E,4S)-2,5-Dimethyl-4-[methyl((2S)-2-{[(2S)-3-methyl-2-(methylamino)-3-phenylbutanoyl]amino}-3-phenylpropanoyl)amino]-2-hexenoic Acid

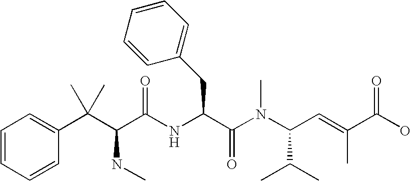

By using a procedure analogous to that described in General Procedure V, ethyl (6S,9S,12S,13E)-9-benzyl-12-isopropyl-2,2,5,11,14-pentamethyl-6-(1-methyl-1-phenylethyl)-4,7,10-trioxo-3-oxa-5,8,11-triazapentadec-13-en-15-oate (56 mg, 0.09 mmol, obtained from Reference Example 108) is treated with lithium hydroxide aqueous solution (0.72 mmol) in water (0.72 mL) and methanol (2.1 mL) at room temperature for 15 hours, to provide (6S,9S,12S,13E)-9-benzyl-12-isopropyl-2,2,5,11,14-pentamethyl-6-(1-methyl-1-phenylethyl)-4,7,10-trioxo-3-oxa-5,8,11-triazapentadec-13-en-15-oic acid (55 mg, 100%) as a colorless oil. This material (55 mg, 0.091 mmol) is then treated with hydrogen chloride (0.34 mL, 1.37 mmol; 4 M para-dioxane solution, Aldrich) at room temperature for 30 minutes, to provide (E,4S)-2,5-dimethyl-4-[methyl((2S)-2-{[(2S)-3-methyl-2-(methylamino)-3-phenylbutanoyl]amino}-3-phenylpropanoyl)amino]-2-hexenoic acid hydrogen chloride salt (50 mg, 100%) as a white solid. MS (ES): m/z 507.9 (M+H). Analytical HPLC: (4.6×150 mm Prodigy ODS3 column eluted with 10 to 90% acetonitrile in water containing 0.02% TFA over 30 minutes-isocratic method): 62.1% of (E,4S)-2,5-dimethyl-4-[methyl((2S)-2-{[(2S)-3-methyl-2-(methylamino)-3-phenylbutanoyl]amino)-3-phenylpropanoyl)amino]-2-hexenoic acid at 9.7 minutes, and two other diastereomers, 12.3% (at 10.1 minutes) and 2.12% (at 10.3 minutes).

### EXAMPLE 75

### N,β,β-Trimethyl-L-phenylalanyl-N¹-[(1S,2E)-1-butyl-3-carboxybut-2-enyl]-N¹,3-dimethyl-L-valinamide

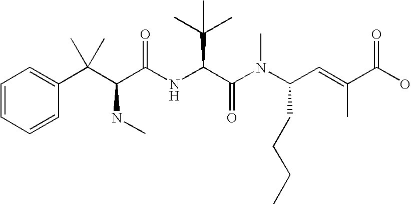

N-(tert-Butoxycarbonyl)-N,β,β-trimethyl-L-phenylalanyl-N¹-[(1S,2E)-3-carboxy-1-n-butyl-2-butenyl]-N¹,3-dimethyl-L-valinamide (94.1 mg, 0.16 mmol, obtained from Reference Example 112) is treated with hydrogen chloride (0.6 mL, 2.4 mmol; 4 M para-dioxane solution, Aldrich) at room temperature for 30 minutes, to provide N,β,β-trimethyl-L-phenylalanyl-N¹-[(1S,2E)-3-carboxy-1-n-butyl-2-butenyl]-N¹,3-dimethyl-L-valinamide hydrogen chloride (83 mg, 100%) as a white powder. MS (ES): m/z 488.6 (M+H). IR cm⁻¹: 3402, 2962, 2873, 1680, 1649. Analytical HPLC (4.6×150 mm Prodigy ODS3 column eluted with a liner gradient of 10-90% actonitrile in water containing 0.02% trifluoroacetic acid over 35 min): 70.9% (at 10.82 minutes) of N,β,β-trimethyl-L-phenylalanyl-N¹-[(1S,2E)-3-carboxy-1-n-butyl-2-butenyl]-N¹,3-dimethyl-L-valinamide, and 0.87% (6.18 minutes), 1.08% (at 6.46 minutes), 22.6% (at 6.62 minutes) and 4.24% (at 7.84minutes) of four other diastereomers.

### EXAMPLE 76

### N,β,β-Trimethyl-L-phenylalanyl-N¹-[(1S,2E)-3-carboxy-1-isobutyl-2-pentenyl]-N¹,3-dimethyl-L-valinamide

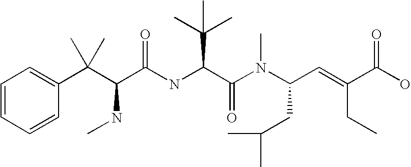

By using a procedure analogous to that described in General Procedure V, N-(tert-butoxycarbonyl)-N,β,β-trimethyl-L-phenylalanyl-N¹-[(1 S ,2E)-3-(ethoxycarbonyl)-1-isobutyl-2-pentenyl]-N¹,3-dimethyl-L-valinamide (57 mg, 0.09 mmol, obtained from Reference Example 116) is treated with lithium hydroxide aqueous solution (0.718 mmol) in water (0.718 mL) and methanol (2.5 mL) at room temperature for 15 hours, to provide N-(tert-butoxycarbonyl)-_N,β,β-trimethyl-L-phenylalanyl-N¹-[(1S,2E)-3-(carbonyl)-1-isobutyl-2-pentenyl]-N¹,3-dimethyl-L-valinamide (54 mg, 96%) as a colorless glass. MS (ES): m/z (M+H). N-(tert-Butoxycarbonyl)- N,β,β-trimethyl-L-phenylalanyl-N¹-[(1S,2E)-3-(carbonyl)-1-isobutyl-2-pentenyl]-N¹,3-dimethyl-L-valinamide (54 mg, 0.087 mmol) is then treated with hydrogen chloride (1 mL, 4 M para-dioxane solution, Aldrich) at room temperature for 45 minutes. The mixture is azeotroped with toluene and dried to provide N,β,β-trimethyl-L-phenylalanyl-N¹-[(1S,2E)-3-carboxy-1-isobutyl-2-pentenyl]-N¹,3-dimethyl-L-valinamide hydrogen chloride (51 mg, 100%) as a white solid. MS (ES): m/z 502.4 (M+H). IR cm⁻¹: 3418.15, 2961.05, 1679.52, 1646.23. Analytical HPLC: (4.6×150 mm YMC Pack Pro C18 column eluted with 10 to 100% acetonitrile in water containing 0.02% formic acid over 35 minutes): 54.91% (at 11.5 minutes) of N,β,β-trimethyl-L-phenylalanyl-N¹-[(1S,2E)-3-carboxy-1-isobutyl-2-pentenyl]-N¹,3-dimethyl-L-valinamide, and 6.56% (at 11.7 minutes), 5.77% (at 12.5 minutes) and 0.73% (at 13.8 minutes) of three other diastereomers.

### EXAMPLE 77

### (E,4S)-2-Butyl-4-[((2S)-3,3-dimethyl-2-{[(2S)-3-methyl-2-(methylamino)-3-phenylbutanoyl]amino}butanoyl)amino]-5-methyl-2-hexenoic Acid

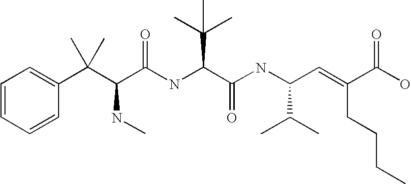

(6S,9S,12S,13E)-14-Butyl-9-(tert-butyl)-12-isopropyl-2,2,5-trimethyl-6-(1-methyl-1-phenylethyl)-4,7,10-trioxo-3-oxa-5,8,11-triazapentadec-13-en-15-oic acid (63 mg, 0.105 mmol, obtained from Reference Example 120) is treated with hydrogen chloride (0.52 mL, 2.1 mL; 4 M para-dioxane solution, Aldrich) at room temperature for 30 minutes, to provide (E,4S)-2-butyl-4-[((2S)-3,3-dimethyl-2-{[(2S)-3-methyl-2-(methylamino)-3-phenylbutanoyl]amino}butanoyl)amino]-5-methyl-2-hexenoic acid hydrogen chloride (55 mg, 97.4%) as a white solid. MS (ES): m/z 502.0 (M+H).

### EXAMPLE 78

### N,β,β-Trimethyl-L-phenylalanyl-N¹-[(1S,2E)-3-carboxy-1-isopropyl-2-pentenyl]-N 1,3-dimethyl-L-valinamide

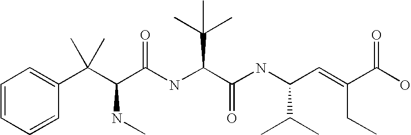

N-(tert-Butoxycarbonyl)- N,β,β-trimethyl-L-phenylalanyl-N¹-[(1S,2E)-3-carboxy-1-isopropyl-2-pentenyl]-N¹,3-dimethyl-L-valinamide (184.6 mg, 0.314 mmol, obtained from Reference Example 124) is treated with hydrogen chloride (1.2 mL, 4.71 mmol; 4 M para-dioxane solution, Aldrich) at room temperature for 30 minutes. The product is purified by using preparative HPLC, to provide N,β,β-trimethyl-L-phenylalanyl-N¹-[(1S,2E)-3-carboxy-1-isopropyl-2-pentenyl]-N¹,3-dimethyl-L-valinamide trifluoroacetic acid a white solid. MS (ES): m/z 974.1 (2M−H), 486.5 (M−H). IR cm⁻¹: 2966, 2877, 1677, 1645. Analytical HPLC (4.6×150 mm Prodigy ODS3 column eluted with a liner gradient of 10-100% actonitrile in water containing 0.02% trifluoroacetic acid over 35 min): 91.1% (at 13.454 minutes)of N,β,β-trimethyl-L-phenylalanyl-N¹-[(1S,2E)-3-carboxy-1-isopropyl-2-pentenyl]-N¹,3-dimethyl-L-valinamide, and 7.5% (at 13.738 minutes) and 0.9% (14.502 minutes) of two other diastereomers.

### EXAMPLE 79

### Ethyl (E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoate

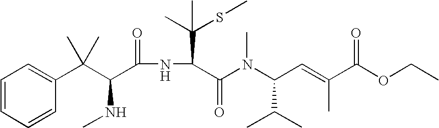

(6S,9R,12S,13E)-12-isopropyl-2,2,5,11,14-pentamethyl-9-[1-methyl-1-(methylsulfanyl)ethyl]-6-(1-methyl-1-phenylethyl)-4,7,10-trioxo-3-oxa-5,8,11-triazapentadec-13-en-15-oic acid (20 mg, 0.03 mmol, from Reference Example 127) is treated with 4N hydrochloric acid/dioxane (0.5 mL) at room temperature for 4 hours and then concentrated to dryness. The residual solid is washed with ether and dried in vacuo to give 14 mg (78%) of the hydrochloric acid salt of the title compound as a white solid. HRMS (ESI) calcd for C₂₉H₄₇N₃O₄S (M+H⁺) 534.3360, found 534.3353.

### EXAMPLE 80

### (E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoic Acid

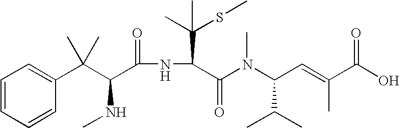

(6S,9R,12S,13E)-12-Isopropyl-2,2,5,11,14-pentamethyl-9-[1-methyl-1-(methylsulfanyl)ethyl]-6-(1-methyl-1-phenylethyl)-4,7,10-trioxo-3-oxa-5,8,11-triazapentadec-13-en-15-oic acid (30 mg, 0.05 mmol, from Reference Example 128) is treated with 4N hydrochloric acid/dioxane (0.6 mL) at room temperature for 2 hours, then concentrated to dryness. The residual solid is washed with ether and dried in vacuo to give 25 mg (93%) of the hydrochloric acid salt of the title compound as a white solid. HRMS (ESI) calcd for C₂₇H₄₃N₃O₄S (M+H⁺) 506.3047, found 506.3044.

### EXAMPLE 81

### N,β,β-trimethyl-L-phenylalanyl-N¹-[(1S,2E)-3-carboxy-1-isopropyl-2-butenyl]-N¹-methyl-3-(methylsulfonyl)-L-valinamide

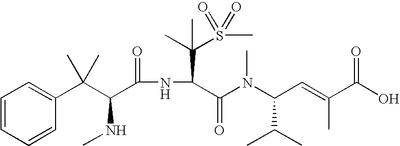

N-(tert-Butoxycarbonyl)-N,β,β-trimethyl-L-phenylalanyl-N¹-[(1S,2E)-3-carboxy-1-isopropyl-2-butenyl]-N¹-methyl-3-(methylsulfonyl)-L-valinamide (40 mg, 0.06 mmol, from Reference Example 129) is treated with 4N hydrochloric acid/dioxane (1.3 mL) at room temperature for 1.2 hours, then concentrated to dryness. The residual solid is washed with ether and purified by preparative reverse phase HPLC to give 20 mg (59%) of the trifluoroacetic acid salt of the title compound as a white solid. HRMS (ESI) calcd for C₂₇H43N₃O₆S (M+H⁺) 538.2945, found 538.2938.

### EXAMPLE 82

### N,β,β-trimethyl-L-phenylalanyl-N¹-[(1S,2E)-3-carboxy-1-isopropyl-2-butenyl]-3-[(4-methoxybenzyl)sulfanyl]-N¹-methyl-L-valinamide

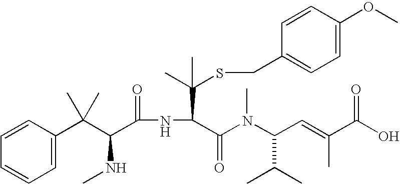

N,β,β-Trimethyl-L-phenylalanyl-N¹-[(1R,2E)-4-ethoxy-1-isopropyl-3-methyl-4-oxo-2-butenyl]-3-[(4-methoxybenzyl)sulfanyl]-N¹-methyl-L-valinamide (35 mg, 0.05 mmol, from Reference Example 133) is treated with 4N hydrochloric acid/dioxane (1.5 mL) to give 26 mg (81%) of the hydrochloric acid salt of the title compound as a white solid. HRMS (ESI) calcd for C₃₄H₄₉N₃O₅S (M+H⁺) 612.3466, found 612.3460.

### EXAMPLE 83

### N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide

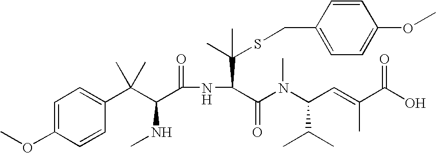

N-(tert-Butoxycarbonyl)-N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide (70 mg, 0.09 mmol, from Reference Example 136) is treated with 4N hydrochloric acid/dioxane (2 mL) at room temperature for 1.5 hours, then concentrated. The residue is washed with ether (3×1 mL) and dried in vacuo. Preparative HPLC separation gave 23 mg (32%) of the trifluoroacetic acid salt of the title compound as a white solid. HRMS (ESI) calcd for C₃₅H₅₁N₃O₆S (M−H⁺) 640.3426, found 640.3426.

### EXAMPLE 84

### N,O,β,β-tetramethyl-L-tyrosyl-N¹-[(1S,2E)-3-carboxy-1-isopropyl-2-butenyl]-N¹-methyl-3-(methylsulfanyl)-L-valinamide

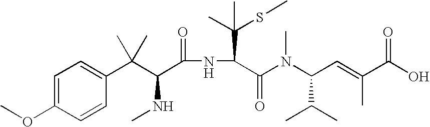

N-(tert-Butoxycarbonyl)-N,O,β,β-tetramethyl-L-tyrosyl-N¹-[(1S,2E)-3-carboxy-1-isopropyl-2-butenyl]-N¹-methyl-3-(methylsulfanyl)-L-valinamide (50 mg, 0.079 mmol, from Reference Example 139) in dichloromethane (1 mL) is treated with trifluoroacetic acid (0.3 mL) at room temperature for 1.5 hours, then concentrated in vacuo. The residue is washed with ether (3×1 mL) and dried in vacuo to give 51 mg (100%) of the trifluoroacetic acid salt of the title compound as a white solid. HRMS (ESI) calcd for C₂₈H₄₅N₃O₅S (M+H⁺) 536.3153, found 536.3149.

### EXAMPLE 85

### N,β,β-trimethyl-L-phenylalanyl-N¹-[(1R,2E)-4-ethoxy-1-isopropyl-3-methyl-4-oxo-2-butenyl]-N¹-methyl-L-allothreoninamide

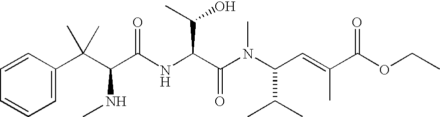

N-(tert-Butoxycarbonyl)-N,β,-trimethyl-L-phenylalanyl-N¹-[(1R,2E)-4-ethoxy-1-isopropyl-3-methyl-4-oxo-2-butenyl]-N¹-methyl-L-allothreoninamide (30 mg, 0.05 mmol, from Reference Example 142) is treated with 4N hydrochloric acid/dioxane (1.5 mL) at room temperature for 3 hours to give 21 mg (79%) of the hydrochloric acid salt of the title compound as a white solid. HRMS (ESI) calcd for C₂₇H₄₃N₃O₅ (M+H⁺) 490.3275, found 490.3261.

### EXAMPLE 86

### N,β,β-trimethyl-L-phenylalanyl-N¹-[(1S,2E)-3-carboxy-1-isopropyl-2-butenyl]-N¹-methyl-L-allothreoninamide

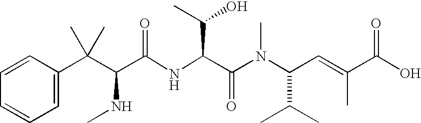

N-(tert-Butoxycarbonyl)-N,β,β-trimethyl-L-phenylalanyl-N¹-[(1R,2E)-3-carboxy-1-isopropyl-2-butenyl]-N¹-methyl-L-allothreoninamide (30 mg, 0.05 mmol, from Reference Example 143) in dichloromethane (1 mL) is treated with trifluoroacetic acid (0.3 mL) at room temperature for 1.5 hours, then concentrated in vacuo. The residue is washed with ether, dried in vacuo to give 22 mg (72%) of the trifluoroacetic acid salt of the title compound as a white solid. HRMS (ESI) calcd for C₂₅H₃₉N₃O₅ (M+H⁺) 462.2963, found 462.2967.

### EXAMPLE 87

### N,β,β-trimethyl-L-phenylalanyl-N-[(1S,2E)-3-carboxy-1-isopropyl-2-butenyl]-N,O,β,β-tetramethyl-L-tyrosinamide

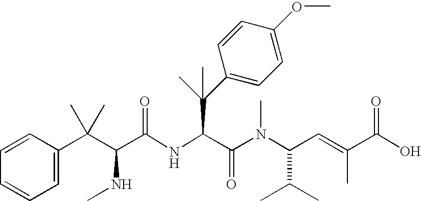

N-(tert-Butoxycarbonyl)-N,β,β-trimethyl-L-phenylalanyl-N-[(1S,2E)-3-carboxy-1-isopropyl-2-butenyl]-N,O,β,β-tetramethyl-L-tyrosinamide (40 mg, 0.06 mmol, from Reference Example 147) is treated with 4N hydrochloric acid/dioxane (1.5 mL) at room temperature for 1.5 hours to give 32 mg (84%) of the hydrochloric acid salt of the title compound as a white solid. HRMS (ESI) calcd for C₃₃H₄₇N₃O₅ (M+H⁺) 566.3589, found 566.3584.

### EXAMPLE 88

### N,β,β-trimethyl-L-phenylalanyl-N¹-[(1S,2E)-3-carboxy-1-isopropyl-2-butenyl]-N¹,O-dimethyl-L-allothreoninamide

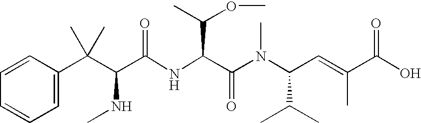

N-(tert-Butoxycarbonyl)-N,β,β-trimethyl-L-phenylananyl-N¹-[(1S,2E)-3-carboxy-1-isopropyl-2-butenyl]-N¹,O-dimethyl-L-allothreoninamide (40 mg, 0.07 mmol, from Reference Example 152) in dichloromethane (1 mL) is treated with trifluoroacetic acid (0.2 mL) at room temperature for 1.5 hours, then concentrated in vacuo. The residue is washed with ether and dried in vacuo to give 39 mg (95%) of the trifluoroacetic acid salt of the title compound as a white solid. HRMS (ESI) calcd for C₂₆H₄₁N₃O₅ (M+H⁺) 476.3119, found 476.3119.

### EXAMPLE 89

### (E ,4S)-2,5-Dimethyl-4-[methyl((2S)-2-{[(2S)-3-methyl-2-(methylamino)-3-phenylbutanoyl]amino}4-phenylbutanoyl)amino]-2-hexenoic Acid

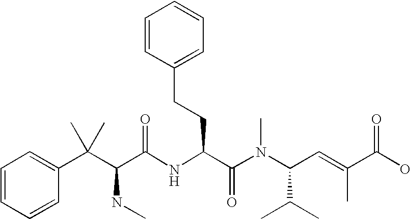

By using a procedure analogous to that described in General Procedure V, ethyl (6S,9S,12S,13E)-12-isopropyl-2,2,5,11,14-pentamethyl-6-(1-methyl-1-phenylethyl)-4,7,10-trioxo-9-(2-phenylethyl)-3-oxa-5,8,11-triazapentadec-13-en-15-oate (130 mg, 0.2 mmol, obtained from Reference Example 155) is treated with lithium hydroxide aqueous solution (1.6 mmol) in water (1.6 mL) and methanol (5.7 mL) at room temperature for 15 hours, to provide , (6S,9S,12S,13E)-12-isopropyl-2,2,5,11,14-pentamethyl-6-(1-methyl-1-phenylethyl)-4,7,10-trioxo-9-(2-phenylethyl)-3-oxa-5,8,11-triazapentadec-13-en-15-oic acid (129 mg, 100%) as a white amorphous solid. This material (106 mg, 0.17 mmol) is then treated with hydrogen chloride (1 mL, 4 mmol; 4 M para-dioxane solution, Aldrich) at room temperature for 1 hour, to provide (E,4S)-2,5-dimethyl4-[methyl((2S)-2-{[(2S)-3-methyl-2-(methylamino)-3-phenylbutanoyl]amino}4-phenylbutanoyl)amino]-2-hexenoic acid hydrochloride (103 mg, 100%) as a white solid. MS (ES): m/z 522.3 (M+H). Analytical HPLC: (4.6×150 mm Luna C18 column eluted with 10 to 90% acetonitrile in water containing 0.02% TFA over 30 minutes): 29.22% (at 8.9 minutes) of E,4S)-2,5-dimethyl4-[methyl((2S)-2-{[(2S)-3-methyl-2-(methylamino)-3-phenylbutanoyl]amino}4-phenylbutanoyl)amino]-2-hexenoic acid, and 2.21% (at 9.4 minutes), 4.33% (at 9.5 minutes), 20.57% (at 9.8 minutes), 0.17% (at 10 minutes), 0.53% (at 10.2 minutes), and 2.76% (at 10.4 minutes) of six other diastereomers.

### EXAMPLE 90

### N,β,β-trimethyl-L-phenylalanyl-4-benzoyl-N-[(1S,2E)-3-carboxy-1-isopropyl-2-butenyl]- N,β,β-trimethyl-L-phenylalaninamide

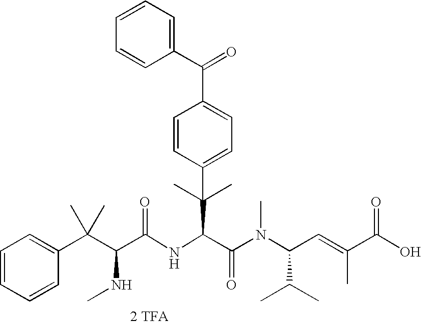

According to General Procedure IV, to a solution of 4-benzoyl-N-(tert-butoxycarbonyl)-β,β-dimethyl-L-phenylalanine (0.13 g, 0.33 mmol, from Reference Example 161) and benzotriazole-1-yl-oxy-tris-pyrrolidinophosphonium hexafluorophosphate (0.26 g, 0.50 mmol) and dimethylaminopyridine (DMAP, 24 mg, 0.20 mmol) in dichloromethane (4 mL, Aldrich) is added diisopropylethylamine (0.17 mL, 0.99 mmol) under a nitrogen atmosphere. To this mixture is added a solution of ethyl (E,4S)-2,5-dimethyl-4-(methylamino)-2-hexenoate (0.38 g, 1.1 mmol) in anhydrous dichloromethane (3 mL). The resulting reaction mixture is stirred at room temperature for 18 hours. Volatiles are evaporated under reduced pressure. The crude product (0.70 g) is purified by semi-preparative HPLC (employing a gradient elution of 5% acetonitrile/95% water to 100% acetonitrile over 1 hour). A hard, white foam (0.16 g, 84%) is obtained after collection and concentration. MS (ES⁺): m/z (M+Na)=601.3 This hard white foam (0.16 g, 0.28 mmol) in anhydrous dichloromethane (3 mL) is cooled to 0° C. in an ice-water bath and hydrochloric acid (4N solution in dioxane, 1 mL, 4 mmol) is added. Stirring at 0° C. is continued for 5 minutes and then the cooling bath is removed. After 1 hour, TLC revealed a preponderance of starting material. An additional 2 mL of 4N hydrochloric acid is added. After 2 hours, the reaction mixture is still composed of mostly starting material. The reaction mixture is left in a -10° C. freezer for 72 hours, following which the TLC showed no change. An additional 2 mL of 4N hydrochloric acid is added and the reaction mixture is allowed to stir for 8 hours at room temperature. LC/MS analysis showed the conversion to product to be nearly complete, so stirring is continued overnight. The ratio is unchanged. Solvent and excess acid are removed under reduced pressure. The resulting light beige foam is triturated with ether, but a gum resulted. Under vacuum, the gum afforded (0.17 g, >100%) of a light beige foam, which is carried on to the next step without further purification. According to General Procedure IV (method lVb), to a solution of N,β,β-trimethyl-L-phenylalanine (0.17 g, 0.56 mmol) and the hydrochloride from the previous step (0.28 mmol max) in anhydrous dimethylformamide (4 mL) under a nitrogen atmosphere is added hydroxybenzotriazole (0.076 g, 0.56 mmol, 1-(3-dimethylaminopropyl)-3-ethylcarbodiimine hydrochloride (0.11 g, 0.56 mmol) and N-methylmorpholine (0.62 μL, 0.56 mmol). After 24 hours the mixture is diluted with water, and the aqueous layer is extracted with diethyl ether (3 times). The combined organic extracts are washed with saturated aqueous sodium hydrogen carbonate and saturated aqueous sodium chloride, dried over sodium sulfate, filtered and concentrated in vacuo. The crude residue is isolated as cloudy beige semi-solid (0.36 g). MS (ES⁺): m/z (M+Na)=790.5

A solution of this material (0.36 g, 0.28 mmol maximum) in dichloromethane (5 mL) is cooled to 0 IC in an ice-water bath. Trifluoroacetic acid (1.5 mL) is added and the mixture is stirred for 10 minutes at 0 IC and then the cooling bath is removed. After an additional 2 hours of stirring, thin-layer chromatography (TLC) showed complete deprotection. Solvent and excess acid are removed under reduced pressure to yield 0.52 g of a reddish oil. The oil is taken up in tetrahydrofuran (2 mL), methanol (2 mL), and water (1 mL). Lithium hydroxide monohydrate (35 mg, 0.84 mmol) is added and the reaction mixture is stirred at 45 IC for 2 hours. LC/MS analysis showed only slight hydrolysis of the ester. An additional 20 mg of lithium hydroxide monohydrate is added and the reaction mixture is heated at 55° C. overnight. The solvent is evaporated under reduced pressure. The residue is purified by semi-preparative reverse-phase HPLC (employing a gradient elution of 5% acetonitrile/95% water/0.1 % trifluoroacetic acid to 100% acetonitrile over 1 hour) to give N,β,β-trimethyl-L-phenylalanyl-4-benzoyl-N-[(1S,2E)-3-carboxy-1-isopropyl-2-butenyl]- N,β,β-trimethyl-L-phenylalaninamide trifluoroacetic acid as a hard, white foam (0.12 g, 50% over 4 steps). TOF MS (ES⁺): m/z (M+H)=640.4

### EXAMPLE 91

### 4-benzoyl-N,β,β-trimethyl-L-phenylalanyl-N¹-[(1S,2E)-3-carboxy-1-isopropylbut-2-enyl]-N¹,3-dimethyl-L-valinamide

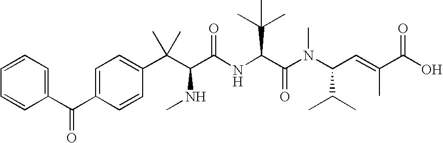

According to general method IV (method IVb), to a solution of N-(tert-butoxycarbonyl)-N,β,β-trimethyl-4-(2-phenyl-1,3-dioxolan-2-yl)-L-phenylalanine (0.23 g, 0.50 mmol, from Reference Example 163) and ethyl (2E,4S)-2,5-dimethyl-4-[methyl(3-methyl-L-valyl)amino]hex-2-enoate (0.24 g, 0.76 mmol) in anhydrous dimethylformamide (3 mL) under a nitrogen atmosphere is added hydroxybenzotriazole (0.14 g, 1.0 mmol, 1-(3-dimethylaminopropyl)-3-ethylcarbodiimine hydrochloride (0.19 g, 1.0 mmol), and N-methylmorpholine (0.11 mL, 1.0 mmol). After 24 hours the mixture is diluted with water, and the aqueous layer is extracted with diethyl ether (3 times). The combined extracts are washed with 2% hydrochloric acid and saturated aqueous sodium chloride, dried over sodium sulfate, filtered and concentrated in vacuo. The residue is isolated as a hard white foam (0.34 g, 92%). MS (ES⁺): m/z (M-Boc+H)=650.4. To a 0° C. solution of this material in tetrahydrofuran (10 mL), is added 10% hydrochloric acid (2.5 mL). The mixture is stirred for 22 hours at room temperature then heated for 28 hours at 45-50° C. After an additional 48 hours at room temperature, the reaction mixture is carefully quenched by the addition of saturated aqueous sodium hydrogen carbonate and then extracted thrice with diethyl ether. The combined organic extracts are washed with saturated aqueous sodium hydrogen carbonate and saturated aqueous sodium chloride, dried over sodium sulfate, decanted, and concentrated under reduced pressure to afford a hard white foam (0.29 g, 91%). MS (ES⁺): m/z (M-Boc+H)=606.4

A solution of this material (0.29 g, 0.42 mmol) in dichloromethane (4 mL) is cooled to 0 IC in an ice-water bath. Trifluoroacetic acid (0.32 mL, 4.2 mmol) is added and the mixture is stirred for 30 minutes at 0° C. and then the cooling bath is removed. After an additional 2 hours of stirring, an additional quantity of trifluoroacetic acid (0.20 mL) is added. After 18 hours of stirring, solvent and excess acid are removed under reduced pressure to yield 0.43 g of a hard brown foam. This material is taken up in tetrahydrofuran (2 mL), methanol (2 mL) and water (1 mL). Lithium hydroxide monohydrate (0.16 mg, 3.8 mmol) is added and the reaction mixture is stirred overnight at room temperature. The solvent is evaporated under reduced pressure. The residue is purified by semi-preparative reverse-phase HPLC (employing a gradient elution of 5% acetonitrile/95% water/0.1% trifluoroacetic acid to 100% acetonitrile over 1 hour) to give a hard, white foam (0.20 g). Subsequent isocratic reverse phase HPLC purifications (employing 60% methanol/40% water (0.02%) trifluoroacetic acid), furnished 4-benzoyl-N,β,β-trimethyl-L-phenylalanyl-N¹-[(1S,2E)-3-carboxy-1-isopropylbut-2-enyl]-N¹,3-dimethyl-L-valinamide trifluoroacetic acid as a white powder (60 mg, 19%). TOF MS (ES⁺): m/z (M+H)=578.4

### EXAMPLE 92

### N,β,β-trimethyl-L-phenylalanyl-N¹-[(1S,2E)-3-carboxy-1-isobutylbut-2-enyl]-N¹-methyl-L-valinamide

Step 1

(2S)-[1-(Methoxy-methyl-carbamoyl)-3-methyl-butyl]-carbamic acid tert-butyl Ester

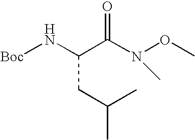

According to the procedure described in the literature (Andersen, R, WO 96/33211) a solution of N-tert-Butoxycarbonyl-L-leucine (30 mmol), O,N-dimethyl hydroxylamine hydrochloride (39.5 mmol), benzotriazole-1-yl-oxy-tris-pyrrolidino-phosphonium hexafluorophosphate (31.5 mmol) in dichloromethane is cooled to 0° C. and diethylisopropylamine (90 mmol) is added. The reaction is stirred at room temperature for 2 hours then concentrated. The residue is diluted with ether and washed with 3 N hydrochloric acid and saturated aqueous sodium bicarbonate, dried over anhydrous magnesium sulfate and concentrated.

Step 2

(2S)-(1—Formyl-3-methyl-butyl)-carbamic Acid Tert-butyl Ester

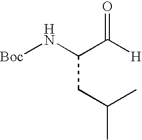

1-(Methoxy-methyl-carbamoyl)-3-methyl-butyl]-carbamic acid tert-butyl ester (15 mmol, from step 1) in tetrahydrofuran is cooled to 0° C. and lithium aluminum hydride (1 M in tetrahydrofuran, 45 mmol) is added. The reaction is stirred at 0° C. for 1 hour. Ethyl acetate (100 mL) and propionic acid (15 mmol, solution in ether) are added dropwise to quench excess lithium aluminum hydride. A 3% solution of hydrochloric acid is then added. The layers are separated and the aqueous layer extracted with ether. The organic layers are washed with saturated aqueous sodium carbonate, then brine, then dried over MgSO4. Concentration in vacuo gave the crude aldehyde, which is used immediately.

Step 3

(2E,4S)-4-tert-Butoxycarbonylamino-2,6-dimethyl-hept-2-enoic Acid Ethyl Ester

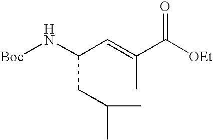

(2E,2S)-(1 —Formyl-3-methyl-butyl)-carbamic acid tert-butyl ester (from step 2) is converted to the title compound by reaction with (carboethoxyethylidene)triphenyl phosphorane as described in Reference Example 114. The product is purified by chromatography over silica gel with hexane: ether (3:1).

Step 4

(2E,4S)4-(tert-Butoxycarbonyl-methylamino)-2,6-dimethyl-hept-2-enoic Acid Ethyl Ester

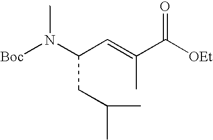

(2E,4S)-4-tert-Butoxycarbonylamino-2,6-dimethyl-hept-2-enoic acid ethyl ester (1.2 mmol, from step 3) is dissolved in tetrahydrofuran and cooled to 0° C. Sodium hydride (6 mmol, 60% dispersion in oil) is added followed after 30 minutes by iodomethane (12 mmol). The reaction is shaken at room temperature for 16 hours then saturated aqueous sodium bicarbonate is added. The reaction is diluted with ethyl acetate and the layers separated. The organic layer is washed with brine, dried over magnesium sulfate and concentrated in vacuo.

Step 5

(2E,4S)4-Methylamino-2,6-dimethyl-hept-2-enoic Acid Ethyl Ester

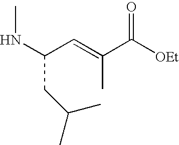

(4S)4-(tert-Butoxycarbonyl-methyl-amino)-2,6-dimethyl-hept-2-enoic acid ethyl ester is dissolved in 4 N hydrochloric acid in dioxane and stirred at room temperature for 2 hours, then concentrated in vacuo. The residue is dissolved in dichloromethane and concentrated in vacuo.

Step 6

4-[(2S)-(2-tert-Butoxycarbonylamino-3,3-dimethyl-butyryl)- (2E,4S)-methylamino]-2,6-dimethyl-hept-2-enoic Acid Ethyl Ester

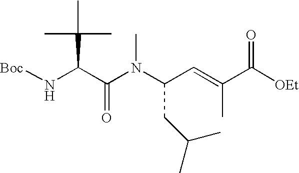

Using General Procedure IV, Method IVa, (2E,4S)-4-methylamino-2,6-dimethyl-hept-2-enoic acid ethyl ester hydrochloride salt (0.4 mmol, from step 5) is treated with N-tert-butoxycarbonyl-tert-butylglycine (0.4 mmol), bromo-tris-pyrrolidino-phosphonium hexafluorophoxphate, diethylisopropyl amine (1.2 mmol) and dimethylaminopyridine (0.2 mmol) in dichloromethane. The reaction is shaken at room temperature for 16 hours, then washed with saturated aqueous sodium bicarbonate and concentrated in vacuo.

Step 7

4-[(2S)- (2-Amino-3,3-dimethyl-butyryl)- (2E,4S)-methylamino]-2,6-dimethyl-hept-2-enoic Acid Ethyl Ester

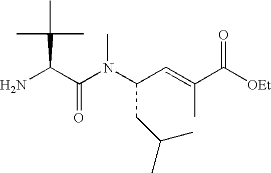

4-[(2S)- (2-tert-Butoxycarbonylamino-3,3-dimethyl-butyryl)- (2E,4S)-methylamino]-2,6-dimethyl-hept-2-enoic acid ethyl ester (0.4 mmol, from step 6) is dissolved in 4 N hydrogen chloride/dioxane solution (1.2 mL) and shaken at room temperature for 1 hour, concentrated in vacuo, suspended in dichloromethane and reconcentated in vacuo.

Step 8

(2S)-4-({2-[2-(tert-Butoxycarbonyl-methyl-amino)-3-methyl-3-phenyl-butyrylamino]-(2S)-3,3-dimethyl-butyryl}-methyl-amino)-(2E,4S)-2,6-dimethyl-hex-2-enoic Acid Ethyl Ester

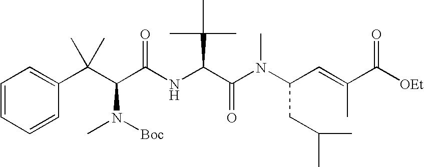

Using the General Procedure IV, Method IVb, 4-[(2S)- (2-amino-3,3-dimethyl-butyryl)-(2E,4S)-methylamino]-2,6-dimethyl-hept-2-enoic acid ethyl ester hydrochloride salt (0.4 mmol, from step 7), N-(tert-butoxycarbonyl)-N,β,β-trimethyl-L-phenylalanine (0.4 mmol), N-hydroxybenzotriazole (1.2 mmol) and 1-ethyl-3(3′-dimethylaminopropyl)carbodiimine hydrochloride (1.2 mmol) in dichloromethane are shaken at room temperature for 16 hours, then washed with saturated sodium bicarbonate and concentrated in vacuo.

Step 9

(2S)-4-({2-[2-methylamino-3-methyl-3-phenyl-butyrylamino]-(2S)-3,3-dimethyl-butyryl}-methyl-amino)-(2E,4S)-2,6-dimethyl-hex-2-enoic Acid Ethyl Ester

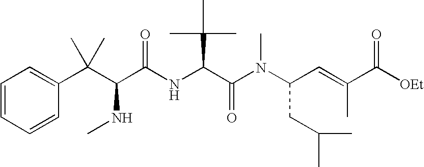

(2S)-4-({2-[2-(tert-Butoxycarbonyl-methyl-amino)-3-methyl-3-phenyl-butyrylamino]-(2S)-3,3-dimethyl-butyryl}-ethyl-amino)-(2E,4S)-2,6-dimethyl-hex-2-enoic acid ethyl ester (from step 8) is dissolved in a mixture of trifluoroacetic acid and dichloromethane (1:1) and shaken at room temperature for 1 hour, then concentrated. The residue is redissolved in dichloromethane and evaporated again in vacuo.

Step 10

4-{[3,3-Dimethyl-2-(3-methyl-2-methylamino-3-phenyl-butyrylamino)-butyryl]-methyl-amino}-2,6-dimethyl-hept-2-enoic Acid

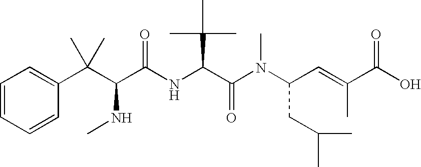

(2S)-4-({2-[2-methylamino-3-methyl-3-phenyl-butyrylamino]-(2S)-3,3-dimethyl-butyryl}-methyl-amino)-(2E,4S)-2,6-dimethyl-hex-2-enoic acid ethyl ester (from step 9) is dissolved in methanol: water (1:1) and lithium hydroxide (5 eq.) is added. The reaction is shaken at room temperature overnight then concentrated in vacuo. The residue is dissolved in a mixture of dimethylsulfoxide/methanol/water and purified by preparative chromatography¹, to give the trifluoroacetic acid salt of the title compound (6.9 mg).

Examples 93-106 are prepared following the procedure described for Example 92 (steps 1-10), and using: either Boc-S-Leu or Boc-S-Val in step 1;

either iodomethane or iodoethane in step 4, or skipping step 4 in the case where R₈ is H;

one of Boc-S-tert-butylglycine, Boc-S- Val, Boc-S-Leu, Boc-S-Nva, Boc-R-Val, Boc-S-Chg, Boc-S-Abu, Boc-S-Ala, Boc-S-Phe, and Boc-S-Phe in step 6;

and either (S)-2-(Boc-methylamino)-3-methyl-3-phenyl-butyric acid or beta-methyl-DL-Boc-phenylalanine in step 8.

### EXAMPLE 93

### N,β,β-trimethyl-L-phenylalanyl-N¹-[(1S,2E)-3-carboxy-1-isobutylbut-2-enyl]-3-methyl-L-valinamide Trifluoroacetic Acid

### EXAMPLE 94

### N,β,β-trimethyl-L-phenylalanyl-N¹-[(1S,2E)-3-carboxy-1-isopropylbut-2-enyl]-N¹-ethyl-3-methyl-L-valinamide Trifluoroacetic Acid

### EXAMPLE 95

### N,β,β-trimethyl-L-phenylananyl-N¹-[(1S,2E)-3-carboxy-1-isopropylbut-2-enyl]-N¹-ethyl-L-valinamide Trifluoroacetic Acid

### EXAMPLE 96

### N,β,β-trimethyl-L-phenylalanyl-N¹-[(1S,2E)-3-carboxy-1-isopropylbut-2-enyl]-N¹-methyl-L-leucinamide Trifluoroacetic Acid

### EXAMPLE 97

### N,β,β-trimethyl-L-phenylalanyl-N¹-[(1S,2E)-3-carboxy-1-isopropylbut-2-enyl]-N¹-methyl-L-norvalinamide Trifluoroacetic Acid

### EXAMPLE 98

### (2E,4S)4-[{(2R)-2-cyclohexyl-2-[(N,β,1-trimethyl-L-phenylalanyl)amino]ethanoyl}(methyl)amino]-2,5-dimethylhex-2-enoic Acid Trifluoroacetic Acid

### EXAMPLE 99

### (2E,4S)-2,5-dimethyl4-(methyl{(2S)-2-[(N,β,1-trimethyl-L-phenylalanyl)amino]butanoyl}amino)hex-2-enoic Acid Trifluoroacetic Acid

### EXAMPLE 100

### 4-{[3,3-Dimethyl-2-(2-methylamino-3-phenyl-butyrylamino)-butyryl]-methyl-amino}-2,5-dimethyl-hex-2-enoic Acid Trifluoroacetic Acid

### EXAMPLE 101

### N,β,β-trimethyl-L-phenylalanyl-N¹-[(1S,2E)-3-carboxy-1-isopropylbut-2-enyl]-3-methyl-L-valinamide Trifluoroacetic Acid

### EXAMPLE 102

### N,β,β-trimethyl-L-phenylalanyl-N¹-[(1S,2E)-3-carboxy-1-isopropylbut-2-enyl]-L-valinamide Trifluoroacetic Acid

### EXAMPLE 103

### 2,5-dimethyl-4-{methyl-[2-(3-methyl-2-methylamino-3-phenyl-butyrylamino)-propionyl]-amino}-hex-2-enoic Acid Trifluoroacetic Acid

### EXAMPLE 104

### 4-{[3,3-Dimethyl-2-(3-methyl-2-methylamino-3-phenyl-butyrylamino)-butyryl]-methyl-amino}-2,6-dimethyl-hept-2-enoic Acid Trifluoroacetic Acid

### EXAMPLE 105

### N,β,β-trimethyl-L-phenylalanyl-N¹-[(1S,2E)-3-carboxy-1-isopropyl-2-butenyl]-N¹-methyl-L-valinamide Trifluoroacetic Acid

### EXAMPLE 106

### N,β,β-trimethyl-L-phenylalanyl-N¹-[(1S,2E)-3-carboxy-1-isopropyl-2-butenyl]-N¹-methyl-L-isoleucinamide Trifluoroacetic Acid

| | HPLC² | |
|---|---|---|
| Ex. # | ret. time | MS³ |
| 92 | 2.73 | 474.4(M + H) |
| 93 | 2.72 | 474.3(M + H) |
| 94 | 2.78 | 488.4(M + H) |
| 95 | 2.68 | 474.4(M + H) |
| 96 | 2.78 | 474.6(M + H) |
| 97 | 2.65 | 460.6(M + H) |
| 98 | 2.85 | 500.7(M + H) |
| 99 | 1.64⁴ | 446.3(M + H) |
| 100 | 1.72⁴ | 460.3(M + H) |
| 101 | 2.66 | 460.3(M + H) |
| 102 | 2.50 | 446.3(M + H) |
| 103 | 2.54 | 432.5(M + H) |
| 104 | 2.80 | 488.4(M + H) |
| 105 | 2.68 | 460.6(M + H) |
| 106 | 2.77 | 474.6(M + H) |

¹Gilson Preparative HPLC conditions: Gilson Preparative HPLC system; YMC Pro C18, 20 mm × 50 mm ID, 5 uM column; 2 mL injection; Solvent A: 0.02% TFA/water; Solvent B: 0.02% TFA/acetonitrile; Gradient: Time 0: 95% A; 2 min: 95% A; 14 min: 10% A, 15 min: 10% A, 16 min: 95% A; Flow rate 22.5 mL/min; Detection: 254 nm DAD.
²HPLC conditions: Hewlett Packard 1100 HPLC system; Waters Xterra C18, 2 mm × 30 mm ID, 3 uM column; 5 uL injection; Solvent A: 0.02% TFA/water; Solvent B:0.02% TFA/acetonitrile; Gradient: Time 0: 95% A; 2 min: 95% A; 3 min: 5% A; Flow rate 1.2 mL/min; Detection: 254 nm DAD.
³Mass Spectrometer: Hewlett Packard MSD; Mass range 105-1000, Fragmentor 140 mV.
⁴HPLC conditions: Waters 2790 HPLC system; Waters Xterra C18, 2 mm × 30 mm ID, 3 uM column; 5 uL injection; Solvent A: 0.02% TFA/water; Solvent B: 0.02% TFA/acetonitrile; Gradient: Time 0: 90% A; 0.3 min 90% A; 3.9 min: 10% A; 5.0 min: 10% A; Flow rate 1.0 mL/min; Detection: 254 nm DAD.

### EXAMPLE 107

### (E,4S)-4-[((2S)-3,3-dimethyl-2-{[(2S)-3-methyl-2-(methylamino)-3-phenylbutanoyI]amino}butanoyl)(methyl)amino]-2,5-dimethyl-2-hexenamide

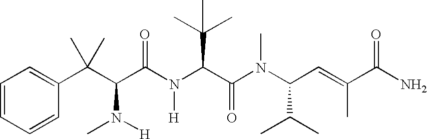

As described in General Procedure Via, N,β,β-trimethyl-L-phenylalanyl-N¹-[(1S,2E)-3-carboxy-1-isopropylbut-2-enyl]-N¹,3-dimethyl-L-valinamide (50 mg, 0.106 mmol) in acetonitrile (5 mL) is treated with 1-hydroxybenzotriazole hydrate (17 mg, 0.127 mmol) and 1-(3-dimethylaminopropyl)-3-ethylcarbodiimide hydrochloride (27 mg, 1.41 mmol). After 2 hours 2.0 M ammonia in methanol is added (0.2 mL, 0.42 mmol). Purification by preparative reverse phase HPLC gave (E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenamide trifluoroacetic acid as a white powder (13 mg). MS (ES): m/z 473.4 (M+H).

### EXAMPLE 108

### (E,4S)-4-[((2S)-3,3-dimethyl-2-{[(2S)-3-methyl-2-(methylamino)-3-phenylbutanoyl]amino}butanoyl)(methyl)amino]-N,2,5-trimethyl-2-hexenamide

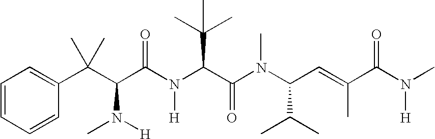

As described in General Procedure Via N,β,β-trimethyl-L-phenylalanyl-N¹-[(1S,2E)-3-carboxy-1-isopropylbut-2-enyl]-N¹,3-dimethyl-L-valinamide (30 mg, 0.064 mmol) in acetonitrile (5 mL) is treated with 1-hydroxybenzotriazole hydrate (10.2 mg, 0.076 mmol) and 1-(3-dimethylaminopropyl)-3-ethylcarbodiimide hydrochloride (16.2 mg, 0.85 mmol). After 2 hours methylamine (0.021 mL, 0.25 mmol) is added. Purification by preparative reverse phase HPLC gave (E,4S)-4-[((2S)-3,3-dimethyl-2-{[(2S)-3-methyl-2-(methylamino)-3-phenylbutanoyl]amino}butanoyl)(methyl)amino]-N,2,5-trimethyl-2-hexenamide trifluoroacetic acid as a white powder (20 mg). MS (ES): m/z 487.6 (M+H).

### EXAMPLE 109

### N,β,β-trimethyl-L-phenylalanyl-N¹-{(1S,2E)-4-[(2-cyanoethyl)amino]-1-isopropyl-3-methyl-4-oxo-2-butenyl}-N¹,3-dimethyl-L-valinamide

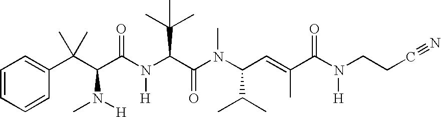

As described in General Procedure Via, N,β,β-trimethyl-L-phenylalanyl-N¹-[(1S,2E)-3-carboxy-1-isopropylbut-2-enyl]-N¹,3-dimethyl-L-valinamide (250 mg, 0.528 mmol) in acetonitrile (25 mL) is treated with 1-hydroxybenzotriazole hydrate (85 mg) and 1-(3-dimethylaminopropyl)-3-ethylcarbodiimide hydrochloride (135 mg). After 18 hours 3-aminopropionitrile fumarate (270 mg) and 1 N aqueous sodium hydroxide (2.0 mL) are added. Purification by preparative reverse phase HPLC gave N,β,β-trimethyl-L-phenylalanyl-N¹-{(1S,2E)-4-[(2-cyanoethyl)amino]-1-isopropyl-3-methyl-4-oxo-2-butenyl}-N¹,3-dimethyl-L-valinamide trifluoroacetic acid as a white powder (278 mg). MS (ES): m/z 526.1 (M+H).

### EXAMPLE 110

### N,β,β-trimethyl-L-phenylalanyl-N¹-{(1S,2E)-4-[(carboxymethyl)amino]-1-isopropyl-3-methyl-4-oxo-2-butenyl}-N¹,3-dimethyl-L-valinamide

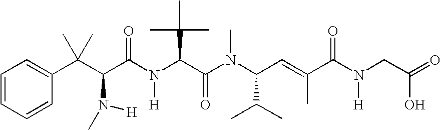

As described in General Procedure Via N,β,β-trimethyl-L-phenylalanyl-N¹-[(1S,2E)-3-carboxy-1-isopropylbut-2-enyl]-N¹,3-dimethyl-L-valinamide (50 mg, 0.106 mmol) in acetonitrile (5 mL) is treated with 1-hydroxybenzotriazole hydrate (17 mg, 0.127 mmol) and 1-(3-dimethylaminopropyl)-3-ethylcarbodiimide hydrochloride (27 mg, 1.41 mmol). After 18 hours glycine (32 mg, 0.83 mmol) in 1N aqueous sodium hydroxide (0.83 mL) is added. Purification by preparative reverse phase HPLC gave N,β,β-trimethyl-L-phenylalanyl-N¹-{(1S,2E)-4-[(carboxymethyl)amino]-1-isopropyl-3-methyl-4-oxo-2-butenyl}-N¹,3-dimethyl-L-valinamide trifluoroacetic acid as a white powder (37 mg). MS (ES): m/z 531.6 (M+H).

### EXAMPLE 111

### N,β,β-trimethyl-L-phenylalanyl-N¹-{(1S,2E)-4-[(4-azidophenyl)amino]-1-isopropyl-3-methyl-4-oxo-2-butenyl}-N¹,3-dimethyl-L-valinamide

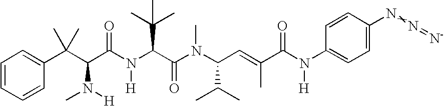

As described in General Procedure Via N,β,β-trimethyl-L-phenylalanyl-N¹-[(1S,2E)-3-carboxy-1-isopropylbut-2-enyl]-N¹,3-dimethyl-L-valinamide (50 mg, 0.106 mmol) in acetonitrile (5 mL) is treated with 1-hydroxybenzotriazole hydrate (17 mg, 0.127 mmol) and 1-(3-dimethylaminopropyl)-3-ethylcarbodiimide hydrochloride (27 mg, 1.41 mmol). After 18 hours 4-Azido-phenylamine hydrochloride (72 mg, 0.424 mmol) and Hunig's base (0.074 mL, 0.424 mmol) is added. After 4 hours 4-dimethylaminopyridine (˜10 mg) is added. Purification by preparative reverse phase HPLC gave N,β,β-trimethyl-L-phenylalanyl-N¹-{(1S,2E)4-[(4-azidophenyl)amino]-1-isopropyl-3-methyl4-oxo-2-butenyl}-N¹,3-dimethyl-L-valinamide trifluoroacetic acid as a tan powder (22 mg). MS (ES): m/z 590.38108 (M+H). (calc'd M+=589.37423).

### EXAMPLE 112

### N,β,β-trimethyl-L-phenylalanyl-N¹-{(1S,2E)-1-isopropyl-3-methyl4-oxo4-[(2-phenylethyl)amino]but-2-enyl}-N¹,3-dimethyl-L-valinamide

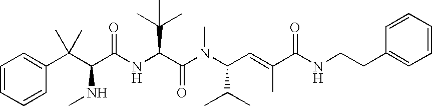

Following General Procedure VIb, N,β,β-trimethyl-L-phenylalanyl-N¹-[(1S,2E)-3-carboxy-1-isopropylbut-2-enyl]-N¹,3-dimethyl-L-valinamide (238 mg, 0.504 mmol) and phenethylamine (73 mg, 0.605 mmol) is coupled to give the title compound as white crystals (62 mg). MS: m/z 575.4 (M−1).

### EXAMPLE 113

### N,β,β-trimethyl-L-phenylalanyl-N¹-{(1S,2E)-4-[[(1S,2E)-4-ethoxy-1-isopropyl-3-methyl-4-oxobut-2-enyl](methyl)amino]-1-isopropyl-3-methyl-4-oxobut-2-enyl}-N¹,3-dimethyl-L-valinamide

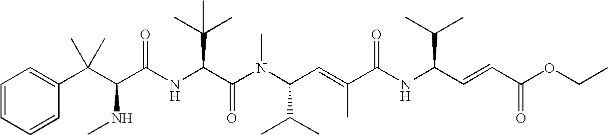

Following General Procedure VIb N,β,β-trimethyl-L-phenylalanyl-N¹-[(1S,2E)-3-carboxy-1-isopropylbut-2-enyl]-N¹,3-dimethyl-L-valinamide (210 mg, 0.443 mmol) and ethyl (2E,4S)-2,5-dimethyl-4-[methyl(3-methyl-L-valyl)amino]hex-2-enoate (106 mg, 0.532 mmol) are coupled to give the title compound as a light yellow solid (329 mg). MS: m/z 655.6 (M+H).

### EXAMPLE 114

### N,β,β-trimethyl-L-phenylalanyl-N¹-{(1S,2E)-4-[[(1S,2E)-3-carboxy-1-isopropylbut-2-enyl](methyl)amino]-1-isopropyl-3-methyl-4-oxobut-2-enyl-N¹,3-dimethyl-L-valinamide

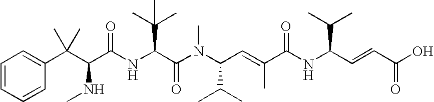

Following General Procedure V, N,β,β-trimethyl-L-phenylalanyl-N¹-{(1S,2E)4-[[(1S,2E)-4-ethoxy-1-isopropyl-3-methyl-4-oxobut-2-enyl](methyl)amino]-1-isopropyl-3-methyl-4-oxobut-2-enyl}-N-1-,3-dimethyl-L-valinamide (133 mg, 0.443 mmol, from Example 113) is hydrolyzed, giving after reverse phase HPLC (0.01% trifluoroacetic acid in water/acetonitrile), the trifluoroacetic acid salt of the title compound as a white solid (87 mg). MS: m/z 625.5 (M−H).

### EXAMPLE 115

### N,β,β-trimethyl-L-phenylalanyl-N¹-[(1S,2E)-1-isopropyl-3-methyl-4-oxo-4-(thien-2-ylmethoxy)but-2-enyl]-N¹,3-dimethyl-L-valinamide

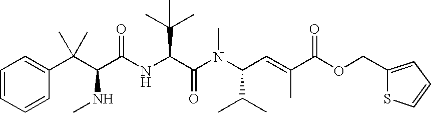

Following General Procedure VII, N,β,β-trimethyl-L-phenylalanyl-N¹-[(1S,2E)-3-carboxy-1-isopropylbut-2-enyl]-N¹,3-dimethyl-L-valinamide (287 mg, 0.607 mmol) and 2-thiophenemethanol (83 mg, 0.728 mmol) are coupled to give the title compound as a light yellow gum (187 mg). MS: m/z 570.3 (M+H).

### EXAMPLE 116

### N,β,β-trimethyl-L-phenylalanyl-N¹-[(1S,2E)-1-isopropyl-3-methyl-4-(octyloxy)-4-oxobut-2-enyl]-N¹,3-dimethyl-L-valinamide

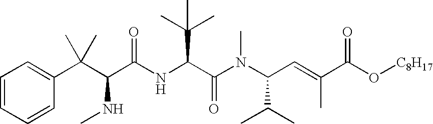

Following General Procedure VII, N,β,β-trimethyl-L-phenylalanyl-N¹-[(1S,2E)-3-carboxy-1-isopropylbut-2-enyl]-N¹,3-dimethyl-L-valinamide (287 mg, 0.607 mmol) and 1-octanol (81 mg, 0.622 mmol) are coupled to give the title compound as a white gum (247 mg). MS: m/z 586.5 (M+H).

### EXAMPLE 117

### N,β,β-trimethyl-L-phenylalanyl-N¹-[(1S,2Z)-3-carboxy-1-isopropyl-2-butenyl]-N¹,3-dimethyl-L-valinamide

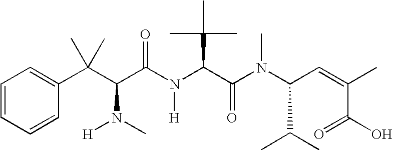

Following the literature procedure (Still, W. C. and Gennari, C. Tetrahedron Letters, 1983, 4405) a solution of 2-[bis-(2,2,2-trifluoro-ethoxy)-phosphoryl]-propionic acid ethyl ester (208 mg, 0.60 mmol) and 18-crown-6 (0.735 g, 2.85 mmol) in tetrahydrofuran (5 mL) at −78° C. is treated with potassium hexamethyldisilazide (1.2 mL, 0.6 mmol, 0.5M in toluene) followed by addition of a solution of N-{1-[(1—Formyl-2-methyl-propyl)-methyl-carbamoyl]-2,2-dimethyl-propyl}-3-methyl-2-methylamino-3-phenyl-butyramide (119 mg, 0.285 mmol, from Reference Example 164) in tetrahydrofuran. After 30 min at −78° C. saturated aqueous ammonium chloride is added. Reverse phase HPLC (0.01% aqueous trifluoroacetic acid/acetonitrile gradient system) gave the desired product (27 mg) which is treated according to General Procedure V, with lithium hydroxide (0.162 mmol) in methanol (0.5 mL), tetrahydrofuran (0.5 mL) and water (0.25 mL) at 25° C. for 18 hours. Reverse phase HPLC (0.01% aqueous trifluoroacetic acid/acetonitrile gradient system) gave the trifluoroacetic acid of the title compound as a white solid (4 mg). MS (ES): m/z 474.33222 (M+H). (calc'd M+=473.32554).

### EXAMPLE 118

### N,β,β-trimethyl-L-phenylalanyl-N¹-[(1S,2E)-3-carboxy-1-isopropylprop-2-enyl]-N¹,3-dimethyl-L-valinamide

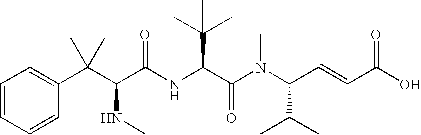

To a solution of N-{1-[(1 —Formyl-2-methyl-propyl)-methyl-carbamoyl]-2,2-dimethyl-propyl}-3-methyl-2-methylamino-3-phenyl-butyramide (340 mg, 0.815 mmol, from Reference Example 164) in dichloromethane (5 mL) is added (triphenyl-λ5-phosphanylidene)-acetic acid methyl ester (273 mg, 0.815 mmol). After 18 hours the reaction mixture is concentrated in vacuo, dissolved in dimethylformamide and purified by reverse phase HPLC (methanol/water) to give a colorless oil (66 mg). According to General Procedure V, this material (60 mg, 0.127 mmol) is treated with lithium hydroxide (0.25 mmol) in methanol (0.5 mL), tetrahydrofuran (0.5 mL) and water (0.25 mL) at 25° C. for 18 hours. Concentration in vacuo to a volume of ˜0.2 mL followed by addition of 0.25 mL of 1 N aqueous citric acid gave a precipitate which is filtered, washed with water and dried in vacuo to give the title compound as a white powder (12 mg). MS (ES): m/z 460.31681 (M+H) (calc'd =459.30988). HPLC and ¹H NMR indicate ˜39% of N,β,β-trimethyl-L-phenylalanyl-N¹-[(1S,2E)-3-carboxy-1-isopropylprop-2-enyl]-N¹,3-dimethyl-D-valinamide is present.

### EXAMPLE 119

### N,β,β-trimethyl-L-phenylalanyl-N¹-[(1S,2E)-1-allyl-3-carboxybut-2-enyl]-N¹,3-dimethyl-L-valinamide

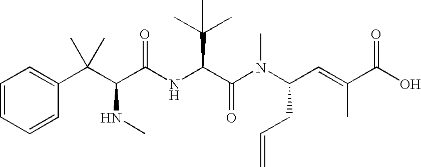

N-(tert-butoxycarbonyl)-N,β,β-trimethyl-L-phenylalanyl-N¹-[(2E)-1-allyl-3-carboxy-2-butenyl]-N¹,3-dimethyl-L-valinamide (55 mg, 0.096 mmol, from Reference Example 173) is dissolved in dichloromethane (0.34 mL, 3.5 mummol) and treated with trifluoroacetic acid (0.074 mL, 0.96 mmol) at room temperature for 30 minutes. The reaction mixture is then concentrated in vacuo and triturated with ether to give a white solid (0.43 g, 95%). The crude material is purified by reverse phase HPLC to give the trifluoroacetic acid salt of the title compound. M.W. 473.67 [M+H] 474.62

### EXAMPLE 120

### (2E,4S)4-[{(2S)-3,3-dimethyl-2-[(N,β,β-trimethyl-L-phenylalanyl)amino]4-pentenoyl}(methyl)amino]-2,5-dimethyl-2-hexenoic Acid

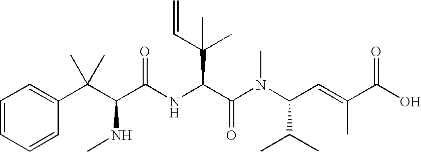

(2E ,4S)4-({2-[2-(tert-butoxycarbonyl-methyl-amino)-3-methyl-3-phenyl-butyrylamino]-(2S)-3,3-dimethyl-pent-4-enoyl}-methyl-amino)-2,5-dimethyl-hex-2-enoic acid (22 mg, 0.04 mmol, from Reference Example 178) is dissolved in dichloromethane (0.5 mL) and treated with 1 N hydrogen chloride/dioxane (0.20 mL) at room temperature for 30 minutes. The reaction mixture is then concentrated in vacuo and purified by reverse phase HPLC to give the trifluoroacetic acid salt of the title compound (18 mg, 95%). M.W. 483.69 [M+H] 484.7

### EXAMPLE 121

### (2E, 4S)-4-[((2S)-2-{[3,3-dimethyl-2-(methylamino)-4-pentenoyl]amino}-3,3-dimethylbutanoyl)(methyl)amino]-2,5-dimethyl-2-hexenoic Acid

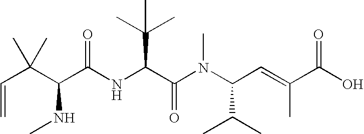

(9S, 1 2S,13E)-9-tert-Butyl-6-(1,1-dimethyl-2-propenyl)-12-isopropyl-2,2,5,11,14-pentamethyl-4,7,10-trioxo-3-oxa-5,8,11-triazapentadec-13-en-15-oic acid (26 mg, 0.05 mmol, from Reference Example 182) is dissolved in dichloromethane (0.5 mL) and treated with 1 N hydrogen chloride/dioxane (0.025 mL, 0.10 mmol) at room temperature for 30 minutes to give the hydrochloric acid salt of the title compound as a white solid (18 mg, 78%). M.W. 421.69 [M+H] 422.62

### EXAMPLE 122

### N,β,β-trimethyl-L-phenylalanyl-N¹-[(1S,2E)-3-carboxy-1-isopropylbut-2-enyl]-N¹,3-dimethyl-L-isoleucinamide

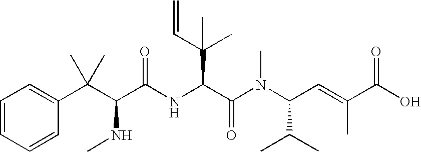

To a solution of N-(tert-butoxycarbonyl)-N,β,β-trimethyl-L-phenylalanyl-N¹-[(1S,2E)-3-carboxy-1-isopropylbut-2-enyl]-N¹,3-dimethylisoleucinamide (50 mg, 0.09 mmol, from Reference Example 187) in dichloromethane is added 4N hydrogen chloride in dioxane (0.044 mL, 0.17 mmol). After 15 hours the solvent is removed. Purification by reverse phase HPLC gave the trifluoroacetic acid salt of the title compound (20 mg). M.W. 428.3 [M+H] 429.3

### EXAMPLES 123 AND 124

### N,β,β-trimethyl-L-phenylalanyl-N¹-[(1R,3S)-3-carboxy-1-isopropylbutyl]-N¹,3-dimethyl-L-valinamide and and Example 124 N,β,β-trimethyl-L-phenylalanyl-N¹-[(1R,3R)-3-carboxy-1-isopropylbutyl]-N¹,3-dimethyl-L-valinamide

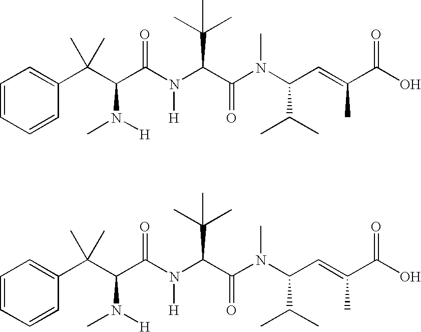

A solution of N,β,β-trimethyl-L-phenylalanyl-N¹-[(1S,2E)-3-carboxy-1-isopropylbut-2-enyl]-N¹,3-dimethyl-L-valinamide (200 mg, Andersen, R. WO 99/32509) in acetic acid (10 mL) is treated with 10% Pd/C under 1 atmosphere of hydrogen for 18 hours. Concentration in vacuo, filtration through diatomaceous earth with 1:1 methanol:water (0.02% trifluoroacetic acid) and purification by reverse phase HPLC (1:1 methanol:water (0.02% trifluoroacetic acid) gave the major isomer (first off of HPLC column) N,β,β-trimethyl-L-phenylalanyl-N¹-[(1R,3S)-3-carboxy-1-isopropylbutyl]-N¹,3-dimethyl-L-valinamide trifluoroacetic acid as a white powder (56 mg). Exact mass +H =476.34796 (calc'd =475.3412). The minor isomer N,β,β-trimethyl-L-phenylalanyl-N¹-[(1R,3R)-3-carboxy-1-isopropylbutyl]-N¹,3-dimethyl-L-valinamide trifluoroacetic acid is obtained as a white powder (7 mg). MS (ES): m/z 476.49 (M+H).

### EXAMPLES 125 and 126

### β,β-diethyl-N-methyl-L-phenylalanyl-N¹-[(1S,2E)-3-carboxy-1-isopropylbut-2-enyl]-N¹,3-dimethyl-L-valinamide and Example 126 β,β-diethyl-N-methyl-D-phenylalanyl-N¹-[(1S,2E)-3-carboxy-1-isopropylbut-2-enyl]-N¹,3-dimethyl-L-valinamide

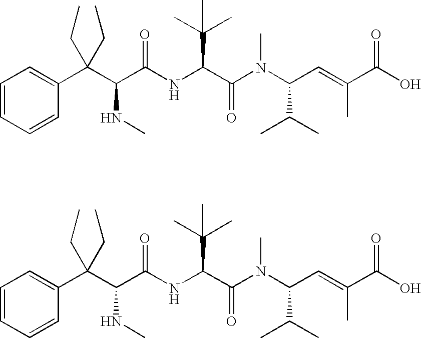

By following the procedures described in General Procedures II, III, IVa, V and substituting iodoethane for iodomethane in General Procedure II, β,β-diethyl-N-methyl-L-phenylalanyl-N¹-[(1S,2E)-3-carboxy-1-isopropylbut-2-enyl]-N¹,3-dimethyl-L-valinamide MS (ES): m/z 502.36402 (M+H) (Calc'd NW=501.35683) and β,β-diethyl-N-methyl-D-phenylalanyl-N¹-[(1S,2E)-3-carboxy-1-isopropylbut-2-enyl]-N¹,3-dimethyl-L-valinamide is obtained MS (ES): m/z 502.36329 (M+H) (Calc'd NW=501.35683).

### EXAMPLE 127

### (BetaS)-N, Beta-dimethyl-L-phenylalanyl-N¹-[(1S,2E)-3-carboxy-1-isopropylbut-2-enyl]-N¹,3-dimethyl-L-valinamide

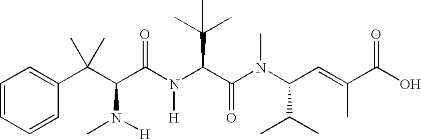

By a procedure analogous to that described in Reference Example 68, methyl (BetaS)-N-(tert-butoxycarbonyl)-N,Beta-dimethyl-L-phenylalaninate (from Reference Example 188) is hydrolysed using aqueous lithium hydroxide. By following General Procedures lVb and V, the carboxylic acid that is obtained is converted to the title compound (white powder). MS (ES): m/z 458.3(M−H)

### EXAMPLE 128

### O-benzyl-N-methyl-L-threonyl-N¹-[(1S,2E)-3-carboxy-1-isopropylbut-2-enyl]-N¹,3-dimethyl-L-valinamide

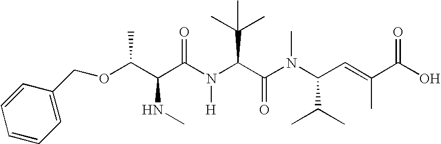

According to General Procedure V, a solution of O-benzyl-N-methyl-L-threonyl-N¹-[(1S,2E)4-ethoxy-1-isopropyl-3-methyl-4-oxobut-2-enyl]-N¹,3-dimethyl-L-valinamide (0.8 g) from Reference Example 190 in methanol (5 mL), tetrahydrofuran (5 mL) and water (2,5 mL) is treated with lithium hydroxide hydrate (92 mg). After the hydrolysis is complete the organic solvents are removed in vacuo and 1 N aqueous citric acid is added dropwise. The resulting precipitate is collected, washed with water and dried in vacuo to give the title compound as a white solid. MS (ES): m/z 490.32768 (M+H) (Calc'd NW=489.32027)

### EXAMPLE 129

### N,β,β-trimethyl-L-phenylalanyl-N¹-[(1S,2E)-3-carboxy-1-isopropylbut-2-enyl]-N¹,3-dimethyl-L-valinamide

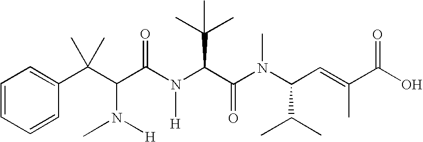

### EXAMPLE 130

### (2E,4S)-4-[((2S)-2-{[(2S)-2-Amino-3-(1-naphthyl)propanoyl]amino}-3,3-dimethylbutanoyl)(methyl)amino]-2,5-dimethyl-2-hexenoic Acid

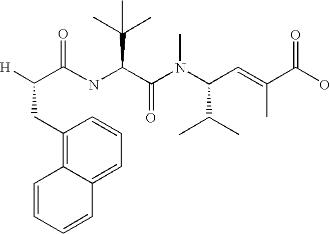

(6S,9S,12S,13E)-9-tert-Butyl-12-isopropyl-2,2,11,14-tetramethyl-6-(1-naphthylmethyl)-4,7,10-trioxo-3-oxa-5,8,11-triazapentadec-13-en-15-oic acid (60 mg, 0.103 mmol, obtained from Reference Example 192) is treated with hydrogen chloride (1 ml, 4 M para-dioxane solution, Aldrich) at room temperature for 1 h, to provide (2E,4S)-4-[((2S)-2-{[(2S)-2-amino-3-(1-naphthyl)propanoyl]amino}-3,3-dimethylbutanoyl)(methyl)amino]-2,5-dimethyl-2-hexenoic acid hydrochloric acid salt (64 mg, 100%) as a white powder. MS (ES): m/z 481.9 (M+H).

Further compounds prepared by procedures described herein or methods described herein and in WO 99/32509, WO 96/33211 and U.S. Pat. No. 6,153,590.

### EXAMPLE 131

### N,β,β-trimethyl-L-phenylalanyl-N¹-[(1S,2E)-3-carboxy-1-isopropylbut-2-enyl]-N¹-methyl-D-valinamide

### EXAMPLE 132

### (E,4S)-4-[((2S)-3,3-dimethyl-2-{[(2S)-3-methyl-2-(methylamino)-3-(1-methyl-1H-ethyl-1H-indol-3-yl)butanoyl]amino}butanoyl)amino]-2,5-dimethyl-2-hexenoic Acid

### EXAMPLE 133

### Ethyl (E,4S)4-[((2S)-3,3-dimethyl-2-{[(2S)-3-methyl-2-(methylamino)-3-phenylbutanoyl]amino}butanoyl)(methyl)amino]-2,5-dimethyl-2-hexenoate

### EXAMPLE 134

### (E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenoic Acid

### EXAMPLE 135

### Ethyl (E,4S)-4-[((2S)-3,3-dimethyl-2-{[(2R)-3-methyl-2-(methylamino)-3-phenylbutanoyl]amino}butanoyl)(methyl)amino]-2,5-dimethyl-2-hexenoate

### EXAMPLE 136

### (E,4S)-4-[((2S)-3,3-dimethyl-2-{[(2S)-3-methyl-2-(methylamino)-3-phenylbutanoyl]amino}butanoyl)(methyl)amino]-2-methyl-5-phenyl-2-pentenoic Acid

### EXAMPLE 137

### (E,4S)-2,5-dimethyl4-[methyl((2S)-2-{[(2S)-3-methyl-2-(methylamino)-3-phenylbutanoyl]amino}-3-phenylpropanoyl)amino]-2-hexenoic Acid

### EXAMPLE 138

### (4R)4-[((2S)-2-{[(2S)-2-amino4-methylpentanoyl]amino}-3,3-dimethylbutanoyl)amino]-2,5-dimethylhexanoic Acid

### EXAMPLE 139

### N,β,β-trimethyl-L-phenylalanyl-N¹-[(1S,2E)-3-carboxy-1-isopropyl-2-butenyl]-N¹-methyl-L-alpha-glutamine

### EXAMPLE 140

### N,3-dimethyl-L-valyl-N¹-[(1S,2E)-3-carboxy-1-isopropyl-2-butenyl]-N¹,3-dimethyl-L-valinamide

### EXAMPLE 141

### N,β,β-trimethyl-L-tryptophyl-N¹-[(1S,2E)-3-carboxy-1-isopropyl-2-butenyl]-N¹,3-dimethyl-L-valinamide

### EXAMPLE 142

### (2E,4S)-2,5-dimethyl-4-(methyl{3-methyl-N-[(2S)-2-(methylamino)-2-(1-phenylcyclopropyl)acetyl]-L-valyl}amino)hex-2-enoic Acid

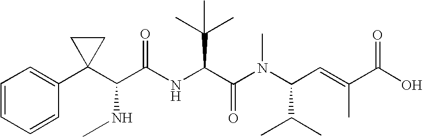

### EXAMPLE 143

### (2E,4S)-2,5-dimethyl-4-(methyl{3-methyl-N-[(2R)-2-(methylamino)-2-(1-phenylcyclopropyl)acetyl]-L-valyl}amino)hex-2-enoic Acid

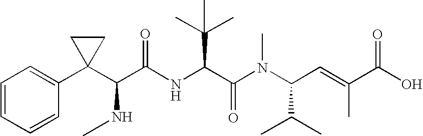

To a solution of (methylamino)(1-phenylcyclopropyl)acetic acid (465 mg, 2.1 mmol, from Reference Example) in dichloromethane (9 mL) and dimethylformamide (9 mL) was added PyBOP (1.09 g, 2.1 mmol), ethyl (2E,4S)-2,5-dimethyl-4-[methyl(3-methyl-L-valyl)amino]hex-2-enoate (660 mg, 2.1 mmole) in dichloromethane (3 mL) and diisopropylethylamine (0.81 mL, 4.5 mmole). After 18 h the reaction mixture was evaporated in vacuo and the residue treated with ethyl acetate and water. The ethyl acetate layer was dried over sodium sulfate, filtered and evaporated in vacuo to give a mixture of esters which was then treated with aqueous 1 M lithium hydroxide solution (10.5 mL, 10.5 mmol), water (6 mL) and methanol (20 mL), to provide after preparative HPLC the title compounds as trifluoroacetic acid salts. Example 174 was isolated as a white solid (129 mg, contains 6.4% of the other isomer by HPLC) MS (ES): m/z 472.3 (M+H). Example 175 was isolated as a white solid (142 mg, contains 4.1% of the other isomer by HPLC) MS (ES): m/z 472.3 (M+H).

### EXAMPLE 144

### 2-(4-{[3,3-Dimethyl-2-(3-methyl-2-methylamino-3-phenyl-butyrylamino)-butyryl]-methyl-amino}-2,5-dimethyl-hex-2-enoylamino)-4-methylsulfanyl-butyric Acid Methyl

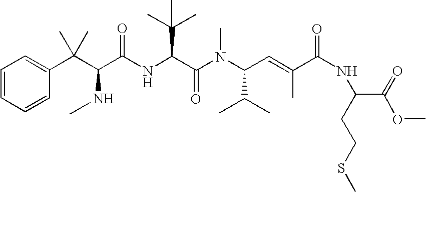

Using General Procedure VI Method B and L-methionine methyl ester hydrochloride (131 mg, 0.654 mmol) the title compound was obtained as a trifluoroacetic acid salt after HPLC (196 mg, light yellow solid). MS: m/z (M+H)=619.3.

### EXAMPLE 145

### N,β,β-trimethyl-L-phenylalanyl-N 1-((1S,2E)4-{[(1S)-1-carboxy-3-(methylthio)propyl]amino}-1-isopropyl-3-methyl-4-oxobut-2-enyl)-N 1,3-dimethyl-L-valinamide

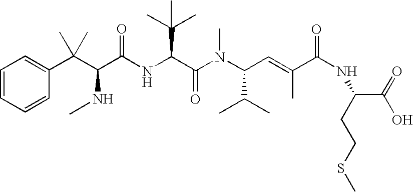

Using General Procedure V, 2-(4-{[3,3-Dimethyl-2-(3-methyl-2-methylamino-3-phenyl-butyrylamino)-butyryl]-methyl-amino}-2,5-dimethyl-hex-2-enoylamino)-4-methylsulfanyl-butyric acid methyl ester (151 mg, 0.243 mmol) was converted to the title compound. The title compound was obtained as a trifluoroacetic acid salt after HPLC (50 mg, white solid). MS: m/z (M−H)=603.1.

### EXAMPLE 146

### N,β,β-trimethyl-4-[(E)-2-phenylvinyl]-L-phenylalanyl-N¹-[(1S,2E)-3-carboxy-1-isopropylbut-2-enyl]-N¹,3-dimethyl-L-valinamide

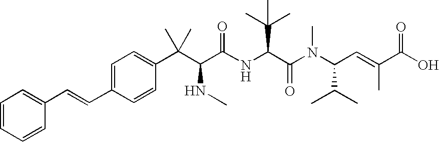

### EXAMPLE 147

### N,β,β-trimethyl4-[(E)-2-phenylvinyl]-D-phenylalanyl-N¹-[(1S,2E)-3-carboxy-1-isopropylbut-2-enyl]-N¹,3-dimethyl-L-valinamide

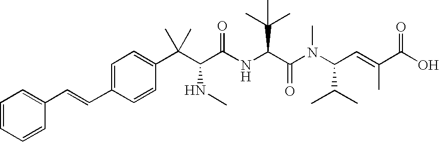

To a solution of 3-bromo-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide and 3-bromo-N,β,β-trimethyl-D-phenylalanyl-N¹-[(1S,2E)-4-ethoxy-1-isopropyl-3-methyl4-oxo-2-butenyl]-N¹,3-dimethyl-L-valinamide (0.62 g, 1.1 mmol, Reference Example 52) in DMF (3 mL) was added styrene (0.17 g, 1.6 mmol), palladium acetate (25 mg, 0.11 mmol) and tri-o-tolylphosphine (67 mg, 0.22 mmol) and triethylamine (0.28 g, 2.8 mmol). The reaction mixture was heated at 100° C. After 17 h styrene (0.17 g, 1.6 mmol), palladium acetate (25 mg, 0.11 mmol), tri-o-tolylphosphine (67 mg, 0.22 mmol) and triethylamine (0.28 g, 2.8 mmol) were added and the reaction mixture heated at 120° C. for 24 h. Aqueous workup and chromatography on silica gel (EtOAc/hexane) gave the ethyl esters of the title compounds. Treatment of each isomer as described in General Procedure V gave N,β,β-trimethyl-4-[(E)-2-phenylvinyl]-L-phenylalanyl-N¹-[(1S,2E)-3-carboxy-1-isopropylbut-2-enyl]-N¹,3-dimethyl-L-valinamide as a white powder after HPLC (190 mg, trifluoroacetic acid salt). MS: m/z =576.4 (M+H) and N,β,β-trimethyl-4-[(E)-2-phenylvinyl]-D-phenylalanyl-N¹-[(1S,2E)-3-carboxy-1-isopropylbut-2-enyl]-N¹,3-dimethyl-L-valinamide as a white powder after HPLC (100 mg, trifluoroacetic acid salt). MS m/z =576.3 (M+H).

### EXAMPLE 148

### N,β,β-trimethyl-L-phenylalanyl-N¹-[(1S,2E)-3-carboxy-1-isopropylbut-2′enyl]-3-fluoro-N¹-methyl-D-valinamide

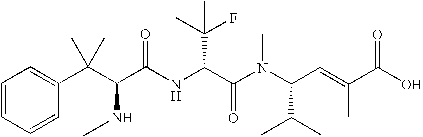

Beginning with commercially available 2-Amino-3-fluoro-3-methyl-butyric acid and using the appropriate procedures described in the above examples the title compound was obtained as the trifluoroacetic acid salt. This compound contained 2% -3% of the SSS isomer. MS: m/z 478.4 (M+H).

### EXAMPLE 149

### N,β,β-trimethyl-L-phenylalanyl-N¹-[(1S,2E)-3-carboxy-1-isopropylbut-2-enyl]-3-fluoro-N¹-methyl-L-valinamide

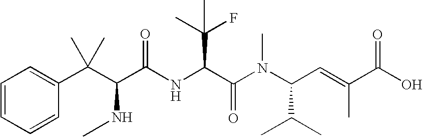

Beginning with commercially available 2-Amino-3-fluoro-3-methyl-butyric acid and using the appropriate procedures described in the above examples the title compound was obtained as the trifluoroacetic acid salt. MS: m/z 478.4 (M+H).

### EXAMPLE 150

### 3-[(4-methoxybenzyl)thio]-N-methyl-L-valyl-N¹-[(1S,2E)-3-carboxy-1-isopropylbut-2-enyl]-N¹,3-dimethyl-L-valinamide

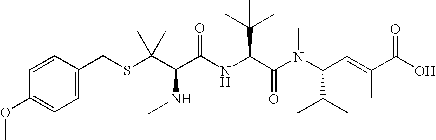

Starting with commercially available BOC-S-(4-methoxybenzyl)-L-penicillamine the title compound was prepared by using the appropriate methods described in the Reference Examples, Examples and the General Methods. The title compound was obtained as a trifluoroacetic acid salt after HPLC. MS: m/z=550.3 (M+H).

### EXAMPLE 151

### N-ethyl-β,β-dimethyl-L-phenylalanyl-N¹-[(1S,2E)-3-carboxy-1-isopropylbut-2-enyl]-N¹,3-dimethyl-L-valinamide

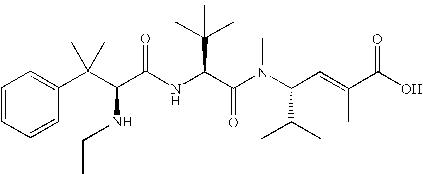

Commercially available (S)-N-BOC-2-amino-3-methyl-3-phenyl-butyric acid was converted to the HCl salt of ethyl (E,4S)-4-[((2S)-2-{[(2S)-2-amino-3-methyl-3-phenylbutanoyl]amino]-3,3-dimethylbutanoyl)(methyl)amino]-2,5-dimethyl-2-hexenoate by the use of General Procedure IV followed by treatment with hydrochloric acid in dioxane to deprotect the amine. Treatment of this compound (143 mg, 0.273 mmol) in DMF (1.5 mL) with iodoethane (0.025 mL, 0.30 mmol) and Hunig's base (100 uL, 0.57 mmol) for 6 h, followed by HPLC gave a solid (32 mg). This material (28 mg) was treated as described in General Procedure V to give the TFA salt of the title compound after HPLC as a white solid (20 mg). MS: m/z 488.3 (M+H).

### EXAMPLE 152

### (2E,4S)-2,5-dimethyl-4-(methyl{3-methyl-N-[(2S)-3-methyl-3-phenyl-2-pyrrolidin-1-ylbutanoyl]-L-valyl}amino)hex-2-enoic Acid

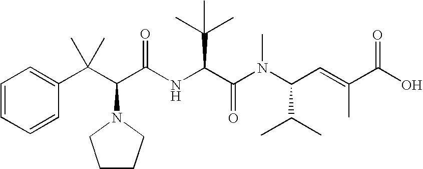

Commercially available (S)-N-BOC-2-amino-3-methyl-3-phenyl-butyric acid was converted to the HCl salt of ethyl (E,4S)-4-[((2S)-2-[(2S)-2-amino-3-methyl-3-phenylbutanoyl]amino}-3,3-dimethylbutanoyl)(methyl)amino]-2,5-dimethyl-2-hexenoate by the use of General Procedure IV followed by treatment with hydrochloric acid in dioxane to deprotect the amine. Treatment of this compound (138 mg, 0.264 mmol) in DMF (2 mL) with 1,4-diiodobutane (0.04 mL, 0.29 mmol) and Hunig's base (115 uL, 0.66 mmol) for 72 h, followed by HPLC gave a solid (67 mg). This material (43 mg) was treated as described in General Procedure V to give the TFA salt of the title compound after HPLC as a white solid (13 mg). MS: m/z 514.5 (M+H).

### EXAMPLE 153

### N-(2-hydroxyethyl)-β,β-dimethyl-L-phenylalanyl-N¹-[(1S,2E)-3-carboxy-1-isopropylbut-2-enyl]-N¹,3-dimethyl-L-valinamide

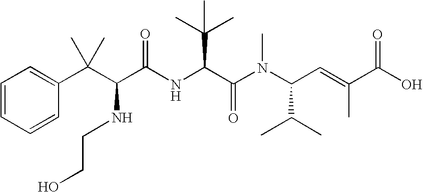

Commercially available (S)-N-BOC-2-amino-3-methyl-3-phenyl-butyric acid was converted to the HCl salt of ethyl (E,4S)-4-[((2S)-2-{[(2S)-2-amino-3-methyl-3-phenylbutanoyl]amino}-3,3-dimethylbutanoyl)(methyl)amino]-2,5-dimethyl-2-hexenoate by the use of General Procedure IV followed by treatment with hydrochloric acid in dioxane to deprotect the amine. Treatment of this compound (112 mg, 0.214 mmol) in DMF (3 mL) with 2-bromoethanol (0.02 mL, 0.26 mmol) and Hunig's base (104 uL, 0.60 mmol) for 18 h, followed by HPLC gave an oil (97 mg). This material (85 mg) was treated as described in General Procedure V to give the TFA salt of the title compound after HPLC as a white solid (42 mg). MS: m/z 504.3 (M+H).

### EXAMPLE 154

### (βR)-N,β-dimethyl-L-phenylalanyl-N¹-[(1S,2E)-3-carboxy-1-isopropylbut-2- enyl]-N¹,3-dimethyl-L-valinamide

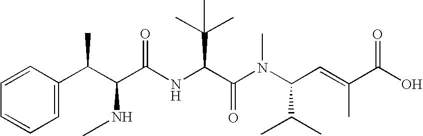

Commercially available (βR)-N-(tert-butoxycarbonyl)-β-methyl-L-phenylalanine was converted to the title compound (trifluoroacetic acid salt) in a manner analogous to that described for the compound of Example 127. MS: m/s (M+H)=460.3.

### EXAMPLE 155

### 3-acetyl-N,β,β-trimethyl-L-phenylalanyl-N¹-[(1S,2E)-3-carboxy-1-isopropylbut-2-enyl]-N¹,3-dimethyl-L-valinamide

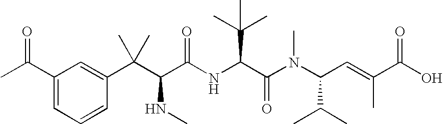

3-Bromo-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide (0.48 g, 0.83 mmol, from Reference Example 52) in toluene (4 mL) was treated with tributyl(1-ethoxyvinyl)tin (0.45 g, 1.2 mmol) and Pd(PPh3)4 (48 mg, 0.04 mmol). The reaction mixture was heated at 120° C. for 36h. Filtration through celite and concentration in vacuo gave a material that was dissolved in THF (5 mL). To this solution was added 2N aqueous HCl. After 18 h the reaction mixture was concentrated in vacuo. The resulting material was treated as described in General Procedure V (100 mg LiOH.H2O in 5 mL of 2:2:1 methanol:THF:water) to give the title compound (180 mg, foam) as a trifluoroacetic acid salt after HPLC. MS: m/z=516.4 (M+H).

### EXAMPLE 156

### N,β,β-trimethyl-L-phenylalanyl-N¹-[(1S,2E)-3-carboxy-1-isopropylbut-2-enyl]-3-hydroxy-N¹-methyl-L-valinamide

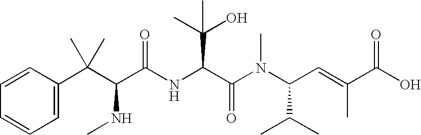

Beginning with commercially available (S)-(+)-2-amino-3-hydroxy-3-methylbutanoic acid and using the appropriate procedures described in the above examples the title compound was prepared as the trifluoroacetic acid salt. MS: m/z 476.4 (M+H).

### EXAMPLE 157

### WAY-188776

### N,β,β-trimethyl-L-phenylalanyl-N¹-[(1R,2E)-3-carboxy-1-isopropylbut-2- enyl]-N¹,3-dimethyl-L-valinamide

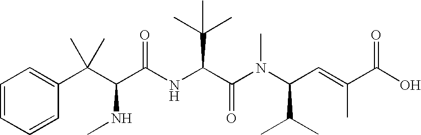

The title compound was prepared in a manner analogous to the compound of Example 129 using the appropriate enantiomerically pure starting materials to obtain the desired stereochemistry in the final product. The title compound was obtained as a trifluoroacetic acid salt after HPLC. MS: m/z=474.4 (M+H).

We claim:

1. A method of treating, inhibiting the growth of, or eradicating a tumor in a mammal in need thereof wherein said tumor is resistant to at least one chemotherapeutic agent which method comprises providing to said mammal an effective amount of a compound of Formula (II): 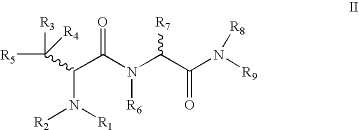
wherein:
R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
or R₁ and R₂ taken together with the nitrogen atom to which they are attached is a three to seven membered ring;
R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
or R₃ and R₄ taken together with the carbon to which they are attached form a three to seven membered ring;
R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀OH, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; aryl-R- and aryl and provided that when R₅ is an indolyl moiety of the formula 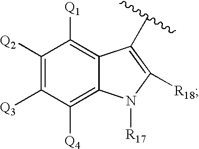
R₁₇ is H or an optionally substituted alkyl or acyl group; and
R₁₈, Q₁, Q₂, Q₃, and Q₄ are independently selected from H, halogen, alkyl, acyl, —OH, —O- alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;
R₆ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₇ is selected from the group consisting of a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₉ is: 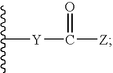
and wherein,
R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁,O —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group;
X is a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;
Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;
Y is a moiety selected from the group consisting of: a linear, saturated or unsaturated, one to six carbon alkyl group, optionally substituted with R, ArylR-, or X; and,
Z is a moiety selected from the group consisting of: —OH, —OR; —SH; —SR; —NH₂; —NHR; —N(R)₂; —NHCH(R₁₁)COOH; and —NRCH(R₁₁)COOH, wherein R₁₁ is a moiety having the formula: R, or —(CH₂)ₙNR₁₂R₁₃, wherein n=1-4 and R₁₂ and R₁₃ are independently selected from the group consisting of: H; R; and —C(NH) (NH₂); with the provisos that:
(1) when R₁ is H and R₂ is CH₃ of the moiety 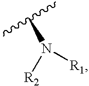
R₃ is CH₃, R₄ is CH₃, R₅ is phenyl, R₆ is H, R₈ is CH₃, and
a) when R₉ is 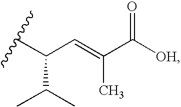
then R₇ is not 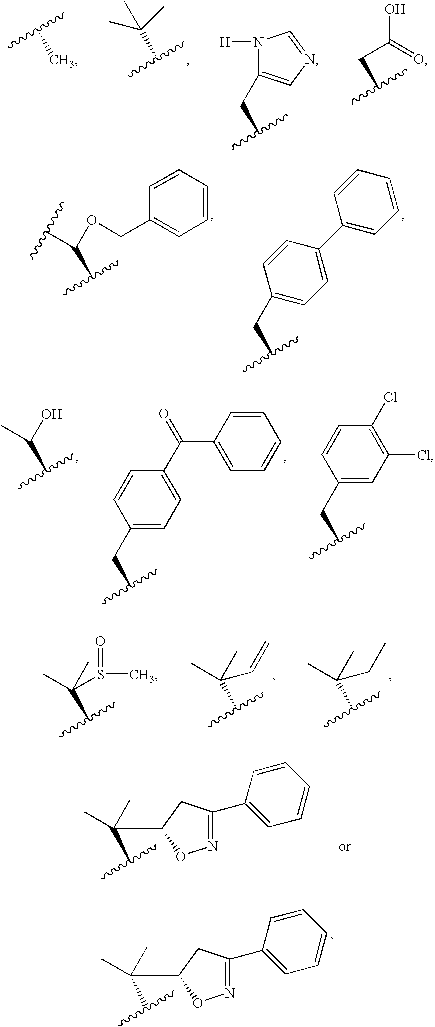
b) when R₉ is 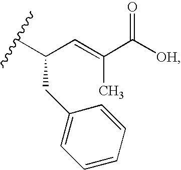
then R₇ is not 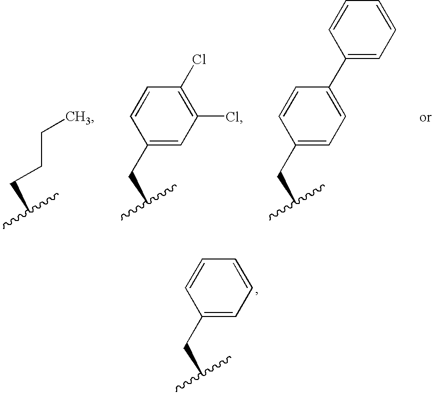
(2) when R₁ is H and R₂ is CH₃, of the moiety 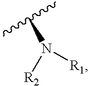
R₃ is CH₃, R₄ is CH₃, R₅ is phenyl, R₆ is H, R₈ is H,
a) R₉ is 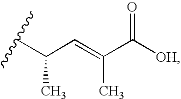
then R₇ is not 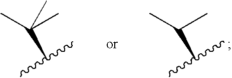
b) when R₉ is 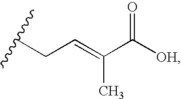
then R₇ is not 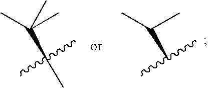
c) when R₉ is 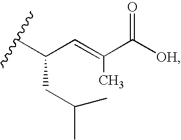
then R₇ is not 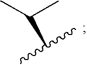
(3) when R₁ is H and R₂ is CH₃, of the moiety 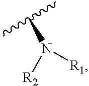
R₃ is CH₃, R₄ is CH₃, R₅ is phenyl, R₆ is H,
R₇ is 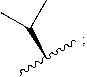
R₈ is 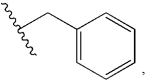
then R₉ is not 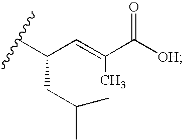
(4) when R₁ is H, R₂ is H, of the moiety 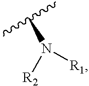
R₃ is CH₃, R₄ is CH₃, R₅ is phenyl, R₆ is H,
R₇ is 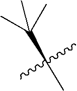
and
R₈ is CH₃,
then R₉ is not 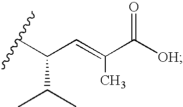
(5) when R₁ is H and R₂ is CH₃ of the moiety 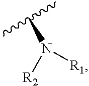
R₃ is CH₃, R₄ is CH₃, R₅ is phenyl, R₆ is H,
R₇ is 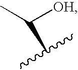
and
R₈ is CH₃,
then R₉ is not 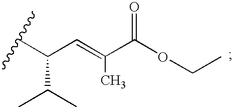
(6) when R₁ is H and R₂ is CH₃ of the moiety 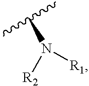
R₃ is CH₃, R₄ is CH₃, R₅ is phenyl, R₆ is H,
R₇ is 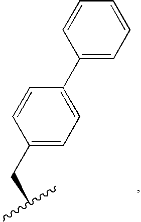
and
R₈ is CH₃,
then R₉ is not 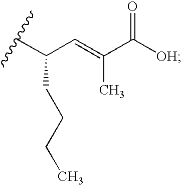
(7) when R₁ is H, R₂ is H, R₃ is CH₃, R₄ is CH₃, R₅ is 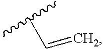
R₆ is H,
R₇ is 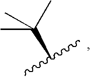
and
R₈ is CH₃,
then R₉ is not 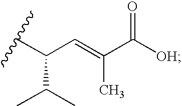
(8) when R₁ is H and R₂ is CH₃, of the moiety 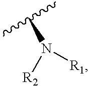
R₃ is CH₃, R₄ is CH₃, R₅ is phenyl, R₆ is H,
R₇ is 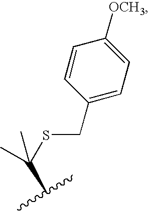
and
R₉ is CH₃,
then R₉ is not 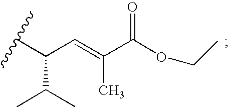
(9) when R₁ is H and R₂ is CH₃ of the moiety 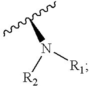
R₃ is CH₃, R₄ is CH₃, R₅ is phenyl,
R₆ is H,
R₇ is 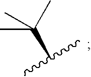
and
R₈ is H,
then R₉ is not 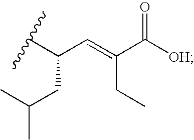
(10) when R₁ is H, R₂ is CH₃,
R₃ is H, R₄ is phenyl, R₅ is phenyl,
R₆ is H,
R₈ is CH₃,
and
R₉ is 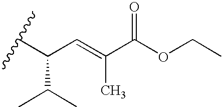
then R₇ is not 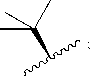
(11) when R₁ is H and R₂ is CH₃ of the moiety 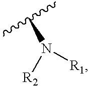
R₃ is CH₃, R₄ is CH₃, R₅ is phenyl,
R₆ is H,
R₈ is CH₃,
and
R₇ is 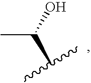
then R₉ is not 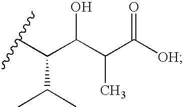
(12) when R₁ is H and R₂ is CH₃ of the moiety 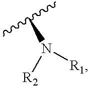
R₃ is CH₃, R₄ is CH₃, R₅ is phenyl,
R₆ is H,
R₇ is 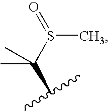
and
R is CH₃,
then R₉ is not 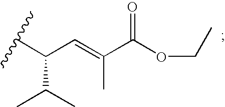
(13) when R₁ is H and R₂ is CH₃ of the moiety 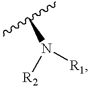
R₃ is CH₃, R₄ is CH₃, R₅ is phenyl,
R₆ is H,
R₇ is 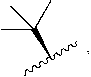
and
R₈ is CH₃,
then R₉ is not 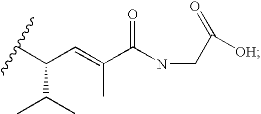
(14) when R₁ is H and R₂ is CH₃ of the moiety 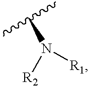
R₃ is CH₃, R₄ is CH₃, R₅ is phenyl,
R₆ is H,
R₇ is 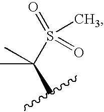
and
R₈ is CH₃,
then R₉ is not 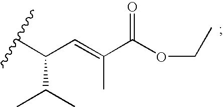
(15) when R₁ is CH₃, R₂ is H,
R₃ is H, R₄ is phenyl, R₅ is phenyl,
R₆ is H,
R₇ is 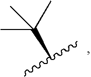
and
R₈ is CH₃,
then R₉ is not 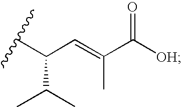
(16) when R₁ is CH₃ and R₂ is H of the moiety 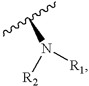
R₃ is CH₃, R₄ is methyl, R₅ is phenyl,
R₆ is H,
R₇ is 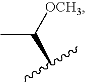
and
R₈ is CH₃,
then R₉ is not 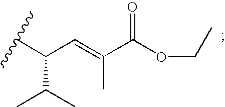
(17) when R₁ is CH₃ and R₂ is H of the moiety 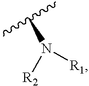
R₃ is CH₃, R₄ is methyl, R₅ is 4-methoxyphenyl,
R₆ is H,
R₇ is 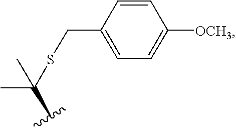
and
R₈ is CH₃,
then R₉ is not 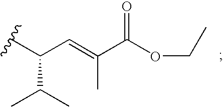
(18) when R₁ is CH₃ and R₂ is H of the moiety 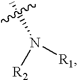
R₃ is CH₃, R₄ is CH₃, R₅ is 3-chlorophenyl,
R₆ is H,
R₇ is 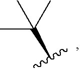
and
R₈ is CH₃,
then R₉ is not 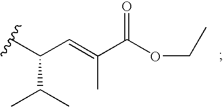
(19) when R₁ is CH₃ and R₂ is H of the moiety 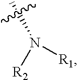
R₃ is CH₃, R₄ is CH₃, R₅ is phenyl,
R₆ is H,
R₇ is 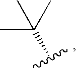
and
R₈ is CH₃,
then R₉ is not 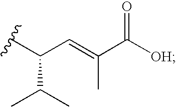
(20) when R₁ is CH₃ and R₂ is CH₃ of the moiety 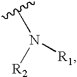
R₃ is H, R₄ is H, R₅ is 3-pyridyl,
R₆ is H,
R₇ is 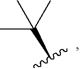
and
R₆ is CH₃,
then R₉ is not 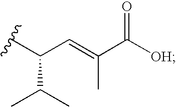
(21) when R₁ is CH₃ and R₂ is H, R₃ is 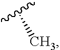
R₄ is H, R₅ is —O—CH2-phenyl,
R₆ is H,
R₇ is 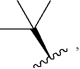
and
R₈ is CH₃,
then R₉ is not 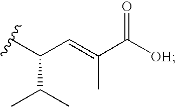
(22) when R₁ is H and R₂ is CH₃ of the moiety 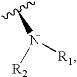
R₃ is CH₃, R₄ is CH₃, R₅ is phenyl, R₆ is CH₃, R₈ is CH₃,
and
R₉ is 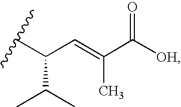
then R₇ is not 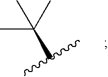
(23) when R₁ is H;
R₃ and R₄ are CH₃;
R₅ is phenyl;
R₆ is H;
R₇ is 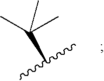
R₆ is CH₃;
R₉ is 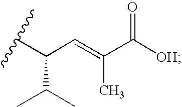
then R₂ of the moiety 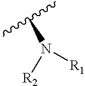
is not 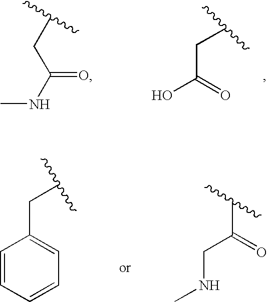
or pharmaceutically acceptable salts thereof.

2. The method according to claim 1 wherein the chemotherapeutic agents are antimicrotubule inhibitors.

3. The method according to claim 2 wherein the antimicrotubule inhibitors are selected from the group consisting of paclitaxel, docetaxel, vinblastine, vincristine and vinorelbine.

4. The method according to claim 1 wherein the tumors are selected from the group consisting of breast, colon, lung, prostate, melanoma, epidermal, leukemia, kidney, bladder, mouth, larynx, esophagus, stomach, ovary, pancreas, liver, skin and brain.

5. The method according to claim 1 wherein the tumors overexpress MDR-1, MXR or MRP.

6. The method according to claim 1 wherein the resistance to chemotherapeutic agents is multiple drug resistance (MDR).

7. The method according to claim 1 wherein the resistance is inherent or acquired.

8. The method according to claim 7 wherein the resistance is acquired.

9. The method according to claim 1 wherein a compound of Formula (II) is administered before, concurrently, or after treatment with the chemotherapeutic agent.

10. The method according to claim 1 wherein:
(a) R₁ and R₂ are independently: H, methyl, ethyl, propyl, or n-butyl; or
(b) R₁ and R₂ taken together with the nitrogen atom to which they are attached form a three to six membered ring;

11. The method according to claim 10 wherein R₁ and R₂ are independently: H or CH₃.

12. The method according to claim 11 wherein R₁ is H and R₂ is CH₃.

13. The method according to claim 10 wherein no more than one of R₁ and R₂ is H.

14. The method according to claim 1 wherein R₃ and R₄are independently: H, methyl, ethyl, n-propyl or n-butyl, provided no more than one of R₃ and R₄ is H or, R₃ and R₄ are joined to form a β-cyclopropyl, β-cyclobutyl, β-cyclopentyl or β-cyclohexyl ring.

15. The method according to claim 14 wherein R₃ and R₄ are each methyl.

16. The method according to claim 1 wherein R₅ is cyclohexyl, phenyl, naphthyl, thienyl, anthracyl, pyrrolyl or indolyl.

17. The method according to claim 16 wherein R₅ is phenyl, or indolyl.

18. The method according to claim 17 wherein R₅is phenyl;

19. The method according to claim 1 wherein R₆ and R₈ are independently: H or methyl.

20. The method according to claim 19 wherein R₆ is H and R₈ is methyl.

21. The method according to claim 1 wherein R₇ is a three to six carbon, branched alkyl group.

22. The method of claim 21 where R₇ is —C(CH₃)₃.

23. The method of claim 1 wherein; Z is OH, or —OR₁₄; R₁₄, is a linear or branched one to six carbon alkyl group, —NHCH(R)₁₁)COOH or —NCH₃CH(R₁₁)COOH; R₁₁ is R, or —(CH₂)ₙ NHC (NH) (NH₂); or
R₉ is —C(R₁₅)—C═C(R₁₆)C(O)—OH wherein R₁₅ is methyl, ethyl, n-propyl, isopropyl, tert-butyl, iso-butyl, or sec-butyl and R₁₆ is H, methyl, ethyl, propyl, iso-propyl, n-butyl, iso-butyl or sec-butyl.

24. The method according to claim 1 wherein R₉ is:

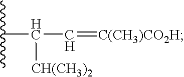

25. The method according to claim 1 wherein
R₉ is

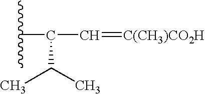

26. The method according to claim 1 wherein R₉ is:

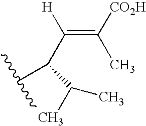

27. The method according to claim 1 wherein the absolute configurations of moieties a, b and c of Formula (II) are: 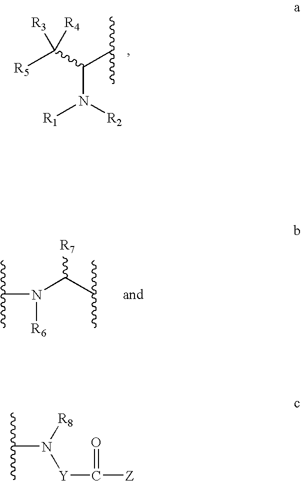
are selected from:

| | a | b | c |
|---|---|---|---|
| | S | S | S |
| | R | S | S |
| and | S | S | R. |

28. The method according to claim 1 wherein said compound of Formula (II) is selected from:
3-Chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide, 4-Chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide,
4-chloro-N,β,β-trimethyl-D-phenylalanyl-N¹-[(1S,2E)4-ethoxy-1-isopropyl-3-methyl-4-oxo-2-butenyl]-N¹,3-dimethyl-L-valinamide,
4-Chloro-N,β,β-triethyl-L-phenylalanyl-N¹-[(1S,2E)-3-carboxy-1-isopropyl-2-butenyl]-N¹,3-dimethyl-L-valinamide,
4-Chloro-N,β,β-trimethyl-D-phenylalanyl-N¹-[(1S,2E)-3-carboxy-1-isopropyl-2-butenyl]-N¹,3-dimethyl-L-valinamide,
N,β,β,3-Tetramethyl-L-phenylalanyl-N¹-[(1S,2E)-4-ethoxy-1-isopropyl-3-methyl-4-oxo-2-butenyl]-N¹,3-dimethyl-L-valinamide,
N,β,β,3-tetramethyl-D-phenylalanyl-N¹-[(1S,2E)4-ethoxy-1-isopropyl-3-methyl-4-oxo-2-butenyl]-N¹,3-dimethyl-L-valinamide,
N,β,β,3-Tetramethyl-L-phenylalanyl-N¹-[(1S,2E)-3-carboxy-1-isopropyl-2-butenyl]-N¹,3-dimethyl-L-valinamide,
N,β,β,3-Tetramethyl-D-phenylalanyl-N¹-[(1S,2E)-3-carboxy-1-isopropyl-2-butenyl]-N¹,3-dimethyl-L-valinamide,
N,β,β,4-Tetramethyl-L-phenylalanyl-N¹-[(1S,2E)4-ethoxy-1-isopropyl-3-methyl-4-oxo-2-butenyl]-N¹,3-dimethyl-L-valinamide,
N,β,β,4-tetramethyl-D-phenylalanyl-N¹-[(1S,2E)-4-ethoxy-1-isopropyl-3-methyl-4-oxo-2-butenyl]-N¹,3-dimethyl-L-valinamide,
N,β,β,4-Tetramethyl-L-phenylalanyl-N¹-[(1S,2E)-3-carboxy-1-isopropyl-2-butenyl]-N¹,3-dimethyl-L-valinamide,
N,β,β,4-Tetramethyl-D-phenylalanyl-N¹-[(1S,2E)-3-carboxy-1-isopropyl-2-butenyl]-N¹,3-dimethyl-L-valinamide,
N,β,β-3,4-Pentamethyl-L-phenylalanyl-N¹-[(1S,2E)4-ethoxy-1-isopropyl-3-methyl-4-oxo-2-butenyl]-N¹,3-dimethyl-L-valinamide,
N,β,β-3,4-pentamethyl-D-phenylalanyl-N¹-[(1S,2E)4-ethoxy-1-isopropyl-3-methyl-4-oxo-2-butenyl]-N¹,3-dimethyl-L-valinamide,
N,β,β,3,4-Pentamethyl-L-phenylalanyl-N¹-[(1S,2E)-3-carboxy-1-isopropyl-2-butenyl]-N¹,3-dimethyl-L-valinamide,
N,β,β,3,4-Pentamethyl-D-phenylalanyl-N¹,[(1S,2E)-3-carboxy-1-isopropyl-2-butenyl]-N¹,3-dimethyl-L-valinamide,
N,β,β,3,5-Pentamethyl-L-phenylalanyl-N¹-[(1S,2E)-4-ethoxy-1-isopropyl-3-methyl-4-oxo-2-butenyl]-N¹,3-dimethyl-L-valinamide,
N,β,β,3,5-pentamethyl-D-phenylalanyl-N¹-[(1S,2E)-4-ethoxy-1-isopropyl-3-methyl4-oxo-2-butenyl]-N¹,3-dimethyl-L-valinamide,
N,β,β,3,5-Pentamethyl-L-phenylalanyl-N¹-[(1S,2E)-3-carboxy-1-isopropyl-2-butenyl]-N¹,3-dimethyl-L-valinamide,
N,β,β,3,5-Pentamethyl-D-phenylalanyl-N¹-[(1S,2E)-3-carboxy-1-isopropyl-2-butenyl]-N¹,3-dimethyl-L-valinamide,
N-Methyl-3-(2-thienyl)-L-valyl-N¹-[(1S,2E)4-ethoxy-1-isopropyl-3-methyl-4-oxo-2-butenyl]-N¹,3-dimethyl-L-valinamide,
N-methyl-3-(2-thienyl)-D-valyl-N¹-[(1S,2E)4-ethoxy-1-isopropyl-3-methyl4-oxo-2-butenyl]-N¹,3-dimethyl-L-valinamide,
N-Methyl-3-(2-thienyl)-L-valyl-N¹-[(1S,2E)-3-carboxy-1-isopropyl-2-butenyl]-N¹,3-dimethyl-L-valinamide,
N-Methyl-3-(2-thienyl)-D-valyl-N¹-[(1S,2E)-3-carboxy-1-isopropyl-2-butenyl]-N¹,3-dimethyl-L-valinamide,
N-Methyl-3-thien-3-yl-L-valyl-N¹-[(1S,2E)4-ethoxy-1-isopropyl-3-methyl4-oxobut-2-enyl]-N¹,3-dimethyl-L-valinamide,
N-methyl-3-thien-3-yl-D-valyl-N¹-[(1S,2E)-4-ethoxy-1-isopropyl-3-methyl-4-oxobut-2-enyl]-N¹,3-dimethyl-L-valinamide,
N-Methyl-3-thien-3-yl-L-valyl-N¹-[(1S,2E)-3-carboxy-1-isopropylbut-2-enyl]-N¹,3-dimethyl-L-valinamide,
N-Methyl-3-thien-3-yl-D-valyl-N¹-[(1S,2E)-3-carboxy-1-isopropylbut-2-enyl]-N¹,3-dimethyl-L-valinamide,
3-(1-Benzothien-3-yl)-N-methylvalyl-N¹-[(1S,2E)4-ethoxy-1-isopropyl-3-methyl4-oxo-2-butenyl]-N¹,3-dimethyl-L-valinamide,
3-(1-Benzothien-3-yl)-N-methylvalyl-N¹-[(1S,2E)-3-carboxy-1-isopropyl-2-butenyl]-N¹,3-dimethyl-L-valinamide,
3-(1-Benzothien-2-yl)-N-methylvalyl-N¹-[(1S,2E)4-ethoxy-1-isopropyl-3-methyl4-oxo-2-butenyl]-N¹,3-dimethyl-L-valinamide,
3-(1-Benzothien-2-yl)-N-methylvalyl-N¹-[(1S,2E)-3-carboxy-1-isopropyl-2-butenyl]-N¹,3-dimethyl-L-valinamide,
4-tert-Butyl-N,β,β-trimethylphenylalanyl-N¹-[(1S,2E)4-ethoxy-1-isopropyl-3-methyl4-oxo-2-butenyl]-N¹,3-dimethyl-L-valinamide,
4-tert-Butyl-N,β,β-trimethylphenylalanyl-N¹-[(1S,2E)-3-carboxy-1-isopropyl-2-butenyl]-N¹,3-dimethyl-L-valinamide,
N-Ethyl-β,β-dimethylphenylalanyl-N¹-[(1S,2E)4-ethoxy-1-isopropyl-3-methyl4-oxo-2-butenyl]-N¹,3-dimethyl-L-valinamide,
N-Ethyl-β,β-dimethylphenylalanyl-N¹-[(1S,2E)-3-carboxy-1-isopropyl-2-butenyl]-N¹,3-dimethyl-L-valinamide,
N-(tert-Butoxycarbonyl)-N-β,β,2-tetramethylphenylalanyl-N¹-[(1S,2E)4-ethoxy-1-isopropyl-3-methyl-4-oxo-2-butenyl]-N¹,3-dimethyl-L-valinamide,
N,β,β,2-tetramethyl-L-phenylalanyl-N¹-[(1S,2E)-4-ethoxy-1-isopropyl-3-methyl4-oxo-2-butenyl]-N¹,3-dimethyl-L-valinamide,
N,β,β,2-Tetramethyl-D-phenylalanyl-N¹-[(1S,2E)4-ethoxy-1-isopropyl-3-methyl-4-oxo-2-butenyl]-N¹,3-dimethyl-L-valinamide,
N,β,β,2-Tetramethyl-L-phenylalanyl-N¹-[(1S,2E)-3-carboxy-1-isopropyl-2-butenyl]-N¹,3-dimethyl-L-valinamide,
N,β,β,2-Tetramethyl-D-phenylalanyl-N¹-[(1S,2E)-3-carboxy-1-isopropyl-2-butenyl]-N¹,3-dimethyl-L-valinamide,
3-bromo-N,β,β-trimethyl-L-phenylalanyl-N¹-[(1S,2E)-3-carboxy-1-isopropylbut-2-enyl]-N¹,3-dimethyl-L-valinamide,
3-bromo-N,β,β-trimethyl-D-phenylalanyl-N¹-[(1S,2E)-3-carboxy-1-isopropylbut-2-enyl]-N¹,3-dimethyl-L-valinamide,
3-phenyl-N,β,β-trimethyl-L-phenylalanyl-N¹-[(1S,2E)-3-carboxy-1-isopropylbut-2-enyl]-N¹,3-dimethyl-L-valinamide,
3-phenyl-N,β,β-trimethyl-D-phenylalanyl-N¹-[(1S,2E)-3-carboxy-1-isopropylbut-2-enyl]-N¹,3-dimethyl-L-valinamide,
N,β,β-trimethyl-3-vinyl-L-phenylalanyl-N¹-[(1S,2E)-3-carboxy-1-isopropylbut-2-enyl]-N¹,3-dimethyl-L-valinamide,
3-ethyl- N,β,β-trimethyl-L-phenylalanyl-N¹-[(1S,2E)-3-carboxy-1-isopropylbut-2-enyl]-N¹,3-dimethyl-L-valinamide,
4-bromo-N,β,β-trimethylphenylalanyl-N¹-[(1S,2E)-3-carboxy-1-isopropyl-2-butenyl]-N¹,3-dimethyl-L-valinamide,
4-phenyl-N,β,β--trimethyl-L-phenylalanyl-N¹-[(1S,2E)-3-carboxy-1-isopropyl-2-butenyl]-N¹,3-dimethyl-L-valinamide,
4-carboxy-N,β,β-trimethylphenylananyl-N¹-[(1S,2E)-3-carboxy-1-isopropylbut-2-enyl]-N¹,3-dimethyl-L-valinamide,
3-Methoxy-N,β,β-trimethylphenylalanyl-N¹-[(1S,2E)-3-carboxy-1-isopropyl-2-butenyl]-N¹,3-dimethyl-L-valinamide,
3-Hydroxy-N,β,β-trimethylphenylalanyl-N¹-[(1S,2E)-3-carboxy-1-isopropyl-2-butenyl]-N¹,3-dimethyl-L-valinamide,
N,3-Dimethyl-4-phenyl-L-valyl-N¹-[(1S,2E)-3-carboxy-1-isopropyl-2-butenyl]-N¹,3-dimethyl-L-valinamide,
N,3-dimethyl4-phenyl-D-valyl-N¹-[(1S,2E)-3-carboxy-1-isopropyl-2-butenyl]-N¹,3-dimethyl-L-valinamide,
(2E,4S)4-[((2S)-2-{[(2S)-3,3-dimethyl-2-(methylamino)octanoyl]amino}-3,3-dimethylbutanoyl)(methyl)amino]-2,5-dimethyl-2-hexenoic acid,
(2E,4S)-4-[((2S)-2-{[(2R)-3,3-dimethyl-2-(methylamino)octanoyl]amino}-3,3-dimethylbutanoyl)(methyl)amino]-2,5-dimethyl-2-hexenoic acid,
N,N,β,β-Tetramethyl-L-phenylalanyl-N¹-[(1S,2E)-3-carboxy-1-isopropyl-2-butenyl]-N¹,3-dimethyl-L-valinamide,
N-(2-hydroxyethyl)-N,β,β-trimethyl-L-phenylalanyl-N¹-[(1S,2E)-3-carboxy-1-isopropylbut-2-enyl]-N¹,3-dimethyl-L-valinamide,
2-Methoxy-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide,
2-Methoxy-N,β,β-trimethyl-L-phenylalanyl-N¹-[(1S,2E)-3-carboxy-1-isopropyl-2-butenyl]-N¹,3-dimethyl-L-valinamide,
N,O,β,β-tetramethyl-L-tyrosyl-N¹-[(1S,2E)4-ethoxy-1-isopropyl-3-methyl4-oxo-2-butenyl]-N¹,3-dimethyl-L-valinamide,
N,O,β,β-tetramethyl-L-tyrosyl-N¹-[(1S,2E)-3-carboxy-1-isopropyl-2-butenyl]-N¹,3-dimethyl-L-valinamide,
2-Methoxy-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide,
2-Methoxy-N,O,β, -tetramethyl-L-tyrosyl-N¹-[(1S,2E)-3-carboxy-1-isopropyl-2-butenyl]-N¹,3-dimethyl-L-valinamide,
3-Fluoro-N,β,β,-trimethyl-L-phenylalanyl-N¹-[(1S,2E)-3-carboxy-1-isopropyl-2-butenyl]-N¹,3-dimethyl-L-valinamide,
3-Fluoro- N,β,β-trimethyl-D-phenylalanyl-N¹-[(1S,2E)-3-carboxy-1-isopropyl-2-butenyl]-N¹,3-dimethyl-L-valinamide,
N,β,β-Trimethyl-3-(trifluoromethyl)-L-phenylalanyl-N¹-[(1S,2E)-3-carboxy-1-isopropyl-2-butenyl]-N¹,3-dimethyl-L-valinamide,
N,β,β-Trimethyl-3-(trifluoromethyl)-D-phenylalanyl-N¹-[(1S,2E)-3-carboxy-1-isopropyl-2-butenyl]-N¹,3-dimethyl-L-valinamide,
3,5-Difluoro-N,β,β3-trimethyl-L-phenylalanyl-N¹-[(1S,2E)-3-carboxy-1-isopropylbut-2-enyl]-N¹,3-dimethyl-L-valinamide,
3,5-Difluoro-N,β,β-trimethyl-D-phenylalanyl-N¹-[(1S,2E)-3-carboxy-1-isopropylbut-2-enyl]-N¹,3-dimethyl-L-valinamide,
N,β,β-trimethyl-3,5-bis(trifluoromethyl)-L-phenylalanyl-N¹-[(1S,2E)-3-carboxy-1-isopropylbut-2-enyl]-N¹,3-dimethyl-L-valinamide,
N,β3,1-trimethyl-3,5-bis(trifluoromethyl)-D-phenylalanyl-N¹-[(1S,2E)-3-carboxy-1-isopropylbut-2-enyl]-N¹,3-dimethyl-L-valinamide,
O-isopropyl-N,β,β-trimethyl-L-tyrosyl-N¹-[(1S,2E)-3-carboxy-1-isopropylbut-2-enyl]-N¹,3-dimethyl-L-valinamide,
O-isopropyl- N,β,β-trimethyl-D-tyrosyl-N¹-[(1S,2E)-3-carboxy-1-isopropylbut-2-enyl]-N¹,3-dimethyl-L-valinamide,
3-Cyclohexyl-N-methyl-L-valyl-N¹-[(1S,2E)-3-carboxy-1-isopropyl-2-butenyl]-N¹,3-dimethyl-L-valinamide,
(2E,4S)-2,5-dimethyl-4-(methyl{3-methyl-N-[(2S)-2-(methylamino)-2-(1-phenylcyclopentyl)ethanoyl]-L-valyl}amino)-2-hexenoic acid,
(2E,4S)-2,5-dimethyl-4-(methyl{3-methyl-N-[(2R)-2-(methylamino)-2-(1-phenylcyclopentyl)ethanoyl]-L-valyl}amino)-2-hexenoic acid,
(2E,4R)-2,5-dimethyl-4-(methyl{3-methyl-N-[(methylamino)(1-phenylcyclohexyl)acetyl]-L-valyl}amino)-2-hexenoic acid,
(E,4S)-2,5-Dimethyl-4-[methyl((2S)-2-{[(2S)-3-methyl-2-(methylamino)-3-phenylbutanoyl]amino}-3-phenylpropanoyl)amino]-2-hexenoic acid,
N,β,β-Trimethyl-L-phenylalanyl-N¹-[(1S,2E)-1-butyl-3-carboxybut-2-enyl]-N¹,3-dimethyl-L-valinamide,
N,β,β-Trimethyl-L-phenylalanyl-N¹-[(1S,2E)-3-carboxy-1-isobutyl-2-pentenyl]-N¹,3-dimethyl-L-valinamide,
(E,4S)-2-Butyl-4-[((2S)-3,3-dimethyl-2-{[(2S)-3-methyl-2-(methylamino)-3-phenylbutanoyl]amino}butanoyl)amino]-5-methyl-2-hexenoic acid,
N,β,β-Trimethyl-L-phenylalanyl-N ′-[(1S,2E)-3-carboxy-1-isopropyl-2-pentenyl]-N¹,3-dimethyl-L-valinamide,
Ethyl (E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}2-hexenoate,
(E,4S)-2,5-dimethyl-4-{methyl[(2R)-3-methyl-2-{[(2S)-3-methyl-2-(methylamino)-3-phenylbutanoyl]amino}-3-(methylsulfanyl)butanoyl]amino}-2-hexenoic acid,
N,β,β-trimethyl-L-phenylalanyl-N¹-[(1S,2E)-3-carboxy-1-isopropyl-2-butenyl]-N¹-methyl-3-(methylsulfonyl)-L-valinamide,
N,β,β-trimethyl-L-phenylalanyl-N¹-[(1S,2E)-3-carboxy-1-isopropyl-2-butenyl]-3-[(4-methoxybenzyl)sulfanyl]-N¹-methyl-L-valinamide,
N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide,
N,O,β,β-tetramethyl-L-tyrosyl-N¹-[(1S,2E)-3-carboxy-1-isopropyl-2-butenyl]-N¹-methyl-3-(methylsulfanyl)-L-valinamide,
N,β,β-trimethyl-L-phenylalanyl-N¹-[(1R,2E)4-ethoxy-1-isopropyl-3-methyl4-oxo-2-butenyl]-N¹-methyl-L-allothreoninamide,
N,β,β-trimethyl-L-phenylalanyl-N¹-[(1S,2E)-3-carboxy-1-isopropyl-2-butenyl]-N¹-methyl-L-allothreoninamide,
N,β,β-trimethyl-L-phenylalanyl-N¹-[(1S,2E)-3-carboxy-1-isopropyl-2-butenyl]-N,O, β,β-tetramethyl-L-tyrosinamide,
N,β,β-trimethyl-L-phenylalanyl-N¹-[(1S,2E)-3-carboxy-1-isopropyl-2-butenyl]-N¹,O-dimethyl-L-allothreoninamide,
(E,4S)-2,5-Dimethyl4-[methyl((2S)-2-{[(2S)-3-methyl-2-(methylamino)-3-phenylbutanoyl]amino}4-phenylbutanoyl)amino]-2-hexenoic acid,
N,β,β-trimethyl-L-phenylalanyl-4-benzoyl-N¹-[(1S,2E)-3-carboxy-1-isopropyl-2-butenyl]- N,β,β-trimethyl-L-phenylalaninamide,
4-benzoyl-N,β,β-trimethyl-L-phenylalanyl-N¹-[(1S,2E)-3-carboxy-1-isopropylbut-2-enyl]-N¹,3-dimethyl-L-valinamide,
N,β,β-trimethyl-L-phenylalanyl-N¹-[(1S,2E)-3-carboxy-1-isobutylbut-2-enyl]-N¹-methyl-L-valinamide,
N,β,β-trimethyl-L-phenylalanyl-N¹-[(1S,2E)-3-carboxy-1-isobutylbut-2-enyl]-3-methyl-L-valinamide,
N,β,β-trimethyl-L-phenylalanyl-N¹-[(1S,2E)-3-carboxy-1-isopropylbut-2-enyl]-N¹-ethyl-3-methyl-L-valinamide,
N,β,β-trimethyl-L-phenylalanyl-N¹-[(1S,2E)-3-carboxy-1-isopropylbut-2-enyl]-N¹-ethyl-L-valinamide,
N,β,β-trimethyl-L-phenylalanyl-N¹-[(1S,2E)-3-carboxy-1-isopropylbut-2-enyl]-N¹-methyl-L-leucinamide,
N,β,β-trimethyl-L-phenylalanyl-N¹-[(1S,2E)-3-carboxy-1-isopropylbut-2-enyl]-N¹-methyl-L-norvalinamide,
(2E,4S)-4-[{(2R)-2-cyclohexyl-2-[(N,β,β-trimethyl-L-phenylalanyl)amino]ethanoyl}(methyl)amino]-2,5-dimethylhex-2-enoic acid,
(2E ,4S)-2,5-dimethyl-4-(methyl{(2S)-2-[(N,β,β-trimethyl-L-phenylalanyl)amino]butanoyl}amino)hex-2-enoic acid,
4-{[3,3-Dimethyl-2-(2-methylamino-3-phenyl-butyrylamino)-butyryl]-methyl-amino}-2,5-dimethyl-hex-2-enoic acid,
N,β,β-trimethyl-L-phenylalanyl-N¹-[(1S,2E)-3-carboxy-1-isopropylbut-2-enyl]-3-methyl-L-valinamide,
N,β,β-trimethyl-L-phenylalanyl-N¹-[(1S,2E)-3-carboxy-1-isopropylbut-2-enyl]-L-valinamide,
2,5-dimethyl4-{methyl-[2-(3-methyl-2-methylamino-3-phenyl-butyrylamino)-propionyl]-amino}-hex-2-enoic acid,
4-{[3,3-Dimethyl-2-(3-methyl-2-methylamino-3-phenyl-butyrylamino)-butyryl]-methyl-amino}-2,6-dimethyl-hept-2-enoic acid,
N,β,β-trimethyl-L-phenylalanyl-N¹-[(1S,2E)-3-carboxy-1-isopropyl-2-butenyl]-N¹-methyl-L-valinamide,
N,β,β-trimethyl-L-phenylalanyl-N¹-[(1S,2E)-3-carboxy-1-isopropyl-2-butenyl]-N¹-methyl-L-isoleucinamide,
(E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenamide,
(E,4S)-4-[((2S)-3,3-dimethyl-2-{[(2S)-3-methyl-2-(methylamino)-3-phenylbutanoylamino}butanoyl)(methyl)amino]-N,2,5-trimethyl-2-hexenamide,
N,β,β-trimethyl-L-phenylalanyl-N¹-{(1S,2E)-4-[(2-cyanoethyl)amino]-1-isopropyl-3-methyl-4-oxo-2-butenyl}-N¹,3-dimethyl-L-valinamide,
N,β,β-trimethyl-L-phenylalanyl-N¹-{(1S,2E)-4-[(carboxymethyl)amino]-1-isopropyl-3-methyl-4-oxo-2-butenyl}-N¹,3-dimethyl-L-valinamide,
N,β,β-trimethyl-L-phenylalanyl-N¹-{(1S,2E)-4-[(4-azidophenyl)amino]-1-isopropyl-3-methyl-4-oxo-2-butenyl}-N¹,3-dimethyl-L-valinamide,
N,β,β-trimethyl-L-phenylalanyl-N¹-{(1S,2E)-1-isopropyl-3-methyl-4-oxo-4-[(2-phenylethyl)amino]but-2-enyl}-N¹-,3-dimethyl-L-valinamide,
N,β,β-trimethyl-L-phenylalanyl-N¹-{(1S,2E)4-[[(1S,2E)-4-ethoxy-1-isopropyl-3-methyl-4-oxobut-2-enyl](methyl)amino]-1-isopropyl-3-methyl-4-oxobut-2-enyl}-N¹-,3-dimethyl-L-valinamide,
N,β,β-trimethyl-L-phenylalanyl-N¹-{(1S,2E)-4-[[(1S,2E)-3-carboxy-1-isopropylbut-2-enyl](methyl)amino]-1-isopropyl-3-methyl-4-oxobut-2-enyl}-N-1-,3-dimethyl-L-valinamide,
N,β,β-trimethyl-L-phenylalanyl-N¹-[(1S,2E)-1-isopropyl-3-methyl-4-oxo-4-(thien-2-ylmethoxy)but-2-enyl]-N¹-,3-dimethyl-L-valinamide,
N,β,β-trimethyl-L-phenylalanyl-N¹-[(1S,2E)-l-isopropyl-3-methyl-4-(octyloxy)-4-oxobut-2-enyl]-N¹-,3-dimethyl-L-valinamide,
N,β,β-trimethyl-L-phenylalanyl-N¹-[(1S,2Z)-3-carboxy-1-isopropyl-2-butenyl]-N¹,3-dimethyl-L-valinamide,
N,β,β-trimethyl-L-phenylalanyl-N¹-[(1S,2E)-3-carboxy-1-isopropylprop-2-enyl]-N¹,3-dimethyl-L-valinamide,
N,β,β-trimethyl-L-phenylalanyl-N¹-[(1S,2E)-1-allyl-3-carboxybut-2-enyl]-N¹,3-dimethyl-L-valinamide,
(2E,4S)-4-[{(2S)-3,3-dimethyl-2-[(N,β,β-trimethyl-L-phenylalanyl)amino]-4-pentenoyl}(methyl)amino]-2,5-dimethyl-2-hexenoic acid,
(2E, 4S)-4-[((2S)-2-{[3,3-dimethyl-2-(methylamino)-4-pentenoyl]amino}-3,3-dimethylbutanoyl)(methyl)amino]-2,5-dimethyl-2-hexenoic acid,
N,β,β-trimethyl-L-phenylalanyl-N¹-[(1S,2E)-3-carboxy-1-isopropylbut-2-enyl]-N¹,3-dimethyl-L-isoleucinamide,
N,β,β-trimethyl-L-phenylalanyl-N¹-[(1R,3S)-3-carboxy-1-isopropylbutyl]-N¹,3-dimethyl-L-valinamide,
N,β,β-trimethyl-L-phenylalanyl-N¹-[(1R,3R)-3-carboxy-1-isopropylbutyl]-N¹,3-dimethyl-L-valinamide,
β,β-diethyl-N-methyl-L-phenylalanyl-N¹-[(1S,2E)-3-carboxy-1-isopropylbut-2-enyl]-N¹,3-dimethyl-L-valinamide,
β,β-diethyl-N-methyl-D-phenylalanyl-N¹-[(1S,2E)-3-carboxy-1-isopropylbut-2-enyl]-N¹,3-dimethyl-L-valinamide,
β,β-dimethyl-L-phenylalanyl-N¹-[(1S,2E)-3-carboxy-1-isopropylbut-2-enyl]-N¹,3-dimethyl-L-valinamide,
O-benzyl-N-methyl-L-threonyl-N¹-[(1S,2E)-3-carboxy-1-isopropylbut-2-enyl]-N¹,3-dimethyl-L-valinamide,
N,β,β-trimethyl-L-phenylalanyl-N¹-[(1S,2E)-3-carboxy-1-isopropylbut-2-enyl]-N¹,3-dimethyl-L-valinamide,
(2E,4S)-4-[((2S)-2-{[(2S)-2-Amino-3-(I -naphthyl)propanoyl]amino}-3,3-dimethylbutanoyl)(methyl)amino]-2,5-dimethyl-2-hexenoic acid,
N,β,β-trimethyl-L-phenylalanyl-N¹-[(1S,2E)-3-carboxy-1-isopropylbut-2-enyl]-N¹-methyl-D-valinamide,
(E,4S)-4-[((2S)-3,3-dimethyl-2-{[(2S)-3-methyl-2-(methylamino)-3-(1-methyl-1H-ethyl-1H-indol-3-yl)butanoyl]amino}butanoyl)amino]-2,5-dimethyl-2-hexenoic acid,
ethyl (E,4S)-4-[((2S)-3,3-dimethyl-2-{[(2S)-3-methyl-2-(methylamino)-3-phenylbutanoyl]amino]butanoyl)(methyl)amino]-2,5-dimethyl-2-hexenoate,
(E,4S)-4-[((2S)-3,3-dimethyl-2-{[(2S)-3-methyl-2-(methylamino)-3-phenylbutanoyl]amino}butanoyl)(methyl)amino]-2,5-dimethyl-2-hexenoic acid,
Ethyl (E,4S)4-[((2S)-3,3-dimethyl-2-{[(2R)-3-methyl-2-(methylamino)-3-phenylbutanoyl]amino]butanoyl)(methyl)amino]-2,5-dimethyl-2-hexenoate,
(E,4S)-4-[((2S)-3,3-dimethyl-2-{[(2S)-3-methyl-2-(methylamino)-3-phenylbutanoyl]amino]butanoyl)(methyl)amino]-2-methyl-5-phenyl-2-pentenoic acid,
(E,4S)-2,5-dimethyl-4-[methyl((2S)-2-{[(2S)-3-methyl-2-(methylamino)-3-phenylbutanoyl]amino}-3-phenylpropanoyl)amino]-2-hexenoic acid,
(4R)-4-[((2S)-2-{[(2S)-2-amino-4-methylpentanoyl]amino}-3,3-dimethylbutanoyl)amino]-2,5-dimethylhexanoic acid,
N,β,β-trimethyl-L-phenylalanyl-N¹-[(1S,2E)-3-carboxy-1-isopropyl-2-butenyl]-N¹-methyl-L-alpha-glutamine,
N,3-dimethyl-L-valyl-N¹-[(1S,2E)-3-carboxy-1-isopropyl-2-butenyl]-N¹,3-dimethyl-L-valinamide,
N,β,β-trimethyl-L-tryptophyl-N¹-[(1S,2E)-3-carboxy-1-isopropyl-2-butenyl]-N¹,3-dimethyl-L-valinamide,
3-cyclohexyl-N-methyl-L-valyl-N¹-[(1S,2E)-4-ethoxy-1-isopropyl-3-methyl-4-oxo-2-butenyl]-N¹,3-dimethyl-L-valinamide,
(2E,4S)-2,5-dimethyl-4-(methyl{3-methyl-N-[(2S)-2-(methylamino)-2-(1-phenylcyclopropyl)acetyl]-L-valyl}amino)hex-2-enoic acid,
(2E,4S)-2,5-dimethyl4-(methyl{3-methyl-N-[(2R)-2-(methylamino)-2-(1-phenylcyclopropyl)acetyl]-L-valyl}amino)hex-2-enoic acid,
2-(4-{[3,3-Dimethyl-2-(3-methyl-2-methylamino-3-phenyl-butyrylamino)-butyryl]-methyl-amino}-2,5-dimethyl-hex-2-enoylamino)-4-methylsulfanyl-butyric acid methyl ester,
N,β,β-trimethyl-L-phenylalanyl-N1-((1S,2E)-4-{[(1S)-1-carboxy-3-(methylthio)propyl]amino}-1-isopropyl-3-methyl-4-oxobut-2-enyl)-N¹,3-dimethyl-L-valinamide,
N,β,β-trimethyl-4-[(E)-2-phenylvinyl]-L-phenylalanyl-N¹-[(1S,2E)-3-carboxy-1-isopropylbut-2-enyl]-N¹,3-dimethyl-L-valinamide,
N,β,β-trimethyl-4-[(E)-2-phenylvinyl]-D-phenylalanyl-N¹-[(1S,2E)-3-carboxy-1-isopropylbut-2-enyl]-N¹,3-dimethyl-L-valinamide,
N,β,β-trimethyl-L-phenylalanyl-N¹-[(1S,2E)-3-carboxy-1-isopropylbut-2′enyl]-3-fluoro-N¹-methyl-D-valinamide,
N,β,β-trimethyl-L-phenylalanyl-N¹-[(1S,2E)-3-carboxy-1-isopropylbut-2-enyl]-3-fluoro-N¹-methyl-L-valinamide,
3-[(4-methoxybenzyl)thio]-N-methyl-L-valyl-N¹-[(1S,2E)-3-carboxy-1-isopropylbut-2-enyl]-N¹,3-dimethyl-L-valinamide,
N-ethyl-β,β-dimethyl-L-phenylalanyl-N¹-[(1S,2E)-3-carboxy-1-isopropylbut-2-enyl]-N¹,3-dimethyl-L-valinamide,
(2E,4S)-2,5-dimethyl-4-(methyl{3-methyl-N-[(2S)-3-methyl-3-phenyl-2-pyrrolidin-1-ylbutanoyl]-L-valyl}amino)hex-2-enoic acid,
N-(2-hydroxyethyl)-β,β-dimethyl-L-phenylalanyl-N¹-[(1S,2E)-3-carboxy-1-isopropylbut-2-enyl]-N¹,3-dimethyl-L-valinamide,
(βR)-N,β,β-dimethyl-L-phenylalanyl-N¹-[(1S,2E)-3-carboxy-1-isopropylbut-2- enyl]-N¹,3-dimethyl-L-valinamide,
3-acetyl-N,β,β-trimethyl-L-phenylalanyl-N¹-[(1S,2E)-3-carboxy-1-isopropylbut-2-enyl]-N¹,3-dimethyl-L-valinamide,
N,β,β-trimethyl-L-phenylalanyl-N¹-[(1S,2E)-3-carboxy-1-isopropylbut-2-enyl]-3-hydroxy-N¹-methyl-L-valinamide, and
N,β,β-trimethyl-L-phenylalanyl-N¹-[(1R,2E)-3-carboxy-1-isopropylbut-2- enyl]-N¹,3-dimethyl-L-valinamide
or pharmaceutically acceptable salts thereof.

29. The method according to claim 28 wherein said compound of Formula (II) is selected from:
3-Chloro-N,β,β-trimethyl-L-phenylalanyl-N¹-[(1S,2E)-4-ethoxy-1-isopropyl-3-methyl-4-oxo-2-butenyl]-N¹,3-dimethyl-L-valinamide,
4-Chloro- N,β,β-trimethyl-L-phenylalanyl-N¹-[(1S,2E)-4-ethoxy-1-isopropyl-3-methyl-4-oxo-2-butenyl]-N¹,3-dimethyl-L-valinamide,
4-chloro- N,β,β-trimethyl-D-phenylalanyl-N¹-[(1S,2E)-4-ethoxy-1-isopropyl-3-methyl-4-oxo-2-butenyl]-N¹,3-dimethyl-L-valinamide,
4-Chloro- N,β,1-triethyl-L-phenylalanyl-N¹-[(1S,2E)-3-carboxy-1-isopropyl-2-butenyl]-N¹,3-dimethyl-L-valinamide,
4-Chloro-N,β,β-trimethyl-D-phenylalanyl-N¹-[(1S,2E)-3-carboxy-1-isopropyl-2-butenyl]-N¹,3-dimethyl-L-valinamide and
3-ethyl- N,β,β-trimethyl-L-phenylalanyl-N¹-[(1S,2E)-3-carboxy-1-isopropylbut-2-enyl]-N¹,3-dimethyl-L-valinamide or pharmaceutically acceptable salts thereof.

30. The method according to claim 28 wherein said compound of Formula (II) is selected from:
N,β,β,3-Tetramethyl-L-phenylalanyl-N¹-[(1S,2E)4-ethoxy-1-isopropyl-3-methyl4-oxo-2-butenyl]-N¹,3-dimethyl-L-valinamide,
N,β,β,3-tetramethyl-D-phenylalanyl-N¹-[(1S,2E)4-ethoxy-1-isopropyl-3-methyl-4-oxo-2-butenyl]-N¹,3-dimethyl-L-valinamide,
N,β,β,3-Tetramethyl-L-phenylalanyl-N¹-[(1S,2E)-3-carboxy-1-isopropyl-2-butenyl]-N¹,3-dimethyl-L-valinamide,
N,β,β,3-Tetramethyl-D-phenylalanyl-N¹-[(1S,2E)-3-carboxy-1-isopropyl-2-butenyl]-N¹,3-dimethyl-L-valinamide,
N,β,β,4-Tetramethyl-L-phenylalanyl-N¹-[(1S,2E)-4-ethoxy-1-isopropyl-3-methyl4-oxo-2-butenyl]-N¹,3-dimethyl-L-valinamide,
N,β,β,4-tetramethyl-D-phenylalanyl-N¹-[(1S,2E)-4-ethoxy-1-isopropyl-3-methyl-4-oxo-2-butenyl]-N¹,3-dimethyl-L-valinamide,
N,β,β,4-Tetramethyl-L-phenylalanyl-N¹-[(1S,2E)-3-carboxy-1-isopropyl-2-butenyl]-N¹,3-dimethyl-L-valinamide,
N,β,β,4-Tetramethyl-D-phenylalanyl-N¹-[(I S,2E)-3-carboxy-1-isopropyl-2-butenyl]-N¹,3-dimethyl-L-valinamide,
N,β,β-3,4-Pentamethyl-L-phenylalanyl-N¹-[(l S,2E)4-ethoxy-1-isopropyl-3-methyl-4-oxo-2-butenyl]-N¹,3-dimethyl-L-valinamide,
N,β,β-3,4-pentamethyl-D-phenylalanyl-N¹-[(I S,2E)4-ethoxy-1-isopropyl-3-methyl4-oxo-2-butenyl]-N¹,3-dimethyl-L-valinamide,
N,β,β3,4-Pentamethyl-L-phenylalanyl-N¹-[(1S,2E)-3-carboxy-1-isopropyl-2-butenyl]-N¹,3-dimethyl-L-valinamide,
N,β,β3,4-Pentamethyl-D-phenylalanyl-N¹-[(1S,2E)-3-carboxy-1-isopropyl-2-butenyl]-N¹,3-dimethyl-L-valinamide,
N,β,β3,5-Pentamethyl-L-phenylalanyl-N¹-[(1S,2E)4-ethoxy-1-isopropyl-3-methyl-4-oxo-2-butenyl]-N¹,3-dimethyl-L-valinamide,
N,β,β,3,5-pentamethyl-D-phenylalanyl-N¹-[(1S,2E)-4-ethoxy-1-isopropyl-3-methyl4-oxo-2-butenyl]-N¹,3-dimethyl-L-valinamide,
N,β,β,3,5-Pentamethyl-L-phenylalanyl-N¹-[(1S,2E)-3-carboxy-1-isopropyl-2-butenyl]-N¹,3-dimethyl-L-valinamide and
N,β,β,3,5-Pentamethyl-D-phenylalanyl-N¹-[(1S,2E)-3-carboxy-1-isopropyl-2-butenyl]-N¹,3-dimethyl-L-valinamide
or pharmaceutically acceptable salts thereof.

31. The method according to claim 28 wherein said compound of Formula (II) is selected from:
N-Methyl-3-(2-thienyl)-L-valyl-N¹-[(1S,2E)-4-ethoxy-1-isopropyl-3-methyl-4-oxo-2-butenyl]-N¹,3-dimethyl-L-valinamide,
N-methyl-3-(2-thienyl)-D-valyl-N¹-[(1S,2E)4-ethoxy-1-isopropyl-3-methyl-4-oxo-2-butenyl]-N¹,3-dimethyl-L-valinamide,
N-Methyl-3-(2-thienyl)-L-valyl-N¹-[(1S,2E)-3-carboxy-1-isopropyl-2-butenyl]-N¹,3-dimethyl-L-valinamide,
N-Methyl-3-(2-thienyl)-D-valyl-N¹-[(1S,2E)-3-carboxy-1-isopropyl-2-butenyl]-N¹,3-dimethyl-L-valinamide,
N-Methyl-3-thien-3-yl-L-valyl-N¹-[(1S,2E)4-ethoxy-1-isopropyl-3-methyl-4-oxobut-2-enyl]-N¹,3-dimethyl-L-valinamide,
N-methyl-3-thien-3-yl-D-valyl-N¹-[(1S,2E)-4-ethoxy-1-isopropyl-3-methyl-4-oxobut-2-enyl]-N¹,3-dimethyl-L-valinamide,
N-Methyl-3-thien-3-yl-L-valyl-N¹-[(1S,2E)-3-carboxy-1-isopropylbut-2-enyl]-N¹,3-dimethyl-L-valinamide and
N-Methyl-3-thien-3-yl-D-valyl-N¹-[(1S,2E)-3-carboxy-1-isopropylbut-2-enyl]-N¹,3-dimethyl-L-valinamide
or pharmaceutically acceptable salts thereof.

32. The method according to claim 28 wherein said compound of Formula (II) is selected from:
3-(1-Benzothien-3-yl)-N-methylvalyl-N¹-[(1S,2E)-4-ethoxy-1-isopropyl-3-methyl4-oxo-2-butenyl]-N¹,3-dimethyl-L-valinamide,
3-(1-Benzothien-3-yl)-N-methylvalyl-N¹-[(1S,2E)-3-carboxy-1-isopropyl-2-butenyl]-N¹,3-dimethyl-L-valinamide,
3-(1-Benzothien-2-yl)-N-methylvalyl-N¹-[(1S,2E)-4-ethoxy-1-isopropyl-3-methyl-4-oxo-2-butenyl]-N¹,3-dimethyl-L-valinamide and
3-(1-Benzothien-2-yl)-N-methylvalyl-N¹-[(1S,2E)-3-carboxy-1-isopropyl-2-butenyl]-N¹,3-dimethyl-L-valinamide
or pharmaceutically acceptable salts thereof.

33. The method according to claim 28 wherein said compound of Formula (II) is selected from:
4-tert-Butyl-N,β,β-trimethylphenylalanyl-N¹-[(1S,2E)-4-ethoxy-1-isopropyl-3-methyl-4-oxo-2-butenyl]-N¹,3-dimethyl-L-valinamide,
4-tert-Butyl-N,β,β-trimethylphenylalanyl-N¹-[(1S,2E)-3-carboxy-1-isopropyl-2-butenyl]-N¹,3-dimethyl-L-valinamide,
N-Ethyl-β,β-dimethylphenylalanyl-N¹-[(1S,2E)-4-ethoxy-1-isopropyl-3-methyl-4-oxo-2-butenyl]-N¹,3-dimethyl-L-valinamide and
N-Ethyl-β,β-dimethylphenylalanyl-N¹-[(1S,2E)-3-carboxy-1-isopropyl-2-butenyl]-N¹,3-dimethyl-L-valinamide
or pharmaceutically acceptable salts thereof.

34. The method according to claim 28 wherein said compound of Formula (II) is selected from:
N-(tert-Butoxycarbonyl)-N-β,β,2-tetramethylphenylalanyl-N¹-[(1S,2E)-4-ethoxy-1-isopropyl-3-methyl-4-oxo-2-butenyl]-N¹,3-dimethyl-L-valinamide,
N,β,β,2-tetramethyl-L-phenylalanyl-N¹-[(1S,2E)-4-ethoxy-1-isopropyl-3-methyl-4-oxo-2-butenyl]-N¹,3-dimethyl-L-valinamide,
N,β,β,2-Tetramethyl-D-phenylalanyl-N¹-[(1S,2E)4-ethoxy-1-isopropyl-3-methyl-4-oxo-2-butenyl]-N¹,3-dimethyl-L-valinamide,
N,β,β-Tetramethyl-L-phenylalanyl-N¹-[(1S,2E)-3-carboxy-1-isopropyl-2-butenyl]-N¹,3-dimethyl-L-valinamide and
N,β,β,2-Tetramethyl-D-phenylalanyl-N¹-[(1S,2E)-3-carboxy-1-isopropyl-2-butenyl]-N¹,3-dimethyl-L-valinamide
or pharmaceutically acceptable salts thereof.

35. The method according to claim 28 wherein said compound of Formula (II) is selected from:
3-bromo-N,β,β-trimethyl-L-phenylalanyl-N¹-[(1S,2E)-3-carboxy-1-isopropylbut-2-enyl]-N¹,3-dimethyl-L-valinamide,
3-bromo-N,β,β-trimethyl-D-phenylalanyl-N¹-[(1S,2E)-3-carboxy-1-isopropylbut-2-enyl]-N¹,3-dimethyl-L-valinamide and
4-bromo-N,β,β-trimethylphenylalanyl-N¹-[(1S,2E)-3-carboxy-1-isopropyl-2-butenyl]-N¹,3-dimethyl-L-valinamide
or pharmaceutically acceptable salts thereof.

36. The method according to claim 28 wherein said compound of Formula (II) is selected from:
3-phenyl-N,β,β-trimethyl-L-phenylalanyl-N¹-[(1S,2E)-3-carboxy-1-isopropylbut-2-enyl]-N¹,3-dimethyl-L-valinamide,
3-phenyl-N,β,β-trimethyl-D-phenylalanyl-N¹-[(1S,2E)-3-carboxy-1-isopropylbut-2-enyl]-N¹,3-dimethyl-L-valinamide and
4-phenyl-N,β,β-trimethyl-L-phenylalanyl-N¹-[(1S,2E)-3-carboxy-I -isopropyl-2-butenyl]-N¹,3-dimethyl-L-valinamide
or pharmaceutically acceptable salts thereof.

37. The method according to claim 28 wherein said compound of Formula (II) is selected from:
4-carboxy-N,β,β-trimethylphenylalanyl-N¹-((1S,2E)-3-carboxy-1-isopropylbut-2-enyl]-N¹,3-dimethyl-L-valinamide,
3-Methoxy- N,β,β-trimethylphenylalanyl-N¹-[(1S,2E)-3-carboxy-1-isopropyl-2-butenyl]-N¹,3-dimethyl-L-valinamide and
3-Hydroxy- N,β,β-trimethylphenylalanyl-N¹-[(1S,2E)-3-carboxy-1-isopropyl-2-butenyl]-N¹,3-dimethyl-L-valinamide
or pharmaceutically acceptable salts thereof.

38. The method according to claim 28 wherein said compound of Formula (II) is selected from:
N,β,β-trimethyl-3-vinyl-L-phenylalanyl-N¹-[(1S,2E)-3-carboxy-1-isopropylbut-2-enyl]-N¹,3-dimethyl-L-valinamide,
N,3-Dimethyl-4-phenyl-L-valyl-N¹-[(1S,2E)-3-carboxy-1-isopropyl-2-butenyl]-N¹,3-dimethyl-L-valinamide,
N,3-dimethyl-4-phenyl-D-valyl-N¹-[(1S,2E)-3-carboxy-1-isopropyl-2-butenyl]-N¹,3-dimethyl-L-valinamide,
(2E,4S)-4-[((2S)-2-{[(2S)-3,3-dimethyl-2-(methylamino)octanoyl]amino}-3,3-dimethylbutanoyl)(methyl)amino]-2,5-dimethyl-2-hexenoic acid,
(2E,4S)-4-[((2S)-2-1[(2R)-3,3-dimethyl-2-(methylamino)octanoyl]amino}-3,3-dimethylbutanoyl)(methyl)amino]-2,5-dimethyl-2-hexenoic acid,
N,N,β,β-Tetramethyl-L-phenylalanyl-N¹-[(1S,2E)-3-carboxy-1-isopropyl-2-butenyl]-N¹,3-dimethyl-L-valinamide and
N-(2-hydroxyethyl)-N,β,β-trimethyl-L-phenylalanyl-N¹-[(1S,2E)-3-carboxy-1-isopropylbut-2-enyl]-N¹,3-dimethyl-L-valinamide
or pharmaceutically acceptable salts thereof.

39. The method according to claim 28 wherein said compound of Formula (II) is selected from:
2-Methoxy-N,β,β-trimethyl-L-phenylalanyl-N¹-[(1S,2E)4-ethoxy-1-isopropyl-3-methyl-4-oxo-2-butenyl]-N¹,3-dimethyl-L-valinamide,
2-Methoxy-N,β,β-trimethyl-L-phenylalanyl-N¹-[(1S,2E)-3-carboxy-1-isopropyl-2-butenyl]-N¹,3-dimethyl-L-valinamide,
N,O,β,β-tetramethyl-L-tyrosyl-N¹-[(1S,2E)4-ethoxy-1-isopropyl-3-methyl4-oxo-2-butenyl]-N¹,3-dimethyl-L-valinamide,
N,O,β,β-tetramethyl-L-tyrosyl-N¹-[(1S,2E)-3-carboxy-1-isopropyl-2-butenyl]-N¹,3-dimethyl-L-valinamide,
2-Methoxy-N,O,β,β-tetramethyl-L-tyrosyl-N¹-[(1S,2E)-4-ethoxy-1-isopropyl-3-methyl-4-oxo-2-butenyl]-N¹,3-dimethyl-L-valinamide,
2-Methoxy-N,O,β,β-tetramethyl-L-tyrosyl-N¹-[(1S,2E)-3-carboxy-1-isopropyl-2-butenyl]-N¹,3-dimethyl-L-valinamide,
O-isopropyl-N,β,β-trimethyl-L-tyrosyl-N¹-[(1S,2E)-3-carboxy-1-isopropylbut-2-enyl]-N¹,3-dimethyl-L-valinamide and
O-isopropyl-N,β,β-trimethyl-D-tyrosyl-N¹-[(1S,2E)-3-carboxy-1-isopropylbut-2-enyl]-N¹,3-dimethyl-L-valinamide
or pharmaceutically acceptable salts thereof.

40. The method according to claim 28 wherein said compound of Formula (II) is selected from:
3-Fluoro-N,β,β-trimethyl-L-phenylalanyl-N¹-[(1S,2E)-3-carboxy-1-isopropyl-2-butenyl]-N¹,3-dimethyl-L-valinamide,
3-Fluoro- N,β,β-trimethyl-D-phenylalanyl-N¹-[(1S,2E)-3-carboxy-1-isopropyl-2-butenyl]-N¹,3-dimethyl-L-valinamide,
N,β,β-Trimethyl-3-(trifluoromethyl)-L-phenylalanyl-N¹-[(1S,2E)-3-carboxy-1-isopropyl-2-butenyl]-N¹,3-dimethyl-L-valinamide,
N,β,β-Trimethyl-3-(trifluoromethyl)-D-phenylalanyl-N¹-[(1S,2E)-3-carboxy-1-isopropyl-2-butenyl]-N¹,3-dimethyl-L-valinamide,
3,5-Difluoro- N,β,β-trimethyl-L-phenylalanyl-N¹-[(1S,2E)-3-carboxy-1-isopropylbut-2-enyl]-N¹,3-dimethyl-L-valinamide,
3,5-Difluoro- N,β,β-trimethyl-D-phenylalanyl-N¹-[(1S,2E)-3-carboxy-1-isopropylbut-2-enyl]-N¹,3-dimethyl-L-valinamide,
N,β,β-trimethyl-3,5-bis(trifluoromethyl)-L-phenylalanyl-N¹-[(1S,2E)-3-carboxy-1-isopropylbut-2-enyl]-N¹,3-dimethyl-L-valinamide and
N,β,β-trimethyl-3,5-bis(trifluoromethyl)-D-phenylalanyl-N¹-[(1S,2E)-3-carboxy-1-isopropylbut-2-enyl]-N¹,3-dimethyl-L-valinamide
or pharmaceutically acceptable salts thereof.

41. The method according to claim 28 wherein said compound of Formula (II) is selected from:
(2E,4S)-2,5-dimethyl-4-(methyl{3-methyl-N-[(2S)-2-(methylamino)-2-(1-phenylcyclopentyl)ethanoyl]-L-valyl}amino)-2-hexenoic acid,
(2E,4S)-2,5-dimethyl4-(methyl{3-methyl-N-[(2R)-2-(methylamino)-2-(1-phenylcyclopentyl)ethanoyl]-L-valyl}amino)-2-hexenoic acid and
(2E,4R)-2,5-dimethyl-4-(methyl{3-methyl-N-[(methylamino)(1-phenylcyclohexyl)acetyl]-L-valyl}amino)-2-hexenoic acid
or pharmaceutically acceptable salts thereof.

42. The method according to claim 28 wherein said compound of Formula (II) is selected from:
(E,4S)-2,5-Dimethyl4-[methyl((2S)-2-{[(2S)-3-methyl-2-(methylamino)-3-phenylbutanoyl]amino}-3-phenylpropanoyl)amino]-2-hexenoic acid,
N,β,β-Trimethyl-L-phenylalanyl-N¹-[(1S,2E)-1-butyl-3-carboxybut-2-enyl]-N¹,3-dimethyl-L-valinamide,
N,β,β-Trimethyl-L-phenylalanyl-N¹-[(1S,2E)-3-carboxy-1-isobutyl-2-pentenyl]-N¹,3-dimethyl-L-valinamide,
(E,4S)-2-Butyl4-[((2S)-3,3-dimethyl-2-{[(2S)-3-methyl-2-(methylamino)-3-phenylbutanoyl]amino}butanoyl)amino]-5-methyl-2-hexenoic acid,
N,β,β-Trimethyl-L-phenylalanyl-N¹-[(1S,2E)-3-carboxy-1-isopropyl-2-pentenyl]-N¹,3-dimethyl-L-valinamide,
Ethyl (E,4S)-2,5-dimethyl4-{methyl[(2R)-3-methyl-2-{[(2S)-3-methyl-2-(methylamino)-3-phenylbutanoyl]amino}-3-(methylsulfanyl)butanoyl]amino}-2-hexenoate,
(E,4S)-2,5-dimethyl4-{methyl[(2R)-3-methyl-2-{[(2S)-3-methyl-2-(methylamino)-3-phenylbutanoyl]amino}-3-(methylsulfanyl)butanoyl]amino}-2-hexenoic acid,
N,β,β-trimethyl-L-phenylalanyl-N¹-[(1S,2E)-3-carboxy-1-isopropyl-2-butenyl]-N¹-methyl-3-(methylsulfonyl)-L-valinamide,
N,β,β-trimethyl-L-phenylalanyl-N¹-[(1S,2E)-3-carboxy-1-isopropyl-2-butenyl]-3-[(4-methoxybenzyl)sulfanyl]-N¹-methyl-L-valinamide,
N,O,β,β-tetramethyl-L-tyrosyl-N¹-[(1S,2E)-3-carboxy-1-isopropyl-2-butenyl]-3-[(4-methoxybenzyl)sulfanyl]-N¹-methyl-L-valinamide and
N,O,β,β-tetramethyl-L-tyrosyl-N¹-[(1S,2E)-3-carboxy-1-isopropyl-2-butenyl]-N¹-methyl-3-(methylsulfanyl)-L-valinamide
or pharmaceutically acceptable salts thereof.

43. The method according to claim 28 wherein said compound of Formula (II) is selected from:
N,β,β-trimethyl-L-phenylalanyl-N¹-[(1R,2E)-4-ethoxy-1-isopropyl-3-methyl-4-oxo-2-butenyl]-N¹-methyl-L-allothreoninamide,
N,β,β-trimethyl-L-phenylalanyl-N¹-[(1S,2E)-3-carboxy-1-isopropyl-2-butenyl]-N¹-methyl-L-allothreoninamide,
N,β,β-trimethyl-L-phenylalanyl-N-[(1S,2E)-3-carboxy-1-isopropyl-2-butenyl]-N,O,β,β-tetramethyl-L-tyrosinamide,
N,β,β-trimethyl-L-phenylalanyl-N¹-[(1S,2E)-3-carboxy-1-isopropyl-2-butenyl]-N¹,O-dimethyl-L-allothreoninamide,
(E,4S)-2,5-Dimethyl-4-[methyl((2S)-2-{[(2S)-3-methyl-2-(methylamino)-3-phenylbutanoyl]amino}-4-phenylbutanoyl)amino]-2-hexenoic acid,
N,β,β-trimethyl-L-phenylalanyl-4-benzoyl-N-[(1S,2E)-3-carboxy-1-isopropyl-2-butenyl]- N,β,β-trimethyl-L-phenylalaninamide and
4-benzoyl-N,β,β-trimethyl-L-phenylalanyl-N1-[(1S,2E)-3-carboxy-1-isopropylbut-2-enyl]-N¹,3-dimethyl-L-valinamide
or pharmaceutically acceptable salts thereof.

44. The method according to claim 28 wherein said compound of Formula (II) is selected from:
N,β,β-trimethyl-L-phenylalanyl-N¹-[(1S,2E)-3-carboxy-1-isobutylbut-2-enyl]-N¹-methyl-L-valinamide,
N,β,β-trimethyl-L-phenylalanyl-N¹-[(1S,2E)-3-carboxy-1-isobutylbut-2-enyl]-3-methyl-L-valinamide,
N,β,β-trimethyl-L-phenylalanyl-N¹-[(1S,2E)-3-carboxy-1-isopropylbut-2-enyl]-N¹-ethyl-3-methyl-L-valinamide,
N,β,β-trimethyl-L-phenylalanyl-N¹-[(1S,2E)-3-carboxy-1-isopropylbut-2-enyl]-N¹-ethyl-L-valinamide,
N,β,β-trimethyl-L-phenylalanyl-N¹-[(1S,2E)-3-carboxy-1-isopropylbut-2-enyl]-N¹-methyl-L-leucinamide,
N,β,β-trimethyl-L-phenylalanyl-N¹-[(1S,2E)-3-carboxy-1-isopropylbut-2-enyl]-N¹-methyl-L-norvalinamide,
(2E,4S)4-[{(2R)-2-cyclohexyl-2-[(N,β,1-trimethyl-L-phenylalanyl)amino]ethanoyl}(methyl)amino]-2,5-dimethylhex-2-enoic acid,
(2E,4S)-2,5-dimethyl-4-(methyl{(2S)-2-[(N,β,1-trimethyl-L-phenylalanyl)amino]butanoyl}amino)hex-2-enoic acid,
4-{[3,3-Dimethyl-2-(2-methylamino-3-phenyl-butyrylamino)-butyryl]-methyl-amino}-2,5-dimethyl-hex-2-enoic acid,
N,β,β-trimethyl-L-phenylalanyl-N¹-[(1S,2E)-3-carboxy-1-isopropylbut-2-enyl]-3-methyl-L-valinamide,
N,β,β-trimethyl-L-phenylalanyl-N¹-[(1S,2E)-3-carboxy-1-isopropylbut-2-enyl]-L-valinamide,
2,5-dimethyl-4-{methyl-[2-(3-methyl-2-methylamino-3-phenyl-butyrylamino)-propionyl]-amino}-hex-2-enoic acid,
4-{[3,3-Dimethyl-2-(3-methyl-2-methylamino-3-phenyl-butyrylamino)-butyryl]-methyl-amino}-2,6-dimethyl-hept-2-enoic acid,
N,β,β-trimethyl-L-phenylalanyl-N¹-[(1S,2E)-3-carboxy-1-isopropyl-2-butenyl]-N¹-methyl-L-valinamide and
N,β,β-trimethyl-L-phenylalanyl-N¹-[(1S,2E)-3-carboxy-1-isopropyl-2-butenyl]-N¹-methyl-L-isoleucinamide
or pharmaceutically acceptable salts thereof.

45. The method according to claim 28 wherein said compound of Formula (II) is selected from:
(E,4S)4-[((2S)-3,3-dimethyl-2-{[(2S)-3-methyl-2-(methylamino)-3-phenylbutanoyl]amino}butanoyl)(methyl)amino]-2,5-dimethyl-2-hexenamide,
(E,4S)-4-[((2S)-3,3-dimethyl-2-{[(2S)-3-methyl-2-(methylamino)-3-phenylbutanoyl]amino]butanoyl)(methyl)amino]-N,2,5-trimethyl-2-hexenamide,
N,β,β-trimethyl-L-phenylalanyl-N¹-{(1S,2E)-4-[(2-cyanoethyl)amino]-1-isopropyl-3-methyl-4-oxo-2-butenyl}-N¹,3-dimethyl-L-valinamide,
N,β,β-trimethyl-L-phenylalanyl-N¹-{(1S,2E)4-[(carboxymethyl)amino]-1-isopropyl-3-methyl-4-oxo-2-butenyl}-N¹,3-dimethyl-L-valinamide,
N,β,β-trimethyl-L-phenylalanyl-N¹-{(1S,2E)4-[(4-azidophenyl)amino]-1-isopropyl-3-methyl-4-oxo-2-butenyl}-N¹,3-dimethyl-L-valinamide,
N,β,β-trimethyl-L-phenylalanyl-N¹-{(1S,2E)-1-isopropyl-3-methyl4-oxo-4-[(2-phenylethyl)amino]but-2-enyl}-N¹-,3-dimethyl-L-valinamide,
N,β,β-trimethyl-L-phenylalanyl-N¹-{(1S,2E)4-[[(1S,2E)4-ethoxy-1-isopropyl-3-methyl-4-oxobut-2-enyl](methyl)amino]-1-isopropyl-3-methyl-4-oxobut-2-enyl}-N¹-,3-dimethyl-L-valinamide,
N,β,β-trimethyl-L-phenylalanyl-N¹-{(1S,2E)4-[[(1S,2E)-3-carboxy-1-isopropylbut-2-enyl](methyl)amino]-1-isopropyl-3-methyl-4-oxobut-2-enyl}-N¹-,3-dimethyl-L-valinamide,
N,β,β-trimethyl-L-phenylalanyl-N¹-[(1S,2E)-1-isopropyl-3-methyl-4-oxo4-(thien-2-ylmethoxy)but-2-enyl]-N¹-,3-dimethyl-L-valinamide,
N,β,β-trimethyl-L-phenylalanyl-N¹-[(1S,2E)-1-isopropyl-3-methyl-4-(octyloxy)-4-oxobut-2-enyl]-N¹-,3-dimethyl-L-valinamide,
N,β,β-trimethyl-L-phenylalanyl-N¹-[(1S,2Z)-3-carboxy-1-isopropyl-2-butenyl]-N¹,3-dimethyl-L-valinamide and
N,β,β-trimethyl-L-phenylalanyl-N¹-[(1S,2E)-3-carboxy-1-isopropylprop-2-enyl]-N¹,3-dimethyl-L-valinamide
or pharmaceutically acceptable salts thereof.

46. The method according to claim 28 wherein said compound of Formula (II) is selected from:
N,β,β-trimethyl-L-phenylalanyl-N¹-[(1S,2E)-1-allyl-3-carboxybut-2-enyl]-N¹,3-dimethyl-L-valinamide,
(2E,4S)4-[{(2S)-3,3-dimethyl-2-[(N,β,β-trimethyl-L-phenylalanyl)amino]4-pentenoyl}(methyl)amino]-2,5-dimethyl-2-hexenoic acid,
N,β,β-trimethyl-L-phenylalanyl-N¹-[(1S,2E)-3-carboxy-1-isopropylbut-2-enyl]-N¹,3-dimethyl-L-isoleucinamide,
N,β,β-trimethyl-L-phenylalanyl-N¹-[(1R,3S)-3-carboxy-1-isopropylbutyl]-N¹,3-dimethyl-L-valinamide,
N,β,β-trimethyl-L-phenylalanyl-N¹-[(1R,3R)-3-carboxy-1-isopropylbutyl]-N¹,3-dimethyl-L-valinamide,
β,β-diethyl-N-methyl-L-phenylalanyl-N¹-[(1S,2E)-3-carboxy-1-isopropylbut-2-enyl]-N¹,3-dimethyl-L-valinamide,
β,β-diethyl-N-methyl-D-phenylalanyl-N¹-[(1S,2E)-3-carboxy-1-isopropylbut-2-enyl]-N¹,3-dimethyl-L-valinamide,
(betaS)-N,beta-dimethyl-L-phenylalanyl-N¹-[(1S,2E)-3-carboxy-1-isopropylbut-2-enyl]-N¹,3-dimethyl-L-valinamide and
O-benzyl-N-methyl-L-threonyl-N¹-[(1S,2E)-3-carboxy-1-isopropylbut-2-enyl]-N¹,3-dimethyl-L-valinamide
or pharmaceutically acceptable salts thereof.

47. The method according to claim 28 wherein said compound of Formula (II) is selected from:
3-Cyclohexyl-N-methyl-L-valyl-N¹-[(1S,2E)-3-carboxy-1-isopropyl-2-butenyl]-N¹,3-dimethyl-L-valinamide and
3-cyclohexyl-N-methyl-L-valyl-N¹-[(1S,2E)-4-ethoxy-1-isopropyl-3-methyl-4-oxo-2-butenyl]-N¹,3-dimethyl-L-valinamide
or pharmaceutically acceptable salts thereof.

48. The method according to claim 28 wherein said compound of Formula (II) is selected from:
(2E,4S)-2,5-dimethyl-4-(methyl{3-methyl-N-[(2S)-2-(methylamino)-2-(1-phenylcyclopropyl)acetyl]-L-valyl}amino)hex-2-enoic acid,
(2E,4S)-2,5-dimethyl-4-(methyl{3-methyl-N-[(2R)-2-(methylamino)-2-(1-phenylcyclopropyl)acetyl]-L-valyl}amino)hex-2-enoic acid,
2-(4-{[3,3-Dimethyl-2-(3-methyl-2-methylamino-3-phenyl-butyrylamino)-butyryl]-methyl-amino}-2,5-dimethyl-hex-2-enoylamino)-4-methylsulfanyl-butyric acid methyl ester,
N,β,β-trimethyl-L-phenylalanyl-N 1-((1S,2E)-4-{[(1S)-1-carboxy-3-(methylthio)propyl]amino}-1-isopropyl-3-methyl-4-oxobut-2-enyl)-N1,3-dimethyl-L-valinamide,
N,β,β-trimethyl-4-[(E)-2-phenylvinyl]-L-phenylalanyl-N¹-[(1S,2E)-3-carboxy-1-isopropylbut-2-enyl]-N¹,3-dimethyl-L-valinamide,
N,β,β-trimethyl-4-[(E)-2-phenylvinyl]-D-phenylalanyl-N¹-[(1S,2E)-3-carboxy-1-isopropylbut-2-enyl]-N¹,3-dimethyl-L-valinamide,
N,β,β-trimethyl-L-phenylalanyl-N¹-[(1S,2E)-3-carboxy-1-isopropylbut-2′enyl]-3-fluoro-N¹-methyl-D-valinamide,
N,β,β-trimethyl-L-phenylalanyl-N¹-[(1S,2E)-3-carboxy-1-isopropylbut-2-enyl]-3-fluoro-N¹-methyl-L-valinamide,
3-[(4-methoxybenzyl)thio]-N-methyl-L-valyl-N¹-[(1S,2E)-3-carboxy-1-isopropylbut-2-enyl]-N¹,3-dimethyl-L-valinamide,
N-ethyl-β,β-dimethyl-L-phenylalanyl-N¹-[(1S,2E)-3-carboxy-1-isopropylbut-2-enyl]-N¹,3-dimethyl-L-valinamide,
(2E ,4S)-2,5-dimethyl-4-(methyl{3-methyl-N-[(2S)-3-methyl-3-phenyl-2-pyrrolidin-1-ylbutanoyl]-L-valyl}amino)hex-2-enoic acid,
N-(2-hydroxyethyl)-β,β-dimethyl-L-phenylalanyl-N¹-[(1S,2E)-3-carboxy-1-isopropylbut-2-enyl]-N¹,3-dimethyl-L-valinamide,
(βR)-N,β-dimethyl-L-phenylalanyl-N¹-[(1S,2E)-3-carboxy-1-isopropylbut-2-enyl]-N¹,3-dimethyl-L-valinamide,
3-acetyl-N,β,β-trimethyl-L-phenylalanyl-N¹-[(1S,2E)-3-carboxy-1-isopropylbut-2-enyl]-N¹,3-dimethyl-L-valinamide,
N,β,β-trimethyl-L-phenylalanyl-N¹-[(1S,2E)-3-carboxy-1-isopropylbut-2-enyl]-3-hydroxy-N¹-methyl-L-valinamide and
N,β,β-trimethyl-L-phenylalanyl-N¹-[(1R,2E)-3-carboxy-1-isopropylbut-2- enyl]-N¹,3-dimethyl-L-valinamide.
or pharmaceutically acceptable salts thereof.

49. The method according to claim 28 wherein said compound of Formula (II) is selected from:
3-Chloro-N,β,β-trimethyl-L-phenylalanyl-N¹-[(1S,2E)-3-carboxy-1-isopropyl-2-butenyl]-N¹,3-dimethyl-L-valinamide,
3-bromo-N,β,β-trimethyl-L-phenylalanyl-N¹-[(1S,2E)-3-carboxy-1-isopropylbut-2-enyl]-N¹,3-dimethyl-L-valinamide,
N,β,β,3-Tetramethyl-L-phenylalanyl-N¹-[(1S,2E)-3-carboxy-1-isopropyl-2-butenyl]-N¹,3-dimethyl-L-valinamide,
3-Cyclohexyl-N-methyl-L-valyl-N¹-[(1S,2E)-3-carboxy-1-isopropyl-2-butenyl]-N¹,3-dimethyl-L-valinamide,
N,O,β,β-tetramethyl-L-tyrosyl-N¹-[(1S,2E)-3-carboxy-1-isopropyl-2-butenyl]-N¹,3-dimethyl-L-valinamide,
N,O,β,β-tetramethyl-L-tyrosyl-N¹-[(1S,2E)-3-carboxy-1-isopropyl-2-butenyl]-N¹-methyl-3-(methylsulfanyl)-L-valinamide, and
N,β,β-3,4-Pentamethyl-L-phenylalanyl-N¹-[(1S,2E)-4-ethoxy-1-isopropyl-3-methyl-4-oxo-2-butenyl]-N¹,3-dimethyl-L-valinamide.

50. The method according to claim 28 wherein said compound of Formula (II) is N,β,β-trimethyl-L-phenylalanyl-N¹-[(1S,2E)-3-carboxy-1-isopropylbut-2-enyl]-N¹,3-dimethyl-L-valinamide.

51. A method of treating, inhibiting the growth of, or eradicating a tumor in a mammal in need thereof wherein said tumor is resistant to at least one chemotherapeutic agent which method comprises providing to said mammal an effective amount of a compound selected from the group:
3-Chloro-N,β,β-trimethyl-L-phenylalanyl-N¹-[(1S,2E)-3-carboxy-1-isopropyl-2-butenyl]-N¹,3-dimethyl-L-valinamide,
3-bromo-N,β,β-trimethyl-L-phenylalanyl-N¹-[(1S,2E)-3-carboxy-1-isopropylbut-2-enyl]-N¹,3-dimethyl-L-valinamide,
N,β,β,3-Tetramethyl-L-phenylalanyl-N¹-[(1S,2E)-3-carboxy-1-isopropyl-2-butenyl]-N¹,3-dimethyl-L-valinamide,
3-Cyclohexyl-N-methyl-L-valyl-N¹-[(1S,2E)-3-carboxy-1-isopropyl-2-butenyl]-N¹,3-dimethyl-L-valinamide,
N,O,β,β-tetramethyl-L-tyrosyl-N¹-[(1S,2E)-3-carboxy-1-isopropyl-2-butenyl]-N¹,3-dimethyl-L-valinamide,
N,O,β,β-tetramethyl-L-tyrosyl-N¹-[(1S,2E)-3-carboxy-1-isopropyl-2-butenyl]-N¹-methyl-3-(methylsulfanyl)-L-valinamide, and
N,β,β-3,4-Pentamethyl-L-phenylalanyl-N¹-[(1S,2E)-4-ethoxy-1-isopropyl-3-methyl-4-oxo-2-butenyl]-N¹,3-dimethyl-L-valinamide
or a pharmaceutically acceptable salt thereof.

52. The method according to claim 51 wherein the chemotherapeutic agents are antimicrotubule inhibitors.

53. The method according to claim 52 wherein the antimicrotubule inhibitors are selected from the group consisting of paclitaxel, docetaxel, vinblastine, vincristine and vinorelbine.

54. The method according to claim 51 wherein the tumors are selected from the group consisting of breast, colon, lung, prostate, melanoma, epidermal, leukemia, kidney, bladder, mouth, larynx, esophagus, stomach, ovary, pancreas, liver, skin and brain.

55. The method according to claim 51 wherein the tumors overexpress MDR-1, MXR or MRP.

56. The method according to claim 51 wherein the resistance to chemotherapeutic agents is multiple drug resistance (MDR).

57. The method according to claim 51 wherein the resistance is inherent or acquired.

58. The method according to claim 57 wherein the resistance is acquired.

59. The method according to claim 51 wherein a compound is administered before, concurrently, or after treatment with the chemotherapeutic agent.

60. A method of treating, inhibiting the growth of, or eradicating a tumor in a mammal in need thereof wherein said tumor is resistant to at least one chemotherapeutic agent which method comprises providing to said mammal an effective amount of the compound
N,β,β-trimethyl-L-phenylalanyl-N¹-[(1S,2E)-3-carboxy-1-isopropylbut-2-enyl]-N¹,3-dimethyl-L-valinamide or a pharmaceutically acceptable salt thereof.

61. The method according to claim 60 wherein the chemotherapeutic agents are antimicrotubule inhibitors.

62. The method according to claim 61 wherein the antimicrotubule inhibitors are selected from the group consisting of paclitaxel, docetaxel, vinblastine, vincristine and vinorelbine.

63. The method according to claim 60 wherein the tumors are selected from the group consisting of breast, colon, lung, prostate, melanoma, epidermal, leukemia, kidney, bladder, mouth, larynx, esophagus, stomach, ovary, pancreas, liver, skin and brain.

64. The method according to claim 60 wherein the tumors overexpress MDR-1, MXR or MRP.

65. The method according to claim 60 wherein the resistance to chemotherapeutic agents is multiple drug resistance (MDR).

66. The method according to claim 60 wherein the resistance is inherent or acquired.

67. The method according to claim 66 wherein the resistance is acquired.

68. The method according to claim 60 wherein the compound is administered before, concurrently, or after treatment with the chemotherapeutic agent.

69. A process for the preparation of a carboxylic acid of the formula 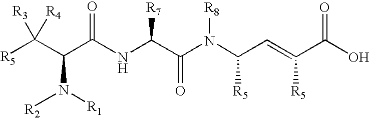
wherein:
R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)2 —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, NO₂, —SO₃H, —SOR₁₀ or —SO₂R₁₀, wherein R₁₀ is a linear, branched or cyclic, one to ten carbon saturated or unsaturated alkyl group; and aryl-R-; or R₁ and R₂ taken together with the nitrogen atom to which they are attached is a three to seven membered ring;
R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
or R₃ and R₄ taken together with the carbon to which they are attached form a three to seven membered ring;
R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀OH, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R- and aryl and provided that when R₅ is an indolyl moiety of the formula 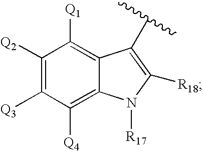
R₁₇ is H or an optionally substituted alkyl or acyl group; and
R₁₈ Q₁, Q₂, Q₃ and Q₄ are independently selected from H, halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;
R₇ is selected from the group consisting of a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
and wherein,
R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group;
X is a moiety selected from the group consisting of —OH, —OR, ═O, ═S, —O₂CR, —SH, —SR, —SOCR, —NH₂, —NHR, —N(R)₂, —NHCOR, NRCOR, —I, Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;
Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;
comprising the steps of:
a) treating a carboxylic acid of the formula 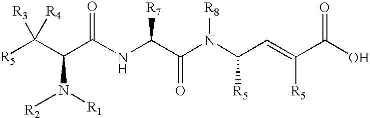
with ozone in the methanol followed by further treating with dimethylsulfide to obtain an aldehyde of the formula 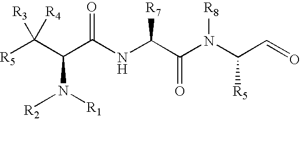
b) reacting said aldehyde with a phosphonate of the formula 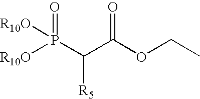
where R₁₀ is optionally fluoro substituted alkyl of 1 to 10 carbon atoms, in the presence of potassium hexamethyldisilazide and 1 8-crown-6 and hydrolyzing with base to obtain a carboxylic acid of the formula

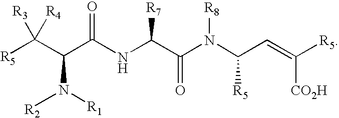

70. A process for the preparation of a carboxylic acid of the formula 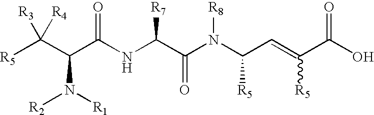
wherein:
R₁ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R-;
R₂ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂ —NHCOR₁₀, —NR₁₀OCOR₁₀, —I, Br, —Cl, —F, —CN, —CO₂H, —CHO, —COR₁₀, —CONH₂, —CONHR₁₀, —CON(R₁₀)₂, —COSH, —COSR₁₀, NO₂, —SO₃H, —SOR₁₀ or —SO₂R₁₀, wherein R₁₀ is a linear, branched or cyclic, one to ten carbon saturated or unsaturated alkyl group; and aryl-R-; or R₁ and R₂ taken together with the nitrogen atom to which they are attached is a three to seven membered ring;
R₃ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
R₄ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀OCOR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
or R₃ and R₄ taken together with the carbon to which they are attached form a three to seven membered ring;
R₅ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R- and aryl and provided that when R₅ is an indolyl moiety of the formula 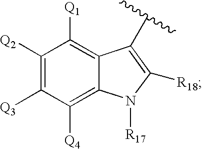
R₁₇ is H or an optionally substituted alkyl or acyl group; and
R₁₈ Q₁, Q₂, Q₃ and Q₄ are independently selected from H, halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl, wherein the alkyl and acyl groups of the substituents are optionally substituted;
R₇ is selected from the group consisting of a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
R₈ is selected from the group consisting of H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, CO₂H, —CO₂R₁₀, —CHO, —COR₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group; and aryl-R-;
and wherein,
R is a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀OH, —N(R₁₀)₂, —NHCOR₁₀, —NR₁₀COR₁₀, —I, Br, —Cl, —F, —CN, CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ wherein R₁₀ is a linear, branched or cyclic, one to ten carbon saturated or unsaturated alkyl group;
X is a moiety selected from the group consisting of —OH, —OR, ═O, ═S, —O₂CR, —SH, —SR, —SOCR, —NH₂, —NHR, —N(R)₂, —NHCOR, NRCOR, —I, Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R;
Aryl is an aromatic ring selected from the group consisting of: phenyl, naphthyl, anthracyl, phenanthryl, thienyl, furyl, indolyl, pyrrolyl, thiophenyl, benzofuryl, benzothiophenyl, quinolyl, isoquinolyl, imidazolyl, thiazolyl, oxazolyl, and pyridyl, optionally substituted with R or X;
comprising the steps of:
b) treating a carboxylic acid of the formula 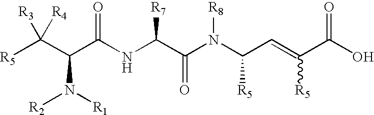
with ozone in methanol followed by further treating with dimethylsulfide to obtain an aldehyde of the formula 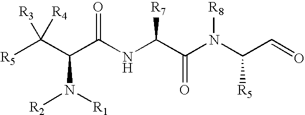
c) reacting said aldehyde of step a) with triphenylphosphorane of the formula 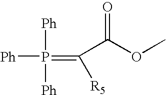
and hydrolyzing with base to obtain said carboxylic acid having the formula

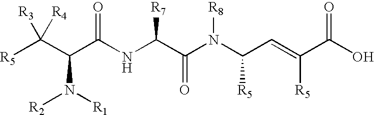

71. The process according to claim 69 wherein the base in step b) is aqueous lithium hydroxide.

72. The process according to claim 70 wherein the base in step b) is aqueous lithium hydroxide.

73. A method of treating, inhibiting the growth of, or eradicating a tumor in a mammal in need thereof wherein said tumor is resistant to at least one chemotherapeutic agent which method comprises providing to said mammal an effective amount of the compound
N,O,β,β-tetramethyl-L-tyrosyl-N¹-[(1S,2E)-3-carboxy-1-isopropyl-2-butenyl]-N¹,3-dimethyl-L-valinamide.

74. The method according to claim 73 wherein the chemotherapeutic agents are antimicrotubule inhibitors.

75. The method according to claim 74 wherein the antimicrotubule inhibitors are selected from the group consisting of paclitaxel, docetaxel, vinblastine, vincristine and vinorelbine.

76. The method according to claim 73 wherein the tumors are selected from the group consisting of breast, colon, lung, prostate, melanoma, epidermal, leukemia, kidney, bladder, mouth, larynx, esophagus, stomach, ovary, pancreas, liver, skin and brain.

77. The method according to claim 73 wherein the tumors overexpress MDR-1, MXR or MRP.

78. The method according to claim 73 wherein the resistance to chemotherapeutic agents is multiple drug resistance (MDR).

79. The method according to claim 73 wherein the resistance is inherent or acquired.

80. The method according to claim 79 wherein the resistance is acquired.

81. The method according to claim 73 wherein the compound is administered before, concurrently, or after treatment with the chemotherapeutic agent.
